(19) United States
(12) Patent Application Publication
(10) Pub. No.: US 2023/0365705 A1
(43) Pub. Date: Nov. 16, 2023

(54) ANTI-CD93 CONSTRUCTS AND USES THEREOF

(71) Applicant: DYNAMICURE BIOTECHNOLOGY LLC, Cambridge, MA (US)

(72) Inventors: Zirong CHEN, Saugus, MA (US); Roxann GUERRETTE, Cambridge, MA (US); Gregory JONES, Cambridge, MA (US); Shigeru KOMABA, Cambridge, MA (US); Jian LI, Cambridge, MA (US); Angela NORTON, Reading, MA (US); Lihua WU, Cambridge, MA (US); Zhinan XIA, Waltham, MA (US)

(21) Appl. No.: 18/028,170

(22) PCT Filed: Sep. 28, 2021
(86) PCT No.: PCT/US2021/052446

§ 371 Date: Mar. 23, 2023

Related U.S. Application Data
(60) Provisional application No. 63/084,474, filed on Sep. 28, 2020.

(51) Int. Cl. C07K 16/28; C12N 15/63; A61P 35/04

(52) U.S. Cl. CPC C07K 16/2896; C12N 15/63; A61P 35/04; C07K 2317/565; C07K 2317/70; C07K 2317/31

(57) ABSTRACT

The present application provides anti-CD93 constructs that bind to CD93 (e.g., anti-CD93 antibodies), nucleic acid molecules encoding an amino acid sequence of the anti-CD93, vectors comprising the nucleic acid molecules, host cells containing the vectors, methods of preparing the anti-CD93 construct, pharmaceutical compositions containing the anti-CD93 construct, and methods of using the anti-CD93 construct or compositions.

### CROSS-REFERENCE TO RELATED APPLICATION

This application claims priority to U.S. provisional application 63/084,474, filed on Sep. 28, 2020, International Application No. PCT/US2021/035542, filed on Jun. 2, 2021, and International Application No. PCT/US2021/043784, filed on Jul. 29, 2021, the contents of which are incorporated by reference in their entirety for all purposes.

### TECHNICAL FIELD

The present disclosure relates to anti-CD93 constructs (such as anti-CD93 antibodies) and the uses thereof.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 193852000246SEQLIST.TXT, date recorded: Sep. 28, 2021, size: 196,372 bytes).

### BACKGROUND OF THE APPLICATION

CD93 (Cluster of Differentiation 93) is a protein that in humans is encoded by the CD93 gene. CD93 is a C-type lectin transmembrane receptor which plays a role not only in cell-cell adhesion processes but also in host defense. CD93 was initially thought to be a receptor for C1q, but now is thought to instead be involved in intercellular adhesion and in the clearance of apoptotic cells. The intracellular cytoplasmic tail of this protein contains two highly conserved domains which may be involved in CD93 function. Indeed, the highly charged juxtamembrane domain has been found to interact with moesin, a protein known to play a role in linking transmembrane proteins to the cytoskeleton and in the remodeling of the cytoskeleton. This process appears crucial for adhesion, migration and phagocytosis.

The disclosures of all publications, patents, patent applications and published patent applications referred to herein are hereby incorporated herein by reference in their entirety.

### BRIEF SUMMARY OF THE APPLICATION

The following summary is illustrative only and is not intended to be limiting in any way. That is, the following summary is provided to introduce highlights, benefits and advantages of the novel molecules and the uses thereof. Thus, the following summary is not intended to identify essential features of the claimed subject matter, nor is it intended for use in determining the scope of the claimed subject matter.

In one aspect, the present application provides an anti-CD93 construct comprising an antibody moiety comprising a heavy chain variable region (V_{H}) and a light chain variable region (V_{L}), wherein the antibody moiety competes for a binding epitope of CD93 with an antibody or antibody fragment comprising a second heavy chain variable region (V_{H-2}) and a second light chain variable region (V_{L-2}), wherein:
a) the V_{H-2} comprising the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the V_{L-2} comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6;
b) the V_{H-2} comprises the HC-CDR1 comprising the amino acid sequence of SEQ ID NO: 17, the HC-CDR2 comprising the amino acid sequence of SEQ ID NO: 18, and the HC-CDR3 comprising the amino acid sequence of SEQ ID NO: 19, and the V_{L-2} comprises the LC-CDR1 comprising the amino acid sequence of SEQ ID NO: 20, the LC-CDR2 comprising the amino acid sequence of SEQ ID NO: 21, and the LC-CDR3 comprising the amino acid sequence of SEQ ID NO: 22;
c) the V_{H-2} comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the V_{L-2} comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38;
d) the V_{H-2} comprises the HC-CDR1 comprising the amino acid sequence of SEQ ID NO: 49, the HC-CDR2 comprising the amino acid sequence of SEQ ID NO: 50, and the HC-CDR3 comprising the amino acid sequence of SEQ ID NO: 51, and the V_{L-2} comprises the LC-CDR1 comprising the amino acid sequence of SEQ ID NO: 52, the LC-CDR2 comprising the amino acid sequence of SEQ ID NO: 53, and the LC-CDR3 comprising the amino acid sequence of SEQ ID NO: 54;
e) the V_{H-2} comprises the HC-CDR1 comprising the amino acid sequence of SEQ ID NO: 65, the HC-CDR2 comprising the amino acid sequence of SEQ ID NO: 66, and the HC-CDR3 comprising the amino acid sequence of SEQ ID NO: 67, and the V_{L-2} comprises the LC-CDR1 comprising the amino acid sequence of SEQ ID NO: 68, the LC-CDR2 comprising the amino acid sequence of SEQ ID NO: 69, and the LC-CDR3 comprising the amino acid sequence of SEQ ID NO: 70;
f) the V_{H-2} comprises the HC-CDR1 comprising the amino acid sequence of SEQ ID NO: 81, the HC-CDR2 comprising the amino acid sequence of SEQ ID NO: 82, and the HC-CDR3 comprising the amino acid sequence of SEQ ID NO: 83, and the V_{L-2} comprises the LC-CDR1 comprising the amino acid sequence of SEQ ID NO: 84, the LC-CDR2 comprising the amino acid sequence of SEQ ID NO: 85, and the LC-CDR3 comprising the amino acid sequence of SEQ ID NO: 86;
g) the V_{H-2} comprises the HC-CDR1 comprising the amino acid sequence of SEQ ID NO: 97, the HC-CDR2 comprising the amino acid sequence of SEQ ID NO: 98, and the HC-CDR3 comprising the amino acid sequence of SEQ ID NO: 99, and the V_{L-2} comprises the LC-CDR1 comprising the amino acid sequence of SEQ ID NO: 100, the LC-CDR2 comprising the amino acid sequence of SEQ ID NO: 101, and the LC-CDR3 comprising the amino acid sequence of SEQ ID NO: 102;
h) the V_{H-2} comprises the HC-CDR1 comprising the amino acid sequence of SEQ ID NO: 113, the HC-CDR2 comprising the amino acid sequence of SEQ ID NO: 114, and the HC-CDR3 comprising the amino acid sequence of SEQ ID NO: 115, and the V_{L-2} comprises the LC-CDR1 comprising the amino acid sequence of SEQ ID NO: 116, the LC-CDR2 comprising the amino acid sequence of SEQ ID NO: 117, and the LC-CDR3 comprising the amino acid sequence of SEQ ID NO: 118;
i) the V_{H-2} comprises the HC-CDR1 comprising the amino acid sequence of SEQ ID NO: 129, the HC-CDR2 comprising the amino acid sequence of SEQ ID NO: 130, and the HC-CDR3 comprising the amino acid sequence of SEQ ID NO: 131, and the V_{L-2} comprises the LC-CDR1 comprising the amino acid sequence of SEQ ID NO: 132, the LC-CDR2 comprising the amino acid sequence of SEQ ID NO: 133, and the LC-CDR3 comprising the amino acid sequence of SEQ ID NO: 134;
j) the V_{H-2} comprises the HC-CDR1 comprising the amino acid sequence of SEQ ID NO: 145, the HC-CDR2 comprising the amino acid sequence of SEQ ID NO: 146, and the HC-CDR3 comprising the amino acid sequence of SEQ ID NO: 147, and the V_{L-2} comprises the LC-CDR1 comprising the amino acid sequence of SEQ ID NO: 148, 355, or 358, the LC-CDR2 comprising the amino acid sequence of SEQ ID NO: 149 or 356, and the LC-CDR3 comprising the amino acid sequence of SEQ ID NO: 150, 357 or 359;
k) the V_{H-2} comprises the HC-CDR1 comprising the amino acid sequence of SEQ ID NO: 161, the HC-CDR2 comprising the amino acid sequence of SEQ ID NO: 162, and the HC-CDR3 comprising the amino acid sequence of SEQ ID NO: 163, and the V_{L-2} comprises the LC-CDR1 comprising the amino acid sequence of SEQ ID NO: 164, the LC-CDR2 comprising the amino acid sequence of SEQ ID NO: 165, and the LC-CDR3 comprising the amino acid sequence of SEQ ID NO: 166;
l) the V_{H-2} comprises the HC-CDR1 comprising the amino acid sequence of SEQ ID NO: 177, the HC-CDR2 comprising the amino acid sequence of SEQ ID NO: 178, and the HC-CDR3 comprising the amino acid sequence of SEQ ID NO: 179, and the V_{L-2} comprises the LC-CDR1 comprising the amino acid sequence of SEQ ID NO: 180 or 353, the LC-CDR2 comprising the amino acid sequence of SEQ ID NO: 181 or 354, and the LC-CDR3 comprising the amino acid sequence of SEQ ID NO: 182;
m) the V_{H-2} comprises the HC-CDR1 comprising the amino acid sequence of SEQ ID NO: 193, the HC-CDR2 comprising the amino acid sequence of SEQ ID NO: 194, and the HC-CDR3 comprising the amino acid sequence of SEQ ID NO: 195, and the V_{L-2} comprises the LC-CDR1 comprising the amino acid sequence of SEQ ID NO: 196, the LC-CDR2 comprising the amino acid sequence of SEQ ID NO: 197, and the LC-CDR3 comprising the amino acid sequence of SEQ ID NO: 198;
n) the V_{H-2} comprises the HC-CDR1 comprising the amino acid sequence of SEQ ID NO: 209, the HC-CDR2 comprising the amino acid sequence of SEQ ID NO: 210, and the HC-CDR3 comprising the amino acid sequence of SEQ ID NO: 211, and the V_{L-2} comprises the LC-CDR1 comprising the amino acid sequence of SEQ ID NO: 212, the LC-CDR2 comprising the amino acid sequence of SEQ ID NO: 213, and the LC-CDR3 comprising the amino acid sequence of SEQ ID NO: 214; or
o) the V_{H-2} comprises the HC-CDR1 comprising the amino acid sequence of SEQ ID NO: 289, the HC-CDR2 comprising the amino acid sequence of SEQ ID NO: 290, and the HC-CDR3 comprising the amino acid sequence of SEQ ID NO: 291, and the V_{L-2} comprises the LC-CDR1 comprising the amino acid sequence of SEQ ID NO: 292, the LC-CDR2 comprising the amino acid sequence of SEQ ID NO: 293, and the LC-CDR3 comprising the amino acid sequence of SEQ ID NO: 294;
p) the V_{H-2} comprises the HC-CDR1 comprising the amino acid sequence of SEQ ID NO: 17 or 304, the HC-CDR2 comprising the amino acid sequence of SEQ ID NO: 18 or 305, and the HC-CDR3 comprising the amino acid sequence of SEQ ID NO: 19, and the V_{L-2} comprises the LC-CDR1 comprising the amino acid sequence of SEQ ID NO: 20, 301, 302, 303, or 306, the LC-CDR2 comprising the amino acid sequence of SEQ ID NO: 21, and the LC-CDR3 comprising the amino acid sequence of SEQ ID NO:22.

In some embodiments, the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 5, 4, 3, 2, or 1 amino acid substitutions in the LC-CDRs.

In some embodiments, the V_{H} comprises i) the HC-CDR1 comprising the amino acid sequence of SEQ ID NO: 17 or 304, ii) the HC-CDR2 comprising the amino acid sequence of SEQ ID NO: 18 or 305, and iii) the HC-CDR3 comprising the amino acid sequence of SEQ ID NO: 19, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 20, 301, 302, 303, or 306, ii) the LC-CDR2 comprising the amino acid sequence of SEQ ID NO: 21, and iii) the LC-CDR3 comprising the amino acid sequence of SEQ ID NO: 22, or a variant thereof comprising up to 5, 4, 3, 2, or 1 amino acid substitutions in the LC-CDRs.

In some embodiments, the V_{H} comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38, or a variant thereof comprising up to 5, 4, 3, 2, or 1 amino acid substitutions in the LC-CDRs.

In some embodiments, the V_{H} comprises i) the HC-CDR1 comprising the amino acid sequence of SEQ ID NO: 49, ii) the HC-CDR2 comprising the amino acid sequence of SEQ ID NO: 50, and iii) the HC-CDR3 comprising the amino acid sequence of SEQ ID NO: 51, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 52, ii) the LC-CDR2 comprising the amino acid sequence of SEQ ID NO: 53, and iii) the LC-CDR3 comprising the amino acid sequence of SEQ ID NO: 54, or a variant thereof comprising up to 5, 4, 3, 2, or 1 amino acid substitutions in the LC-CDRs.

In some embodiments, the V_{H} comprises i) the HC-CDR1 comprising the amino acid sequence of SEQ ID NO: 65, ii) the HC-CDR2 comprising the amino acid sequence of SEQ ID NO: 66, and iii) the HC-CDR3 comprising the amino acid sequence of SEQ ID NO: 67, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 68, ii) the LC-CDR2 comprising the amino acid sequence of SEQ ID NO: 69, and iii) the LC-CDR3 comprising the amino acid sequence of SEQ ID NO: 70, or a variant thereof comprising up to 5, 4, 3, 2, or 1 amino acid substitutions in the LC-CDRs.

In some embodiments, the V_{H} comprises i) the HC-CDR1 comprising the amino acid sequence of SEQ ID NO: 81, ii) the HC-CDR2 comprising the amino acid sequence of SEQ ID NO: 82, and iii) the HC-CDR3 comprising the amino acid sequence of SEQ ID NO: 83, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 84, ii) the LC-CDR2 comprising the amino acid sequence of SEQ ID NO: 85, and iii) the LC-CDR3 comprising the amino acid sequence of SEQ ID NO: 86, or a variant thereof comprising up to 5, 4, 3, 2, or 1 amino acid substitutions in the LC-CDRs.

In some embodiments, the V_{H} comprises i) the HC-CDR1 comprising the amino acid sequence of SEQ ID NO: 97, ii) the HC-CDR2 comprising the amino acid sequence of SEQ ID NO: 98, and iii) the HC-CDR3 comprising the amino acid sequence of SEQ ID NO: 99, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 100, ii) the LC-CDR2 comprising the amino acid sequence of SEQ ID NO: 101, and iii) the LC-CDR3 comprising the amino acid sequence of SEQ ID NO: 102, or a variant thereof comprising up to 5, 4, 3, 2, or 1 amino acid substitutions in the LC-CDRs.

In some embodiments, the V_{H} comprises i) the HC-CDR1 comprising the amino acid sequence of SEQ ID NO: 113, ii) the HC-CDR2 comprising the amino acid sequence of SEQ ID NO: 114, and iii) the HC-CDR3 comprising the amino acid sequence of SEQ ID NO: 115, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 116, ii) the LC-CDR2 comprising the amino acid sequence of SEQ ID NO: 117, and iii) the LC-CDR3 comprising the amino acid sequence of SEQ ID NO: 118, or a variant thereof comprising up to 5, 4, 3, 2, or 1 amino acid substitutions in the LC-CDRs.

In some embodiments, the V_{H} comprises i) the HC-CDR1 comprising the amino acid sequence of SEQ ID NO: 129, ii) the HC-CDR2 comprising the amino acid sequence of SEQ ID NO: 130, and iii) the HC-CDR3 comprising the amino acid sequence of SEQ ID NO: 131, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 132, ii) the LC-CDR2 comprising the amino acid sequence of SEQ ID NO: 133, and iii) the LC-CDR3 comprising the amino acid sequence of SEQ ID NO: 134, or a variant thereof comprising up to 5, 4, 3, 2, or 1 amino acid substitutions in the LC-CDRs.

In some embodiments, the V_{H} comprises i) the HC-CDR1 comprising the amino acid sequence of SEQ ID NO: 145, ii) the HC-CDR2 comprising the amino acid sequence of SEQ ID NO: 146, and iii) the HC-CDR3 comprising the amino acid sequence of SEQ ID NO: 147, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 148, 355, or 358, ii) the LC-CDR2 comprising the amino acid sequence of SEQ ID NO: 149 or 356, and iii) the LC-CDR3 comprising the amino acid sequence of SEQ ID NO: 150, 357 or 359, or a variant thereof comprising up to 5, 4, 3, 2, or 1 amino acid substitutions in the LC-CDRs.

In some embodiments, the V_{H} comprises i) the HC-CDR1 comprising the amino acid sequence of SEQ ID NO: 161, ii) the HC-CDR2 comprising the amino acid sequence of SEQ ID NO: 162, and iii) the HC-CDR3 comprising the amino acid sequence of SEQ ID NO: 163, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 164, ii) the LC-CDR2 comprising the amino acid sequence of SEQ ID NO: 165, and iii) the LC-CDR3 comprising the amino acid sequence of SEQ ID NO: 166, or a variant thereof comprising up to 5, 4, 3, 2, or 1 amino acid substitutions in the LC-CDRs.

In some embodiments, the V_{H} comprises i) the HC-CDR1 comprising the amino acid sequence of SEQ ID NO: 177, ii) the HC-CDR2 comprising the amino acid sequence of SEQ ID NO: 178, and iii) the HC-CDR3 comprising the amino acid sequence of SEQ ID NO: 179, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 180 or 353, ii) the LC-CDR2 comprising the amino acid sequence of SEQ ID NO: 181 or 354, and iii) the LC-CDR3 comprising the amino acid sequence of SEQ ID NO: 182, or a variant thereof comprising up to 5, 4, 3, 2, or 1 amino acid substitutions in the LC-CDRs.

In some embodiments, the V_{H} comprises i) the HC-CDR1 comprising the amino acid sequence of SEQ ID NO: 193, ii) the HC-CDR2 comprising the amino acid sequence of SEQ ID NO: 194, and iii) the HC-CDR3 comprising the amino acid sequence of SEQ ID NO: 195, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 196, ii) the LC-CDR2 comprising the amino acid sequence of SEQ ID NO: 197, and iii) the LC-CDR3 comprising the amino acid sequence of SEQ ID NO: 198, or a variant thereof comprising up to 5, 4, 3, 2, or 1 amino acid substitutions in the LC-CDRs.

In some embodiments, the V_{H} comprises i) the HC-CDR1 comprising the amino acid sequence of SEQ ID NO: 209, ii) the HC-CDR2 comprising the amino acid sequence of SEQ ID NO: 210, and iii) the HC-CDR3 comprising the amino acid sequence of SEQ ID NO: 211, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 212, ii) the LC-CDR2 comprising the amino acid sequence of SEQ ID NO: 213, and iii) the LC-CDR3 comprising the amino acid sequence of SEQ ID NO: 214, or a variant thereof comprising up to 5, 4, 3, 2, or 1 amino acid substitutions in the LC-CDRs.

In some embodiments, the V_{H} comprises i) the HC-CDR1 comprising the amino acid sequence of SEQ ID NO: 289, ii) the HC-CDR2 comprising the amino acid sequence of SEQ ID NO: 290, and iii) the HC-CDR3 comprising the amino acid sequence of SEQ ID NO: 291, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 292, ii) the LC-CDR2 comprising the amino acid sequence of SEQ ID NO: 293, and iii) the LC-CDR3 comprising the amino acid sequence of SEQ ID NO: 294, or a variant thereof comprising up to 5, 4, 3, 2, or 1 amino acid substitutions in the LC-CDRs.

In some embodiments, the V_{H} comprises the HC-CDR1 comprising the amino acid sequence of SEQ ID NO: 17 or 304, the HC-CDR2 comprising the amino acid sequence of SEQ ID NO: 18 or 305, and the HC-CDR3 comprising the amino acid sequence of SEQ ID NO: 19, or a variant thereof comprising up to 5, 4, 3, 2, or 1 amino acid substitutions in the HC-CDRs; and the V_{L} comprises the LC-CDR1 comprising the amino acid sequence of SEQ ID NO: 20, 301, 302, 303, or 306, the LC-CDR2 comprising the amino acid sequence of SEQ ID NO: 21, and the LC-CDR3 comprising the amino acid sequence of SEQ ID NO:22, or a variant thereof comprising up to 5, 4, 3, 2, or 1 amino acid substitutions in the LC-CDRs.

The present application in another aspect comprises an anti-CD93 construct comprising an antibody moiety that specifically binds to CD93, comprising:
a) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 13, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 14;
b) a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 29 and 307-312, and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 30, and 313-318;
c) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 45, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 46;
d) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 61, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 62;
e) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 77, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 78;
f) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 93, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 94;
g) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 109, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 110;
h) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 125, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 126;
i) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 141, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 142;
j) a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 157 and 360-362, and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 158, and 363-365;
k) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 173, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 174;
l) a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 189 and 347-349, and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 190, and 350-352;
m) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 205, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 206;
n) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 221, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 222;
o) a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 287 and 319-321, and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 288, and 322-324;
p) a HC-CDR1, a HC-CDR2, and a HC-CDR3, respectively comprising the amino acid sequences of a CDR1, a CDR2, and a CDR3 within a V_{H} chain region having the sequence set forth in any one of SEQ ID NOs: 307-312, and a LC-CDR1, a LC-CDR2, and a LC-CDR3, respectively comprising the amino acid sequences of a CDR1, a CDR2, and a CDR3 within a V_{L} chain region having the sequence set forth in any one of SEQ ID NOs: 313-318; or
q) a HC-CDR1, a HC-CDR2, and a HC-CDR3, respectively comprising the amino acid sequences of a CDR1, a CDR2, and a CDR3 within a V_{H} chain region having the sequence set forth in any one of SEQ ID NOs: 319-321, and a LC-CDR1, a LC-CDR2, and a LC-CDR3, respectively comprising the amino acid sequences of a CDR1, a CDR2, and a CDR3 within a V_{L} chain region having the sequence set forth in any one of SEQ ID NOs: 322-324.

In some embodiments according to any of the anti-CD93 constructs described above, wherein the V_{H} comprises an amino acid sequence of any one of SEQ ID NOs: 13, 29, 45, 61, 77, 93, 109, 125, 141, 157, 173, 189, 205, 221, 287, 307-312 and 319-321, or a variant comprising an amino acid sequence having at least about 80% (such as at least about any one of 80%, 85%, 90%, 95%, 96%, 97%, 98%, or 99%) sequence identity, and/or wherein the V_{L} comprises an amino acid sequence of any one of SEQ ID NOs: 14, 30, 46, 62, 78, 94, 110, 126, 142, 158, 174, 190, 206, 222, 288, 313-318 and 322-324 or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 13, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 14, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of any of SEQ ID NO: 29 and 307-312, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of any of SEQ ID NO: 30, and 313-318, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 45, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of SEQ ID NO: 46, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 61, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 62, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 77, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 78, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 93, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of SEQ ID NO: 94, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 109, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 110, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 125, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 126, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 141, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of SEQ ID NO: 142, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 157, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of SEQ ID NO: 158, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 173, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 174, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of any of SEQ ID NO: 189 and 347-349, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of any of SEQ ID NO: 190, and 350-352, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 205, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of SEQ ID NO: 206, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 221, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 222, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of any of SEQ ID NO: 287 and 319-321, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of any of SEQ ID NO: 288, and 322-324, or a variant comprising an amino acid sequence having at least about 80% (such as at least about any one of 80%, 85%, 90%, 95%, 96%, 97%, 98%, or 99%) sequence identity.

In some embodiments according to any of the anti-CD93 constructs described above, the antibody moiety is an antibody or antigen-binding fragment thereof selected from the group consisting of a full-length antibody, a bispecific antibody, a single-chain Fv (scFv) fragment, a Fab fragment, a Fab′ fragment, a F(ab′)2, an Fv fragment, a disulfide stabilized Fv fragment (dsFv), a (dsFv)2, a Fv-Fc fusion, a scFv-Fc fusion, a scFv-Fv fusion, a diabody, a tribody, and a tetrabody. In some embodiments, the antibody moiety is a full-length antibody.

In some embodiments according to any of the anti-CD93 constructs described above, the antibody moiety has an Fc fragment is selected from the group consisting of Fc fragments form IgG, IgA, IgD, IgE, IgM, and combinations and hybrids thereof. In some embodiments, the Fc fragment is selected from the group consisting of Fc fragments from IgG1, IgG2, IgG3, IgG4, and combinations and hybrids thereof. In some embodiments, the Fc fragment has a reduced effector function as compared to the corresponding wildtype Fc fragment. In some embodiments, the Fc fragment has an enhanced effector function as compared to the corresponding wildtype Fc fragment. In some embodiments the Fc fragment has extended serum half-life. In some embodiments the Fc fragment has reduced serum half-life.

In some embodiments according to any of the anti-CD93 constructs described above, the antibody moiety blocks the binding of CD93 to IGFBP7 (such as human IGFBP7).

In some embodiments according to any of the anti-CD93 constructs described above, the antibody moiety blocks the binding of CD93 to MMRN2 (such as human MMRN2).

In some embodiments according to any of the anti-CD93 constructs described above, the antibody moiety blocks a) the binding of CD93 to IGFBP7 and/or b) the binding of CD93 to MMRN2.

In some embodiments according to any of the anti-CD93 constructs described above, the CD93 is a human CD93.

In some embodiments, there is provided a fusion protein comprising any of the anti-CD93 constructs described above. In some embodiments, the anti-CD93 constructs is fused to one of more cellular signaling peptides or proteins. In some embodiments, the anti-CD93 construct is fused to one or more VEGF binding moieties. In some embodiments, the anti-CD93 construct is fused to one or more VEGF-A binding moieties. In some embodiments, the VEGF-A binding moieties is Aflibercept. In one embodiment, the fusion protein comprises a heavy chain fusion polypeptide comprising the amino acid sequence set forth in SEQ ID NO: 366, or a variant comprising an amino acid sequence having at least about 80% (such as at least about any one of 80%, 85%, 90%, 95%, 96%, 97%, 98%, or 99%) sequence identity, and a light chain polypeptide comprising the amino acid sequence set forth in SEQ ID NO: 367, or a variant comprising an amino acid sequence having at least about 80% (such as at least about any one of 80%, 85%, 90%, 95%, 96%, 97%, 98%, or 99%) sequence identity.

The present application in another aspect provides a pharmaceutical composition comprising any of the anti-CD93 constructs described above, and a pharmaceutical acceptable carrier.

The present application in another aspect provides an isolated nucleic acid encoding any of the anti-CD93 constructs described above.

The present application in another aspect provides a vector comprising any of the isolated nucleic acids described above.

The present application in another aspect provides an isolated host cell comprising any of the isolated nucleic acids or vectors described above.

The present application in another aspect provides an immunoconjugate comprising the any of the anti-CD93 constructs described above, linked to a therapeutic agent or a label.

The present application in another aspect provides a method of producing an anti-CD93 construct comprising: a) culturing the isolated host cell of claim 25 under conditions effective to express the anti-CD93 construct; and b) obtaining the expressed anti-CD93 construct from the host cell.

The present application in another aspect provides a method of treating a disease or condition in an individual, comprising administering to the individual an effective mount of any of the anti-CD93 constructs or pharmaceutical compositions described above. In some embodiments, the disease or condition is associated with an abnormal vascular structure. In some embodiments, the disease or condition is a cancer. In some embodiments, the cancer is a solid tumor. In some embodiments, the cancer comprises CD93+ endothelial cells. In some embodiments, the cancer comprises IGFBP7+ blood vessels. In some embodiments, the cancer is characterized by tumor hypoxia. In some embodiments, the cancer is a locally advanced or metastatic cancer. In some embodiments, the cancer is selected from the group consisting of a lymphoma, colon cancer, brain cancer, breast cancer, ovarian cancer, endometrial cancer, esophageal cancer, prostate cancer, cervical cancer, renal cancer, bladder cancer, gastric cancer, non-small cell lung cancer, melanoma, and pancreatic cancer. In some embodiments, the anti-CD93 construct is administered parenterally into the individual. In some embodiments, the method further comprises administering a second therapy. In some embodiments, the second therapy is selected from the group consisting of surgery, radiation, gene therapy, immunotherapy, bone marrow transplantation, stem cell transplantation, hormone therapy, targeted therapy, cryotherapy, ultrasound therapy, photodynamic therapy, and chemotherapy.

In some embodiments, the second therapy is an immunotherapy. In some embodiments, the immunotherapy comprises administering an immunomodulatory agent. In some embodiments, the immunomodulatory agent is an immune checkpoint inhibitor. In some embodiments, the immune checkpoint inhibitor comprises an anti-PD-L1 antibody or an anti-PD-1 antibody. In some embodiments, the individual is a human.

### BRIEF DESCRIPTION OF THE DRAWINGS

FIG. 1 shows binding affinity of 16E4 and MM01 against human or cynomolgus CD93.

FIG. 2 shows binding of various anti-CD93 antibodies to CD93-expressing CHO cells.

FIGS. 3A-3D show that the inhibition of the interaction between CD93 and IGFBP7 by 16E4 and MM01 as compared to mIgG isotype at various concentrations.

FIGS. 4A-4F show the inhibition of HUVEC tube formation by various anti-CD93 antibodies as compared to control.

FIGS. 5A-5B show results of epitope binning of various anti-CD93 antibodies by Octet competition.

FIGS. 6A-6B show cross-binding activities of various anti-CD93 antibodies against human and cynomolgus CD93 measured by bio-layer interferometry (BLI) assay.

FIGS. 7A-7B show alignment of V_{H} and V_{L} CDRs according to Kabat numbering. From top to bottom, sequences in FIG. 7A are SEQ ID NO: 393-406, and sequences in FIG. 7B are SEQ ID NO: 407-420.

FIGS. 8A-8B show alignment of V_{H} and V_{L} CDRs determined by the VBASE2 tool. From top to bottom, sequences in FIG. 8A are SEQ ID NO: 393-406, and sequences in FIG. 8B are SEQ ID NO: 407-420.

FIG. 9 shows binding affinity of 10B1 and 7F3 to human CD93.

FIG. 10 shows binding of 16E4, 10B1 and 7F3 to human CD93-expressing CHO cells and lack of binding to CHO-K1 cells.

FIGS. 11A-11B show that the inhibition of the interaction between CD93 and MMRN2 by 16E4, 10B1, and 7F3 as compared to mIgG isotype at 50 sg/mL.

FIG. 12 shows the inhibition of the interaction between CD93 and MMRN2 by 7F3 at different MMRN2 concentrations as compared to control (IgG2a)

FIG. 13 shows the inhibition of the interaction between CD93 and MMRN2 by 7F3 as compared to control (IgG1).

FIG. 14 show that the inhibition of the interaction between CD93 and IGFBP7 by 7F3 as compared to mIgG1 isotype at various concentrations.

FIGS. 15A-15B shows the inhibition of HUVEC tube formation by 16E4 and 7F3 at two concentrations as compared to control.

FIG. 16 shows exemplary multispecific anti-CD93 constructs that also recognize VEGF.

FIG. 17 shows tumor volume in mice treated with exemplary anti-CD93 constructs.

FIG. 18 shows tumor volume in mice treated with humanized 17B10 anti-CD93 antibody.

FIG. 19 shows binding of anti-CD93 antibodies to primary HUVEC cells in the presence of human serum determined by flow cytometry.

FIG. 20 shows binding of anti-CD93 antibodies to primary HUVEC cells in the absence of human serum determined by flow cytometry.

FIG. 21 shows binding of anti-CD93 antibodies to hCD93 CHO cells in the presence of human serum determined by flow cytometry assay.

FIG. 22 shows binding of anti-CD93 antibodies to U937 cells determined by flow cytometry assay.

FIGS. 23-24 show the inhibition effect of an exemplary humanized 17B10 antibody in HUVEC tube formation.

FIGS. 25A-25B show binding of exemplary humanized 17B10 antibodies to overexpressing human CD93 CHO cells.

FIGS. 26A-26B show binding of exemplary humanized 17B10 antibodies to KG1a and U937 cells.

FIG. 27 shows binding of humanized anti-CD93 antibody 17B10 to cell surface expressing mouse CD93 CHO cells determined by fluorescence activated cell sorting (FACS) assay.

FIG. 28 shows binding of an exemplary humanized 17B10 antibody to cell surface expressing mouse CD93 HEK cells determined by fluorescence activated cell sorting (FACS) assay.

FIG. 29 shows SDS-PAGE analysis of exemplary humanized 16E4 antibody and humanized 7F3 antibody.

FIG. 30 shows ELISA analysis of the binding of exemplary humanized 16E4 and 7F3 antibodies to human CD93 (hCD93).

FIG. 31 shows ELISA analysis of the binding of exemplary h7F3 (humanized 7F3) antibodies to human CD93 (hCD93).

FIG. 32 shows ELISA analysis the binding of exemplary hybridoma or humanized 16E4 antibodies to hCD93.

FIG. 33 shows ELISA analysis of the binding of exemplary hybridoma or humanized 17B10 antibodies to hCD93.

FIG. 34 shows ELISA analysis of the binding of exemplary humanized 17B10 to hCD93.

FIG. 35 shows FACS analysis of the binding of 16E4-hIgG1 and 7F3-hIgG1 antibodies to CHO-hCD93 cells.

FIG. 36 shows FACS analysis of the binding of humanized 7F3 to CHO-hCD93 cells.

FIG. 37 shows FACS analysis of the binding of h16E4 (humanized 16E4) to CHO-hCD93 cells.

FIG. 38 shows FACS analysis of the binding of humanized 7F3 to HUVEC cells.

FIG. 39 shows FACS analysis of the binding of humanized 7F3 KG1a cells.

FIG. 40 shows FACS analysis of the binding of humanized 16E4 to KG1a cells.

FIG. 41 shows kinetic characterization of the binding of exemplary 16E4 and 7F3 antibodies to hCD93.

FIG. 42 shows kinetic characterization of the binding of exemplary humanized 16E4 antibodies to hCD93

FIG. 43 shows a summary of the binding affinities of exemplary 16E4 and 7F3 antibodies to human CD93 by octet, and human CD93 expressing CHO cells, HUVEC cells, or KG1a cells measured by Flow cytometry.

FIG. 44 shows FACS analysis of the blocking effect of humanized 7F3 on the binding of human MMRN2 to CHO-hCD93 cells.

FIG. 45 shows FACS analysis of the blocking effect of humanized 16E4 and 7F3 antibodies on the binding of MMRN2 to CHO-hCD93 cells.

FIG. 46 shows FACS analysis of the blocking effect of an exemplary humanized 7F3 antibody on the binding of human IGFBP7 to HUVEC cells.

FIG. 47 shows Octet analysis of the blocking effect of exemplary 7F3 or 16E4 antibodies on the binding of human IGFBP7 to human CD93.

FIG. 48 shows Octet analysis of the blocking effect of exemplary 16E4 antibodies on the binding of human IGFBP7 to human CD93.

FIGS. 49-50 show the effects of exemplary humanized 7F3 and 16E4 antibodies on HUVEC tube formation.

FIG. 51 shows a summary of properties of exemplary anti-CD93 antibodies.

FIG. 52A shows the results of in vivo anti-tumor efficacy of 7F3, 16E4, and 17B10 chimeric in B16F10 mouse model as well as the body weight change of the treated mice. FIG. 52C shows the results of in vivo anti-tumor efficacy of 7F3, 16E4, 17B10 and 7F3/VEGFRFc in B16F10 mouse model as well as the body weight change of the treated mice.

FIG. 53A shows the schematic design of h7F3/VEGFR constructs.

FIG. 53B shows the results of FACS binding assay between CD93 and chimeric 7F3-hIgG1 or an exemplary chimeric 7F3/VEGFR construct (i.e., 7F3-Aflibercept).

FIG. 53C-53D show the results of FACS blocking assay. FIG. 53C shows that original 7F3-mIgG1, humanized 7F3-hIgG1 and the exemplary 7F3/VEGFR construct humanized 7F3-Aflibercept all block the interaction between CD93 and IGFBP7. FIG. 53D shows that original 7F3-mIgG1, humanized 7F3-hIgG1 and the exemplary 7F3/VEGFR construct humanized 7F3-Aflibercept all block the interaction between CD93 and MMRN2.

FIG. 53E-F show the results of ELISA binding assay. FIG. 53E shows that chimeric 7F3-hIgG1 and humanized 7F3-Aflibercept both bind to human CD93 while humanized 7F3-Aflibercept and Avastin both bind to VEGFA. FIG. 53F shows that chimeric 7F3-hIgG1, chimeric 7F3-Aflibercept, and humanized 7F3-Aflibercept bind to both human CD93 and cynoCD93. Avastin, chimeric 7F3-Aflibercept and humanized 7F3-Aflibercept all bind to human VEGFA, while only chimeric 7F3-Aflibercept and humanized 7F3-Aflibercept bind to mouse VEGFA.

FIG. 53G shows the results of Octet binding assay that tested the binding between VEGFA and hFc-VEGF trap, h7F3-VEGF trap or Avastin.

### DETAILED DESCRIPTION OF THE APPLICATION

The present application provides novel anti-CD93 constructs that specifically bind to CD93 (such as anti-CD93 monoclonal or multispecific antibodies), methods of preparing the anti-CD93 constructs, methods of using the constructs (e.g., methods of treating a disease or condition).

Anti-CD93 antibodies (e.g., anti-CD93 antibodies that block interaction between CD93 and IGFBP7) may effectively treat a tumor or cancer, block abnormal tumor vascular angiogenesis, normalize immature and leaky tumor blood vessel, promote functional vascular network in a tumor, promote vascular maturation, promote a favorable tumor microenvironment, increase immune cell infiltration in a tumor, increase tumor perfusion, reduce hyperplasia in a tumor, sensitize tumor to a second therapy, and/or facilitating delivery of a second agent. See e.g., WO2021062128A1, the disclosure of which is herein incorporated by reference in its entirety. In some embodiments, the anti-CD93 construct described herein reduces the size of a tumor. In some embodiments, the anti-CD93 construct described herein promotes immune cell infiltration in a tumor. In some embodiments, the anti-CD93 construct described herein promotes vascular maturation in a tumor. In some embodiments, the anti-CD93 construct described herein sensitizes a tumor to a second therapy or facilitates delivery of a second agent.

### I. Definitions

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antibody moiety” refers to a full-length antibody or an antigen-binding fragment thereof.

A full-length antibody comprises two heavy chains and two light chains. The variable regions of the light and heavy chains are responsible for antigen binding. The variable domains of the heavy chain and light chain may be referred to as “V_{H}” and “V_{L}”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein maybe defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasmme such as 1gG1 (γ1 heavy chain), 1gG2 (γ2 heavy chain), 1gG3 (γ3 heavy chain), 1gG4 (γ4 heavy chain), 1gA1 (α1 heavy chain), or 1gA2 (α2 heavy chain).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelid single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above-cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein. In some embodiments, the CDR sequences provided herein are based on IMGT definition. For example, the CDR sequences may be determined by the VBASE2 tool (http://www.vbase2.org/vbase2.php, see also Retter I, Althaus H H, Münch R, Müller W: VBASE2, an integrative V gene database. Nucleic Acids Res. 2005 Jan. 1; 33 (Database issue): D671-4, which is incorporated herein by reference in its entirety).

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or hypervariable region (HVR) of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

“Framework” or “FR” residues are those variable-domain residues other than the CDR residues as herein defined.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, See Jones et al., Nature 321:522-525 (1986); Riechmann et al., Nature 332:323-329 (1988); and Presta, Curr. Op. Struct. Biol. 2:593-596 (1992).

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

“Homologous” refers to the sequence similarity or sequence identity between two polypeptides or between two nucleic acid molecules. When a position in both of the two compared sequences is occupied by the same base or amino acid monomer subunit, e.g., if a position in each of two protein molecules is occupied by lysine, or if a position in each of two DNA molecules is occupied by adenine, then the molecules are homologous at that position.

The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the protein sequences SGTSTD (SEQ ID NO: 421) and TGTSDA (SEQ ID NO: 422) share 50% homology. Generally, a comparison is made when two sequences are aligned to give maximum homology.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the CHL (or C_{L}) domain of the light chain.

The “light chains” of antibodies (immunoglobulins) from any mammalian species can be assigned to one of two clearly distinct types, called kappa (“κ”) and lambda (“λ”), based on the amino acid sequences of their constant domains.

The “CH1 domain” (also referred to as “C1” of “H1” domain) usually extends from about amino acid 118 to about amino acid 215 (EU numbering system).

“Hinge region” is generally defined as a region in IgG corresponding to Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region (also referred to as “C2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” (also referred to as “C2” domain) comprises the stretch of residues C-terminal to a CH2 domain in an Fc region (i.e. from about amino acid residue 341 to the C-terminal end of an antibody sequence, typically at amino acid residue 446 or 447 of an IgG).

The term “Fc region” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody.

Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Fc receptor” or “FcR” describes a receptor that binds the Fc region of an antibody. The preferred FcR is a native sequence human FcR. Moreover, a preferred FcR is one which binds an IgG antibody (a gamma receptor) and includes receptors of the FcγRI, FcγRII, FcRN, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIB contains an immunoreceptor tyrosine-based inhibition motif (ITIM) in its cytoplasmic domain. (See M. Daëron, Annu. Rev. Immunol. 15:203-234 (1997). FcRN is critical to the recycling of an antibody to the blood allowing for increased serum half-life of the antibodies. FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody or fragment thereof “competes” for binding to a target antigen with a second antibody or fragment thereof when the first antibody or fragment thereof inhibits the target antigen binding of the second antibody of fragment thereof by at least about 50% (such as at least about any one of 55%, 60%, 65%, 70%, 75%, 80%, 85%, 90%, 95%, 98% or 99%) in the presence of an equimolar concentration of the first antibody or fragment thereof, or vice versa. A high throughput process for “binning” antibodies based upon their cross-competition is described in PCT Publication No. WO 03/48731.

As used herein, the terms “specifically binds,” “specifically recognizing,” and “is specific for” refer to measurable and reproducible interactions, such as binding between a target and an antibody or antibody moiety, which is determinative of the presence of the target in the presence of a heterogeneous population of molecules, including biological molecules. For example, an antibody or antibody moiety that specifically recognizes a target (which can be an epitope) is an antibody or antibody moiety that binds this target with greater affinity, avidity, more readily, and/or with greater duration than its bindings to other targets. In some embodiments, the extent of binding of an antibody to an unrelated target is less than about 10% of the binding of the antibody to the target as measured, e.g., by a radioimmunoassay (RIA). In some embodiments, an antibody that specifically binds a target has a dissociation constant (K_{D}) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA-, BLI, RIA-, ECL-, IRMA-, EIA-, BIACORE™-tests and peptide scans.

As used herein, molecule A (e.g., an anti-CD93 construct as described herein) “blocks” the binding of molecule B (e.g., CD93) and molecule C (e.g., IGFBP7 or MMRN2) refers to both direct blocking and indirect blocking. For example, instead of directly blocking the binding of CD93 and IGFBP7 or MMRN2 by occupying at least a portion of the binding site on CD93 that is responsible for IGFBP7 or MMRN2 binding, an anti-CD93 construct as described herein may block the binding of CD93 and IGFBP7 or MMRN2 by altering the structure of CD93 such that CD93 and IGFBP7/MMRN2 cannot bind.

An “isolated” or “purified” antibody (or construct) is one that has been identified, separated and/or recovered from a component of its production environment (e.g., natural or recombinant). Preferably, the isolated polypeptide is free of association with all other components from its production environment.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, and may contain mutations. Mutant progeny that have the same function or biological activity as screened or selected for in the originally transformed cell are included herein.

The term “immunoconjugate” includes reference to a covalent linkage of a therapeutic agent or a detectable label to an antibody such as an antibody moiety described herein. The linkage can be direct or indirect through a linker (such as a peptide linker).

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment.

In the context of cancer, the term “treating” includes any or all of: inhibiting growth of cancer cells, inhibiting replication of cancer cells, lessening of overall tumor burden and ameliorating one or more symptoms associated with the disease.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to that of a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or patient.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing” as used herein, includes providing prophylaxis with respect to the occurrence or recurrence of a disease in an individual that may be predisposed to the disease but has not yet been diagnosed with the disease.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

The terms “subject,” “individual,” and “patient” are used interchangeably herein to refer to a mammal, including, but not limited to, human, bovine, horse, feline, canine, rodent, or primate. In some embodiments, the individual is a human.

An “effective amount” of an agent refers to an amount effective, at dosages and for periods of time necessary, to achieve the desired therapeutic or prophylactic result. The specific dose may vary depending on one or more of: the particular agent chosen, the dosing regimen to be followed, whether it is administered in combination with other compounds, timing of administration, the tissue to be imaged, and the physical delivery system in which it is carried.

A “therapeutically effective amount” of a substance/molecule of the application, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

The terms “pharmaceutical formulation” and “pharmaceutical composition” refer to a preparation which is in such form as to permit the biological activity of the active ingredient(s) to be effective, and which contains no additional components which are unacceptably toxic to an individual to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to an individual. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about 60 minutes, such as no more than about any of 30, 15, 10, 5, or 1 minutes.

The term “sequentially” is used herein to refer to administration of two or more therapeutic agents where the administration of one or more agent(s) continues after discontinuing the administration of one or more other agent(s). For example, administration of the two or more therapeutic agents are administered with a time separation of more than about 15 minutes, such as about any of 20, 30, 40, 50, or 60 minutes, 1 day, 2 days, 3 days, 1 week, 2 weeks, or 1 month, or longer.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker described herein. In certain embodiments, the manufacture or kit is promoted, distributed, or sold as a unit for performing the methods described herein.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### II. Anti-CD93 Constructs

The present application provides anti-CD93 constructs comprising an anti-CD93 antibody moiety that specifically binds to CD93 as described herein.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the V_{L-2} comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6.

In some embodiments, the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 7, ii) the HC-CDR2 comprising the amino acid sequence of SEQ ID NO: 8, and iii) the HC-CDR3 comprising the amino acid sequence of SEQ ID NO: 9, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 10, ii) the LC-CDR2 comprising the amino acid sequence of SEQ ID NO: 11, and iii) the LC-CDR3 comprising the amino acid sequence of SEQ ID NO: 12, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 13; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 14.

In some embodiments, the V_{H} comprises an amino acid sequence of SEQ ID NO: 13, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 14, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 17, the HC-CDR2 comprising the amino acid sequence of SEQ ID NO: 18, and the HC-CDR3 comprising the amino acid sequence of SEQ ID NO: 19, and the V_{L-2} comprises the LC-CDR1 comprising the amino acid sequence of SEQ ID NO: 20, the LC-CDR2 comprising the amino acid sequence of SEQ ID NO: 21, and the LC-CDR3 comprising the amino acid sequence of SEQ ID NO: 22.

In some embodiments, the V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 23, ii) the HC-CDR2 comprising the amino acid sequence of SEQ ID NO: 24, and iii) the HC-CDR3 comprising the amino acid sequence of SEQ ID NO: 25, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 26, ii) the LC-CDR2 comprising the amino acid sequence of SEQ ID NO: 27, and iii) the LC-CDR3 comprising the amino acid sequence of SEQ ID NO: 28, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, and the V_{L} comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 29 and 307-312; and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 30, and 313-318.

In some embodiments, the V_{H} comprises an amino acid sequence of any of SEQ ID NO: 29 and 307-312, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of any of SEQ ID NO: 30, and 313-318, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the V_{L-2} comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38.

In some embodiments, the V_{H} comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 39, ii) the HC-CDR2 comprising the amino acid sequence of SEQ ID NO: 40, and iii) the HC-CDR3 comprising the amino acid sequence of SEQ ID NO: 41, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 42, ii) the LC-CDR2 comprising the amino acid sequence of SEQ ID NO: 43, and iii) the LC-CDR3 comprising the amino acid sequence of SEQ ID NO: 44, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, and the V_{L} comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 45; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 46.

In some embodiments, the V_{H} comprises an amino acid sequence of SEQ ID NO: 45, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 46, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 49, the HC-CDR2 comprising the amino acid sequence of SEQ ID NO: 50, and the HC-CDR3 comprising the amino acid sequence of SEQ ID NO: 51, and the V_{L-2} comprises the LC-CDR1 comprising the amino acid sequence of SEQ ID NO: 52, the LC-CDR2 comprising the amino acid sequence of SEQ ID NO: 53, and the LC-CDR3 comprising the amino acid sequence of SEQ ID NO: 54.

In some embodiments, the V_{H} comprises i) the HC-CDR1 comprising the amino acid sequence of SEQ ID NO: 49, ii) the HC-CDR2 comprising the amino acid sequence of SEQ ID NO: 50, and iii) the HC-CDR3 comprising the amino acid sequence of SEQ ID NO: 51, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 52, ii) the LC-CDR2 comprising the amino acid sequence of SEQ ID NO: 53, and iii) the LC-CDR3 comprising the amino acid sequence of SEQ ID NO: 54, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 55, ii) the HC-CDR2 comprising the amino acid sequence of SEQ ID NO: 56, and iii) the HC-CDR3 comprising the amino acid sequence of SEQ ID NO: 57, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 58, ii) the LC-CDR2 comprising the amino acid sequence of SEQ ID NO: 59, and iii) the LC-CDR3 comprising the amino acid sequence of SEQ ID NO: 60, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 49, ii) the HC-CDR2 comprising the amino acid sequence of SEQ ID NO: 50, and iii) the HC-CDR3 comprising the amino acid sequence of SEQ ID NO: 51, and the V_{L} comprises i) the LC-CDR1 comprising the amino acid sequence of SEQ ID NO: 52, ii) the LC-CDR2 comprising the amino acid sequence of SEQ ID NO: 53, and iii) the LC-CDR3 comprising the amino acid sequence of SEQ ID NO: 54.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 61; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 62.

In some embodiments, the V_{H} comprises an amino acid sequence of SEQ ID NO: 61, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 62, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 65, the HC-CDR2 comprising the amino acid sequence of SEQ ID NO: 66, and the HC-CDR3 comprising the amino acid sequence of SEQ ID NO: 67, and the V_{L-2} comprises the LC-CDR1 comprising the amino acid sequence of SEQ ID NO: 68, the LC-CDR2 comprising the amino acid sequence of SEQ ID NO: 69, and the LC-CDR3 comprising the amino acid sequence of SEQ ID NO: 70.

In some embodiments, the V_{H} comprises i) the HC-CDR1 comprising the amino acid sequence of SEQ ID NO: 65, ii) the HC-CDR2 comprising the amino acid sequence of SEQ ID NO: 66, and iii) the HC-CDR3 comprising the amino acid sequence of SEQ ID NO: 67, or a variant thereof comprising up to 5, 4, 3, 2, or 1 amino acid substitutions in the HC-CDRs, and the VI, comprises i) the LC-CDR1 comprising the amino acid sequence of SEQ ID NO: 68, ii) the LC-CDR2 comprising the amino acid sequence of SEQ ID NO: 69, and iii) the LC-CDR3 comprising the amino acid sequence of SEQ ID NO: 70, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 71, ii) the HC-CDR2 comprising the amino acid sequence of SEQ ID NO: 72, and iii) the HC-CDR3 comprising the amino acid sequence of SEQ ID NO: 73, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 74, ii) the LC-CDR2 comprising the amino acid sequence of SEQ ID NO: 75, and iii) the LC-CDR3 comprising the amino acid sequence of SEQ ID NO: 76, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 65, ii) the HC-CDR2 comprising the amino acid sequence of SEQ ID NO: 66, and iii) the HC-CDR3 comprising the amino acid sequence of SEQ ID NO: 67, and the V_{L} comprises i) the LC-CDR1 comprising the amino acid sequence of SEQ ID NO: 68, ii) the LC-CDR2 comprising the amino acid sequence of SEQ ID NO: 69, and iii) the LC-CDR3 comprising the amino acid sequence of SEQ ID NO: 70.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 77; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 78.

In some embodiments, the V_{H} comprises an amino acid sequence of SEQ ID NO: 77, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 78, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 81, the HC-CDR2 comprising the amino acid sequence of SEQ ID NO: 82, and the HC-CDR3 comprising the amino acid sequence of SEQ ID NO: 83, and the V_{L-2} comprises the LC-CDR1 comprising the amino acid sequence of SEQ ID NO: 84, the LC-CDR2 comprising the amino acid sequence of SEQ ID NO: 85, and the LC-CDR3 comprising the amino acid sequence of SEQ ID NO: 86.

In some embodiments, the V_{H} comprises i) the HC-CDR1 comprising the amino acid sequence of SEQ ID NO: 81, ii) the HC-CDR2 comprising the amino acid sequence of SEQ ID NO: 82, and iii) the HC-CDR3 comprising the amino acid sequence of SEQ ID NO: 83, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 84, ii) the LC-CDR2 comprising the amino acid sequence of SEQ ID NO: 85, and iii) the LC-CDR3 comprising the amino acid sequence of SEQ ID NO: 86, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 87, ii) the HC-CDR2 comprising the amino acid sequence of SEQ ID NO: 88, and iii) the HC-CDR3 comprising the amino acid sequence of SEQ ID NO: 89, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 90, ii) the LC-CDR2 comprising the amino acid sequence of SEQ ID NO: 91, and iii) the LC-CDR3 comprising the amino acid sequence of SEQ ID NO: 92, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 81, ii) the HC-CDR2 comprising the amino acid sequence of SEQ ID NO: 82, and iii) the HC-CDR3 comprising the amino acid sequence of SEQ ID NO: 83, and the V_{L} comprises i) the LC-CDR1 comprising the amino acid sequence of SEQ ID NO: 84, ii) the LC-CDR2 comprising the amino acid sequence of SEQ ID NO: 85, and iii) the LC-CDR3 comprising the amino acid sequence of SEQ ID NO: 86.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 93; and a LC-CDR1, a LC-CDR2, and a LC-CDR3, respectively comprising the amino acid sequences of a CDR1, a CDR2, and a CDR3 within a VI, chain region having the sequence set forth in SEQ ID NO: 94.

In some embodiments, the V_{H} comprises an amino acid sequence of SEQ ID NO: 93, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 94, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 97, the HC-CDR2 comprising the amino acid sequence of SEQ ID NO: 98, and the HC-CDR3 comprising the amino acid sequence of SEQ ID NO: 99, and the V_{L-2} comprises the LC-CDR1 comprising the amino acid sequence of SEQ ID NO: 100, the LC-CDR2 comprising the amino acid sequence of SEQ ID NO: 101, and the LC-CDR3 comprising the amino acid sequence of SEQ ID NO: 102.

In some embodiments, the V_{H} comprises i) the HC-CDR1 comprising the amino acid sequence of SEQ ID NO: 97, ii) the HC-CDR2 comprising the amino acid sequence of SEQ ID NO: 98, and iii) the HC-CDR3 comprising the amino acid sequence of SEQ ID NO: 99, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 100, ii) the LC-CDR2 comprising the amino acid sequence of SEQ ID NO: 101, and iii) the LC-CDR3 comprising the amino acid sequence of SEQ ID NO: 102, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 103, ii) the HC-CDR2 comprising the amino acid sequence of SEQ ID NO: 104, and iii) the HC-CDR3 comprising the amino acid sequence of SEQ ID NO: 105, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 106, ii) the LC-CDR2 comprising the amino acid sequence of SEQ ID NO: 107, and iii) the LC-CDR3 comprising the amino acid sequence of SEQ ID NO: 108, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 97, ii) the HC-CDR2 comprising the amino acid sequence of SEQ ID NO: 98, and iii) the HC-CDR3 comprising the amino acid sequence of SEQ ID NO: 99, and the V_{L} comprises i) the LC-CDR1 comprising the amino acid sequence of SEQ ID NO: 100, ii) the LC-CDR2 comprising the amino acid sequence of SEQ ID NO: 101, and iii) the LC-CDR3 comprising the amino acid sequence of SEQ ID NO: 102.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 109; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 110.

In some embodiments, the V_{H} comprises an amino acid sequence of SEQ ID NO: 109, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of SEQ ID NO: 110, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 113, the HC-CDR2 comprising the amino acid sequence of SEQ ID NO: 114, and the HC-CDR3 comprising the amino acid sequence of SEQ ID NO: 115, and the V_{L-2} comprises the LC-CDR1 comprising the amino acid sequence of SEQ ID NO: 116, the LC-CDR2 comprising the amino acid sequence of SEQ ID NO: 117, and the LC-CDR3 comprising the amino acid sequence of SEQ ID NO: 118.

In some embodiments, the V_{H} comprises i) the HC-CDR1 comprising the amino acid sequence of SEQ ID NO: 113, ii) the HC-CDR2 comprising the amino acid sequence of SEQ ID NO: 114, and iii) the HC-CDR3 comprising the amino acid sequence of SEQ ID NO: 115, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 116, ii) the LC-CDR2 comprising the amino acid sequence of SEQ ID NO: 117, and iii) the LC-CDR3 comprising the amino acid sequence of SEQ ID NO: 118, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 119, ii) the HC-CDR2 comprising the amino acid sequence of SEQ ID NO: 120, and iii) the HC-CDR3 comprising the amino acid sequence of SEQ ID NO: 121, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 122, ii) the LC-CDR2 comprising the amino acid sequence of SEQ ID NO: 123, and iii) the LC-CDR3 comprising the amino acid sequence of SEQ ID NO: 124, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 113, ii) the HC-CDR2 comprising the amino acid sequence of SEQ ID NO: 114, and iii) the HC-CDR3 comprising the amino acid sequence of SEQ ID NO: 115, and the V_{L} comprises i) the LC-CDR1 comprising the amino acid sequence of SEQ ID NO: 116, ii) the LC-CDR2 comprising the amino acid sequence of SEQ ID NO: 117, and iii) the LC-CDR3 comprising the amino acid sequence of SEQ ID NO: 118.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 125; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 126.

In some embodiments, the V_{H} comprises an amino acid sequence of SEQ ID Na 125, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 126, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 129, the HC-CDR2 comprising the amino acid sequence of SEQ ID NO: 130, and the HC-CDR3 comprising the amino acid sequence of SEQ ID NO: 131, and the V_{L-2} comprises the LC-CDR1 comprising the amino acid sequence of SEQ ID NO: 132, the LC-CDR2 comprising the amino acid sequence of SEQ ID NO: 133, and the LC-CDR3 comprising the amino acid sequence of SEQ ID NO: 134.

In some embodiments, the V_{H} comprises i) the HC-CDR1 comprising the amino acid sequence of SEQ ID NO: 129, ii) the HC-CDR2 comprising the amino acid sequence of SEQ ID NO: 130, and iii) the HC-CDR3 comprising the amino acid sequence of SEQ ID NO: 131, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 132, ii) the LC-CDR2 comprising the amino acid sequence of SEQ ID NO: 133, and iii) the LC-CDR3 comprising the amino acid sequence of SEQ ID NO: 134, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 135, ii) the HC-CDR2 comprising the amino acid sequence of SEQ ID NO: 136, and iii) the HC-CDR3 comprising the amino acid sequence of SEQ ID NO: 137, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 138, ii) the LC-CDR2 comprising the amino acid sequence of SEQ ID NO: 139, and iii) the LC-CDR3 comprising the amino acid sequence of SEQ ID NO: 140, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 129, ii) the HC-CDR2 comprising the amino acid sequence of SEQ ID NO: 130, and iii) the HC-CDR3 comprising the amino acid sequence of SEQ ID NO: 131, and the V_{L} comprises i) the LC-CDR1 comprising the amino acid sequence of SEQ ID NO: 132, ii) the LC-CDR2 comprising the amino acid sequence of SEQ ID NO: 133, and iii) the LC-CDR3 comprising the amino acid sequence of SEQ ID NO: 134.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 141; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 142.

In some embodiments, the V_{H} comprises an amino acid sequence of SEQ ID NO: 141, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of SEQ ID NO: 142, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 145, the HC-CDR2 comprising the amino acid sequence of SEQ ID NO: 146, and the HC-CDR3 comprising the amino acid sequence of SEQ ID NO: 147, and the V_{L-2} comprises the LC-CDR1 comprising the amino acid sequence of SEQ ID NO: 148, 355, or 358, the LC-CDR2 comprising the amino acid sequence of SEQ ID NO: 149 or 356, and the LC-CDR3 comprising the amino acid sequence of SEQ ID NO: 150, 357 or 359.

In some embodiments, the V_{H} comprises i) the HC-CDR1 comprising the amino acid sequence of SEQ ID NO: 145, ii) the HC-CDR2 comprising the amino acid sequence of SEQ ID NO: 146, and iii) the HC-CDR3 comprising the amino acid sequence of SEQ ID NO: 147, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 148, 355, or 358, ii) the LC-CDR2 comprising the amino acid sequence of SEQ ID NO: 149 or 356, and iii) the LC-CDR3 comprising the amino acid sequence of SEQ ID NO: 150, 357 or 359, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 151, ii) the HC-CDR2 comprising the amino acid sequence of SEQ ID NO: 152, and iii) the HC-CDR3 comprising the amino acid sequence of SEQ ID NO: 153, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 154, ii) the LC-CDR2 comprising the amino acid sequence of SEQ ID NO: 155, and iii) the LC-CDR3 comprising the amino acid sequence of SEQ ID NO: 156, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 145, ii) the HC-CDR2 comprising the amino acid sequence of SEQ ID NO: 146, and iii) the HC-CDR3 comprising the amino acid sequence of SEQ ID NO: 147, and the V_{L} comprises i) the LC-CDR1 comprising the amino acid sequence of SEQ ID NO: 148, 355, or 358, ii) the LC-CDR2 comprising the amino acid sequence of SEQ ID NO: 149 or 356, and iii) the LC-CDR3 comprising the amino acid sequence of SEQ ID NO: 150, 357 or 359.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 157 and 360-362; and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 158, and 363-365.

In some embodiments, the V_{H} comprises an amino acid sequence of any of SEQ ID NO: 157 and 360-362, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of any of SEQ ID NO: 158, and 363-365, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID Na 157, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 158, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID Na 360, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 363, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 360, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 364, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 360, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 365, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 361, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 363, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 361, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 364, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 361, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 365, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 362, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 363, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 362, or a variant comprising an amino acid sequence having at least about 80% (such as at least about any one of 80%, 85%, 90%, 95%, 96%, 97%, 98% or 99%) sequence identity; and the V_{L} comprises an amino acid sequence of SEQ ID NO: 364, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 362, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 365, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 161, the HC-CDR2 comprising the amino acid sequence of SEQ ID NO: 162, and the HC-CDR3 comprising the amino acid sequence of SEQ ID NO: 163, and the V_{L-2} comprises the LC-CDR1 comprising the amino acid sequence of SEQ ID NO: 164, the LC-CDR2 comprising the amino acid sequence of SEQ ID NO: 165, and the LC-CDR3 comprising the amino acid sequence of SEQ ID NO: 166.

In some embodiments, the V_{H} comprises i) the HC-CDR1 comprising the amino acid sequence of SEQ ID NO: 161, ii) the HC-CDR2 comprising the amino acid sequence of SEQ ID NO: 162, and iii) the HC-CDR3 comprising the amino acid sequence of SEQ ID NO: 163, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 164, ii) the LC-CDR2 comprising the amino acid sequence of SEQ ID NO: 165, and iii) the LC-CDR3 comprising the amino acid sequence of SEQ ID NO: 166, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 167, ii) the HC-CDR2 comprising the amino acid sequence of SEQ ID NO: 168, and iii) the HC-CDR3 comprising the amino acid sequence of SEQ ID NO: 169, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 170, ii) the LC-CDR2 comprising the amino acid sequence of SEQ ID NO: 171, and iii) the LC-CDR3 comprising the amino acid sequence of SEQ ID NO: 172, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 161, ii) the HC-CDR2 comprising the amino acid sequence of SEQ ID NO: 162, and iii) the HC-CDR3 comprising the amino acid sequence of SEQ ID NO: 163, and the V_{L} comprises i) the LC-CDR1 comprising the amino acid sequence of SEQ ID NO: 164, ii) the LC-CDR2 comprising the amino acid sequence of SEQ ID NO: 165, and iii) the LC-CDR3 comprising the amino acid sequence of SEQ ID NO: 166.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 173; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 174.

In some embodiments, the V_{H} comprises an amino acid sequence of SEQ ID NO: 173, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 174, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 177, the HC-CDR2 comprising the amino acid sequence of SEQ ID NO: 178, and the HC-CDR3 comprising the amino acid sequence of SEQ ID NO: 179, and the V_{L-2} comprises the LC-CDR1 comprising the amino acid sequence of SEQ ID NO: 180 or 353, the LC-CDR2 comprising the amino acid sequence of SEQ ID NO: 181 or 354, and the LC-CDR3 comprising the amino acid sequence of SEQ ID NO: 182.

In some embodiments, the V_{H} comprises i) the HC-CDR1 comprising the amino acid sequence of SEQ ID NO: 177, ii) the HC-CDR2 comprising the amino acid sequence of SEQ ID NO: 178, and iii) the HC-CDR3 comprising the amino acid sequence of SEQ ID NO: 179, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 180 or 353, ii) the LC-CDR2 comprising the amino acid sequence of SEQ ID NO: 181 or 354, and iii) the LC-CDR3 comprising the amino acid sequence of SEQ ID NO: 182, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 177, ii) the HC-CDR2 comprising the amino acid sequence of SEQ ID NO: 178, and iii) the HC-CDR3 comprising the amino acid sequence of SEQ ID NO: 179, and the V_{L} comprises i) the LC-CDR1 comprising the amino acid sequence of SEQ ID NO: 180, ii) the LC-CDR2 comprising the amino acid sequence of SEQ ID NO: 181, and iii) the LC-CDR3 comprising the amino acid sequence of SEQ ID NO: 182. In some embodiments, the V_{H} comprises i) the HC-CDR1 comprising the amino acid sequence of SEQ ID NO: 183, ii) the HC-CDR2 comprising the amino acid sequence of SEQ ID NO: 184, and iii) the HC-CDR3 comprising the amino acid sequence of SEQ ID NO: 185, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 186, ii) the LC-CDR2 comprising the amino acid sequence of SEQ ID NO: 187, and iii) the LC-CDR3 comprising the amino acid sequence of SEQ ID NO: 188, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 177, ii) the HC-CDR2 comprising the amino acid sequence of SEQ ID NO: 178, and iii) the HC-CDR3 comprising the amino acid sequence of SEQ ID NO: 179, and the VI, comprises i) the LC-CDR1 comprising the amino acid sequence of SEQ ID NO: 180 or 353, ii) the LC-CDR2 comprising the amino acid sequence of SEQ ID NO: 181 or 354, and iii) the LC-CDR3 comprising the amino acid sequence of SEQ ID NO: 182.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 189 and 347-349; and a LC-CDR1, a LC-CDR2, and a LC-CDR3, respectively comprising the amino acid sequences of a CDR1, a CDR2, and a CDR3 within a VI, chain region having the sequence set forth in any of SEQ ID NO: 190, and 350-352.

In some embodiments, the V_{H} comprises an amino acid sequence of any of SEQ ID NO: 189 and 347-349, or a variant comprising an amino acid sequence having at least about 80% (such as at least about any one of 80%, 85%, 90%, 95%, 96%, 97%, 98%, or 99%) sequence identity; and the VI, comprises an amino acid sequence of any of SEQ ID NO: 190, and 350-352, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID Na 189, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 190, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 347, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 350, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 347, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 351, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of any of SEQ ID NO: 347, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of any of SEQ ID NO: 352, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 348, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 350, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 348, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 351, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 348, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 352, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 349, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 350, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 349, or a variant comprising an amino acid sequence having at least about 80% (such as at least about any one of 80%, 85%, 90%, 95%, 96%, 97%, 98% or 99%) sequence identity, and the V_{L} comprises an amino acid sequence of SEQ ID NO: 351, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 349, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 352, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 193, the HC-CDR2 comprising the amino acid sequence of SEQ ID NO: 194, and the HC-CDR3 comprising the amino acid sequence of SEQ ID NO: 195, and the V_{L-2} comprises the LC-CDR1 comprising the amino acid sequence of SEQ ID NO: 196, the LC-CDR2 comprising the amino acid sequence of SEQ ID NO: 197, and the LC-CDR3 comprising the amino acid sequence of SEQ ID NO: 198.

In some embodiments, the V_{H} comprises i) the HC-CDR1 comprising the amino acid sequence of SEQ ID NO: 193, ii) the HC-CDR2 comprising the amino acid sequence of SEQ ID NO: 194, and iii) the HC-CDR3 comprising the amino acid sequence of SEQ ID NO: 195, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 196, ii) the LC-CDR2 comprising the amino acid sequence of SEQ ID NO: 197, and iii) the LC-CDR3 comprising the amino acid sequence of SEQ ID NO: 198, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 199, ii) the HC-CDR2 comprising the amino acid sequence of SEQ ID NO: 200, and iii) the HC-CDR3 comprising the amino acid sequence of SEQ ID NO: 201, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 202, ii) the LC-CDR2 comprising the amino acid sequence of SEQ ID NO: 203, and iii) the LC-CDR3 comprising the amino acid sequence of SEQ ID NO: 204, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 193, ii) the HC-CDR2 comprising the amino acid sequence of SEQ ID NO: 194, and iii) the HC-CDR3 comprising the amino acid sequence of SEQ ID NO: 195, and the V_{L} comprises i) the LC-CDR1 comprising the amino acid sequence of SEQ ID NO: 196, ii) the LC-CDR2 comprising the amino acid sequence of SEQ ID NO: 197, and iii) the LC-CDR3 comprising the amino acid sequence of SEQ ID NO: 198.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 205; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 206.

In some embodiments, the V_{H} comprises an amino acid sequence of SEQ ID NO: 205, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of SEQ ID NO: 206, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 209, the HC-CDR2 comprising the amino acid sequence of SEQ ID NO: 210, and the HC-CDR3 comprising the amino acid sequence of SEQ ID NO: 211, and the V_{L-2} comprises the LC-CDR1 comprising the amino acid sequence of SEQ ID NO: 212, the LC-CDR2 comprising the amino acid sequence of SEQ ID NO: 213, and the LC-CDR3 comprising the amino acid sequence of SEQ ID NO: 214.

In some embodiments, the V_{H} comprises i) the HC-CDR1 comprising the amino acid sequence of SEQ ID NO: 209, ii) the HC-CDR2 comprising the amino acid sequence of SEQ ID NO: 210, and iii) the HC-CDR3 comprising the amino acid sequence of SEQ ID NO: 211, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 212, ii) the LC-CDR2 comprising the amino acid sequence of SEQ ID NO: 213, and iii) the LC-CDR3 comprising the amino acid sequence of SEQ ID NO: 214, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 215, ii) the HC-CDR2 comprising the amino acid sequence of SEQ ID NO: 216, and iii) the HC-CDR3 comprising the amino acid sequence of SEQ ID NO: 217, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 218, ii) the LC-CDR2 comprising the amino acid sequence of SEQ ID NO: 219, and iii) the LC-CDR3 comprising the amino acid sequence of SEQ ID NO: 220, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 209, ii) the HC-CDR2 comprising the amino acid sequence of SEQ ID NO: 210, and iii) the HC-CDR3 comprising the amino acid sequence of SEQ ID NO: 211, and the V_{L} comprises i) the LC-CDR1 comprising the amino acid sequence of SEQ ID NO: 212, ii) the LC-CDR2 comprising the amino acid sequence of SEQ ID NO: 213, and iii) the LC-CDR3 comprising the amino acid sequence of SEQ ID NO: 214.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 221; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 222.

In some embodiments, the V_{H} comprises an amino acid sequence of SEQ ID NO: 221, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 222, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 289, the HC-CDR2 comprising the amino acid sequence of SEQ ID NO: 290, and the HC-CDR3 comprising the amino acid sequence of SEQ ID NO: 291, and the V_{L-2} comprises the LC-CDR1 comprising the amino acid sequence of SEQ ID NO: 292, the LC-CDR2 comprising the amino acid sequence of SEQ ID NO: 293, and the LC-CDR3 comprising the amino acid sequence of SEQ ID NO: 294.

In some embodiments, the V_{H} comprises i) the HC-CDR1 comprising the amino acid sequence of SEQ ID NO: 289, ii) the HC-CDR2 comprising the amino acid sequence of SEQ ID NO: 290, and iii) the HC-CDR3 comprising the amino acid sequence of SEQ ID NO: 291, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 292, ii) the LC-CDR2 comprising the amino acid sequence of SEQ ID NO: 293, and iii) the LC-CDR3 comprising the amino acid sequence of SEQ ID NO: 294, or a variant thereof comprising up to 5, 4, 3, 2, or 1 amino acid substitutions in the LC-CDRs. In some embodiments, the V_{H} comprises i) the HC-CDR1 comprising the amino acid sequence of SEQ ID NO: 295, ii) the HC-CDR2 comprising the amino acid sequence of SEQ ID NO: 296, and iii) the HC-CDR3 comprising the amino acid sequence of SEQ ID NO: 297, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 298, ii) the LC-CDR2 comprising the amino acid sequence of SEQ ID NO: 299, and iii) the LC-CDR3 comprising the amino acid sequence of SEQ ID NO: 300, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 289, ii) the HC-CDR2 comprising the amino acid sequence of SEQ ID NO: 290, and iii) the HC-CDR3 comprising the amino acid sequence of SEQ ID NO: 291, and the V_{L} comprises i) the LC-CDR1 comprising the amino acid sequence of SEQ ID NO: 292, ii) the LC-CDR2 comprising the amino acid sequence of SEQ ID NO: 293, and iii) the LC-CDR3 comprising the amino acid sequence of SEQ ID NO: 294.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 287 and 319-321; and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 288, and 322-324.

In some embodiments, the V_{H} comprises an amino acid sequence of any of SEQ ID NO: 287 and 319-321, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of any of SEQ ID NO: 288, and 322-324, or a variant comprising an amino acid sequence having at least about 80% (such as at least about any one of 80%, 85%, 90%, 95%, 96%, 97%, 98%, or 99%) sequence identity.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NOs: 287, and 319-321; and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 288, and 322-324.

In some embodiments, the V_{H} comprises an amino acid sequence of any one of SEQ ID NOs: 319-321, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of any one of SEQ ID NOs: 322-324, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 319, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 322, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 319, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 323, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 319, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 324, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 320, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 322, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 320, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 323, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 320, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 324, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 321, or a variant comprising an amino acid sequence having at least about 80% (such as at least about any one of 80%, 85%, 90%, 95%, 96%, 97%, 98% or 99%) sequence identity; and the V_{L} comprises an amino acid sequence of SEQ ID NO: 322, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 321, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 323, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 321, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 324, or a variant comprising an amino acid sequence having at least about 80% (such as at least about any one of 80%, 85%, 90%, 95%, 96%, 97%, 98%, or 99%) sequence identity.

In some embodiments, the anti-CD93 construct comprises an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H}_2) and a second light chain variable region (V_{L-2}), wherein the V_{H-2} comprises the HC-CDR1 comprising the amino acid sequence of SEQ ID NO: 17 or 304, the HC-CDR2 comprising the amino acid sequence of SEQ ID NO: 18 or 305, and the HC-CDR3 comprising the amino acid sequence of SEQ ID NO: 19, and the V_{L-2} comprises the LC-CDR1 comprising the amino acid sequence of SEQ ID NO: 20, 301, 302, 303, or 306, the LC-CDR2 comprising the amino acid sequence of SEQ ID NO: 21, and the LC-CDR3 comprising the amino acid sequence of SEQ ID NO: 22.

In some embodiments, the V_{H} comprises i) the HC-CDR1 comprising the amino acid sequence of SEQ ID NO: 17 or 304, ii) the HC-CDR2 comprising the amino acid sequence of SEQ ID NO: 18 or 305, and iii) the HC-CDR3 comprising the amino acid sequence of SEQ ID NO: 19, or a variant thereof comprising up to 5, 4, 3, 2, or 1 amino acid substitutions in the HC-CDRs, and the VI, comprises i) the LC-CDR1 comprising the amino acid sequence of SEQ ID NO: 20, 301, 302, 303, or 306, ii) the LC-CDR2 comprising the amino acid sequence of SEQ ID NO: 21, and iii) the LC-CDR3 comprising the amino acid sequence of SEQ ID NO: 22, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-CD93 V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, and the anti-CD93 VI, comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22.

In some embodiments, the anti-CD93 V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, and the anti-CD93 V_{L} comprises i) the LC-CDR1 comprising the amino acid sequence of SEQ ID NO: 301, ii) the LC-CDR2 comprising the amino acid sequence of SEQ ID NO: 21, and iii) the LC-CDR3 comprising the amino acid sequence of SEQ ID NO: 22.

In some embodiments, the anti-CD93 V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, and the anti-CD93 VI, comprises i) the LC-CDR1 comprising the amino acid sequence of SEQ ID NO: 302, ii) the LC-CDR2 comprising the amino acid sequence of SEQ ID NO: 21, and iii) the LC-CDR3 comprising the amino acid sequence of SEQ ID NO: 22.

In some embodiments, the anti-CD93 V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, and the anti-CD93 V_{L} comprises i) the LC-CDR1 comprising the amino acid sequence of SEQ ID NO: 303, ii) the LC-CDR2 comprising the amino acid sequence of SEQ ID NO: 21, and iii) the LC-CDR3 comprising the amino acid sequence of SEQ ID NO: 22.

In some embodiments, the anti-CD93 V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, and the anti-CD93 V_{L} comprises i) the LC-CDR1 comprising the amino acid sequence of SEQ ID NO: 306, ii) the LC-CDR2 comprising the amino acid sequence of SEQ ID NO: 21, and iii) the LC-CDR3 comprising the amino acid sequence of SEQ ID NO: 22.

In some embodiments, the anti-CD93 V_{H} comprises i) the HC-CDR1 comprising the amino acid sequence of SEQ ID NO: 304, ii) the HC-CDR2 comprising the amino acid sequence of SEQ ID NO: 305, and iii) the HC-CDR3 comprising the amino acid sequence of SEQ ID NO: 19, and the anti-CD93 V_{L} comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22.

In some embodiments, the anti-CD93 V_{H} comprises i) the HC-CDR1 comprising the amino acid sequence of SEQ ID NO: 304, ii) the HC-CDR2 comprising the amino acid sequence of SEQ ID NO: 305, and iii) the HC-CDR3 comprising the amino acid sequence of SEQ ID NO: 19, and the anti-CD93 V_{L} comprises i) the LC-CDR1 comprising the amino acid sequence of SEQ ID NO: 301, ii) the LC-CDR2 comprising the amino acid sequence of SEQ ID NO: 21, and iii) the LC-CDR3 comprising the amino acid sequence of SEQ ID NO: 22.

In some embodiments, the anti-CD93 V_{H} comprises i) the HC-CDR1 comprising the amino acid sequence of SEQ ID NO: 304, ii) the HC-CDR2 comprising the amino acid sequence of SEQ ID NO: 305, and iii) the HC-CDR3 comprising the amino acid sequence of SEQ ID NO: 19, and the anti-CD93 V_{L} comprises i) the LC-CDR1 comprising the amino acid sequence of SEQ ID NO: 302, ii) the LC-CDR2 comprising the amino acid sequence of SEQ ID NO: 21, and iii) the LC-CDR3 comprising the amino acid sequence of SEQ ID NO: 22.

In some embodiments, the anti-CD93 V_{H} comprises i) the HC-CDR1 comprising the amino acid sequence of SEQ ID NO: 304, ii) the HC-CDR2 comprising the amino acid sequence of SEQ ID NO: 305, and iii) the HC-CDR3 comprising the amino acid sequence of SEQ ID NO: 19, and the anti-CD93 V_{L} comprises i) the LC-CDR1 comprising the amino acid sequence of SEQ ID NO: 303, ii) the LC-CDR2 comprising the amino acid sequence of SEQ ID NO: 21, and iii) the LC-CDR3 comprising the amino acid sequence of SEQ ID NO: 22.

In some embodiments, the anti-CD93 V_{H} comprises i) the HC-CDR1 comprising the amino acid sequence of SEQ ID NO: 304, ii) the HC-CDR2 comprising the amino acid sequence of SEQ ID NO: 305, and iii) the HC-CDR3 comprising the amino acid sequence of SEQ ID NO: 19, and the anti-CD93 V_{L} comprises i) the LC-CDR1 comprising the amino acid sequence of SEQ ID NO: 306, ii) the LC-CDR2 comprising the amino acid sequence of SEQ ID NO: 21, and iii) the LC-CDR3 comprising the amino acid sequence of SEQ ID NO: 22.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NOs: 29, and 307-312; and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NOs: 30, and 313-318.

In some embodiments, the V_{H} comprises an amino acid sequence of any one of SEQ ID NOs: 307-312, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of any one of SEQ ID NOs: 313-318, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 307, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of SEQ ID NO: 313, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 307, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 314, or a variant comprising an amino acid sequence having at least about 80% (such as at least about any one of 80⁰/oy 85%, 90%, 95%, 96%, 97%, 98%, or 99%) sequence identity.

In some embodiments, the V_{H} comprises an amino acid sequence of SEQ ID NO: 307, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 315, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 307, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 316, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 307, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 317, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 307, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 318, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 308, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 313, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 308, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 314, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 308, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 315, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 308, or a variant comprising an amino acid sequence having at least about 80% (such as at least about any one of 80%, 85%, 90%, 95%, 96%, 97%, 98% or 99%) sequence identity, and the V_{L} comprises an amino acid sequence of SEQ ID NO: 316, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 308, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 317, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 308, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 318, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 309, or a variant comprising an amino acid sequence having at least about 80% (such as at least about any one of 80%, 85%, 90%, 95%, 96%, 97%, 98% or 99%) sequence identity, and the V_{L} comprises an amino acid sequence of SEQ ID NO: 313, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 309, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 314, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 309, or a variant comprising an amino acid sequence having at least about 80% (such as at least about any one of 80%, 85%, 90%, 95%, 96%, 97%, 98% or 99%) sequence identity; and the V_{L} comprises an amino acid sequence of SEQ ID NO: 315, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 309, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 316, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 309, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 317, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 309, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 318, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 310, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 313, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 310, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 314, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 310, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 315, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 310, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 316, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 310, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 317, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 310, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 318, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 311, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 313, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 311, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 314, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 311, or a variant comprising an amino acid sequence having at least about 80% (such as at least about any one of 80%, 85%, 90%, 95%, 96%, 97%, 98% or 99%) sequence identity, and the V_{L} comprises an amino acid sequence of SEQ ID NO: 315, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 311, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 316, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 311, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 317, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 311, or a variant comprising an amino acid sequence having at least about 80% (such as at least about any one of 80%, 85%, 90%, 95%, 96%, 97%, 98% or 99%) sequence identity; and the V_{L} comprises an amino acid sequence of SEQ ID NO: 318, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 312, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 313, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 312, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 314, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 312, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 315, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 312, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 316, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 312, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 317, or a variant comprising an amino acid sequence having at least about 80% (such as at least about any one of 80%, 85%, 90%, 95%, 96%, 97%, 98%, or 99%) sequence identity.

In some embodiments, the V_{H} comprises an amino acid sequence of SEQ ID NO: 312, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 318, or a variant comprising an amino acid sequence having at least about 80% (such as at least about any one of 80%, 85%, 90%, 95%, 96%, 97%, 98%, or 99%) sequence identity.

In some embodiments, the antibody moiety comprises a heavy chain variable region (V_{H}) and a light chain variable region (V_{L}), wherein the V_{H} comprises i) the HC-CDR1 comprising the amino acid sequence of SEQ ID NO: 33, ii) the HC-CDR2 comprising the amino acid sequence RIFPGDGDX₁X₂YX₃GKFKG (SEQ ID NO: 233), wherein X₁X₂ are AN or TD, and/or X₃ is N or D, and iii) the HC-CDR3 comprising the amino acid sequence of TGAAYX₁FDPFPY (SEQ ID NO: 234), wherein X₁ is D or E; and the V_{L} comprises i) the LC-CDR1 comprising the amino acid sequence SSX₁KSLLHSX₂GX₃TYLY (SEQ ID NO: 235), wherein X₁ is S or T, X₂ is N or S, and/or X₃ is V or I, ii) the LC-CDR2 comprising the amino acid sequence of SEQ ID NO: 37, and iii) the LC-CDR3 comprising the amino acid sequence of SEQ ID NO: 38.

In some embodiments, the antibody moiety comprises a heavy chain variable region (V_{H}) and a light chain variable region (V_{L}), wherein the V_{H} comprises i) the HC-CDR1 comprising the amino acid sequence X₁YWX₂N (SEQ ID NO: 236), wherein X₁ is S or T, and/or X₂ is L or M, ii) the HC-CDR2 comprising the amino acid sequence RIX₁PGDGDX₂X₃YX₄GKFKG (SEQ ID NO: 237), wherein X₁ is Y or F, X₂X₃ are TD or AN, and/or X₄ is N or D, and iii) the HC-CDR3 comprising the amino acid sequence selected from the group consisting of SEQ ID NOs: 35, 163, and 179; and the V_{L} comprises i) the LC-CDR1 comprising the amino acid sequence of X₁X₂X₃KSLLHSX₄GX₅TYLY (SEQ ID NO: 238), wherein X₁X₂X₃ are SSS, SST, or RFS, X₄ is N or S, and/or X₅═V or I, ii) the LC-CDR2 comprising the amino acid sequence X₁MSNLAS (SEQ ID NO: 239), wherein X₁ is R or Q, and iii) the LC-CDR3 comprising the amino acid sequence AQX₁LEX₂PX₃T (SEQ ID NO: 240), wherein X₁ is M or N, X₂ is R or L, and/or X₃ is F or W. In some embodiments, the LC-CDR3 comprises the amino acid sequence selected from the group consisting of SEQ ID NOS: 38, 166, and 182.

In some embodiments, the antibody moiety comprises a heavy chain variable region (V_{H}) and a light chain variable region (V_{L}), wherein the V_{H} comprises i) the HC-CDR1 comprising the amino acid sequence X₁YVX₂H (SEQ ID NO: 241), wherein X₁ is A or S, and/or X₂ is M or I, ii) the HC-CDR2 comprising the amino acid sequence YIX₁PYX₂DX₃TX₄YNEKFKG (SEQ ID NO: 242), wherein X₁ is F or N, X₂ is N or S, X₃ is G or Y, and/or X₄ is E or Q, and iii) the HC-CDR3 comprising the amino acid sequence RX₁DGNPYX₂MDY (SEQ ID NO: 243), wherein X₁ is T or A, and/or X₂ is T or A; and the V_{L} comprises i) the LC-CDR1 comprising the amino acid sequence of KASQDVSTAVX₁ (SEQ ID NO: 244), wherein X₁ is A or V, ii) the LC-CDR2 comprising the amino acid sequence of SEQ ID NO: 117, and iii) the LC-CDR3 comprising the amino acid sequence of SEQ ID NO: 118. In some embodiments, the LC-CDR3 comprises the amino acid sequence set forth in SEQ ID NO: 115 or 221.

In some embodiments, the antibody moiety comprises a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 5, 4, 3, 2, or 1 amino acid substitutions in the HC-CDRs; and wherein the V_{L} comprises i) the LC-CDR1 comprising the amino acid sequence of KASQX₁VX₂TX₃VX₄(SEQ ID NO: 245), wherein X₁ is N or D, X₂ is G or S, X₃ is N or A, and/or X₄ is A or V, ii) the LC-CDR2 comprising the amino acid sequence of SASYRX₁X₂ (SEQ ID NO: 246), wherein a) X₁ is F or Y, X₂ is I or T, or b) X₁X₂ are FI or YT, and iii) the LC-CDR3 comprising the amino acid sequence QQX₁X₂X₃X₄PX₅T (SEQ ID NO: 247), wherein X₁X₂X₃X₄ are YNRN or HYST, and/or X₅ and I or F. In some embodiments, the LC-CDR3 comprises the amino acid sequence set forth in SEQ ID NO: 6, 118, or 214. In some embodiments, the LC-CDR3 comprises the amino acid sequence set forth in SEQ ID NO: 6.

In some embodiments, the antibody moiety comprises a heavy chain variable region (V_{H}) and a light chain variable region (V_{L}), wherein the V_{H} comprises i) the HC-CDR1 comprising the amino acid sequence of SEQ ID NO: 113, ii) the HC-CDR2 comprising the amino acid sequence of SEQ ID NO: 114, and iii) the HC-CDR3 comprising the amino acid sequence of SEQ ID NO: 115, or a variant thereof comprising up to 5, 4, 3, 2, or 1 amino acid substitutions in the HC-CDRs; and wherein the VI, comprises i) the LC-CDR1 comprising the amino acid sequence of KASQX₁VX₂TX₃VX₄(SEQ ID NO: 245), wherein X₁ is N or D, X₂ is G or S, X₃ is N or A, and/or X₄ is A or V, ii) the LC-CDR2 comprising the amino acid sequence of SASYRX₁X₂ (SEQ ID NO: 246), wherein a) X₁ is F or Y, X₂ is I or T, or b) X₁X₂ are FI or YT, and iii) the LC-CDR3 comprising the amino acid sequence QQX₁X₂X₃X₄PX₅T (SEQ ID NO: 247), wherein X₁X₂X₃X₄ are YNRN or HYST, and/or X₅ and I or F. In some embodiments, the LC-CDR3 comprises the amino acid sequence set forth in SEQ ID NO: 6, 118, or 214. In some embodiments, the LC-CDR3 comprises the amino acid sequence set forth in SEQ ID NO: 118.

In some embodiments, the antibody moiety comprises a heavy chain variable region (V_{H}) and a light chain variable region (V_{L}), wherein the V_{H} comprises i) the HC-CDR1 comprising the amino acid sequence of SEQ ID NO: 209, ii) the HC-CDR2 comprising the amino acid sequence of SEQ ID NO: 210, and iii) the HC-CDR3 comprising the amino acid sequence of SEQ ID NO: 211, or a variant thereof comprising up to 5, 4, 3, 2, or 1 amino acid substitutions in the HC-CDRs; and wherein the V_{L} comprises i) the LC-CDR1 comprising the amino acid sequence of KASQX₁VX₂TX₃VX₄(SEQ ID NO: 245), wherein X₁ is N or D, X₂ is G or S, X₃ is N or A, and/or X₄ is A or V, ii) the LC-CDR2 comprising the amino acid sequence of SASYRX₁X₂ (SEQ ID NO: 246), wherein a) X₁ is F or Y, X₂ is I or T, or b) X₁X₂ are FI or YT, and iii) the LC-CDR3 comprising the amino acid sequence QQX₁X₂X₃X₄PX₅T (SEQ ID NO: 247), wherein X₁X₂X₃X₄ are YNRN or HYST, and/or X₅ and I or F. In some embodiments, the LC-CDR3 comprises the amino acid sequence set forth in SEQ ID NO: 6, 118, or 214. In some embodiments, the LC-CDR3 comprises the amino acid sequence set forth in SEQ ID NO: 214.

In some embodiments, the antibody moiety comprises a heavy chain variable region (V_{H}) and a light chain variable region (V_{L}), wherein the V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, or a variant thereof comprising up to 5, 4, 3, 2, or 1 amino acid substitutions in the HC-CDRs; and wherein the VI, comprises i) the LC-CDR1 comprising the amino acid sequence of X₁ASQSVX₃X₄X₅X₆SYMX₇ (SEQ ID NO: 248), wherein X₁ is K or R, X₂X₃X₄X₅X₆ are DYAGD or STSSY, and/or X₇ is N or H, ii) the LC-CDR2 comprising the amino acid sequence of X₁ASNLES (SEQ ID NO: 249), wherein X₁ is A or Y, and iii) the LC-CDR3 comprising the amino acid sequence QX₁X₂X₃X₄X₅PX₆T (SEQ ID NO: 250), wherein X₁X₂X₃X₄X₅ are QTNED or HSWEI, and/or X₆ is R or F. In some embodiments, the LC-CDR3 comprises the amino acid sequence set forth in SEQ ID NO: 22 or 54. In some embodiments, the LC-CDR3 comprises the amino acid sequence set forth in SEQ ID NO: 22.

In some embodiments, the antibody moiety comprises a heavy chain variable region (V_{H}) and a light chain variable region (V_{L}), wherein the V_{H} comprises i) the HC-CDR1 comprising the amino acid sequence of SEQ ID NO: 49, ii) the HC-CDR2 comprising the amino acid sequence of SEQ ID NO: 50, and iii) the HC-CDR3 comprising the amino acid sequence of SEQ ID NO: 51, or a variant thereof comprising up to 5, 4, 3, 2, or 1 amino acid substitutions in the HC-CDRs; and wherein the VI, comprises i) the LC-CDR1 comprising the amino acid sequence of X₁ASQSVX₃X₄X₅X₆SYMX₇ (SEQ ID NO: 248), wherein X₁ is K or R, X₂X₃X₄X₅X₆ are DYAGD or STSSY, and/or X₇ is N or H, ii) the LC-CDR2 comprising the amino acid sequence of X₁ASNLES (SEQ ID NO: 249), wherein X₁ is A or Y, and iii) the LC-CDR3 comprising the amino acid sequence QX₁X₂X₃X₄X₅PX₆T (SEQ ID NO: 250), wherein X₁X₂X₃X₄X₅ are QTNED or HSWEI, and/or X₆ is R or F. In some embodiments, the LC-CDR3 comprises the amino acid sequence set forth in SEQ ID NO: 22 or 54. In some embodiments, the LC-CDR3 comprises the amino acid sequence set forth in SEQ ID NO: 54.

In some embodiments, the construct comprises or is an antibody or antigen-binding fragment thereof selected from the group consisting of a full-length antibody, a bispecific antibody, a single-chain Fv (scFv) fragment, a Fab fragment, a Fab′ fragment, a F(ab′)2, an Fv fragment, a disulfide stabilized Fv fragment (dsFv), a (dsFv)₂, a V_{H}H, a Fv-Fc fusion, a scFv-Fc fusion, a scFv-Fv fusion, a diabody, a tribody, and a tetrabody.

In some embodiments, the anti-CD93 antibody moiety is a full-length antibody.

In some embodiments, the anti-CD93 antibody moiety is an scFv.

In some embodiments, the anti-CD93 antibody moiety described above comprises an Fc fragment of an immunoglobulin selected from the group consisting of IgG, IgA, IgD, IgE, IgM, and combinations and hybrids thereof. In some embodiments, the anti-CD93 antibody moiety or the full-length antibody described above comprises an Fc fragment of an immunoglobulin selected from the group consisting of IgG1, IgG2, IgG3, IgG4, and combinations and hybrids thereof. In some embodiments, the Fc fragment has a reduced effector function as compared to the corresponding wildtype Fc fragment. In some embodiments, the Fc fragment has an enhanced effector function as compared to the corresponding wildtype Fc fragment. In some embodiments the Fc fragment has been altered for increased serum half-life compared to the corresponding wildtype Fc fragment. In some embodiments the Fc fragment has been altered for decreased serum half-life compared to the corresponding wildtype Fc fragment.

In some embodiments, the antibody moiety comprises a humanized antibody of any of the antibody moiety described herein.

In some embodiments, the anti-CD93 construct comprises or is an anti-CD93 fusion protein.

In some embodiments, the anti-CD93 construct comprises or is a multispecific anti-CD93 construct (such as a bispecific antibody).

In some embodiments, the anti-CD93 construct comprises or is an anti-CD93 immunoconjugate.

In some embodiments, the anti-CD93 construct blocks the binding of CD93 and IGFBP7. In some embodiments, the IGFBP7 is a human IGFBP7. In some embodiments, the binding of CD93 to IGFBP7 is at least blocked by 10%, 20%, 30%, 40%, 50%, 60%, 70%, 80%, 90% or more after a pre-incubation of the anti-CD93 antibody with CD93 or CD93-expressing cells. In some embodiments, the dose of anti-CD93 antibody and CD93 is at a ratio of about 1:10, 1:6, 1:3, 1:1.5, 1:1, 4:3, 2:1, or 5:1. In some embodiments, the binding of CD93 to IGFBP7 is at least blocked by 10%, 20%, 30%, 40%, 50%, 60%, 70%, 80%, 90% or more after a pre-incubation of the anti-CD93 antibody at a concentration of about 50 μg/ml, 25 μg/ml, 10 μg/ml, 5 μg/ml, 2 μg/ml, 1 μg/ml, 0.8 μg/ml, 0.6 μg/ml, or 0.4 μg/ml.

In some embodiments, the anti-CD93 construct blocks the binding of CD93 and MMRN2. In some embodiments, the MMRN2 is a human MMRN2. In some embodiments, the MMRN2 is a MMRN2⁴⁹⁵⁻⁶⁷⁴ fragment. In some embodiments, the binding of CD93 to MMRN2 is at least blocked by 10%, 20%, 30%, 40%, 50%, 60%, 70%, 80%, 90% or more after a pre-incubation of the anti-CD93 antibody with CD93 or CD93-expressing cells. In some embodiments, the anti-CD93 construct does not block the binding of CD93 and MMRN2.

In some embodiments, the anti-CD93 construct blocks the binding of CD93 to both IGFBP7 and MMRN2.

In some embodiments, the anti-CD93 construct does not block the interaction between CD93 and IGFBP7. In some embodiments, the anti-CD93 construct does not block the interaction between CD93 and MMRN2. In some embodiments, the anti-CD93 construct does not block the interaction between either IGFBP7 or MMRN2.

In some embodiments, the CD93 is a human CD93.

a) Antibody Affinity

Binding specificity of the antibody moieties can be determined experimentally by methods known in the art. Such methods comprise, but are not limited to Western blots, ELISA-, RIA-, ECL-, IRMA-, EIA-, BLI, BIACORE™-tests, flow cytometry and peptide scans.

In some embodiments, the K_{D} of the binding between the antibody moiety and CD93 is about 10⁻⁷ M to about 10⁻¹² M, about 10⁻⁷ M to about 10⁻⁸ M, about 10⁻⁸ M to about 10⁻⁹ M, about 10⁻⁹ M to about 10⁻¹⁰ M, about 10⁻¹⁰ M to about 10⁻¹¹ M, about 10⁻¹¹ M to about 10⁻¹² M, about 10⁻⁷ M to about 10⁻¹² M, about 10⁻⁸ M to about 10⁻¹² M, about 10⁻⁹ M to about 10⁻¹² M, about 10⁻¹⁰ M to about 10⁻¹² M, about 10⁻⁷ M to about 10⁻¹¹ M, about 10⁻⁸ M to about 10⁻¹¹ M, about 10⁻⁹ M to about 10⁻¹¹ M, about 10⁻⁷ M to about 10⁻¹⁰ M, about 10⁻⁸ M to about 10⁻¹⁰ M, or about 10⁻⁷ M to about 10⁻⁹ M. In some embodiments, the K_{D} of the binding between the antibody moiety and CD93 is stronger than about any one of 10⁻⁷ M, 10⁻⁸ M, 10⁻⁹ M, 10⁻¹⁰ M, 10⁻¹¹ M, or 10⁻¹² M. In some embodiments, the CD93 is a human CD93.

In some embodiments, the Kₒₙ of the binding between the antibody moiety and CD93 is about 10³ M⁻¹s⁻¹ to about 10⁸ M⁻¹s⁻¹, about 10³M⁻¹s⁻¹ to about 10⁴ M⁻¹s⁻¹, about 10⁴ M⁻¹s⁻¹ to about 10⁵ M⁻¹ s⁻¹, about 10⁵ M⁻¹ s⁻¹ to about 10⁶ M⁻¹ s⁻¹, about 10⁶ M⁻¹ s⁻¹ to about 10⁷ M⁻¹ s⁻¹, or about 10⁷ M⁻¹ s⁻¹ to about 10⁸ M⁻¹ s⁻¹. In some embodiments, the Kₒₙ of the binding between the antibody moiety and CD93 is about 10³ M⁻¹ s⁻¹ to about 10⁵ M⁻¹ s⁻¹, about 10⁴ M⁻¹ s⁻¹ to about 10⁶M⁻¹ s⁻¹, about 10⁵ M⁻¹ s⁻¹ to about 10⁷ M⁻¹ s⁻¹, about 10⁶ M⁻¹ s⁻¹ to about 10⁸M⁻¹ s⁻¹, about 10⁴ M⁻¹ s⁻¹ to about 10⁷ M⁻¹ s⁻¹, or about 10⁵ M⁻¹ s⁻¹ to about 10⁸ M⁻¹ s⁻¹. In some embodiments, the Kₒₙ of the binding between the antibody moiety and CD93 is no more than about any one of 10³ M⁻¹ s⁻¹, 10⁴ M⁻¹ s⁻¹, 10⁵ M⁻¹ s⁻¹, 10⁶ M⁻¹ s⁻¹, 10⁷ M⁻¹ s⁻¹ or 10⁸ M⁻¹ s⁻¹. In some embodiments, CD93 is human CD93.

In some embodiments, the K_{off} of the binding between the antibody moiety and CD93 is about 1 s⁻¹ to about 10⁻⁶ s⁻¹, about 1 s⁻¹ to about 10⁻² s⁻¹, about 10⁻²s⁻¹ to about 10⁻³ s⁻¹, about 10⁻³ s⁻¹ to about 10⁴ s⁻¹, about 10⁴ s⁻¹ to about 10⁻⁵ s⁻¹, about 10⁻⁵ s⁻¹ to about 10⁻⁶ s⁻¹, about 1 s⁻¹ to about 10 s⁻¹, about 10⁻² s⁻¹ to about 10⁻⁶ s⁻¹, about 10 s⁻¹ to about 10⁻⁶ s⁻¹, about 10⁴ s⁻¹ to about 10⁻⁶ s⁻¹, about 10⁻² s⁻¹ to about 10‘ s’¹, or about 10⁻³ s⁻¹ to about 10⁻⁵ s⁻¹. In some embodiments, the K_{off} of the binding between the antibody moiety and CD93 is at least about any one of 1 s⁻¹, 10⁻² s⁻¹, 10⁻³ s⁻¹, 10⁴ s⁻¹, 10⁻⁵ s⁻¹ or 10⁻⁶ s⁻¹. In some embodiments, CD93 is human CD93.

In some embodiments, the binding affinity of the anti-CD93 antibody moiety or anti-CD93 construct are higher (for example, has a smaller K_{D} value) than an existing anti-CD93 antibody (e.g., anti-human CD93 antibody, e.g., MM01).

B) Chimeric or Humanized Antibodies

In some embodiments, the anti-CD93 antibody moiety is a chimeric antibody. Certain chimeric antibodies are described, e.g., in U.S. Pat. No. 4,816,567; and Morrison et al., Proc. Natl. Acad. Sci. USA, 81:6851-6855 (1984)). In some embodiments, a chimeric antibody comprises a non-human variable region (e.g., a variable region derived from mouse) and a human constant region. In some embodiments, a chimeric antibody is a “class switched” antibody in which the class or subclass has been changed from that of the parent antibody. Chimeric antibodies include antigen-binding fragments thereof.

In some embodiments, the anti-CD93 antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); Framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

It is understood that the humanization of mouse derived antibodies is a common and routinely used art. It is therefore understood that a humanized format of any and all of the anti-CD93 antibodies disclosed in Sequence Table can be used in a preclinical or clinical setting. In cases where a humanized format of any of the referenced anti-CD93 antibodies or their antigen-binding regions thereof is used in such a preclinical or clinical setting, the then humanized format is expected to bear the same or similar biological activities and profiles as the original non-humanized format.

c) Human Antibodies

In some embodiments, the anti-CD93 antibody moiety is a human antibody (known as human domain antibody, or human DAb). Human antibodies can be produced using various techniques known in the art. Human antibodies are described generally in van Dijk and van de Winkel, Curr. Opin. Pharmacol. 5: 368-74 (2001), Lonberg, Curr. Opin. Immunol. 20:450-459 (2008), and Chen, Mol. Immunol. 47(4):912-21 (2010). Transgenic mice or rats capable of producing fully human single-domain antibodies (or DAb) are known in the art. See, e.g., US20090307787A1, U.S. Pat. No. 8,754,287, US20150289489A1, US20100122358A1, and WO2004049794.

Human antibodies (e.g., human DAbs) may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies (e.g., human DAbs) can also be made by hybridoma-based methods. Human myeloma and mouse-human heteromyeloma cell lines for the production of human monoclonal antibodies have been described (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies (e.g., human DAbs) may also be generated by isolating Fv clone variable domain sequences selected from human-derived phage display libraries. Such variable domain sequences may then be combined with a desired human constant domain. Techniques for selecting human antibodies from antibody libraries are described below.

d) Library-Derived Antibodies

The anti-CD93 antibody moieties described herein may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N J, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N J, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004). Methods for constructing single-domain antibody libraries have been described, for example, See U.S. Pat. No. 7,371,849.

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically displays antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

e) Substitution, Insertion, Deletion and Variants

In some embodiments, antibody variants having one or more amino acid substitutions are provided. Sites of interest for substitutional mutagenesis include the HVRs (or CDRs) and FRs. Conservative substitutions are shown in Table 2 under the heading of “Preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2
Amino acid substitutions

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-1% (2008)), and/or SDRs (a-CDRs), with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity or molecular behavior. Another method to introduce diversity involves HVR-directed approaches, in which several HVR residues (e.g., 4-6 residues at a time) are randomized. HVR residues involved in antigen binding may be specifically identified, e.g., using alanine or histidine scanning mutagenesis or modeling. HC-CDR3 and LC-CDR3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or CDRs.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties for the antibody.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

f) Glycosylation Variants

In some embodiments, the anti-CD93 antibody moiety is altered to increase or decrease the extent to which the construct is glycosylated. Addition or deletion of glycosylation sites to an antibody may be conveniently accomplished by altering the amino acid sequence such that one or more glycosylation sites is created or removed.

Where the antibody moiety comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (G1cNAc), galactose, and sialic acid, as well as a fucose attached to a G1cNAc in the “stem” of the biantennary oligosaccharide structure. In some embodiments, modifications of the oligosaccharide in the antibody moiety may be made in order to create antibody variants with certain improved properties.

In some embodiments, the anti-CD93 antibody moiety has a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L); US 2004/0093621 (Kyowa Hakk) Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In some embodiments, the anti-CD93 antibody moiety has bisected oligosaccharides, e.g., in which a biantennary oligosaccharide attached to the Fc region of the antibody is bisected by G1cNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

g) Fc Region Variants

In some embodiments, the anti-CD93 antibody moiety comprises an Fc fragment.

The term “Fc region,” “Fc domain,” “Fc fragment” or “Fc” refers to a C-terminal non-antigen binding region of an immunoglobulin heavy chain that contains at least a portion of the constant region. The term includes native Fc regions and variant Fc regions. In some embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. Unless otherwise specified herein, numbering of amino acid residues in the IgG or Fc region is according to the EU numbering system for antibodies, also called the EU index, as described in Kabat et al., Sequences of Proteins of Immunological Interest, 5th Ed. Public Health Service, National Institutes of Health, Bethesda, M D, 1991.

In some embodiments, the Fc fragment is from an immunoglobulin selected from the group consisting of IgG, IgA, IgD, IgE, IgM, and combinations and hybrids thereof. In some embodiments, the Fc fragment is from an immunoglobulin selected from the group consisting of IgG1, IgG2, IgG3, IgG4, and combinations and hybrids thereof.

In some embodiments, the Fc fragment has a reduced effector function as compared to corresponding wildtype Fc fragment (such as at least about 30%, 40%, 50%, 60%, 70%, 80%, 85%, 90%, or 95% reduced effector function as measured by the level of antibody-dependent cellular cytotoxicity (ADCC)).

In some embodiments, the Fc fragment is an IgG1 Fc fragment. In some embodiments, the IgG1 Fc fragment comprises a L234A mutation and/or a L235A mutation. In some embodiments, the Fc fragment is an IgG2 or IgG4 Fc fragment. In some embodiments, the Fc fragment is an IgG4 Fc fragment comprising a S228P, F234A, and/or a L235A mutation. In some embodiments, the Fc fragment comprises a N297A mutation. In some embodiments, the Fc fragment comprises a N297G mutation.

In some embodiments, one or more amino acid modifications may be introduced into the Fc region of the antibody moiety, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the Fc fragment possesses some but not all effector functions, which make it a desirable candidate for applications in which the half-life of the antibody moiety in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (See Bruggemann, M. et al., J. Exp. Med. 166:1351-1361 (1987)). Alternatively, non-radioactive assays methods may be employed (see, for example, ACTIT™ non-radioactive cytotoxicity assay for flow cytometry (Cell Technology, Inc. Mountain View, CA; and CytoTox 96® non radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Intl. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). In some embodiments, the Fc fragment comprises a N297A mutation. In some embodiments, the Fc fragment comprises a N297G mutation.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, the Fc fragment is an IgG1 Fc fragment. In some embodiments, the IgG1 Fc fragment comprises a L234A mutation and/or a L235A mutation. In some embodiments, the IgG1 Fc fragment comprises a L235A mutation and/or a G237A mutation. In some embodiments, the Fc fragment is an IgG2 or IgG4 Fc fragment. In some embodiments, the Fc fragment is an IgG4 Fc fragment comprising a S228P, F234A, and/or a L235A mutation.

In some embodiments, the antibody moiety comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In some embodiments, the antibody moiety variant comprising a variant Fc region comprising one or more amino acid substitutions which alters half-life and/or changes binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which alters binding of the Fc region to FcRn. Such Fc variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

h) Cysteine Engineered Antibody Variants

In some embodiments, it may be desirable to create cysteine engineered antibody moieties, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In some embodiments, any one or more of the following residues may be substituted with cysteine: A118 (EU numbering) of the heavy chain; and S400 (EU numbering) of the heavy chain Fc region. Cysteine engineered antibody moieties may be generated as described, e.g., in U.S. Pat. No. 7,521,541.

i) Antibody Derivatives

In some embodiments, the antibody moiety described herein may be further modified to comprise additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in diagnosis under defined conditions, etc.

In some embodiments, the antibody moiety may be further modified to comprise one or more biologically active protein, polypeptides or fragments thereof. “Bioactive” or “biologically active”, as used herein interchangeably, means showing biological activity in the body to carry out a specific function. For example, it may mean the combination with a particular biomolecule such as protein, DNA, etc., and then promotion or inhibition of the activity of such biomolecule. In some embodiments, the bioactive protein or fragments thereof include proteins and polypeptides that are administered to patients as the active drug substance for prevention of or treatment of a disease or condition, as well as proteins and polypeptides that are used for diagnostic purposes, such as enzymes used in diagnostic tests or in vitro assays, as well as proteins and polypeptides that are administered to a patient to prevent a disease such as a vaccine.

### Multispecific Anti-CD93 Constructs

The anti-CD93 constructs in some embodiments comprise a multispecific (e.g., bispecific) anti-CD93 construct comprising an anti-CD93 antibody moiety according to any one of the anti-CD93 antibody moieties described herein, and a second binding moiety (such as a second antibody moiety) specifically recognizing a second antigen.

In some embodiments, the multispecific anti-CD93 molecule comprises an anti-CD93 antibody moiety and a second moiety (such as a second antibody moiety) specifically recognizing a second antigen.

In some embodiments, the second antigen is an immune checkpoint molecule. In some embodiments, the second antigen is PD-1 or PD-L1.

In some embodiment, the second moiety is an extracellular domain (ECD) of PD-1 or PD-L1. In some embodiments, the second moiety is a PD-L1 trap or PD-1 trap. See e.g., Nat Commun. 2018 Jun. 8; 9(1):2237.

In some embodiments, the second antigen is a tumor antigen.

In some embodiments, the second antigen is an angiogenic agent. In some embodiments, the angiogenic agent is a VEGF (e.g., a human VEGF) antibody. In some embodiments, the angiogenic agent is a VEGF receptor. In some embodiments, the angiogenic agent is a VEGFR1 (e.g., a human VEGFR1). In some embodiments, the angiogenic agent is a VEGFR2 (e.g., a human VEGFR2).

In some embodiments, the second moiety comprises an extracellular domain (ECD) of a VEGF receptor. In some embodiments, the second moiety comprises an ECD of VEGFR1 and/or VEGFR2. In some embodiments, the second moiety comprises a VEGF-trap. See e.g., Proc Natl Acad Sci USA. 2002 Aug. 20; 99(17):11393-8.

In some embodiments, the second antibody moiety and the anti-CD93 antibody moiety are fused with each other via a linker such as any of the linkers described herein with any operable form that allows the proper function of the binding moieties. In some embodiments, the linker is a GS linker. In some embodiments, the linker is selected from the group consisting of SEQ ID NOs: 225-232 and 338.

In some embodiments, the anti-CD93 construct is a multispecific (e.g., bispecific) anti-CD93 construct comprising a) an anti-CD93 antibody moiety according to any one of the anti-CD93 antibody moieties described herein; b) a second antibody moiety specifically recognizing PD-L1 (an anti-PD-L1 antibody moiety).

In some embodiments, the anti-CD93 construct is a multispecific (e.g., bispecific) anti-CD93 construct comprising a) an anti-CD93 full-length antibody comprising two heavy chains and two light chains, wherein the two heavy chains each comprises a heavy chain variable region (V_{H}) and the two light chains each comprises a light chain variable region (V_{L}), b) an anti-PD-L1 antibody moiety (such as any of the antibody moiety described herein) fused to at least one or both of the heavy chains of the anti-CD93 full-length antibody. In some embodiments, the anti-PD-L1 antibody moiety is fused to N-terminus of both heavy chains. In some embodiments, the anti-PD-L1 antibody moiety is fused to C-terminus of both heavy chains.

In some embodiments, the anti-CD93 construct is a multispecific (e.g., bispecific) anti-CD93 construct comprising a) an anti-PD-L1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-CD93 antibody moiety (such as any of the anti-CD93 antibody moiety described herein) fused to at least one or both of the heavy chains of the anti-PD-L1 full-length antibody. In some embodiments, the anti-CD93 antibody moiety is fused to N-terminus of both heavy chains. In some embodiments, the anti-CD93 antibody moiety is fused to C-terminus of both heavy chains.

In some embodiments, the anti-CD93 construct is a multispecific (e.g., bispecific) anti-CD93 construct comprising a) an anti-CD93 full-length antibody comprising two heavy chains and two light chains, wherein the two heavy chains each comprises a heavy chain variable region (V_{H}) and the two light chains each comprises a light chain variable region (V_{L}), b) an anti-PD-L1 antibody moiety (such as any of the antibody moiety described herein) fused to at least one or both of the light chains of the anti-CD93 full-length antibody. In some embodiments, the anti-PD-L1 antibody moiety is fused to N-terminus of both light chains. In some embodiments, the anti-PD-L1 antibody moiety is fused to C-terminus of both light chains.

In some embodiments, the anti-CD93 construct is a multispecific (e.g., bispecific) anti-CD93 construct comprising a) an anti-PD-L1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-CD93 antibody moiety (such as any of the antibody moiety described herein) fused to at least one or both of the light chains of the anti-PD-L1 full-length antibody. In some embodiments, the anti-CD93 antibody moiety is fused to N-terminus of both light chains. In some embodiments, the anti-CD93 antibody moiety is fused to C-terminus of both light chains.

In some embodiments, the anti-CD93 construct is a multispecific (e.g., bispecific) anti-CD93 construct comprising a) an anti-CD93 antibody moiety according to any one of the anti-CD93 antibody moieties described herein; b) a second antibody moiety specifically recognizing PD-1 (an anti-PD-1 antibody moiety).

In some embodiments, the anti-CD93 construct is a multispecific (e.g., bispecific) anti-CD93 construct comprising a) an anti-CD93 full-length antibody comprising two heavy chains and two light chains, wherein the two heavy chains each comprises a heavy chain variable region (V_{H}) and the two light chains each comprises a light chain variable region (V_{L}), b) an anti-PD-1 antibody moiety (such as any of the antibody moiety described herein) fused to at least one or both of the heavy chains of the anti-CD93 full-length antibody. In some embodiments, the anti-PD-antibody moiety is fused to N-terminus of both heavy chains. In some embodiments, the anti-PD-1 antibody moiety is fused to C-terminus of both heavy chains.

In some embodiments, the anti-CD93 construct is a multispecific (e.g., bispecific) anti-CD93 construct comprising a) an anti-PD-1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-CD93 antibody moiety (such as any of the anti-CD93 antibody moiety described herein) fused to at least one or both of the heavy chains of the anti-PD-1 full-length antibody. In some embodiments, the anti-CD93 antibody moiety is fused to N-terminus of both heavy chains. In some embodiments, the anti-CD93 antibody moiety is fused to C-terminus of both heavy chains.

In some embodiments, the anti-CD93 construct is a multispecific (e.g., bispecific) anti-CD93 construct comprising a) an anti-CD93 full-length antibody comprising two heavy chains and two light chains, wherein the two heavy chains each comprises a heavy chain variable region (V_{H}) and the two light chains each comprises a light chain variable region (V_{L}), b) an anti-PD-1 antibody moiety (such as any of the antibody moiety described herein) fused to at least one or both of the light chains of the anti-CD93 full-length antibody. In some embodiments, the anti-PD-1 antibody moiety is fused to N-terminus of both light chains. In some embodiments, the anti-PD-1 antibody moiety is fused to C-terminus of both light chains.

In some embodiments, the anti-CD93 construct is a multispecific (e.g., bispecific) anti-CD93 construct comprising a) an anti-PD-1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-CD93 antibody moiety (such as any of the antibody moiety described herein) fused to at least one or both of the light chains of the anti-PD-1 full-length antibody. In some embodiments, the anti-CD93 antibody moiety is fused to N-terminus of both light chains. In some embodiments, the anti-CD93 antibody moiety is fused to C-terminus of both light chains.

In some embodiments, the anti-CD93 construct is a multispecific (e.g., bispecific) anti-CD93 construct comprising a) an anti-CD93 antibody moiety according to any one of the anti-CD93 antibody moieties described herein; b) a second binding moiety specifically recognizing VEGF.

In some embodiments, the anti-CD93 construct is a multispecific (e.g., bispecific) anti-CD93 construct comprising a) an anti-CD93 full-length antibody comprising two heavy chains and two light chains, wherein the two heavy chains each comprises a heavy chain variable region (V_{H}) and the two light chains each comprises a light chain variable region (V_{L}), b) a second binding moiety specifically recognizing VEGF fused to at least one or both of the heavy chains of the anti-CD93 full-length antibody. In some embodiments, the second binding moiety is fused to N-terminus of both heavy chains. In some embodiments, the second binding moiety is fused to C-terminus of both heavy chains.

In some embodiments, the anti-CD93 construct is a multispecific (e.g., bispecific) anti-CD93 construct comprising a) an anti-VEGF antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-CD93 antibody moiety (such as any of the anti-CD93 antibody moiety described herein) fused to at least one or both of the heavy chains of the anti-VEGF full-length antibody. In some embodiments, the anti-CD93 antibody moiety is fused to N-terminus of both heavy chains. In some embodiments, the anti-CD93 antibody moiety is fused to C-terminus of both heavy chains.

In some embodiments, the anti-CD93 construct is a multispecific (e.g., bispecific) anti-CD93 construct comprising a) an anti-CD93 full-length antibody comprising two heavy chains and two light chains, wherein the two heavy chains each comprises a heavy chain variable region (V_{H}) and the two light chains each comprises a light chain variable region (V_{L}), b) a second binding moiety specifically recognizing VEGF fused to at least one or both of the light chains of the anti-CD93 full-length antibody. In some embodiments, the second binding moiety is fused to N-terminus of both light chains. In some embodiments, a second binding moiety specifically recognizing VEGF is fused to C-terminus of both light chains.

In some embodiments, the anti-CD93 construct is a multispecific (e.g., bispecific) anti-CD93 construct comprising a) an anti-VEGF antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-CD93 antibody moiety (such as any of the antibody moiety described herein) fused to at least one or both of the light chains of the anti-VEGF full-length antibody. In some embodiments, the anti-CD93 antibody moiety is fused to N-terminus of both light chains. In some embodiments, the anti-CD93 antibody moiety is fused to C-terminus of both light chains.

In some embodiments, there is provided an anti-CD93 construct comprising a) a full-length antibody that specifically recognizes CD93 comprising two heavy chains and two light chains, wherein the two heavy chains each comprises a heavy chain variable region (V_{H}) comprising the HC-CDR1 comprising the amino acid sequence of SEQ ID NO: 289, the HC-CDR2 comprising the amino acid sequence of SEQ ID NO: 290, and the HC-CDR3 comprising the amino acid sequence of SEQ ID NO: 291, and wherein the two light chains each comprises a light chain variable region (V_{L}) comprises the LC-CDR1 comprising the amino acid sequence of SEQ ID NO: 292, the LC-CDR2 comprising the amino acid sequence of SEQ ID NO: 293, and the LC-CDR3 comprising the amino acid sequence of SEQ ID NO: 294, and b) a VEGF binding moiety comprising the amino acid sequence of SEQ ID NO: 325, wherein the VEGF binding moiety is fused to one or both of the heavy chains of the full-length antibody. In some embodiments, the VEGF binding moiety is fused to C-terminus of both heavy chains of the full-length antibody. In some embodiments, the VEGF binding moiety is fused to the full-length antibody without a linker. In some embodiments, the VEGF binding moiety is fused to the full-length antibody via a linker. In some embodiments, the linker is GS linker or selected from the group consisting of SEQ ID NOs: 225-232 and 338. In some embodiments, the linker comprises the amino acid sequence of SEQ ID NO: 338. In some embodiments, the anti-CD93 V_{H} comprises the amino acid sequence of any one of SEQ ID NOs: 287, and 319-321, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of any one of SEQ ID NOs: 288, and 322-324, or a variant comprising an amino acid sequence having at least about 80% (such as at least about any one of 80%, 85%, 90%, 95%, 96%, 97%, 98%, or 99%) sequence identity. In some embodiments, the full-length antibody has an IgG1 isotype (such as a human IgG1 isotype). In some embodiments, the heavy chain comprises the amino acid sequence of SEQ ID NO: 342, or a variant comprising an amino acid sequence having at least about 80% (such as at least about any one of 80%, 85%, 90%, 95%, 96%, 97%, 98%, or 99%) sequence identity. In some embodiments, the light chain comprises the amino acid sequence of SEQ ID NO: 343, or a variant comprising an amino acid sequence having at least about 80% (such as at least about any one of 80%, 85%, 90%, 95%, 96%, 97%, 98%, or 99%) sequence identity. In some embodiments, there is provided an anti-CD93 construct comprising a heavy chain fusion polypeptide comprising the amino acid sequence set forth in SEQ ID NO: 366, or a variant comprising an amino acid sequence having at least about 80% (such as at least about any one of 80%, 85%, 90%, 95%, 96%, 97%, 98%, or 99%) sequence identity, and a light chain comprising an amino acid sequence set forth in SEQ ID NO: 367, or a variant comprising an amino acid sequence having at least about 80% (such as at least about any one of 80%, 85%, 90%, 95%, 96%, 97%, 98%, or 99%) sequence identity.

In some embodiments, the anti-CD93 V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 5, 4, 3, 2, or 1 amino acid substitutions in the HC-CDRs, and the anti-CD93 V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 5, 4, 3, 2, or 1 amino acid substitutions in the LC-CDRs.

In some embodiments, the anti-CD93 V_{H} comprises i) the HC-CDR1 comprising the amino acid sequence of SEQ ID NO: 17 or 304, ii) the HC-CDR2 comprising the amino acid sequence of SEQ ID NO: 18 or 305, and iii) the HC-CDR3 comprising the amino acid sequence of SEQ ID NO: 19, or a variant thereof comprising up to 5, 4, 3, 2, or 1 amino acid substitutions in the HC-CDRs, and the anti-CD93 V_{L} comprises i) the LC-CDR1 comprising the amino acid sequence of SEQ ID NO: 20, 301, 302, 303, or 306, ii) the LC-CDR2 comprising the amino acid sequence of SEQ ID NO: 21, and iii) the LC-CDR3 comprising the amino acid sequence of SEQ ID NO: 22, or a variant thereof comprising up to 5, 4, 3, 2, or 1 amino acid substitutions in the LC-CDRs.

In some embodiments, the anti-CD93 V_{H} comprises i) the HC-CDR1 comprising the amino acid sequence of SEQ ID NO: 289, ii) the HC-CDR2 comprising the amino acid sequence of SEQ ID NO: 290, and iii) the HC-CDR3 comprising the amino acid sequence of SEQ ID NO: 291, or a variant thereof comprising up to 5, 4, 3, 2, or 1 amino acid substitutions in the HC-CDRs, and the anti-CD93 V_{L} comprises i) the LC-CDR1 comprising the amino acid sequence of SEQ ID NO: 292, ii) the LC-CDR2 comprising the amino acid sequence of SEQ ID NO: 293, and iii) the LC-CDR3 comprising the amino acid sequence of SEQ ID NO: 294, or a variant thereof comprising up to 5, 4, 3, 2, or 1 amino acid substitutions in the LC-CDRs. In some embodiments, the anti-CD93 V_{H} comprises i) the HC-CDR1 comprising the amino acid sequence of SEQ ID NO: 295, ii) the HC-CDR2 comprising the amino acid sequence of SEQ ID NO: 296, and iii) the HC-CDR3 comprising the amino acid sequence of SEQ ID NO: 297, or a variant thereof comprising up to 5, 4, 3, 2, or 1 amino acid substitutions in the HC-CDRs, and the anti-CD93 V_{L} comprises i) the LC-CDR1 comprising the amino acid sequence of SEQ ID NO: 298, ii) the LC-CDR2 comprising the amino acid sequence of SEQ ID NO: 299, and iii) the LC-CDR3 comprising the amino acid sequence of SEQ ID NO: 300, or a variant thereof comprising up to 5, 4, 3, 2, or 1 amino acid substitutions in the LC-CDRs.

In some embodiments, the amino acid substitutions described above are limited to “exemplary substitutions” shown in Table 2 of this application. In some embodiments, the amino acid substitutions are limited to “preferred substitutions” shown in Table 2 of this application.

### Exemplary Anti-PD-L1 Antibody Moieties

Exemplary anti-PD-L1 antibody moieties include, but not are limited to those described in WO2019228514A1, WO2019227490A1 and WO2020019232A1.

In some embodiments, the anti-PD-L1 antibody moiety (such as an scFv) used in multispecific anti-CD93 constructs comprises an antibody moiety comprising a heavy chain variable region (V_{H}) and a light chain variable region (V_{L}), wherein the antibody moiety competes for a binding epitope of PD-L1 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 251, the HC-CDR2 comprising the amino acid sequence of SEQ ID NO: 252, and the HC-CDR3 comprising the amino acid sequence of SEQ ID NO: 253, and the V_{L-2} comprises the LC-CDR1 comprising the amino acid sequence of SEQ ID NO: 254, the LC-CDR2 comprising the amino acid sequence of SEQ ID NO: 255, and the LC-CDR3 comprising the amino acid sequence of SEQ ID NO: 256.

In some embodiments, the anti-PD-L1 moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 281, 282, or 283; and a LC-CDR1, a LC-CDR2, and a LC-CDR3, respectively comprising the amino acid sequences of a CDR1, a CDR2, and a CDR3 within a VI, chain region having the sequence set forth in SEQ ID NO: 284, 285, or 286.

In some embodiments, the anti-PD-L1 antibody moiety (such as an scFv) used in multispecific anti-CD93 constructs comprises a heavy chain variable region (V_{H}) and a light chain variable region (V_{L}), wherein: a) the V_{H} comprises an HC-CDR1 comprising the amino acid sequence of SEQ ID NO: 251, an HC-CDR2 comprising the amino acid sequence of SEQ ID NO: 252, and an HC-CDR3 comprising the amino acid sequence of SEQ ID NO: 253, or a variant thereof comprising up to a total of about 5, 4, 3, 2, or 1 amino acid substitutions in the HC-CDRs; and b) the V_{L} comprises an LC-CDR1 comprising the amino acid sequence of SEQ ID NO: 254, an LC-CDR2 comprising the amino acid sequence of any one of SEQ ID NO: 255, and an LC-CDR3 comprising the amino acid sequence of any one of SEQ ID NO: 256, or a variant thereof comprising up to a total of about 5, 4, 3, 2, or 1 amino acid substitutions in the LC-CDRs.

In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the V_{H} comprises an amino acid sequence of SEQ ID NO: 281, 282, or 283, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of SEQ ID NO: 284, 285 or 286, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 281, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 284, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 282, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 285, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 283, or a variant comprising an amino acid sequence having at least about 80% (such as at least about any one of 80%, 85%, 90%, 95%, 96%, 97%, 98%, or 99%) sequence identity; and the V_{L}. comprises an amino acid sequence of SEQ ID NO: 286, or a variant comprising an amino acid sequence having at least about 80% (such as at least about any one of 80%, 85%, 90%, 95%, 96%, 97%, 98%, or 99%) sequence identity.

In some embodiments, the second antibody moiety and the anti-CD93 antibody moiety are fused with each other via a linker such as any of the linkers described herein with any operable form that allows the proper function of the binding moieties.

### Exemplary Anti-PD-1 Antibody Moieties

Exemplary anti-PD-1 antibody moieties include, but not are limited to those described in WO2018133842 and WO2018133837.

In some embodiments, the anti-PD-1 antibody moiety (such as an scFv) used in multispecific anti-CD93 constructs comprises an antibody moiety comprising a heavy chain variable region (V_{H}) and a light chain variable region (V_{L}), wherein the antibody moiety competes for a binding epitope of PD-1 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 257, the HC-CDR2 comprising the amino acid sequence of SEQ ID NO: 258, and the HC-CDR3 comprising the amino acid sequence of SEQ ID NO: 259, and the V_{L-2} comprises the LC-CDR1 comprising the amino acid sequence of SEQ ID NO: 260, the LC-CDR2 comprising the amino acid sequence of SEQ ID NO: 261, and the LC-CDR3 comprising the amino acid sequence of SEQ ID NO: 262.

In some embodiments, the anti-PD-1 moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 275; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 276.

In some embodiments, the anti-PD-1 antibody moiety (such as an scFv) used in multispecific anti-CD93 constructs comprises a heavy chain variable region (V_{H}) and a light chain variable region (V_{L}), wherein: a) the V_{H} comprises an HC-CDR1 comprising the amino acid sequence of SEQ ID NO: 257, an HC-CDR2 comprising the amino acid sequence of SEQ ID NO: 258, and an HC-CDR3 comprising the amino acid sequence of SEQ ID NO: 259, or a variant thereof comprising up to a total of about 5, 4, 3, 2, or 1 amino acid substitutions in the HC-CDRs; and b) the V_{L} comprises an LC-CDR1 comprising the amino acid sequence of SEQ ID NO: 260, an LC-CDR2 comprising the amino acid sequence of any one of SEQ ID NO: 261, and an LC-CDR3 comprising the amino acid sequence of any one of SEQ ID NO: 262, or a variant thereof comprising up to a total of about 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the second antibody moiety comprises a humanized antibody moiety derived from a murine antibody comprising a heavy chain variable region (V_{H}) comprising the amino acid sequence set forth in SEQ ID NO: 275 and a light chain variable region (V_{L}) comprising the amino acid sequence forth in SEQ ID NO: 276.

In some embodiments, the anti-PD-1 antibody moiety (such as an scFv) used in multispecific anti-CD93 constructs comprises an antibody moiety comprising a heavy chain variable region (V_{H}) and a light chain variable region (V_{L}), wherein the antibody moiety competes for a binding epitope of PD-1 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 263, the HC-CDR2 comprising the amino acid sequence of SEQ ID NO: 264, and the HC-CDR3 comprising the amino acid sequence of SEQ ID NO: 265, and the V_{L-2} comprises the LC-CDR1 comprising the amino acid sequence of SEQ ID NO: 266, the LC-CDR2 comprising the amino acid sequence of SEQ ID NO: 267, and the LC-CDR3 comprising the amino acid sequence of SEQ ID NO: 268.

In some embodiments, the anti-PD-1 moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 277; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 278.

In some embodiments, the anti-PD-1 antibody moiety (such as an scFv) used in multispecific anti-CD93 constructs comprises a heavy chain variable region (V_{H}) and a light chain variable region (V_{L}), wherein: a) the V_{H} comprises an HC-CDR1 comprising the amino acid sequence of SEQ ID NO: 263, an HC-CDR2 comprising the amino acid sequence of SEQ ID NO: 264, and an HC-CDR3 comprising the amino acid sequence of SEQ ID NO: 265, or a variant thereof comprising up to a total of about 5, 4, 3, 2, or 1 amino acid substitutions in the HC-CDRs; and b) the V_{L} comprises an LC-CDR1 comprising the amino acid sequence of SEQ ID NO: 266, an LC-CDR2 comprising the amino acid sequence of any one of SEQ ID NO: 267, and an LC-CDR3 comprising the amino acid sequence of any one of SEQ ID NO: 268, or a variant thereof comprising up to a total of about 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-PD-1 antibody moiety comprises a humanized antibody moiety derived from a murine antibody comprising a heavy chain variable region (V_{H}) comprising the amino acid sequence set forth in SEQ ID NO: 277 and a light chain variable region (V_{L}) comprising the amino acid sequence forth in SEQ ID NO: 278.

In some embodiments, the anti-PD-1 antibody moiety (such as an scFv) used in multispecific anti-CD93 constructs comprises an antibody moiety comprising a heavy chain variable region (V_{H}) and a light chain variable region (V_{L}), wherein the antibody moiety competes for a binding epitope of PD-1 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 269, the HC-CDR2 comprising the amino acid sequence of SEQ ID NO: 270, and the HC-CDR3 comprising the amino acid sequence of SEQ ID NO: 271, and the V_{L-2} comprises the LC-CDR1 comprising the amino acid sequence of SEQ ID NO: 272, the LC-CDR2 comprising the amino acid sequence of SEQ ID NO: 273, and the LC-CDR3 comprising the amino acid sequence of SEQ ID NO: 274.

In some embodiments, the anti-PD-1 moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 279; and a LC-CDR1, a LC-CDR2, and a LC-CDR3, respectively comprising the amino acid sequences of a CDR1, a CDR2, and a CDR3 within a VI, chain region having the sequence set forth in SEQ ID NO: 280.

In some embodiments, the anti-PD-1 antibody moiety (such as an scFv) used in multispecific anti-CD93 constructs comprises a heavy chain variable region (V_{H}) and a light chain variable region (V_{L}), wherein: a) the V_{H} comprises an HC-CDR1 comprising the amino acid sequence of SEQ ID NO: 269, an HC-CDR2 comprising the amino acid sequence of SEQ ID NO: 270, and an HC-CDR3 comprising the amino acid sequence of SEQ ID NO: 271, or a variant thereof comprising up to a total of about 5, 4, 3, 2, or 1 amino acid substitutions in the HC-CDRs; and b) the V_{L} comprises an LC-CDR1 comprising the amino acid sequence of SEQ ID NO: 272, an LC-CDR2 comprising the amino acid sequence of any one of SEQ ID NO: 273, and an LC-CDR3 comprising the amino acid sequence of any one of SEQ ID NO: 274, or a variant thereof comprising up to a total of about 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the second antibody moiety comprises a humanized antibody moiety derived from a murine antibody comprising a heavy chain variable region (V_{H}) comprising the amino acid sequence set forth in SEQ ID NO: 279 and a light chain variable region (V_{L}) comprising the amino acid sequence forth in SEQ ID NO: 280.

In some embodiments, the second antibody moiety and the anti-CD93 antibody moiety are fused with each other via a linker such as any of the linkers described herein with any operable form that allows the proper function of the binding moieties.

### Exemplary Binding Moieties Specifically Recognizing VEGF

Exemplary binding moieties specifically recognizing VEGF include, but not are limited to avastin, ramucirumab, or VEGF-trap (Aflibercept), or a variant or a functional portion thereof.

In some embodiments, the binding moiety that specifically recognizes VEGF used in multispecific anti-CD93 constructs is an antibody moiety (such as an scFv) comprising an antibody moiety comprising a heavy chain variable region (V_{H}) and a light chain variable region (V_{L}), wherein the V_{H} comprises the HC-CDR1 comprising the amino acid sequence of SEQ ID NO: 326, the HC-CDR2 comprising the amino acid sequence of SEQ ID NO: 327, and the HC-CDR3 comprising the amino acid sequence of SEQ ID NO: 328, and the V_{L} comprises the LC-CDR1 comprising the amino acid sequence of SEQ ID NO: 329, the LC-CDR2 comprising the amino acid sequence of SEQ ID NO: 330, and the LC-CDR3 comprising the amino acid sequence of SEQ ID NO: 331.

In some embodiments, the binding moiety that specifically recognizes VEGF used in multispecific anti-CD93 constructs is an antibody moiety (such as an scFv) comprising an antibody moiety comprising a heavy chain variable region (V_{H}) and a light chain variable region (V_{L}), wherein the V_{H} comprises the HC-CDR1 comprising the amino acid sequence of SEQ ID NO: 332, the HC-CDR2 comprising the amino acid sequence of SEQ ID NO: 333, and the HC-CDR3 comprising the amino acid sequence of SEQ ID NO: 334, and the V_{L} comprises the LC-CDR1 comprising the amino acid sequence of SEQ ID NO: 335, the LC-CDR2 comprising the amino acid sequence of SEQ ID NO: 336, and the LC-CDR3 comprising the amino acid sequence of SEQ ID NO: 337.

In some embodiments, the binding moiety that specifically recognizes VEGF used in multispecific anti-CD93 constructs is an antibody moiety (such as an scFv) comprising an antibody moiety comprising a heavy chain variable region (V_{H}) and a light chain variable region (V_{L}), wherein the V_{H} comprises the HC-CDR1 comprising the amino acid sequence of SEQ ID NO: 289, the HC-CDR2 comprising the amino acid sequence of SEQ ID NO: 290, and the HC-CDR3 comprising the amino acid sequence of SEQ ID NO: 291, and the V_{L} comprises the LC-CDR1 comprising the amino acid sequence of SEQ ID NO: 292, the LC-CDR2 comprising the amino acid sequence of SEQ ID NO: 293, and the LC-CDR3 comprising the amino acid sequence of SEQ ID NO: 294.

In some embodiments, the binding moiety that specifically recognizes VEGF used in multispecific anti-CD93 constructs comprises the amino acid sequence of SEQ ID NO: 325.

In some embodiments, the anti-CD93 construct is a multispecific (e.g., bispecific) anti-CD93 construct comprising a) an anti-CD93 full-length antibody comprising two heavy chains and two light chains, wherein the two heavy chains each comprises a heavy chain variable region (V_{H}) and the two light chains each comprises a light chain variable region (V_{L}), wherein the V_{H} comprises the HC-CDR1 comprising the amino acid sequence of SEQ ID NO: 289, the HC-CDR2 comprising the amino acid sequence of SEQ ID NO: 290, and the HC-CDR3 comprising the amino acid sequence of SEQ ID NO: 291, and the V_{L} comprises the LC-CDR 1 comprising the amino acid sequence of SEQ ID NO: 292, the LC-CDR2 comprising the amino acid sequence of SEQ ID NO: 293, and the LC-CDR3 comprising the amino acid sequence of SEQ ID NO: 294, and b) a binding moiety that specifically recognizes VEGF fused to the C-terminus of the two heavy chains of the anti-CD93 full-length antibody. In some embodiments, the binding moiety that specifically recognizes VEGF used in multispecific anti-CD93 constructs comprises the amino acid sequence of SEQ ID NO: 325, or a variant comprising an amino acid sequence having at least about 80% (such as at least about any one of 80%, 85%, 90%, 95%, 96%, 97%, 98%, or 99%) sequence identity. In some embodiments, there is provided an anti-CD93 construct comprising a heavy chain fusion polypeptide comprising the amino acid sequence set forth in SEQ ID NO: 366, or a variant comprising an amino acid sequence having at least about 80% (such as at least about any one of 80%, 85%, 90%, 95%, 96%, 97%, 98%, or 99%) sequence identity, and a light chain comprising the amino acid sequence set forth in SEQ ID NO: 367, or a variant comprising an amino acid sequence having at least about 80% (such as at least about any one of 80%, 85%, 90%, 95%, 96%, 97%, 98%, or 99%) sequence identity.

In some embodiments, the multispecific anti-CD93 construct is capable of blocking the interaction between CD93 and IGFBP7. In some embodiments, the multispecific anti-CD93 construct is capable of blocking the interaction between CD93 and IGFBP7 by at least 5%, 10%, 20%, 30%, 40%, 50%, 60%, 70%, 80%, or 90%.

In some embodiments, the multispecific anti-CD93 construct is capable of blocking the interaction between CD93 and MMRN2. In some embodiments, the multispecific anti-CD93 construct is capable of blocking the interaction between CD93 and MMRN2 by at least 5%, 10%, 20%, 30%, 40%, 50%, 60%, 70%, 80%, or 90%.

In some embodiments, the multispecific anti-CD93 construct is capable of binding to VEGFA with an dissociation constant measure by biolayer interferometry of less than 1 nM, about 1 nM, about 2 nM, about 3 nM, about 4 nM, about 5 nM, about 10 nM, about 20 nM, about 30 nM, about 40 nM, about 50 nM, or higher than about 50 nM. In some embodiments, multispecific anti-CD93 construct is capable of binding to VEGFA with an dissociation constant measure by biolayer interferometry of about 2 nM.

### Additional Anti-CD93 Fusion Proteins

The anti-CD93 constructs in some embodiments comprise an anti-CD93 antibody moiety (e.g., an anti-CD93 scFv) and a second moiety.

In some embodiments, the second moiety comprises a half-life extending moiety. In some embodiments, the half-life extending moiety is an albumin binding moiety (e.g., an albumin binding antibody moiety). In some embodiments, the anti-CD93 antibody moiety and the half-life extending moiety is linked via a linker (such as any of the linkers described in the “Linkers” section).

In some embodiments, the second moiety comprises an extracellular domain of a receptor. In some embodiment, the second moiety is an extracellular domain (ECD) of PD-1 or PD-L1. In some embodiments, the second moiety is a PD-L1 trap or PD-1 trap. See e.g., Nat Commun. 2018 Jun. 8; 9(1):2237. In some embodiments, the second moiety comprises an extracellular domain (ECD) of a VEGF receptor. In some embodiments, the second moiety comprises an ECD of VEGFR1 and/or VEGFR2. In some embodiments, the second moiety comprises a VEGF-trap. See e.g., Proc Natl Acad Sci USA. 2002 Aug. 20; 99(17):11393-8.

### Anti-CD93 Immunoconjugates

The present application also provides anti-CD93 immunoconjugates comprising an anti-CD93 antibody moiety (such as any of the CD93 antibody moieties described herein) and a second agent. In some embodiments, the second agent is a therapeutic agent. In some embodiments, the second agent is a label.

### Linkers

In some embodiments, the anti-CD93 constructs described herein comprise one or more linkers between two moieties (e.g., the anti-CD93 antibody moiety and the half-life extending moiety, the anti-CD93 antibody moiety and the second binding moiety in the multispecific constructs described above). The length, the degree of flexibility and/or other properties of the linker(s) used in the anti-CD93 constructs may have some influence on properties, including but not limited to the affinity, specificity or avidity for one or more particular antigens or epitopes. For example, longer linkers may be selected to ensure that two adjacent domains do not sterically interfere with one another. In some embodiment, a linker (such as peptide linker) comprises flexible residues (such as glycine and serine) so that the adjacent domains are free to move relative to each other. For example, a glycine-serine doublet can be a suitable peptide linker. In some embodiments, the linker is a non-peptide linker. In some embodiments, the linker is a peptide linker. In some embodiments, the linker is a non-cleavable linker. In some embodiments, the linker is a cleavable linker.

Other linker considerations include the effect on physical or pharmacokinetic properties of the resulting compound, such as solubility, lipophilicity, hydrophilicity, hydrophobicity, stability (more or less stable as well as planned degradation), rigidity, flexibility, immunogenicity, modulation of antibody binding, the ability to be incorporated into a micelle or liposome, and the like.

### Peptide Linkers

The peptide linker may have a naturally occurring sequence, or a non-naturally occurring sequence. For example, a sequence derived from the hinge region of heavy chain only antibodies may be used as the linker. See, for example, WO1996/34103.

The peptide linker can be of any suitable length. In some embodiments, the peptide linker is at least about any of 1, 2, 3, 4, S, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acid to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

An essential technical feature of such peptide linker is that said peptide linker does not comprise any polymerization activity. The characteristics of a peptide linker, which comprise the absence of the promotion of secondary structures, are known in the art and described, e.g., in Dall'Acqua et al. (Biochem. (1998) 37, 9266-9273), Cheadle et al. (Mol Immunol (1992) 29, 21-30) and Raag and Whitlow (FASEB (1995) 9(1), 73-80). A particularly preferred amino acid in context of the “peptide linker” is Gly. Furthermore, peptide linkers that also do not promote any secondary structures are preferred. The linkage of the domains to each other can be provided by, e.g., genetic engineering. Methods for preparing fused and operatively linked bispecific single chain constructs and expressing them in mammalian cells or bacteria are well-known in the art (e.g. WO 99/54440, Ausubel, Current Protocols in Molecular Biology, Green Publishing Associates and Wiley Interscience, N. Y. 1989 and 1994 or Sambrook et al., Molecular Cloning: A Laboratory Manual, Cold Spring Harbor Laboratory Press, Cold Spring Harbor, N. Y., 2001).

The peptide linker can be a stable linker, which is not cleavable by proteases, especially by Matrix metalloproteinases (MMPs).

The linker can also be a flexible linker. Exemplary flexible linkers include glycine polymers (G)ₙ (SEQ ID NO: 225), glycine-serine polymers (including, for example, (GS)ₙ (SEQ ID NO: 226), (GSGGS)ₙ (SEQ ID NO: 227), (GGGGS)ₙ (SEQ ID NO: 228), and (GGGS)ₙ (SEQ ID NO: 229), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (See Scheraga, Rev. Computational Chem. 11 173-142 (1992)). The ordinarily skilled artisan will recognize that design of an antibody fusion protein can include linkers that are all or partially flexible, such that the linker can include a flexible linker portion as well as one or more portions that confer less flexible structure to provide a desired antibody fusion protein structure.

Furthermore, exemplary linkers also include the amino acid sequence of such as (GGGGS) (SEQ ID NO: 228), wherein n is an integer between 1 and 8, e.g. (GGGGS)₃ (SEQ ID NO: 230; hereinafter referred to as “(G4S)3” or “GS3”), or (GGGGS)₆ (SEQ ID NO: 231; hereinafter referred to as “(G4S)₆” or “GS6”). In some embodiments, the peptide linker comprises the amino acid sequence of (GSTSGSGKPGSGEGS)ₙ (SEQ ID NO: 232), wherein n is an integer between 1 and 3.

### Non Peptide Linkers

Coupling of two moieties may be accomplished by any chemical reaction that will bind the two molecules so long as both components retain their respective activities, e.g., binding to CD93 and a second agent in an anti-CD93 multispecific antibody, respectively. This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents may be useful in coupling protein molecules in this context. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (See Killen and Lindstrom, Jour. Immun. 133:1335-2549 (1984); Jansen et al., Immunological Reviews 62:185-216 (1982); and Vitetta et al., Science 238:1098 (1987)).

Linkers that can be applied in the present application are described in the literature (see, for example, Ramakrishnan, S. et al., Cancer Res. 44:201-208 (1984) describing use of MBS (M-maleimidobenzoyl-N-hydroxysuccinimide ester). In some embodiments, non-peptide linkers used herein include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido] hexanoate (Pierce Chem. Co., Cat #216510); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC. In some embodiments, the linker is a PEG containing linker.

The linkers described above contain components that have different attributes, thus may lead to bispecific antibodies with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form antibody fusion protein with increased stability. Disulfide linkages, are in general, less stable than other linkages because the disulfide linkage is cleaved in vitro, resulting in less antibody fusion protein available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

### III. Methods of Preparation

In some embodiments, there is provided a method of preparing an anti-CD93 construct or antibody moiety that specifically binds to CD93 and a composition such as polynucleotide, nucleic acid construct, vector, host cell, or culture medium that is produced during the preparation of the anti-CD93 construct or antibody moiety. The anti-CD93 construct or antibody moiety or composition described herein may be prepared by a number of processes as generally described below and more specifically in the Examples.

### Antibody Expression and Production

The antibodies (including anti-CD93 monoclonal antibodies, anti-CD93 bispecific antibodies, and anti-CD93 antibody moieties) described herein can be prepared using any known methods in the art, including those described below and in the Examples.

### Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or a llama, is immunized as hereinabove described to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986). Also See Example 1 for immunization in Camels.

The immunizing agent will typically include the antigenic protein or a fusion variant thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof, e.g., X63-Ag8-653) available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as flow cytometry, radioimmunoassay (RIA) or enzyme-linked immunosorbent assay (ELISA).

The culture medium in which the hybridoma cells are cultured can be assayed for the presence of monoclonal antibodies directed against the desired antigen. Preferably, the binding affinity and specificity of the monoclonal antibody can be determined by immunoprecipitation or by an in vitro binding assay, such as radioimmunoassay (RIA), enzyme-linked assay (ELISA), or BLL Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, ion exchange chromatography, gel electrophoresis, dialysis, or affinity chromatography.

Monoclonal antibodies may also be made by recombinant DNA methods, such as those described in U.S. Pat. No. 4,816,567, and as described above. mRNA encoding the monoclonal antibodies is readily isolated and sequenced using conventional procedures (e.g., by using oligonucleotide probes that are capable of binding specifically to cDNA encoding the heavy and light chains of murine antibodies). The hybridoma cells serve as a preferred source of such mRNA. Once isolated, the cDNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Pluckthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad Sci. USA, 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Nucleic Add Molecules Encoding Antibody Moieties

In some embodiments, there is provided a polynucleotide encoding any one of the anti-CD93 constructs or antibody moieties described herein. In some embodiments, there is provided a polynucleotide prepared using any one of the methods as described herein. In some embodiments, a nucleic acid molecule comprises a polynucleotide that encodes a heavy chain or a light chain of an antibody moiety (e.g., anti-CD93 antibody moiety). In some embodiments, a nucleic acid molecule comprises both a polynucleotide that encodes a heavy chain and a polynucleotide that encodes a light chain, of an antibody moiety (e.g., anti-CD93 antibody moiety). In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody moiety (e.g., anti-CD93 antibody moiety) comprises a nucleotide sequence that encodes a leader sequence, which, when translated, is located at the N terminus of the heavy chain or light chain. As discussed above, the leader sequence may be the native heavy or light chain leader sequence, or may be another heterologous leader sequence.

In some embodiments, the polynucleotide is a DNA. In some embodiments, the polynucleotide is an RNA. In some embodiments, the RNA is an mRNA.

Nucleic acid molecules may be constructed using recombinant DNA techniques conventional in the art. In some embodiments, a nucleic acid molecule is an expression vector that is suitable for expression in a selected host cell.

### Nucleic Acid Construct

In some embodiments, there is provided a nucleic acid construct comprising any one of the polynucleotides described herein. In some embodiments, there is provided a nucleic acid construct prepared using any method described herein.

In some embodiments, the nucleic acid construct further comprises a promoter operably linked to the polynucleotide. In some embodiments, the polynucleotide corresponds to a gene, wherein the promoter is a wild-type promoter for the gene.

### Vectors

In some embodiments, there is provided a vector comprising any polynucleotides that encode the heavy chains and/or light chains of any one of the antibody moieties described herein (e.g., anti-CD93 antibody moieties) or nucleic acid construct described herein. In some embodiments, there is provided a vector prepared using any method described herein. Vectors comprising polynucleotides that encode any of anti-CD93 constructs such as antibodies, scFvs, fusion proteins or other forms of constructs described herein (e.g., anti-CD93 scFv) are also provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a fast vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004).

### Host Cells

In some embodiments, there is provided a host cell comprising any polypeptide, nucleic acid construct and/or vector described herein. In some embodiments, there is provided a host cell prepared using any method described herein. In some embodiments, the host cell is capable of producing any of antibody moieties described herein under a fermentation condition.

In some embodiments, the antibody moieties described herein (e.g., anti-CD93 antibody moieties) may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, CHOZN® and FUT8 CHO cells; PER.C6® cells (Crucell); and NSO cells. In some embodiments, the antibody moieties described herein (e.g., anti-CD93 antibody moieties) may be expressed in yeast. See, e.g., U.S. Publication No. US 2006/0270045 A1. In some embodiments, a particular eukaryotic host cell is selected based on its ability to make desired post-translational modifications to the heavy chains and/or light chains of the antibody moiety. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Non-limiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

The present application also provides host cells comprising any of the polynucleotides or vectors described herein. In some embodiments, the invention provides a host cell comprising an anti-CD93 antibody. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe, or K. lactis).

In some embodiments, the antibody moiety is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### Culture Medium

In some embodiments, there is provided a culture medium comprising any antibody moiety, polynucleotide, nucleic acid construct, vector, and/or host cell described herein. In some embodiments, there is provided a culture medium prepared using any method described herein.

In some embodiments, the medium comprises hypoxanthine, aminopterin, and/or thymidine (e.g., HAT medium). In some embodiments, the medium does not comprise serum. In some embodiments, the medium comprises serum. In some embodiments, the medium is a D-MEM or RPMI-1640 medium. In some embodiments, the medium is a chemically defined medium. In some embodiments, the chemically defined medium is optimized for the host cell line.

### Purification of Antibody Moieties

The anti-CD93 constructs (e.g., anti-CD93 monoclonal antibodies or multispecific antibodies) may be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A, Protein G, Protein A/G, or an antibody affinity column may be used to bind the constant region and to purify an anti-CD93 construct comprising an Fc fragment. Hydrophobic interactive chromatography, for example, a butyl or phenyl column, may also suitable for purifying some polypeptides such as antibodies. Ion exchange chromatography (e.g. anion exchange chromatography and/or cation exchange chromatography) may also suitable for purifying some polypeptides such as antibodies. Mixed-mode chromatography (e.g. reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

### V. Methods of Treatments

Also provided here are methods of treating a disease or condition in an individual or modulating an immune response in an individual. The methods comprise administering the anti-CD93 construct described herein into individuals (e.g., mammals such as humans). It is to be understood that discussion related to anti-CD93 constructs in this section applies to any anti-CD93 constructs described in this application, such as multispecific anti-CD93 constructs, such as anti-CD93 fusion proteins, such as anti-CD93/VEGFR fusion proteins including anti-CD93/Aflibercept fusion proteins.

In some embodiments, there is provided a method of treating a disease or condition or modulating an immune response in an individual, comprising administering to the individual an effective amount of an anti-CD93 construct described herein. Exemplary diseases or conditions include but are not limited to age-related macular degeneration (AMD), diabetic macular edema (DME), choroidal neovascularization (CNV) and cancer.

In some embodiments, there is provided a method of treating a disease or condition (such as an AMD, DME, CNV, or cancer) in an individual, comprising administering to the individual an effective mount of the anti-CD93 construct comprising an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the V_{L-2} comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6. In some embodiments, the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 5, 4, 3, 2, or 1 amino acid substitutions in the LC-CDRs. In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6. In some embodiments, the V_{H} comprises an amino acid sequence of SEQ ID NO: 13, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 14, or a variant comprising an amino acid sequence having at least about 80% (such as at least about any one of 80%, 85%, 90%, 95%, 96%, 97%, 98%, or 99%) sequence identity.

In some embodiments, there is provided a method of treating a disease or condition (such as an AMD, DME, CNV, or cancer) in an individual, comprising administering to the individual an effective mount of the anti-CD93 construct comprising an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 17, the HC-CDR2 comprising the amino acid sequence of SEQ ID NO: 18, and the HC-CDR3 comprising the amino acid sequence of SEQ ID NO: 19, and the V_{L-2} comprises the LC-CDR1 comprising the amino acid sequence of SEQ ID NO: 20, the LC-CDR2 comprising the amino acid sequence of SEQ ID NO: 21, and the LC-CDR3 comprising the amino acid sequence of SEQ ID NO: 22. In some embodiments, the anti-CD93 V_{H} comprises i) the HC-CDR1 comprising the amino acid sequence of SEQ ID NO: 17 or 304, ii) the HC-CDR2 comprising the amino acid sequence of SEQ ID NO: 18 or 305, and iii) the HC-CDR3 comprising the amino acid sequence of SEQ ID NO: 19, or a variant thereof comprising up to 5, 4, 3, 2, or 1 amino acid substitutions in the HC-CDRs, and the anti-CD93 V_{L} comprises i) the LC-CDR1 comprising the amino acid sequence of SEQ ID NO: 20, 301, 302, 303, or 306, ii) the LC-CDR2 comprising the amino acid sequence of SEQ ID NO: 21, and iii) the LC-CDR3 comprising the amino acid sequence of SEQ ID NO: 22, or a variant thereof comprising up to 5, 4, 3, 2, or 1 amino acid substitutions in the LC-CDRs.

In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, and the V_{L} comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22. In some embodiments, the V_{H} comprises an amino acid sequence of any of SEQ ID NO: 29 and 307-312, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of any of SEQ ID NO: 30, and 313-318, or a variant comprising an amino acid sequence having at least about 80% (such as at least about any one of 80%, 85%, 90%, 95%, 96%, 97%, 98%, or 99%) sequence identity.

In some embodiments, there is provided a method of treating a disease or condition (such as an AMD, DME, CNV, or cancer) in an individual, comprising administering to the individual an effective mount of the anti-CD93 construct comprising an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the V_{L-2} comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38. In some embodiments, the V_{H} comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38, or a variant thereof comprising up to 5, 4, 3, 2, or 1 amino acid substitutions in the LC-CDRs. In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, and the V_{L} comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38. In some embodiments, the V_{H} comprises an amino acid sequence of SEQ ID NO: 45, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 46, or a variant comprising an amino acid sequence having at least about 80% (such as at least about any one of 80%, 85%, 90%, 95%, 96%, 97%, 98%, or 99%) sequence identity.

In some embodiments, there is provided a method of treating a disease or condition (such as an AMD, DME, CNV, or cancer) in an individual, comprising administering to the individual an effective mount of the anti-CD93 construct comprising an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 65, the HC-CDR2 comprising the amino acid sequence of SEQ ID NO: 66, and the HC-CDR3 comprising the amino acid sequence of SEQ ID NO: 67, and the V_{L-2} comprises the LC-CDR1 comprising the amino acid sequence of SEQ ID NO: 68, the LC-CDR2 comprising the amino acid sequence of SEQ ID NO: 69, and the LC-CDR3 comprising the amino acid sequence of SEQ ID NO: 70. In some embodiments, the V_{H} comprises i) the HC-CDR1 comprising the amino acid sequence of SEQ ID NO: 65, ii) the HC-CDR2 comprising the amino acid sequence of SEQ ID NO: 66, and iii) the HC-CDR3 comprising the amino acid sequence of SEQ ID NO: 67, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 68, ii) the LC-CDR2 comprising the amino acid sequence of SEQ ID NO: 69, and iii) the LC-CDR3 comprising the amino acid sequence of SEQ ID NO: 70, or a variant thereof comprising up to 5, 4, 3, 2, or 1 amino acid substitutions in the LC-CDRs. In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 65, ii) the HC-CDR2 comprising the amino acid sequence of SEQ ID NO: 66, and iii) the HC-CDR3 comprising the amino acid sequence of SEQ ID NO: 67, and the V_{L} comprises i) the LC-CDR1 comprising the amino acid sequence of SEQ ID NO: 68, ii) the LC-CDR2 comprising the amino acid sequence of SEQ ID NO: 69, and iii) the LC-CDR3 comprising the amino acid sequence of SEQ ID NO: 70. In some embodiments, the V_{H} comprises an amino acid sequence of SEQ ID NO: 77, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of SEQ ID NO: 78, or a variant comprising an amino acid sequence having at least about 80% (such as at least about any one of 80%, 85%, 90%, 95%, 96%, 97%, 98%, or 99%) sequence identity.

In some embodiments, there is provided a method of treating a disease or condition (such as an AMD, DME, CNV, or cancer) in an individual, comprising administering to the individual an effective mount of the anti-CD93 construct comprising an antibody moiety comprising a heavy chain variable region (V_{H}) and a light chain variable region (V_{L}), wherein the antibody moiety competes for a binding epitope of CD93 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 289, the HC-CDR2 comprising the amino acid sequence of SEQ ID NO: 290, and the HC-CDR3 comprising the amino acid sequence of SEQ ID NO: 291, and the V_{L-2} comprises the LC-CDR1 comprising the amino acid sequence of SEQ ID NO: 292, the LC-CDR2 comprising the amino acid sequence of SEQ ID NO: 293, and the LC-CDR3 comprising the amino acid sequence of SEQ ID NO: 294. In some embodiments, the V_{H} comprises i) the HC-CDR1 comprising the amino acid sequence of SEQ ID NO: 289, ii) the HC-CDR2 comprising the amino acid sequence of SEQ ID NO: 290, and iii) the HC-CDR3 comprising the amino acid sequence of SEQ ID NO: 291, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 292, ii) the LC-CDR2 comprising the amino acid sequence of SEQ ID NO: 293, and iii) the LC-CDR3 comprising the amino acid sequence of SEQ ID NO: 294, or a variant thereof comprising up to 5, 4, 3, 2, or 1 amino acid substitutions in the LC-CDRs. In some embodiments, the anti-CD93 antibody moiety is a humanized antibody derived from an anti-CD93 antibody comprising a heavy chain variable region (V_{H}) and a light chain variable region (V_{L}), wherein the V_{H} comprises i) the HC-CDR1 comprising the amino acid sequence of SEQ ID NO: 289, ii) the HC-CDR2 comprising the amino acid sequence of SEQ ID NO: 290, and iii) the HC-CDR3 comprising the amino acid sequence of SEQ ID NO: 291, and the V_{L} comprises i) the LC-CDR1 comprising the amino acid sequence of SEQ ID NO: 292, ii) the LC-CDR2 comprising the amino acid sequence of SEQ ID NO: 293, and iii) the LC-CDR3 comprising the amino acid sequence of SEQ ID NO: 294. In some embodiments, the V_{H} comprises an amino acid sequence of any of SEQ ID NO: 287 and 319-321, or a variant comprising an amino acid sequence having at least about 80% (such as at least about any one of 80%, 85%, 90%, 95%, 96%, 97%, 98%, or 99%) sequence identity, and the V_{L} comprises an amino acid sequence of any of SEQ ID NO: 288, and 322-324, or a variant comprising an amino acid sequence having at least about 80% (such as at least about any one of 80%, 85%, 90%, 95%, 96%, 97%, 98%, or 99%) sequence identity.

In some embodiments, there is provided a method of treating a disease or condition (such as an AMD, DME, CNV, or cancer) in an individual, comprising administering to the individual an effective mount of an anti-CD93 construct comprising a) a full-length antibody that specifically recognizes CD93 comprising two heavy chains and two light chains, wherein the two heavy chains each comprises a heavy chain variable region (V_{H}) comprising the HC-CDR1 comprising the amino acid sequence of SEQ ID NO: 289, the HC-CDR2 comprising the amino acid sequence of SEQ ID NO: 290, and the HC-CDR3 comprising the amino acid sequence of SEQ ID NO: 291, and wherein the two light chains each comprises a light chain variable region (V_{L}) comprises the LC-CDR1 comprising the amino acid sequence of SEQ ID NO: 292, the LC-CDR2 comprising the amino acid sequence of SEQ ID NO: 293, and the LC-CDR3 comprising the amino acid sequence of SEQ ID NO: 294, and b) a VEGF binding moiety comprising the amino acid sequence of SEQ ID NO: 325, wherein the VEGF binding moiety is fused to one or both of the heavy chains of the full-length antibody. In some embodiments, the VEGF binding moiety is fused to C-terminus of both heavy chains of the full-length antibody. In some embodiments, the VEGF binding moiety is fused to the full-length antibody via a linker. In some embodiments, the linker is GS linker or selected from the group consisting of SEQ ID NOs: 225-232 and 338. In some embodiments, the linker comprises the amino acid sequence of SEQ ID NO: 338. In some embodiments, the anti-CD93 V_{H} comprises the amino acid sequence of any one of SEQ ID NOs: 287, and 319-321, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of any one of SEQ ID NOs: 288, and 322-324, or a variant comprising an amino acid sequence having at least about 80% (such as at least about any one of 80%, 85%, 90%, 95%, 96%, 97%, 98%, or 99%) sequence identity. In some embodiments, the full-length antibody has an IgG1 isotype (such as a human IgG1 isotype). In some embodiments, the heavy chain comprises the amino acid sequence of SEQ ID NO: 342, or a variant comprising an amino acid sequence having at least about 80% (such as at least about any one of 80%, 85%, 90%, 95%, 96%, 97%, 98%, or 99%) sequence identity. In some embodiments, the light chain comprises the amino acid sequence of SEQ ID NO: 343, or a variant comprising an amino acid sequence having at least about 80% (such as at least about any one of 80%, 85%, 90%, 95%, 96%, 97%, 98%, or 99%) sequence identity. In some embodiments, there is provided a method of treating a disease or condition (such as an AMD, DME, CNV, or cancer) in an individual, comprising administering to the individual an effective mount of an anti-CD93 construct comprising a heavy chain fusion polypeptide comprising the amino acid sequence set forth in SEQ ID NO: 366, or a variant comprising an amino acid sequence having at least about 80% (such as at least about any one of 80%, 85%, 90%, 95%, 96%, 97%, 98%, or 99%) sequence identity and a light chain polypeptide comprising the amino acid sequence set forth in SEQ ID NO: 367, or a variant comprising an amino acid sequence having at least about 80% (such as at least about any one of 80%, 85%, 90%, 95%, 96%, 97%, 98%, or 99%) sequence identity.

In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, there is provided a method of treating a tumor, comprising administering to the subject any one of the anti-CD93 constructs described herein. In some embodiments, the method retards tumor growth by at least about 5%, about 10%, about 20%, about 30%, about 40%, about 50%, about 60%, about 70%, about 80%, about 90%, or more than 90%, compared to the tumor growth in the absence of the anti-CD93 constructs.

In some embodiments, there is provided a method of reducing size of a tumor in a subject, comprising administering to the subject any one of the anti-CD93 constructs described herein. In some embodiments, reducing size of a tumor refers to reducing tumor volume in a subject. In some embodiments, reducing size of a tumor refers to reducing tumor dimensions (e.g., diameter) in a subject. In some embodiments, the tumor size is reduced by at least about 2%, about 5%, about 10%, about 20%, about 30%, about 40%, about 50%, about 60%, about 70%, about 80%, about 90%, or more than about 90% compared to the size of a counterpart tumor in a subject without the administration of the anti-CD93 construct. In some embodiments, the tumor size is reduced to about 50%, about 60%, about 70%, about 80%, about 90%, or about 90% compared to the size of a counterpart tumor in a subject without the administration of the anti-CD93 construct.

In some embodiments, there is provided a method of eliminating one or more tumors in a subject, comprising administering to the subject any one of the anti-CD93 constructs described herein. In some embodiments, tumor elimination occurs after about 3 days, about 1 week, about 2 weeks, about 3 weeks, about 4 weeks, about 5 weeks, about 6 weeks, about 7 weeks, about 8 weeks, or more than about 8 weeks after anti-CD93 construct.

In some embodiments, there is provided a method of promoting immune cell infiltration into tumors in a subject, comprising administering to the subject any one of the anti-CD93 constructs described herein. In some embodiments, the method increases immune cell penetration into tumors by at least about 2%, about 5%, about 10%, about 20%, about 30%, about 40%, about 50%, about 60%, about 70%, about 80%, about 90%, or more than about 90% compared to that in a subject without the administration of the anti-CD93 construct.

In some embodiments, there is provided a method of eliminating one or more tumors, reducing size of a tumor in a subject, and/or promoting immune cell infiltration into tumors in a subject, comprising administering an anti-CD93 construct comprising a heavy chain fusion polypeptide comprising the amino acid sequence set forth in SEQ ID NO: 366, or a variant comprising an amino acid sequence having at least about 80% (such as at least about any one of 80%, 85%, 90%, 95%, 96%, 97%, 98%, or 99%) sequence identity, and a light chain polypeptide comprising the amino acid sequence set forth in SEQ ID NO: 367.

In some embodiments, there is provided a method of eliminating one or more tumors, reducing size of a tumor in a subject, and/or promoting immune cell infiltration into tumors in a subject, comprising administering an anti-CD93 construct (e.g., any one of the multispecific anti-CD93 construct described herein), wherein the anti-CD93 construct is capable of blocking the interaction between CD93 and IGFBP7. In some embodiments, the multispecific anti-CD93 construct is capable of blocking the interaction between CD93 and IGFBP7 by at least 5%, 10%, 20%, 30%, 40%, 50%, 60%, 70%, 80%, or 90%. In some embodiments, the anti-CD93 construct is capable of blocking the interaction between CD93 and MMRN2. In some embodiments, the multispecific anti-CD93 construct is capable of blocking the interaction between CD93 and MMRN2 by at least 5%, 10%, 20%, 30%, 40%, 50%, 60%, 70%, 80%, or 90%.

In some embodiments, there is provided a method of eliminating one or more tumors, reducing size of a tumor in a subject, and/or promoting immune cell infiltration into tumors in a subject, comprising administering any one of the multispecific anti-CD93 construct described herein, wherein the multispecific anti-CD93 construct is capable of blocking the interaction between CD93 and MMRN2. In some embodiments, the multispecific anti-CD93 construct is capable of blocking the interaction between CD93 and MMRN2 by at least 5%, 10%, 20%, 30%, 40%, 50%, 60%, 70%, 80%, or 90%.

In some embodiments, there is provided a method of eliminating one or more tumors, reducing size of a tumor in a subject, and/or promoting immune cell infiltration into tumors in a subject, comprising administering any one of the multispecific anti-CD93 construct described herein, wherein the multispecific anti-CD93 construct is capable of binding to VEGFA with an dissociation constant measure by biolayer interferometry of less than 1 nM, about 1 nM, about 2 nM, about 3 nM, about 4 nM, about 5 nM, about 10 nM, about 20 nM, about 30 nM, about 40 nM, about 50 nM, or higher than about 50 nM. In some embodiments, the multispecific anti-CD93 construct is capable of binding to VEGFA with an dissociation constant measure by biolayer interferometry of about 2 nM.

### Disease or Condition

The methods described herein are applicable to any disease or conditions associated with an abnormal vascular structure. In some embodiments, the disease or condition is associated with neovascularization. In some embodiments, the disease or condition is a cutaneous psoriasis. In some embodiments, the disease or condition is a benign tumor. In some embodiments, the disease or condition is a cancer.

Diseases Associated with Neovascularization

In some embodiments, the disease or condition is associated with neovascularization. “Neovascularization” described herein refers to a phenomenon that a new vasculature is developed from an existing vasculature.

In some embodiments, the disease of condition is associated with neovascularization of the eye.

In some embodiments, the disease or condition is choroidal neovascularization (CNV), also known as wet AMD. Choroidal neovascularization can involve the growth of new blood vessels that originate from the choroid through a break in the Bruch membrane into the sub-retinal pigment epithelium (sub-RPE) or subretinal space, which can be a major cause of visual loss. CNV can create a sudden deterioration of central vision, noticeable within a few weeks. Other symptoms which can occur include color disturbances, and metamorphopsia (distortions in which straight lines appears wavy). Hemorrhaging of the new blood vessels can accelerate the onset of symptoms of CNV. CNV may also include the feeling of pressure behind the eye. In some embodiments, methods and pharmaceutical compositions as disclosed herein are used to treat CNV or an eye condition associated with neovascularization.

The advanced “wet” form (neovascular or exudative) of AMD is less common, but may frequently cause a rapid and often substantial loss of central vision in patients. In the wet form of AMD, choroidal neovascularization forms and develops into a network of vessels that may grow under and through the retinal pigment epithelium. As this is accompanied by leakage of plasma and/or hemorrhage into the subretinal space, there could be severe sudden loss of central vision if this occurs in the macula. The term “AMD”, if not otherwise specified, can be either dry AMD or wet AMD. The present application contemplates treatment or prevention of AMD, wet AMD and/or dry AMD.

In some embodiments, the disease or condition is a macular edema following retinal vein occlusion (RVO).

In some embodiments, the disease or condition is a diabetic macular edema (DME). Diabetic macular edema (DME) is a swelling of the retina in diabetes mellitus due to leaking of fluid from blood vessels within the macula. The macula is the central portion of the retina, a small area rich in cones, the specialized nerve endings that detect color and upon which daytime vision depends. As macular edema develops, blurring occurs in the middle or just to the side of the central visual field. Visual loss from diabetic macular edema can progress over a period of months and make it impossible to focus clearly. Common symptoms of DME are blurry vision, floaters, double vision, and eventually blindness if it goes untreated. In some embodiments, methods and pharmaceutical compositions as disclosed herein are used to treat DME.

In some embodiments, the disease or condition is a retinal vein occlusion. Retinal vein occlusion is a blockage of the small veins that carry blood away from the retina. The retina is the layer of tissue at the back of the inner eye that converts light images to nerve signals and sends them to the brain. Retinal vein occlusion is most often caused by hardening of the arteries (atherosclerosis) and the formation of a blood clot. Blockage of smaller veins (branch veins or BRVO) in the retina often occurs in places where retinal arteries that have been thickened or hardened by atherosclerosis cross over and place pressure on a retinal vein. Symptoms of retinal vein occlusion can include a sudden blurring or vision loss in all or part of one eye. In some embodiments, methods and pharmaceutical compositions as disclosed herein are used to treat retinal vein occlusion.

In some embodiments, the disease or condition is a diabetic retinopathy (DR) in patients with DME.

### Cancer

In some embodiments, the disease or condition described herein is a cancer. Cancers that may be treated using any of the methods described herein include any types of cancers. Types of cancers to be treated with the agent as described in this application include, but are not limited to, carcinoma, blastoma, sarcoma, benign and malignant tumors, and malignancies e.g., sarcomas, carcinomas, and melanomas. Adult tumors/cancers and pediatric tumors/cancers are also included.

In various embodiments, the cancer is early stage cancer, non-metastatic cancer, primary cancer, advanced cancer, locally advanced cancer, metastatic cancer, cancer in remission, recurrent cancer, cancer in an adjuvant setting, cancer in a neoadjuvant setting, or cancer substantially refractory to a therapy.

In some embodiments, the cancer is a solid tumor.

In some embodiments, the cancer comprises CD93+ tumor endothelial cells. In some embodiments, at least 10%, 20%, 30%, 40%, S0%, 60%, 70%, 80%, or 90% of the endothelial cells in the tumor are CD93 positive. In some embodiments, the cancer comprises at least 20%, 40%, 60%, 80%, or 100% more CD93+ endothelial cells than that of a normal tissue in the subject. In some embodiments, the cancer comprises at least 20%, 40%, 60%, 80%, or 100% more CD93+ endothelial cells than that of a corresponding organ in a subject or a group of subjects who do not have the cancer.

In some embodiments, the cancer comprises IGFBP7+ blood vessels. In some embodiments, the cancer comprises at least 20%, 40%, 60%, 80%, or 100% more IGFBP7+ blood vessels than that of a normal tissue in the subject. In some embodiments, the cancer comprises at least 20%, 40%, 60%, 80%, or 100% more IGFBP7+ blood vessels than that of a corresponding organ in a subject or a group of subjects who do not have the cancer.

In some embodiments, the cancer (e.g., a solid tumor) is characterized by tumor hypoxia. In some embodiments, the cancer is characterized by a pimonidazole positive percentage (i.e., pimonidazole positive area divided by total tumor area) of at least about 1%, 2%, 3%, 4%, or 5%.

Examples of cancers that may be treated by the methods of this application include, but are not limited to, anal cancer, astrocytoma (e.g., cerebellar and cerebral), basal cell carcinoma, bladder cancer, bone cancer, (osteosarcoma and malignant fibrous histiocytoma), brain tumor (e.g., glioma, brain stem glioma, cerebellar or cerebral astrocytoma (e.g., astrocytoma, malignant glioma, medulloblastoma, and glioblastoma), breast cancer, cervical cancer, colon cancer, brain cancer, colorectal cancer, endometrial cancer (e.g., uterine cancer), esophageal cancer, eye cancer (e.g., intraocular melanoma and retinoblastoma), gastric (stomach) cancer, gastrointestinal stromal tumor (GIST), head and neck cancer, hepatocellular (liver) cancer (e.g., hepatic carcinoma and heptoma), liver cancer, lung cancer (e.g., small cell lung cancer, non-small cell lung cancer, adenocarcinoma of the lung, and squamous carcinoma of the lung), medulloblastoma, melanoma, mesothelioma, myelodysplastic syndromes, nasopharyngeal cancer, neuroblastoma, ovarian cancer, pancreatic cancer, parathyroid cancer, cancer of the peritoneal, pituitary tumor, rectal cancer, renal cancer, renal pelvis and ureter cancer (transitional cell cancer), rhabdomyosarcoma, skin cancer (e.g., non-melanoma (e.g., squamous cell carcinoma), melanoma, and Merkel cell carcinoma), small intestine cancer, squamous cell cancer, testicular cancer, thyroid cancer, and tuberous sclerosis. Additional examples of cancers can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

### Subject

In some embodiments, the subject is a mammal (such as a human).

In some embodiments, the subject has a tissue comprising abnormal vascular comprising CD93+ endothelial cells. In some embodiments, at least 10%, 20%, 30%, 40%, 50%, 60%, 70%, 80%, or 90% of the endothelial cells in the tissue with abnormal vascular are CD93 positive. In some embodiments, the tissue with abnormal vascular comprises at least 20%, 40%, 60%, 80%, or 100% more CD93+ endothelial cells than that of a normal tissue in the subject. In some embodiments, the tissue with abnormal vascular comprises at least 20%, 40%, 60%, 80%, or 100% more CD93+ endothelial cells than that of a corresponding organ in a subject or a group of subjects who do not have the abnormal vascular.

In some embodiments, the subject has a tissue comprising abnormal vascular comprising IGFBP7+ blood vessels. In some embodiments, the tissue comprises at least 20%, 40%, 60%, 80%, or 100% more IGFBP7+ blood vessels than that of a normal tissue in the subject. In some embodiments, the tissue comprises at least 20%, 40%, 60%, 80%, or 100% more IGFBP7+ blood vessels than that of a corresponding organ in a subject or a group of subjects who do not have the abnormal vascular.

In some embodiments, the subject is selected for treatment based upon an abnormal vascular structure. In some embodiments, the abnormal vascular structure is characterized by CD93+ endothelial cells (for example, by measuring CD93+ CD31+ cells). In some embodiments, at least 10%, 20%, 30%, 40%, 50%, 60%, 70%, 80%, or 90% of the endothelial cells in the tissue with abnormal vascular are CD93 positive. In some embodiments, the tissue with abnormal vascular comprises at least 20%, 40%, 60%, 80%, or 100% more CD93+ endothelial cells than that of a normal tissue in the subject. In some embodiments, the tissue with abnormal vascular comprises at least 20%, 40%, 60%, 80%, or 100% more CD93+ endothelial cells than that of a corresponding organ in a subject or a group of subjects who do not have the abnormal vascular.

In some embodiments, the abnormal vascular structure is characterized by an abnormal level of IGFBP7+ blood vessels. In some embodiments, the tissue comprises at least 20%, 40%, 60%, 80%, or 100% more IGFBP7+ blood vessels than that of a normal tissue in the subject. In some embodiments, the tissue comprises at least 20%, 40%, 60%, 80%, or 100% more IGFBP7+ blood vessels than that of a corresponding organ in a subject or a group of subjects who do not have the abnormal vascular.

In some embodiments, the subject has at least one prior therapy. In some embodiments, the prior therapy comprises a radiation therapy, a chemotherapy and/or an immunotherapy. In some embodiments, the subject is resistant, refractory, or recurrent to the prior therapy.

Dosing and Method of Administering the anti-CD93 Construct

The dosing regimen of the anti-CD93 construct (such as the specific dosages and frequencies) used for treating a disease or disorder as described herein administered into the individual may vary with the particular anti-CD93 construct (such as anti-CD93 monoclonal or multispecific antibodies, such as anti-CD93 fusion proteins), the mode of administration, and the type of disease or condition being treated. In some embodiments, the type of disease or condition is a cancer. In some embodiments, the effective amount of the anti-CD93 construct (such as anti-CD93 monoclonal or multispecific antibodies) is an amount that is effective to result in an objective response (such as a partial response or a complete response). In some embodiments, the effective amount of the anti-CD93 construct (such as anti-CD93 monoclonal or multispecific antibodies) is an amount that is sufficient to result in a complete response in the individual. In some embodiments, the effective amount of the anti-CD93 construct (such as anti-CD93 monoclonal or multispecific antibodies) is an amount that is sufficient to result in a partial response in the individual. In some embodiments, the effective amount of anti-CD93 construct (such as anti-CD93 monoclonal or multispecific antibodies) is an amount that is sufficient to produce an overall response rate of more than about any of 20%, 25%, 30%, 35%, 40%, 45%, 50%, 55%, 60%, 64%, 65%, 70%, 75%, 80%, 85%, or 90% among a population of individuals treated with the anti-CD93 construct (such as anti-CD93 monoclonal or multispecific antibodies). Responses of an individual to the treatment of the methods described herein can be determined, for example, based on RECIST levels.

In some embodiments, the effective amount of the anti-CD93 construct (such as anti-CD93 monoclonal or multispecific antibodies) is an amount that is sufficient to prolong progress-free survival of the individual. In some embodiments, the effective amount of the anti-CD93 construct (such as anti-CD93 monoclonal or multispecific antibodies) is an amount that is sufficient to prolong overall survival of the individual. In some embodiments, the effective amount of the anti-CD93 construct (such as anti-CD93 monoclonal or multispecific antibodies) is an amount that is sufficient to produce clinical benefit of more than about any of 50%, 60%, 70%, 80%, or 90% among a population of individuals treated with the anti-CD93 construct (such as anti-CD93 monoclonal or multispecific antibodies).

In some embodiments, the effective amount of the anti-CD93 construct (such as anti-CD93 monoclonal or multispecific antibodies) alone or in combination with a second, third, and/or fourth agent, is an amount sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment (e.g., receiving a placebo treatment). Standard methods can be used to measure the magnitude of this effect, such as in vitro assays with purified enzyme, cell-based assays, animal models, or human testing.

In some embodiments, the effective amount of the anti-CD93 construct (such as anti-CD93 monoclonal or multispecific antibodies) is an amount that is below the level that induces a toxicological effect (i.e., an effect above a clinically acceptable level of toxicity) or is at a level where a potential side effect can be controlled or tolerated when the composition is administered to the individual.

In some embodiments, the effective amount of the anti-CD93 construct (such as anti-CD93 monoclonal or multispecific antibodies) is an amount that is close to a maximum tolerated dose (MTD) of the composition following the same dosing regimen. In some embodiments, the effective amount of the anti-CD93 construct (such as anti-CD93 monoclonal or multispecific antibodies) is more than about any of 80%, 90%, 95%, or 98% of the MTD.

In some embodiments, the effective amount of the anti-CD93 construct (such as anti-CD93 monoclonal or multispecific antibodies) is an amount that slows or inhibits the progression of the disease or condition (for example, by at least about 5%, 10%, 15%, 20%, 30%, 40%, 50%) as compared to that of the individual not receiving the treatment. In some embodiments, the disease or condition is an autoimmune disease. In some embodiments, the disease or condition is an infection.

In some embodiments, the effective amount of the anti-CD93 construct (such as anti-CD93 monoclonal or multispecific antibodies) is an amount that reduces the side effects (auto-immune response) of a condition (e.g., transplantation) (for example, by at least about 5%, 10%, 15%, 20%, 30%, 40%, or 50%) as compared to that of the individual not receiving the treatment.

In some embodiments of any of the above aspects, the effective amount of an anti-CD93 construct (such as anti-CD93 monoclonal or multispecific antibodies) is in the range of about 0.001 μg/kg to about 100 mg/kg of total body weight, for example, about 0.005 μg/kg to about 50 mg/kg, about 0.01 μg/kg to about 10 mg/kg, or about 0.01 μg/kg to about 1 mg/kg.

In some embodiments, the treatment comprises more than one administration of the anti-CD93 constructs (such as about two, three, four, five, six, seven, eight, night, or ten administrations of anti-CD93 constructs). In some embodiments, two administrations are carried out within about a week. In some embodiments, a second administration is carried out at least about 1, 2, 3, 4, 5, 6, or 7 days after the completion of the first administration. In some embodiments, a second administration is carried out about 1-14 days, 1-10 days, 1-7 days, 2-6 days, or 3-5 days after the completion of the first administration. In some embodiments, the anti-CD93 construct is administered about 1-3 times a week (such as about once a week, about twice a week, or about three times a week).

The anti-CD93 construct can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, the anti-CD93 construct is included in a pharmaceutical composition while administered into the individual. In some embodiments, sustained continuous release formulation of the composition may be used. In some embodiments, the composition is administered intravenously. In some embodiments, the composition is administered intraperitoneally. In some embodiments, the composition is administered intravenously. In some embodiments, the composition is administered intraperitoneally. In some embodiments, the composition is administered intramuscularly. In some embodiments, the composition is administered subcutaneously. In some embodiments, the composition is administered intravenously. In some embodiments, the composition is administered orally.

### Combination Therapy

This application also provides methods of administering an anti-CD93 construct into an individual for treating a disease or condition (such as cancer), wherein the method further comprises administering a second agent or therapy. In some embodiments, the second agent or therapy is a standard or commonly used agent or therapy for treating the disease or condition. In some embodiments, the second agent or therapy comprises a chemotherapeutic agent. In some embodiments, the second agent or therapy comprises a surgery. In some embodiments, the second agent or therapy comprises a radiation therapy. In some embodiments, the second agent or therapy comprises an immunotherapy. In some embodiments, the second agent or therapy comprises a cell therapy (such as a cell therapy comprising an immune cell (e.g., CAR T cell)). In some embodiments, the second agent or therapy comprises an angiogenesis inhibitor.

In some embodiments, the second agent is a chemotherapeutic agent. In some embodiments, the second agent is antimetabolite agent. In some embodiments, the antimetabolite agent is 5-FU.

In some embodiments, the second agent is an immune checkpoint modulator. In some embodiments, the immune checkpoint modulator is an inhibitor of an immune checkpoint protein selected from the group consisting of PD-L1, PD-L2, CTLA4, PD-L2, PD-1, CD47, TIGIT, GITR, TIM3, LAG3, CD27, 4-1 BB, and B7H4. In some embodiments, the immune checkpoint protein is PD-1. In some embodiments, the second agent is an anti-PD-1 antibody or fragment thereof.

In some embodiments, the second therapy is an immunotherapy. In some embodiments, the immunotherapy comprises administering an immune cell expressing a chimeric antigen receptor. In some embodiments, the immune cell is a T cell (such as a CD4+ T cell or a CD8+ T cell). In some embodiments, the chimeric antigen receptor binds to a tumor antigen.

In some embodiments, the anti-CD93 construct is administered simultaneously with the second agent or therapy. In some embodiments, the anti-CD93 construct is administered concurrently with the second agent or therapy. In some embodiments, the anti-CD93 construct is administered sequentially with the second agent or therapy. In some embodiments, the anti-CD93 construct is administered prior to the second agent or therapy. In some embodiments, the anti-CD93 construct is administered after the second agent or therapy. In some embodiments, the anti-CD93 construct is administered in the same unit dosage form as the second agent or therapy. In some embodiment, the anti-CD93 construct is administered in a different unit dosage form from the second agent or therapy. In some embodiments, the anti-CD93 construct is administered in the same unit dosage form as the second agent or therapy. In some embodiment, the anti-CD93 construct is administered in a different unit dosage form from the second agent or therapy.

### VI. Compositions, Kits and Articles of Manufacture

Also provided herein are compositions (such as formulations) comprising any one of the anti-CD93 construct or anti-CD93 antibody moiety described herein, nucleic acid encoding the antibody moieties, vector comprising the nucleic acid encoding the antibody moieties, or host cells comprising the nucleic acid or vector.

Suitable formulations of the anti-CD93 construct described herein can be obtained by mixing the anti-CD93 construct or anti-CD93 antibody moiety having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be imaged, diagnosed, or treated herein.

The formulations to be used for in vivo administration must be sterile. This is readily accomplished by, e.g., filtration through sterile filtration membranes.

Also provided are kits comprising any one of the anti-CD93 construct or anti-CD93 antibody moiety described herein. The kits may be useful for any of the methods of modulating cell composition or treatment described herein.

In some embodiments, there is provided a kit comprising an anti-CD93 construct specifically binding to CD93.

In some embodiments, the kit further comprises a device capable of delivering the anti-CD93 construct into an individual. One type of device, for applications such as parenteral delivery, is a syringe that is used to inject the composition into the body of a subject. Inhalation devices may also be used for certain applications.

In some embodiments, the kit further comprises a therapeutic agent for treating a disease or condition, e.g., cancer, infectious disease, autoimmune disease, or transplantation.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information.

The present application thus also provides articles of manufacture. The article of manufacture can comprise a container and a label or package insert on or associated with the container. Suitable containers include vials (such as sealed vials), bottles, jars, flexible packaging, and the like. Generally, the container holds a composition, and may have a sterile access port (for example the container may be an intravenous solution bag or a vial having a stopper pierceable by a hypodermic injection needle). The label or package insert indicates that the composition is used for imaging, diagnosing, or treating a particular condition in an individual. The label or package insert will further comprise instructions for administering the composition to the individual and for imaging the individual. The label may indicate directions for reconstitution and/or use. The container holding the composition may be a multi-use vial, which allows for repeat administrations (e.g. from 2-6 administrations) of the reconstituted formulation. Package insert refers to instructions customarily included in commercial packages of diagnostic products that contain information about the indications, usage, dosage, administration, contraindications and/or warnings concerning the use of such diagnostic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the compositions and instructions for use, packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXEMPLARY EMBODIMENTS

Embodiment 1. An anti-CD93 construct comprising an antibody moiety comprising a heavy chain variable region (V_{H}) and a light chain variable region (V_{L}), wherein the antibody moiety competes for a binding epitope of CD93 with an antibody or antibody fragment comprising a second heavy chain variable region (V_{H-2}) and a second light chain variable region (V_{L-2}), wherein:
a) the V_{H-2} comprising the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the V_{L-2} comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6;
b) the V_{H-2} comprises the HC-CDR1 comprising the amino acid sequence of SEQ ID NO: 17, the HC-CDR2 comprising the amino acid sequence of SEQ ID NO: 18, and the HC-CDR3 comprising the amino acid sequence of SEQ ID NO: 19, and the V_{L-2} comprises the LC-CDR1 comprising the amino acid sequence of SEQ ID NO: 20, the LC-CDR2 comprising the amino acid sequence of SEQ ID NO: 21, and the LC-CDR3 comprising the amino acid sequence of SEQ ID NO: 22;
c) the V_{H-2} comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the V_{L-2} comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38;
d) the V_{H-2} comprises the HC-CDR1 comprising the amino acid sequence of SEQ ID NO: 49, the HC-CDR2 comprising the amino acid sequence of SEQ ID NO: 50, and the HC-CDR3 comprising the amino acid sequence of SEQ ID NO: 51, and the V_{L-2} comprises the LC-CDR1 comprising the amino acid sequence of SEQ ID NO: 52, the LC-CDR2 comprising the amino acid sequence of SEQ ID NO: 53, and the LC-CDR3 comprising the amino acid sequence of SEQ ID NO: 54;
e) the V_{H-2} comprises the HC-CDR1 comprising the amino acid sequence of SEQ ID NO: 65, the HC-CDR2 comprising the amino acid sequence of SEQ ID NO: 66, and the HC-CDR3 comprising the amino acid sequence of SEQ ID NO: 67, and the V_{L-2} comprises the LC-CDR1 comprising the amino acid sequence of SEQ ID NO: 68, the LC-CDR2 comprising the amino acid sequence of SEQ ID NO: 69, and the LC-CDR3 comprising the amino acid sequence of SEQ ID NO: 70;
f) the V_{H-2} comprises the HC-CDR1 comprising the amino acid sequence of SEQ ID NO: 81, the HC-CDR2 comprising the amino acid sequence of SEQ ID NO: 82, and the HC-CDR3 comprising the amino acid sequence of SEQ ID NO: 83, and the V_{L-2} comprises the LC-CDR1 comprising the amino acid sequence of SEQ ID NO: 84, the LC-CDR2 comprising the amino acid sequence of SEQ ID NO: 85, and the LC-CDR3 comprising the amino acid sequence of SEQ ID NO: 86;
g) the V_{H-2} comprises the HC-CDR1 comprising the amino acid sequence of SEQ ID NO: 97, the HC-CDR2 comprising the amino acid sequence of SEQ ID NO: 98, and the HC-CDR3 comprising the amino acid sequence of SEQ ID NO: 99, and the V_{L-2} comprises the LC-CDR1 comprising the amino acid sequence of SEQ ID NO: 100, the LC-CDR2 comprising the amino acid sequence of SEQ ID NO: 101, and the LC-CDR3 comprising the amino acid sequence of SEQ ID NO: 102;
h) the V_{H-2} comprises the HC-CDR1 comprising the amino acid sequence of SEQ ID NO: 113, the HC-CDR2 comprising the amino acid sequence of SEQ ID NO: 114, and the HC-CDR3 comprising the amino acid sequence of SEQ ID NO: 115, and the V_{L-2} comprises the LC-CDR1 comprising the amino acid sequence of SEQ ID NO: 116, the LC-CDR2 comprising the amino acid sequence of SEQ ID NO: 117, and the LC-CDR3 comprising the amino acid sequence of SEQ ID NO: 118;
i) the V_{H-2} comprises the HC-CDR1 comprising the amino acid sequence of SEQ ID NO: 129, the HC-CDR2 comprising the amino acid sequence of SEQ ID NO: 130, and the HC-CDR3 comprising the amino acid sequence of SEQ ID NO: 131, and the V_{L-2} comprises the LC-CDR1 comprising the amino acid sequence of SEQ ID NO: 132, the LC-CDR2 comprising the amino acid sequence of SEQ ID NO: 133, and the LC-CDR3 comprising the amino acid sequence of SEQ ID NO: 134;
j) the V_{H-2} comprises the HC-CDR1 comprising the amino acid sequence of SEQ ID NO: 145, the HC-CDR2 comprising the amino acid sequence of SEQ ID NO: 146, and the HC-CDR3 comprising the amino acid sequence of SEQ ID NO: 147, and the V_{L-2} comprises the LC-CDR1 comprising the amino acid sequence of SEQ ID NO: 148, 355, or 358, the LC-CDR2 comprising the amino acid sequence of SEQ ID NO: 149 or 356, and the LC-CDR3 comprising the amino acid sequence of SEQ ID NO: 150, 357 or 359;
k) the V_{H-2} comprises the HC-CDR1 comprising the amino acid sequence of SEQ ID NO: 161, the HC-CDR2 comprising the amino acid sequence of SEQ ID NO: 162, and the HC-CDR3 comprising the amino acid sequence of SEQ ID NO: 163, and the V_{L-2} comprises the LC-CDR1 comprising the amino acid sequence of SEQ ID NO: 164, the LC-CDR2 comprising the amino acid sequence of SEQ ID NO: 165, and the LC-CDR3 comprising the amino acid sequence of SEQ ID NO: 166;
l) the V_{H-2} comprises the HC-CDR1 comprising the amino acid sequence of SEQ ID NO: 177, the HC-CDR2 comprising the amino acid sequence of SEQ ID NO: 178, and the HC-CDR3 comprising the amino acid sequence of SEQ ID NO: 179, and the V_{L-2} comprises the LC-CDR1 comprising the amino acid sequence of SEQ ID NO: 180 or 353, the LC-CDR2 comprising the amino acid sequence of SEQ ID NO: 181 or 354, and the LC-CDR3 comprising the amino acid sequence of SEQ ID NO: 182;
m) the V_{H-2} comprises the HC-CDR1 comprising the amino acid sequence of SEQ ID NO: 193, the HC-CDR2 comprising the amino acid sequence of SEQ ID NO: 194, and the HC-CDR3 comprising the amino acid sequence of SEQ ID NO: 195, and the V_{L-2} comprises the LC-CDR1 comprising the amino acid sequence of SEQ ID NO: 196, the LC-CDR2 comprising the amino acid sequence of SEQ ID NO: 197, and the LC-CDR3 comprising the amino acid sequence of SEQ ID NO: 198;
n) the V_{H-2} comprises the HC-CDR1 comprising the amino acid sequence of SEQ ID NO: 209, the HC-CDR2 comprising the amino acid sequence of SEQ ID NO: 210, and the HC-CDR3 comprising the amino acid sequence of SEQ ID NO: 211, and the V_{L-2} comprises the LC-CDR1 comprising the amino acid sequence of SEQ ID NO: 212, the LC-CDR2 comprising the amino acid sequence of SEQ ID NO: 213, and the LC-CDR3 comprising the amino acid sequence of SEQ ID NO: 214;
o) the V_{H-2} comprises the HC-CDR1 comprising the amino acid sequence of SEQ ID NO: 289, the HC-CDR2 comprising the amino acid sequence of SEQ ID NO: 290, and the HC-CDR3 comprising the amino acid sequence of SEQ ID NO: 291, and the V_{L-2} comprises the LC-CDR1 comprising the amino acid sequence of SEQ ID NO: 292, the LC-CDR2 comprising the amino acid sequence of SEQ ID NO: 293, and the LC-CDR3 comprising the amino acid sequence of SEQ ID NO: 294; or
p) the V_{H-2} comprises the HC-CDR1 comprising the amino acid sequence of SEQ ID NO: 17 or 304, the HC-CDR2 comprising the amino acid sequence of SEQ ID NO: 18 or 305, and the HC-CDR3 comprising the amino acid sequence of SEQ ID NO: 19, and the V_{L-2} comprises the LC-CDR1 comprising the amino acid sequence of SEQ ID NO: 20, 301, 302, 303, or 306, the LC-CDR2 comprising the amino acid sequence of SEQ ID NO: 21, and the LC-CDR3 comprising the amino acid sequence of SEQ ID NO:22.

Embodiment 2. The anti-CD93 construct of embodiment 1, wherein:
a) the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 5, 4, 3, 2, or 1 amino acid substitutions in the LC-CDRs,
b) the V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22, or a variant thereof comprising up to 5, 4, 3, 2, or 1 amino acid substitutions in the LC-CDRs,
c) the V_{H} comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38, or a variant thereof comprising up to 5, 4, 3, 2, or 1 amino acid substitutions in the LC-CDRs,
d) the V_{H} comprises i) the HC-CDR1 comprising the amino acid sequence of SEQ ID NO: 49, ii) the HC-CDR2 comprising the amino acid sequence of SEQ ID NO: 50, and iii) the HC-CDR3 comprising the amino acid sequence of SEQ ID NO: 51, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 52, ii) the LC-CDR2 comprising the amino acid sequence of SEQ ID NO: 53, and iii) the LC-CDR3 comprising the amino acid sequence of SEQ ID NO: 54, or a variant thereof comprising up to 5, 4, 3, 2, or 1 amino acid substitutions in the LC-CDRs,
e) the V_{H} comprises i) the HC-CDR1 comprising the amino acid sequence of SEQ ID NO: 65, ii) the HC-CDR2 comprising the amino acid sequence of SEQ ID NO: 66, and iii) the HC-CDR3 comprising the amino acid sequence of SEQ ID NO: 67, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 68, ii) the LC-CDR2 comprising the amino acid sequence of SEQ ID NO: 69, and iii) the LC-CDR3 comprising the amino acid sequence of SEQ ID NO: 70, or a variant thereof comprising up to 5, 4, 3, 2, or 1 amino acid substitutions in the LC-CDRs,
f) the V_{H} comprises i) the HC-CDR1 comprising the amino acid sequence of SEQ ID NO: 81, ii) the HC-CDR2 comprising the amino acid sequence of SEQ ID NO: 82, and iii) the HC-CDR3 comprising the amino acid sequence of SEQ ID NO: 83, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 84, ii) the LC-CDR2 comprising the amino acid sequence of SEQ ID NO: 85, and iii) the LC-CDR3 comprising the amino acid sequence of SEQ ID NO: 86, or a variant thereof comprising up to 5, 4, 3, 2, or 1 amino acid substitutions in the LC-CDRs,
g) the V_{H} comprises i) the HC-CDR1 comprising the amino acid sequence of SEQ ID NO: 97, ii) the HC-CDR2 comprising the amino acid sequence of SEQ ID NO: 98, and iii) the HC-CDR3 comprising the amino acid sequence of SEQ ID NO: 99, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 100, ii) the LC-CDR2 comprising the amino acid sequence of SEQ ID NO: 101, and iii) the LC-CDR3 comprising the amino acid sequence of SEQ ID NO: 102, or a variant thereof comprising up to 5, 4, 3, 2, or 1 amino acid substitutions in the LC-CDRs,
h) the V_{H} comprises i) the HC-CDR1 comprising the amino acid sequence of SEQ ID NO: 113, ii) the HC-CDR2 comprising the amino acid sequence of SEQ ID NO: 114, and iii) the HC-CDR3 comprising the amino acid sequence of SEQ ID NO: 115, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 116, ii) the LC-CDR2 comprising the amino acid sequence of SEQ ID NO: 117, and iii) the LC-CDR3 comprising the amino acid sequence of SEQ ID NO: 118, or a variant thereof comprising up to 5, 4, 3, 2, or 1 amino acid substitutions in the LC-CDRs,
i) the V_{H} comprises i) the HC-CDR1 comprising the amino acid sequence of SEQ ID NO: 129, ii) the HC-CDR2 comprising the amino acid sequence of SEQ ID NO: 130, and iii) the HC-CDR3 comprising the amino acid sequence of SEQ ID NO: 131, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 132, ii) the LC-CDR2 comprising the amino acid sequence of SEQ ID NO: 133, and iii) the LC-CDR3 comprising the amino acid sequence of SEQ ID NO: 134, or a variant thereof comprising up to 5, 4, 3, 2, or 1 amino acid substitutions in the LC-CDRs,
j) the V_{H} comprises i) the HC-CDR1 comprising the amino acid sequence of SEQ ID NO: 145, ii) the HC-CDR2 comprising the amino acid sequence of SEQ ID NO: 146, and iii) the HC-CDR3 comprising the amino acid sequence of SEQ ID NO: 147, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 148, 355, or 358, ii) the LC-CDR2 comprising the amino acid sequence of SEQ ID NO: 149 or 356, and iii) the LC-CDR3 comprising the amino acid sequence of SEQ ID NO: 150, 357 or 359, or a variant thereof comprising up to 5, 4, 3, 2, or 1 amino acid substitutions in the LC-CDRs,
k) the V_{H} comprises i) the HC-CDR1 comprising the amino acid sequence of SEQ ID NO: 161, ii) the HC-CDR2 comprising the amino acid sequence of SEQ ID NO: 162, and iii) the HC-CDR3 comprising the amino acid sequence of SEQ ID NO: 163, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 164, ii) the LC-CDR2 comprising the amino acid sequence of SEQ ID NO: 165, and iii) the LC-CDR3 comprising the amino acid sequence of SEQ ID NO: 166, or a variant thereof comprising up to 5, 4, 3, 2, or 1 amino acid substitutions in the LC-CDRs,
l) the V_{H} comprises i) the HC-CDR1 comprising the amino acid sequence of SEQ ID NO: 177, ii) the HC-CDR2 comprising the amino acid sequence of SEQ ID NO: 178, and iii) the HC-CDR3 comprising the amino acid sequence of SEQ ID NO: 179, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 180 or 353, ii) the LC-CDR2 comprising the amino acid sequence of SEQ ID NO: 181 or 354, and iii) the LC-CDR3 comprising the amino acid sequence of SEQ ID NO: 182, or a variant thereof comprising up to 5, 4, 3, 2, or 1 amino acid substitutions in the LC-CDRs,
m) the V_{H} comprises i) the HC-CDR1 comprising the amino acid sequence of SEQ ID NO: 193, ii) the HC-CDR2 comprising the amino acid sequence of SEQ ID NO: 194, and iii) the HC-CDR3 comprising the amino acid sequence of SEQ ID NO: 195, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 196, ii) the LC-CDR2 comprising the amino acid sequence of SEQ ID NO: 197, and iii) the LC-CDR3 comprising the amino acid sequence of SEQ ID NO: 198, or a variant thereof comprising up to 5, 4, 3, 2, or 1 amino acid substitutions in the LC-CDRs,
n) the V_{H} comprises i) the HC-CDR1 comprising the amino acid sequence of SEQ ID NO: 209, ii) the HC-CDR2 comprising the amino acid sequence of SEQ ID NO: 210, and iii) the HC-CDR3 comprising the amino acid sequence of SEQ ID NO: 211, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 212, ii) the LC-CDR2 comprising the amino acid sequence of SEQ ID NO: 213, and iii) the LC-CDR3 comprising the amino acid sequence of SEQ ID NO: 214, or a variant thereof comprising up to 5, 4, 3, 2, or 1 amino acid substitutions in the LC-CDRs,
o) the V_{H} comprises i) the HC-CDR1 comprising the amino acid sequence of SEQ ID NO: 289, ii) the HC-CDR2 comprising the amino acid sequence of SEQ ID NO: 290, and iii) the HC-CDR3 comprising the amino acid sequence of SEQ ID NO: 291, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 292, ii) the LC-CDR2 comprising the amino acid sequence of SEQ ID NO: 293, and iii) the LC-CDR3 comprising the amino acid sequence of SEQ ID NO: 294, or a variant thereof comprising up to 5, 4, 3, 2, or 1 amino acid substitutions in the LC-CDRs, or
p) the V_{H} comprises the HC-CDR1 comprising the amino acid sequence of SEQ ID NO: 17 or 304, the HC-CDR2 comprising the amino acid sequence of SEQ ID NO: 18 or 305, and the HC-CDR3 comprising the amino acid sequence of SEQ ID NO: 19, or a variant thereof comprising up to 5, 4, 3, 2, or 1 amino acid substitutions in the HC-CDRs; and the V_{L} comprises the LC-CDR1 comprising the amino acid sequence of SEQ ID NO: 20, 301, 302, 303, or 306, the LC-CDR2 comprising the amino acid sequence of SEQ ID NO: 21, and the LC-CDR3 comprising the amino acid sequence of SEQ ID NO:22, or a variant thereof comprising up to 5, 4, 3, 2, or 1 amino acid substitutions in the LC-CDRs.

Embodiment 3. The anti-CD93 construct of embodiment 2, wherein the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 5, 4, 3, 2, or 1 amino acid substitutions in the LC-CDRs.

Embodiment 4. The anti-CD93 construct of embodiment 2, wherein the V_{H} comprises i) the HC-CDR1 comprising the amino acid sequence of SEQ ID NO: 17 or 304, ii) the HC-CDR2 comprising the amino acid sequence of SEQ ID NO: 18 or 305, and iii) the HC-CDR3 comprising the amino acid sequence of SEQ ID NO: 19, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 20, 301, 302, 303, or 306, ii) the LC-CDR2 comprising the amino acid sequence of SEQ ID NO: 21, and iii) the LC-CDR3 comprising the amino acid sequence of SEQ ID NO: 22, or a variant thereof comprising up to 5, 4, 3, 2, or 1 amino acid substitutions in the LC-CDRs.

Embodiment 5. The anti-CD93 construct of embodiment 2, wherein the V_{H} comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38, or a variant thereof comprising up to 5, 4, 3, 2, or 1 amino acid substitutions in the LC-CDRs.

Embodiment 6. The anti-CD93 construct of embodiment 2, wherein the V_{H} comprises i) the HC-CDR1 comprising the amino acid sequence of SEQ ID NO: 49, ii) the HC-CDR2 comprising the amino acid sequence of SEQ ID NO: 50, and iii) the HC-CDR3 comprising the amino acid sequence of SEQ ID NO: 51, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 52, ii) the LC-CDR2 comprising the amino acid sequence of SEQ ID NO: 53, and iii) the LC-CDR3 comprising the amino acid sequence of SEQ ID NO: 54, or a variant thereof comprising up to 5, 4, 3, 2, or 1 amino acid substitutions in the LC-CDRs.

Embodiment 7. The anti-CD93 construct of embodiment 2, wherein the V_{H} comprises i) the HC-CDR1 comprising the amino acid sequence of SEQ ID NO: 65, ii) the HC-CDR2 comprising the amino acid sequence of SEQ ID NO: 66, and iii) the HC-CDR3 comprising the amino acid sequence of SEQ ID NO: 67, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 68, ii) the LC-CDR2 comprising the amino acid sequence of SEQ ID NO: 69, and iii) the LC-CDR3 comprising the amino acid sequence of SEQ ID NO: 70, or a variant thereof comprising up to 5, 4, 3, 2, or 1 amino acid substitutions in the LC-CDRs.

Embodiment 8. The anti-CD93 construct of embodiment 2, wherein the V_{H} comprises i) the HC-CDR1 comprising the amino acid sequence of SEQ ID NO: 81, ii) the HC-CDR2 comprising the amino acid sequence of SEQ ID NO: 82, and iii) the HC-CDR3 comprising the amino acid sequence of SEQ ID NO: 83, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 84, ii) the LC-CDR2 comprising the amino acid sequence of SEQ ID NO: 85, and iii) the LC-CDR3 comprising the amino acid sequence of SEQ ID NO: 86, or a variant thereof comprising up to 5, 4, 3, 2, or 1 amino acid substitutions in the LC-CDRs.

Embodiment 9. The anti-CD93 construct of embodiment 2, wherein the V_{H} comprises i) the HC-CDR1 comprising the amino acid sequence of SEQ ID NO: 97, ii) the HC-CDR2 comprising the amino acid sequence of SEQ ID NO: 98, and iii) the HC-CDR3 comprising the amino acid sequence of SEQ ID NO: 99, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 100, ii) the LC-CDR2 comprising the amino acid sequence of SEQ ID NO: 101, and iii) the LC-CDR3 comprising the amino acid sequence of SEQ ID NO: 102, or a variant thereof comprising up to 5, 4, 3, 2, or 1 amino acid substitutions in the LC-CDRs.

Embodiment 10. The anti-CD93 construct of embodiment 2, wherein the V_{H} comprises i) the HC-CDR1 comprising the amino acid sequence of SEQ ID NO: 113, ii) the HC-CDR2 comprising the amino acid sequence of SEQ ID NO: 114, and iii) the HC-CDR3 comprising the amino acid sequence of SEQ ID NO: 115, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 116, ii) the LC-CDR2 comprising the amino acid sequence of SEQ ID NO: 117, and iii) the LC-CDR3 comprising the amino acid sequence of SEQ ID NO: 118, or a variant thereof comprising up to 5, 4, 3, 2, or 1 amino acid substitutions in the LC-CDRs.

Embodiment 11. The anti-CD93 construct of embodiment 2, wherein the V_{H} comprises i) the HC-CDR1 comprising the amino acid sequence of SEQ ID NO: 129, ii) the HC-CDR2 comprising the amino acid sequence of SEQ ID NO: 130, and iii) the HC-CDR3 comprising the amino acid sequence of SEQ ID NO: 131, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 132, ii) the LC-CDR2 comprising the amino acid sequence of SEQ ID NO: 133, and iii) the LC-CDR3 comprising the amino acid sequence of SEQ ID NO: 134, or a variant thereof comprising up to 5, 4, 3, 2, or 1 amino acid substitutions in the LC-CDRs.

Embodiment 12. The anti-CD93 construct of embodiment 2, wherein the V_{H} comprises i) the HC-CDR1 comprising the amino acid sequence of SEQ ID NO: 145, ii) the HC-CDR2 comprising the amino acid sequence of SEQ ID NO: 146, and iii) the HC-CDR3 comprising the amino acid sequence of SEQ ID NO: 147, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 148, 355, or 358, ii) the LC-CDR2 comprising the amino acid sequence of SEQ ID NO: 149 or 356, and iii) the LC-CDR3 comprising the amino acid sequence of SEQ ID NO: 150, 357 or 359, or a variant thereof comprising up to 5, 4, 3, 2, or 1 amino acid substitutions in the LC-CDRs.

Embodiment 13. The anti-CD93 construct of embodiment 2, wherein the V_{H} comprises i) the HC-CDR1 comprising the amino acid sequence of SEQ ID NO: 161, ii) the HC-CDR2 comprising the amino acid sequence of SEQ ID NO: 162, and iii) the HC-CDR3 comprising the amino acid sequence of SEQ ID NO: 163, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 164, ii) the LC-CDR2 comprising the amino acid sequence of SEQ ID NO: 165, and iii) the LC-CDR3 comprising the amino acid sequence of SEQ ID NO: 166, or a variant thereof comprising up to 5, 4, 3, 2, or 1 amino acid substitutions in the LC-CDRs.

Embodiment 14. The anti-CD93 construct of embodiment 2, wherein the V_{H} comprises i) the HC-CDR1 comprising the amino acid sequence of SEQ ID NO: 177, ii) the HC-CDR2 comprising the amino acid sequence of SEQ ID NO: 178, and iii) the HC-CDR3 comprising the amino acid sequence of SEQ ID NO: 179, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 180 or 353, ii) the LC-CDR2 comprising the amino acid sequence of SEQ ID NO: 181 or 354, and iii) the LC-CDR3 comprising the amino acid sequence of SEQ ID NO: 182, or a variant thereof comprising up to 5, 4, 3, 2, or 1 amino acid substitutions in the LC-CDRs.

Embodiment 15. The anti-CD93 construct of embodiment 2, wherein the V_{H} comprises i) the HC-CDR1 comprising the amino acid sequence of SEQ ID NO: 193, ii) the HC-CDR2 comprising the amino acid sequence of SEQ ID NO: 194, and iii) the HC-CDR3 comprising the amino acid sequence of SEQ ID NO: 195, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 196, ii) the LC-CDR2 comprising the amino acid sequence of SEQ ID NO: 197, and iii) the LC-CDR3 comprising the amino acid sequence of SEQ ID NO: 198, or a variant thereof comprising up to 5, 4, 3, 2, or 1 amino acid substitutions in the LC-CDRs.

Embodiment 16. The anti-CD93 construct of embodiment 2, wherein the V_{H} comprises i) the HC-CDR1 comprising the amino acid sequence of SEQ ID NO: 209, ii) the HC-CDR2 comprising the amino acid sequence of SEQ ID NO: 210, and iii) the HC-CDR3 comprising the amino acid sequence of SEQ ID NO: 211, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 212, ii) the LC-CDR2 comprising the amino acid sequence of SEQ ID NO: 213, and iii) the LC-CDR3 comprising the amino acid sequence of SEQ ID NO: 214, or a variant thereof comprising up to 5, 4, 3, 2, or 1 amino acid substitutions in the LC-CDRs.

Embodiment 17. The anti-CD93 construct of embodiment 2, wherein the V_{H} comprises i) the HC-CDR1 comprising the amino acid sequence of SEQ ID NO: 289, ii) the HC-CDR2 comprising the amino acid sequence of SEQ ID NO: 290, and iii) the HC-CDR3 comprising the amino acid sequence of SEQ ID NO: 291, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 292, ii) the LC-CDR2 comprising the amino acid sequence of SEQ ID NO: 293, and iii) the LC-CDR3 comprising the amino acid sequence of SEQ ID NO: 294, or a variant thereof comprising up to 5, 4, 3, 2, or 1 amino acid substitutions in the LC-CDRs

Embodiment 18. An anti-CD93 construct comprising an antibody moiety that specifically binds to CD93, comprising:
a) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 13, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 14;
b) a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 29 and 307-312, and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 30, and 313-318;
c) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 45, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 46;
d) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 61, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 62;
e) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 77, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 78;
f) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 93, and a LC-CDR1, a LC-CDR2, and a LC-CDR3, respectively comprising the amino acid sequences of a CDR1, a CDR2, and a CDR3 within a V_{L}. chain region having the sequence set forth in SEQ ID NO: 94;
g) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 109, and a LC-CDR1, a LC-CDR2, and a LC-CDR3, respectively comprising the amino acid sequences of a CDR1, a CDR2, and a CDR3 within a V_{L}. chain region having the sequence set forth in SEQ ID NO: 110;
h) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 125, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 126;
i) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 141, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 142;
j) a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 157 and 360-362, and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 158, and 363-365;
k) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 173, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 174;
l) a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 189 and 347-349, and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 190, and 350-352;
m) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 205, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 206;
n) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 221, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 222;
o) a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 287 and 319-321, and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 288, and 322-324;
p) a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NOs: 307-312, and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NOs: 313-318; or
q) a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NOs: 319-321, and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NOs: 322-324.

Embodiment 19. The anti-CD93 construct of any one of embodiments 1-18, wherein the V_{H} comprises an amino acid sequence of any one of SEQ ID NOs: 13, 29, 45, 61, 77, 93, 109, 125, 141, 157, 173, 189, 205, 221, 287, 307-312 and 319-321, or a variant comprising an amino acid sequence having at least about 80% sequence identity, and/or wherein the V_{L} comprises an amino acid sequence of any one of SEQ ID NOs: 14, 30, 46, 62, 78, 94, 110, 126, 142, 158, 174, 190, 206, 222, 288, 313-318 and 322-324 or a variant comprising an amino acid sequence having at least about 80% sequence identity.

Embodiment 20. The anti-CD93 construct of embodiment 19, wherein:
a) the V_{H} comprises an amino acid sequence of SEQ ID NO: 13, or a variant comprising an amino acid sequence having at least about 80% sequence identity; and the V_{L} comprises an amino acid sequence of SEQ ID NO: 14, or a variant comprising an amino acid sequence having at least about 80% sequence identity,
b) the V_{H} comprises an amino acid sequence of any of SEQ ID NO: 29 and 307-312, or a variant comprising an amino acid sequence having at least about 80% sequence identity, and the V_{L} comprises an amino acid sequence of any of SEQ ID NO: 30, and 313-318, or a variant comprising an amino acid sequence having at least about 80% sequence identity,
c) the V_{H} comprises an amino acid sequence of SEQ ID NO: 45, or a variant comprising an amino acid sequence having at least about 80% sequence identity; and the V_{L} comprises an amino acid sequence of SEQ ID NO: 46, or a variant comprising an amino acid sequence having at least about 80% sequence identity,
d) the V_{H} comprises an amino acid sequence of SEQ ID NO: 61, or a variant comprising an amino acid sequence having at least about 80% sequence identity; and the V_{L} comprises an amino acid sequence of SEQ ID NO: 62, or a variant comprising an amino acid sequence having at least about 80% sequence identity,
e) the V_{H} comprises an amino acid sequence of SEQ ID NO: 77, or a variant comprising an amino acid sequence having at least about 80% sequence identity; and the V_{L} comprises an amino acid sequence of SEQ ID NO: 78, or a variant comprising an amino acid sequence having at least about 80% sequence identity,
f) the V_{H} comprises an amino acid sequence of SEQ ID NO: 93, or a variant comprising an amino acid sequence having at least about 80% sequence identity; and the V_{L} comprises an amino acid sequence of SEQ ID NO: 94, or a variant comprising an amino acid sequence having at least about 80% sequence identity,
g) the V_{H} comprises an amino acid sequence of SEQ ID NO: 109, or a variant comprising an amino acid sequence having at least about 80% sequence identity, and the V_{L} comprises an amino acid sequence of SEQ ID NO: 110, or a variant comprising an amino acid sequence having at least about 80% sequence identity,
h) the V_{H} comprises an amino acid sequence of SEQ ID NO: 125, or a variant comprising an amino acid sequence having at least about 80% sequence identity; and the V_{L} comprises an amino acid sequence of SEQ ID NO: 126, or a variant comprising an amino acid sequence having at least about 80% sequence identity,
i) the V_{H} comprises an amino acid sequence of SEQ ID NO: 141, or a variant comprising an amino acid sequence having at least about 80% sequence identity, and the V_{L} comprises an amino acid sequence of SEQ ID NO: 142, or a variant comprising an amino acid sequence having at least about 80% sequence identity,
j) the V_{H} comprises an amino acid sequence of SEQ ID NO: 157, or a variant comprising an amino acid sequence having at least about 80% sequence identity, and the V_{L} comprises an amino acid sequence of SEQ ID NO: 158, or a variant comprising an amino acid sequence having at least about 80% sequence identity,
k) the V_{H} comprises an amino acid sequence of SEQ ID NO: 173, or a variant comprising an amino acid sequence having at least about 80% sequence identity; and the V_{L} comprises an amino acid sequence of SEQ ID NO: 174, or a variant comprising an amino acid sequence having at least about 80% sequence identity,
l) the V_{H} comprises an amino acid sequence of any of SEQ ID NO: 189 and 347-349, or a variant comprising an amino acid sequence having at least about 80% sequence identity, and the V_{L} comprises an amino acid sequence of any of SEQ ID NO: 190, and 350-352, or a variant comprising an amino acid sequence having at least about 80% sequence identity,
m) the V_{H} comprises an amino acid sequence of SEQ ID NO: 205, or a variant comprising an amino acid sequence having at least about 80% sequence identity; and the V_{L} comprises an amino acid sequence of SEQ ID NO: 206, or a variant comprising an amino acid sequence having at least about 80% sequence identity,
n) the V_{H} comprises an amino acid sequence of SEQ ID NO: 221, or a variant comprising an amino acid sequence having at least about 80% sequence identity; and the V_{L} comprises an amino acid sequence of SEQ ID NO: 222, or a variant comprising an amino acid sequence having at least about 80% sequence identity,
o) the V_{H} comprises an amino acid sequence of any of SEQ ID NO: 287 and 319-321, or a variant comprising an amino acid sequence having at least about 80% sequence identity, and the V_{L} comprises an amino acid sequence of any of SEQ ID NO: 288, and 322-324, or a variant comprising an amino acid sequence having at least about 80% sequence identity,
p) the V_{H} comprises an amino acid sequence of any one of SEQ ID NOs: 307-312, or a variant comprising an amino acid sequence having at least about 80% sequence identity; and the V_{L} comprises an amino acid sequence of any one of SEQ ID NOs: 313-318, or a variant comprising an amino acid sequence having at least about 80% sequence identity, or
q) the V_{H} comprises an amino acid sequence of any one of SEQ ID NOs: 319-321, or a variant comprising an amino acid sequence having at least about 80% sequence identity; and the V_{L} comprises an amino acid sequence of any one of SEQ ID NOs: 322-324, or a variant comprising an amino acid sequence having at least about 80% sequence identity.

Embodiment 21. The anti-CD93 construct of any one of embodiments 1-20, wherein the antibody moiety is an antibody or antigen-binding fragment thereof selected from the group consisting of a full-length antibody, a bispecific antibody, a single-chain Fv (scFv) fragment, a Fab fragment, a Fab′ fragment, a F(ab′)₂, an Fv fragment, a disulfide stabilized Fv fragment (dsFv), a (dsFv)₂, a Fv-Fc fusion, a scFv-Fc fusion, a scFv-Fv fusion, a diabody, a tribody, and a tetrabody.

Embodiment 22. The anti-CD93 construct of embodiment 21, wherein the antibody moiety is a full-length antibody.

Embodiment 23. The anti-CD93 construct of any one of embodiments 1-22, wherein the antibody moiety has an Fc fragment is selected from the group consisting of Fc fragments form IgG, IgA, IgD, IgE, IgM, and combinations and hybrids thereof.

Embodiment 24. The anti-CD93 construct of embodiment 23, wherein the Fc fragment is selected from the group consisting of Fc fragments from IgG1, IgG2, IgG3, IgG4, and combinations and hybrids thereof.

Embodiment 25. The anti-CD93 construct of embodiment 23 or embodiment 24, wherein the Fc fragment has a reduced effector function as compared to the corresponding wildtype Fc fragment.

Embodiment 26. The anti-CD93 construct of embodiment 23 or embodiment 24, wherein the Fc fragment has an enhanced effector function as compared to the corresponding wildtype Fc fragment.

Embodiment 27. The anti-CD93 construct of any one of embodiments 1-26, wherein the antibody moiety blocks the binding of CD93 to IGFBP7.

Embodiment 28. The anti-CD93 construct of any one of embodiments 1-26, wherein the antibody moiety blocks the binding of CD93 to MMRN2

Embodiment 29. The anti-CD93 construct of any one of embodiments 1-22, wherein the CD93 is a human CD93.

Embodiment 30. A pharmaceutical composition comprising the anti-CD93 construct of any one of embodiments 1-29, and a pharmaceutical acceptable carrier.

Embodiment 31. An isolated nucleic acid encoding the anti-CD93 construct of any one of embodiments 1-28.

Embodiment 32. A vector comprising the isolated nucleic acid of embodiment 31.

Embodiment 33. An isolated host cell comprising the isolated nucleic acid of embodiment 31, or the vector of embodiment 32.

Embodiment 34. An immunoconjugate comprising the anti-CD93 construct of any one of embodiments 1-29, linked to a therapeutic agent or a label.

Embodiment 35. A method of producing an anti-CD93 construct comprising:
a) culturing the isolated host cell of embodiment 33 under conditions effective to express the anti-CD93 construct; and
b) obtaining the expressed anti-CD93 construct from the host cell.

Embodiment 36. A method of treating a disease or condition in an individual, comprising administering to the individual an effective mount of the anti-CD93 construct of any one of embodiments 1-29, or the pharmaceutical composition of embodiment 30.

Embodiment 37. The method of embodiment 36, wherein the disease or condition is associated with an abnormal vascular structure.

Embodiment 38. The method of embodiment 36 or embodiment 37, wherein the disease or condition is a cancer.

Embodiment 39. The method of embodiment 38, wherein the cancer is a solid tumor.

Embodiment 40. The method of embodiment 38 or embodiment 39, wherein the cancer comprises CD93+ endothelial cells.

Embodiment 41. The method of any one of embodiments 38-40, wherein the cancer comprises IGFBP7+ blood vessels.

Embodiment 42. The method of any one of embodiments 38-41, wherein the cancer comprises MMRN2+ blood vessels

Embodiment 43. The method of any one of embodiments 38-42, wherein the cancer is characterized by tumor hypoxia.

Embodiment 44. The method of any one of embodiments 38-43, wherein the cancer is a locally advanced or metastatic cancer.

Embodiment 45. The method of any one of embodiments 38-44, wherein the cancer is selected from the group consisting of a lymphoma, colon cancer, brain cancer, breast cancer, ovarian cancer, endometrial cancer, esophageal cancer, prostate cancer, cervical cancer, renal cancer, bladder cancer, gastric cancer, non-small cell lung cancer, melanoma, and pancreatic cancer.

Embodiment 46. The method of any one of embodiments 36-45, wherein the anti-CD93 construct is administered parenterally into the individual.

Embodiment 47. The method of any one of embodiments 36-46, wherein the method further comprises administering a second therapy.

Embodiment 48. The method of embodiment 47, wherein the second therapy is selected from the group consisting of surgery, radiation, gene therapy, immunotherapy, bone marrow transplantation, stem cell transplantation, hormone therapy, targeted therapy, cryotherapy, ultrasound therapy, photodynamic therapy, and chemotherapy.

Embodiment 49. The method of embodiment 48, wherein the second therapy is an immunotherapy.

Embodiment 50. The method of embodiment 49, wherein the immunotherapy comprises administering an immunomodulatory agent.

Embodiment 51. The method of embodiment 50, wherein the immunomodulatory agent is an immune checkpoint inhibitor.

Embodiment 52. The method of embodiment 51, wherein the immune checkpoint inhibitor comprises an anti-PD-L1 antibody or an anti-PD-1 antibody.

Embodiment 53. The method of any one of embodiments 36-52, wherein the individual is a human.

### EXAMPLES

The examples below are intended to be purely exemplary of the application and should therefore not be considered to limit the application in any way. The following examples and detailed description are offered by way of illustration and not by way of limitation.

### Example 1. Generation of Mouse Anti-Human CD93 Monoclonal Antibodies

Four NZBWF1 mice were immunized with human CD93 recombinant protein (Sino Biologicals). Mice received one prime immunization with a mixture of 100 ug antigen and 100 μL Complete Freund Adjuvant intraperitoneally, followed by 2 boosts of 100 ug antigen mixed with 100 μL of Incomplete Freund Adjuvant intraperitoneally. The serum titer was tested and confirmed by ELISA and FACS assays. A final IP boost with 80 ug of antigen was delivered to mice 5 days before spleen harvest. Single cell suspension of spleen cells from the immunized mice were fused to the mouse myeloma cell line. Fused hybridoma supernatants were screened for specific binding to human CD93 protein by ELISA assay, followed by FACS screen with CD93 expressing CHO cells. Briefly, for FACS screening, the presence of CD93 binding antibodies in the hybridoma supernatant was revealed by goat anti-mouse polyclonal antibody labeled with PE. FACS-positive CD93 specific hybridomas were subcloned and further confirmed by ELISA and FACS assays. Purified monoclonal antibodies were characterized by functional IGFBP7/CD93 blockade and HUVEC tube formation assays. The resulting hybridoma 16E4, 17B10 and 7F3 were identified as representative antibody clones.

### Example 2. Cloning and Sequencing of CD93 Monoclonal Antibodies

Sample Preparation

Total RNA was isolated from the hybridoma cell line culture (2×10⁶ cells). RNA was treated to remove aberrant transcripts and reverse transcribed using oligo (dT) primers. Samples of the resulting cDNA were amplified in separate PCRs using framework 1 and constant region primer pairs specific for either the heavy or light chain. Reaction products were separated on an agarose gel, size-evaluated and recovered. In some cases, a second, nested PCR was performed to increase yield of the desired fragment(s). Amplicons were cloned into pCR®4-TOPO vector using the TA cloning strategy. Fifteen colonies were selected and plasmid DNA was amplified using primers specific for vector DNA sequences. PCR product size for each cloned insert was evaluated by gel electrophoresis, and six reactions were prepared for sequencing using a PCR clean up kit and using cycle sequencing with fluorescent dye terminators and capillary-based electrophoresis. Both PCR products and TA cloned multiple plasmid DNA were subjected to Sanger sequencing.

Sequence Analysis

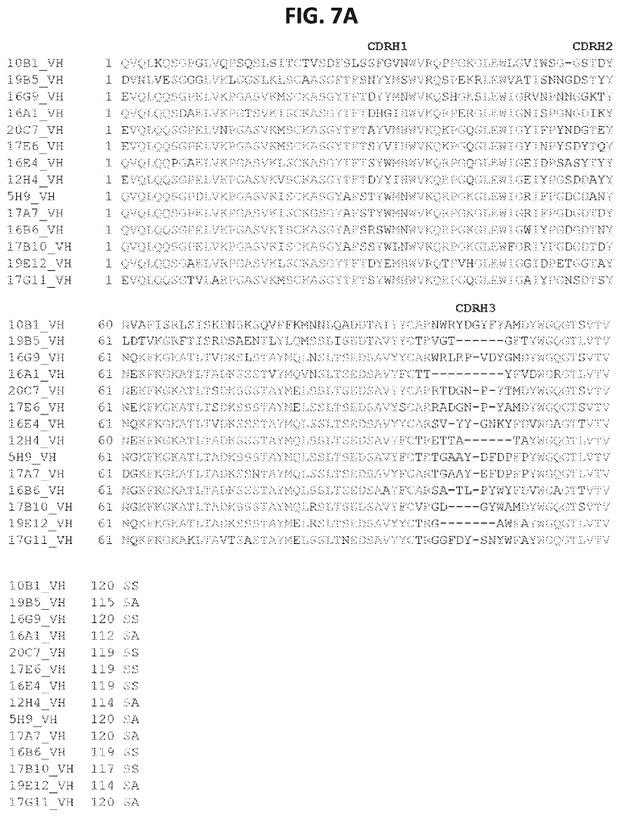

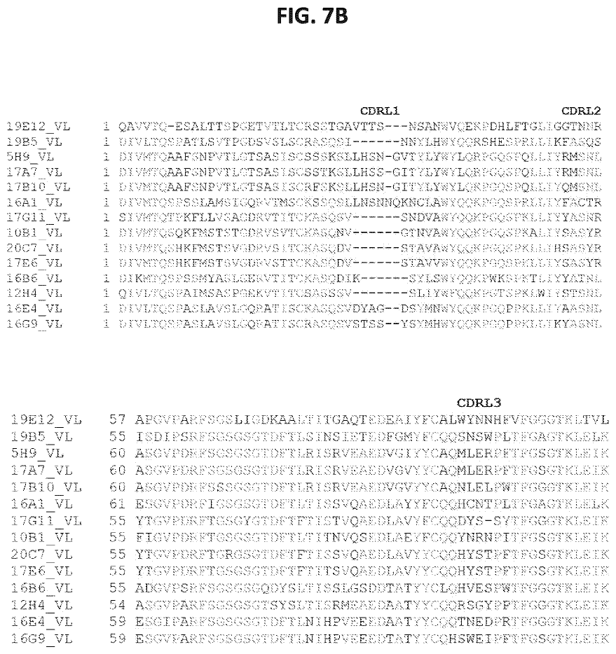

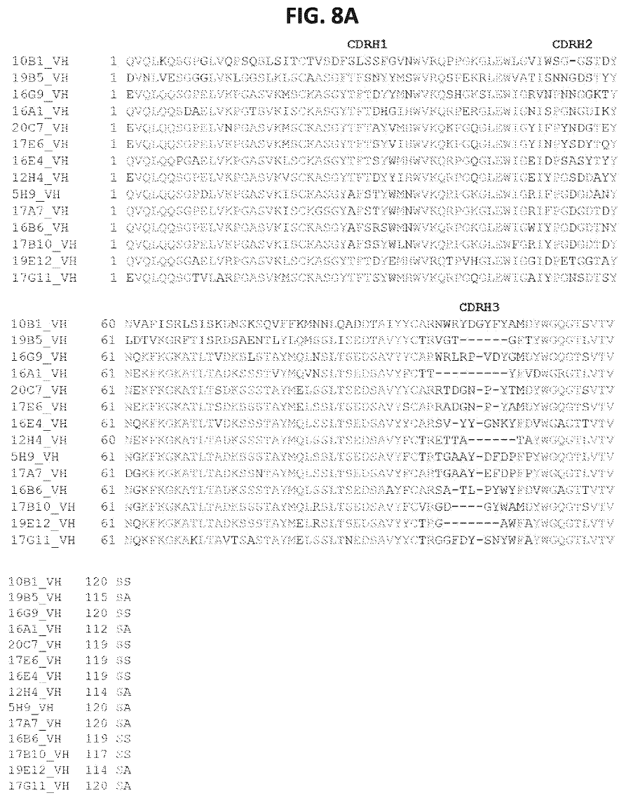

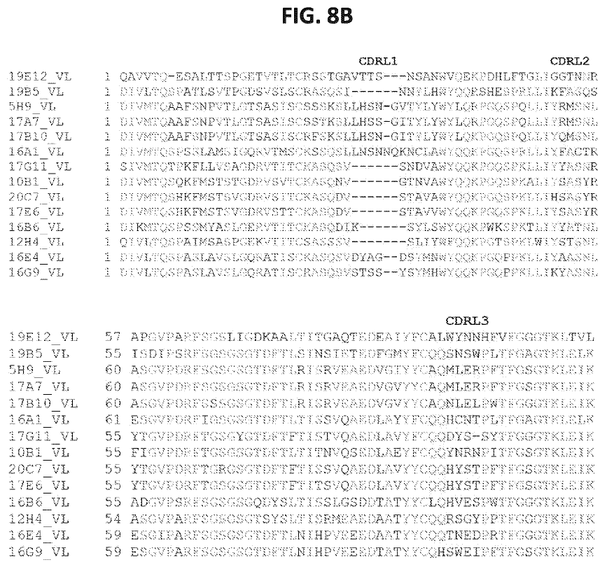

DNA sequence data from all constructs were analyzed and consensus sequences for heavy and light chain were determined. See FIGS. 7A-7B and 8A-8B for alignment of V_{H} and V_{L} CDRs according to Kabat numbering or determined based upon VBASE2 tool. Tables 3 and 4 list V_{H} and VI. CDRs of various antibodies and consensus sequences.

TABLE 3
VH CDRs of various antibodies and consensus sequences.

| | CDRH1 | CDRH2 | CDRH3 |
|---|---|---|---|
| 10B1 | SFGVN | VIWSGGSTDYNVAFIS | NWRYDGYFYAMDY |
| | (SEQ ID NO: 1) | (SEQ ID NO: 2) | (SEQ ID NO: 3) |
| 19B5 | NYYMS | TISNNGDSTYYLDTV | VGTGFTY |
| | (SEQ ID NO: 193) | KG | (SEQ ID NO: 195) |
| | | (SEQ ID NO: 194) | |
| 16G9 | DYYMN | RVNPNNGGKTYNQKF | WRLRP-VDYGMDY |
| | (SEQ ID NO: 49) | KG | (SEQ ID NO: 51) |
| | | (SEQ ID NO: 50) | |
| 16A1 | DHGIH | NISPGNGDIKYNEKFK | YFVD |
| | (SEQ ID NO: 145) | G | (SEQ ID NO: 147) |
| | | (SEQ ID NO: 146) | |
| 20C7 | AYVMH | YIFPYNDGTEYNEKFK | RTDGNPYTMDY |
| | (SEQ ID NO: 113) | G | (SEQ ID NO: 115) |
| | | (SEQ ID NO: 114) | |
| 17E6 | SYVIH | YINPYSDYTQYNEKF | RADGNPY AMDY |
| | (SEQ ID NO: 209) | KG | (SEQ ID NO: 211) |
| | | (SEQ ID NO: 210) | |
| 16E4 | SYWMH | EIDPSASYTYYNQKFK | SVYYGNKYFDV |
| | (SEQ ID NO: 17) | G | (SEQ ID NO: 19) |
| | | (SEQ ID NO: 18) | |
| 12H4 | DYYIH | EIYPGSDDAYYNEKF | ETTATAY |
| | (SEQ ID NO: 129) | KG | (SEQ ID NO: 131) |
| | | (SEQ ID NO: 130) | |
| 5H9 | TYWMN | RIFPGDGDANYNGKF | TGAAYDFDPFPY |
| | (SEQ ID NO: 33) | KG | (SEQ ID NO: 35) |
| | | (SEQ ID NO: 34) | |
| 17A7 | TYWMN | RIFPGDGDTDYDGKF | TGAAYEFDPFPY |
| | (SEQ ID NO: 161) | KG | (SEQ ID NO: 163) |
| | | (SEQ ID NO: 162) | |
| 16B6 | RSWMN | WIYPGDGDTNYNGKF | SATLPYWYFDV |
| | (SEQ ID NO: 97) | KG | (SEQ ID NO: 99) |
| | | (SEQ ID NO: 98) | |
| 17B10 | SYWLN | RIYPGDGDTDYNGKF | GDGYWAMDY |
| | (SEQ ID NO: 177) | KG | (SEQ ID NO: 179) |
| | | (SEQ ID NO: 178) | |
| 19E12 | DYEMH | GIDPETGGTAYNQKF | GAWFAY |
| | (SEQ ID NO: 65) | KG | (SEQ ID NO: 67) |
| | | (SEQ ID NO: 66) | |
| 17G11 | SYWMH | AIYPGNSDTSYNQKF | GGFDYSNYWFAY |
| | (SEQ ID NO: 81) | KG | (SEQ ID NO: 83) |
| | | (SEQ ID NO: 82) | |
| 7F3 | DYEMH | GIDPETGDTAYNQNF | YGNLYYYAMDY |
| | (SEQ ID NO: 289) | KG | (SEQ ID NO: 291) |
| | | (SEQ ID NO: 290) | |
| Consensus | TYWMN | RIFPGDGDX₁X₂YX₃GK | TGAAYX₁FDPFPY |
| sequence | (SEQ ID NO: 33) | FKG | X₁ = D or E |
| based upon | | X₁X₂ = AN or TD, X₃ = | (SEQ ID NO: 234) |
| 5H9/17A7 | | N or D | |
| | | (SEQ ID NO: 233) | |
| Consensus | X₁YWX₂N | RIX₁PGDGDX₂X₃YX₄G | |
| sequence | X₁ = S or T, | KFKG | |
| based upon | X₂ = L or M | X₁ = Y or F, X₂X₃ = TD or | |
| 5H9/17A7/ | (SEQ ID NO: 236) | AN, X₄ = N or D | |
| 17B10 | | (SEQ ID NO: 237) | |
| Consensus | X₁YVX₂H | YIX₁PYX₂DX₃TX₄YNE | RX₁DGNPYX₂MDY |
| sequence | X₁ = A or S, X₂ = M | KFKG | X₁ = T or A, X₂ = T or A |
| based upon | or I | X₁ = F or N, X₂ = N or S, | (SEQ ID NO: 243) |
| 20C7/17E6 | (SEQ ID NO: 241) | X₃ = G or Y, X₄ = E or Q | |
| | | (SEQ ID NO: 242) | |

TABLE 4
VL CDRs of various antibodies and consensus sequences.

| | CDRL1 | CDRL2 | CDRL3 |
|---|---|---|---|
| 19E12 | RSSTGAVTTSNSAN | GTNNRAP | ALWYNNHFV |
| | (SEQ ID NO: 68) | (SEQ ID NO: 69) | (SEQ ID NO: 70) |
| 19B5 | RASQSINNYLH | FASQSIS | QQSNSWPLT |
| | (SEQ ID NO: 196) | (SEQ ID NO: 197) | (SEQ ID NO: 198) |
| 5H9 | SSSKSLLHSNGVTYLY | RMSNLAS | AQMLERPFT |
| | (SEQ ID NO: 36) | (SEQ ID NO: 37) | (SEQ ID NO: 38) |
| 17A7 | SSTKSLLHSSGITYLY | RMSNLAS | AQMLERPFT |
| | (SEQ ID NO: 164) | (SEQ ID NO: 165) | (SEQ ID NO: 166) |
| 17B10 | RFSKSLLHSNGITYLY | QMSNLAS | AQNLELPWT |
| | (SEQ ID NO: 180) | (SEQ ID NO: 181) | (SEQ ID NO: 182) |
| 16A1 | KSSQSLLNSNNQKNCLA | FACTRES | QQHCNTPLT |
| | (SEQ ID NO: 148) | (SEQ ID NO: 149) | (SEQ ID NO: 150) |
| 17G11 | KASQSVSNDVA | YASNRYT | QQDYSSYT |
| | (SEQ ID NO: 84) | (SEQ ID NO: 85) | (SEQ ID NO: 86) |
| 10B1 | KASQNVGTNVA | SASYRFI | QQYNRNPIT |
| | (SEQ ID NO: 4) | (SEQ ID NO: 5) | (SEQ ID NO: 6) |
| 20C7 | KASQDVSTAVA | SASYRYT | QQHYSTPFT |
| | (SEQ ID NO: 116) | (SEQ ID NO: 117) | (SEQ ID NO: 118) |
| 17E6 | KASQDVSTAVV | SASYRYT | QQHYSTPFT |
| | (SEQ ID NO: 212) | (SEQ ID NO: 213) | (SEQ ID NO: 214) |
| 16B6 | KASQDIKSYLS | YATNLAD | LOHVESPWT |
| | (SEQ ID NO: 100) | (SEQ ID NO: 101) | (SEQ ID NO: 102) |
| 12H4 | SASSSVSLIY | STSNLAS | QQRSGYPPT |
| | (SEQ ID NO: 132) | (SEQ ID NO: 133) | (SEQ ID NO: 134) |
| 16E4 | KASQSVDYAGDSYMN | AASNLES | QQTNEDPRT |
| | (SEQ ID NO: 20) | (SEQ ID NO: 21) | (SEQ ID NO: 22) |
| 16G9 | RASQSVSTSSYSYMH | YASNLES | QHSWEIPFT |
| | (SEQ ID NO: 52) | (SEQ ID NO: 53) | (SEQ ID NO: 54) |
| 7F3 | RASSSVSSSYLH | STSNLAF | QQYSGYPLT |
| | (SEQ ID NO: 292) | (SEQ ID NO: 293) | (SEQ ID NO: 294) |
| Consensus | SSX₁KSLLHSX₂GX₃TYLY | RMSNLAS | AQMLERPFT |
| sequence | X₁ = S or T, X₂ = N or S, | (SEQ ID NO: 37) | (SEQ ID NO: 38) |
| based upon | X₃ = V or I | | |
| 5H9/17A7 | (SEQ ID NO: 235) | | |
| Consensus | X₁X₂X₃KSLLHSX₄GX₅TYLY | X₁MSNLAS | AQX₁LEX₂PX₃T |
| sequence | X₁X₂X₃ = SSS, SST, or RFS, | X₁ = R or Q | X₁ = M or N, X₂ = R or |
| based upon | X₄ = N or S, X₅ = V or I | (SEQ ID NO: 239) | L, X₃ = F or W |
| 5H9/17A7/ | (SEQ ID NO: 238) | | (SEQ ID NO: 240) |
| 17B10 | | | |
| Consensus | KASQDVSTAVX₁ | SASYRYT | QQHYSTPFT |
| sequence | X₁ = A or V | (SEQ ID NO: 117) | (SEQ ID NO: 118) |
| based upon | (SEQ ID NO: 244) | | |
| 20C7/17E6 | | | |
| Consensus | KASQX₁VX₂TX₃VX₄ | SASYRX₁X₂ | QQX₁X₂X₃X₄PX₅T |
| sequence | X₁ = N or D, X₂ = G or S, | X₁ = F or Y, X₂ = I or T | X₁X₂X₃X₄ = YNRN |
| based upon | X₃ = N or A, X₄ = A or V | X₁X₂ = FI or YT | or HYST, X₅ = I or F |
| 10B1/20C7/ | (SEQ ID NO: 245) | (SEQ ID NO: 246) | (SEQ ID NO: 247) |
| 17E6 | | | |
| Consensus | X₁ASQSVX₂X₃X₄X₅X₆SYMX₇ | X₁ASNLES | QX₁X₂X₃X₄X₅PX₆T |
| sequence | X₁ = K or R, | X₁ = A or Y | X₁X₂X₃X₄X₅ = QTN |
| based upon | X₂X₃X₄X₅X₆ = DYAGD or | (SEQ ID NO: 249) | ED or HSWEI, |
| 16E4/16G9 | STSSY, X₇ = N or H | | X₆ = R or F |
| | (SEQ ID NO: 248) | | (SEQ ID NO: 250) |

The consensus sequences are compared to known variable region sequences to rule out artifacts and/or process contamination. Consensus sequences are then analyzed using an online tool to verify that the sequences could encode a productive immunoglobulin.

### Example 3. Binding Affinity of Anti-CD93 Antibodies for Human and Cynomolgus CD93 Measured by Bio-Layer Interferometry (BLI) Assay

The binding affinity of anti-CD93 antibodies were determined with bio-layer interferometry using Octet QKe (Fortebio). Human CD93 recombinant protein (Sino Biological Inc, Catalog #12589-H08H) or cynomolgus CD93 protein (made in-house) were biotinylated using EZ-LINK NHS-PEG4 biotin (Thermo Fisher Scientific). Streptavidin biosensors (Fortebio) were used to load biotinylated CD93 protein (300 seconds at 5 μg/ml). The baseline was stabilized for 60 seconds in a 1× kinetics buffer (Fortebio) before serially diluted anti-CD93 antibodies were allowed to associate for 300 seconds with captured protein. The sensors were dissociated in a 1× kinetics buffer for 600 seconds. Data analysis was performed on ForteBio Data Analysis HT 11.1 software.

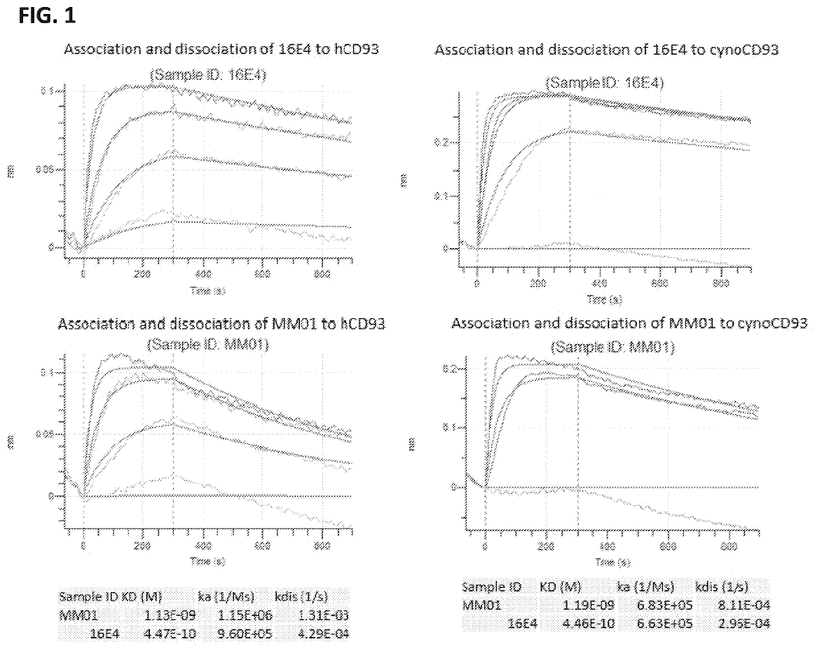

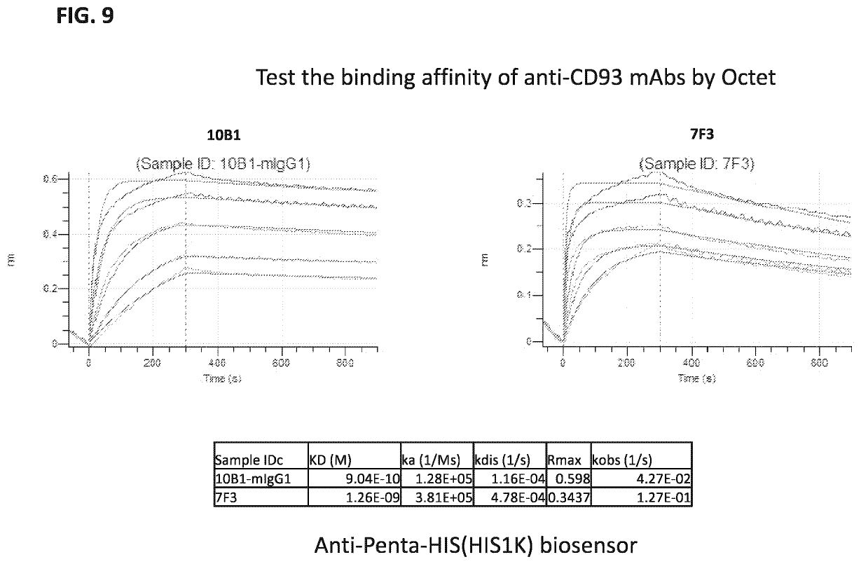

As shown in FIG. 1 and FIG. 9, 16E4, 10B1, 7F3, and reference antibody MM01 all effectively bind to human CD93. 16E4 and MM01 bind to cynomolgus CD93 as well (FIG. 1). 10B1 and 7F3 also bind to cynomolgus CD93 (data not shown).

### Example 4. Binding of Anti-CD93 Antibodies to Cell Surface Expressing Human CD93 CHO Cells Determined by Fluorescence Activated Cell Sorting (FACS) Assay

Human CD93 expressing CHO cells were detached by incubation with TrypLE reagents (Thermos Fisher), which preserves the integrity of CD93 on the cell surface. The cells were then incubated with anti-CD93 antibodies and reference antibody MM01 (Sino Biological Inc, Catalog #12589-MM01) at 10 μg/ml for 30 minutes in 4° C. After washing with FACS buffer, the cells were incubated with Alexa Fluor 488 conjugated anti-human IgG or anti-mouse IgG antibodies (Jackson ImmunoResearch) for 30 minutes at 4° C. After washing with FACS buffer twice, the samples were acquired in NovoCyte Flow Cytometer and analyzed by NovoExpress software. Antibodies 16E4, 10B1, and 7F3 were tested similarly for binding to CHO-K1 cells.

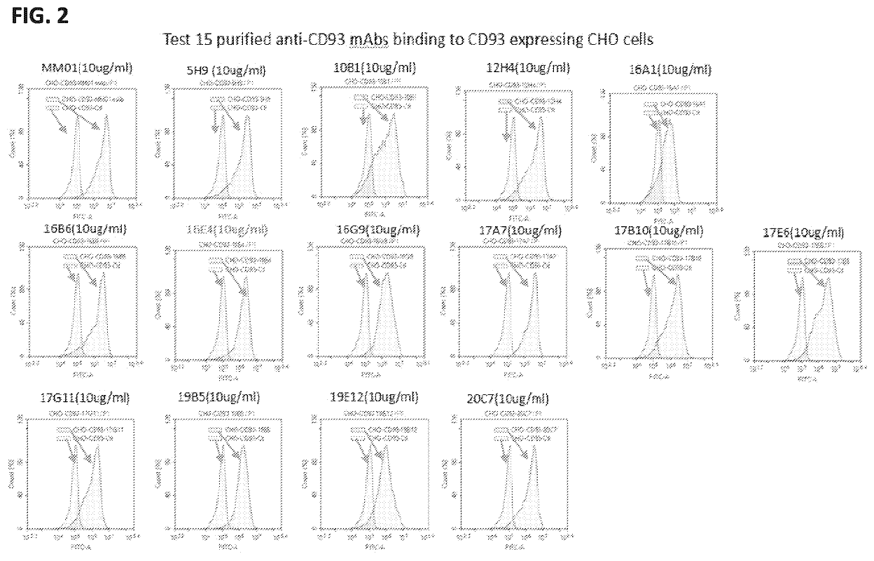

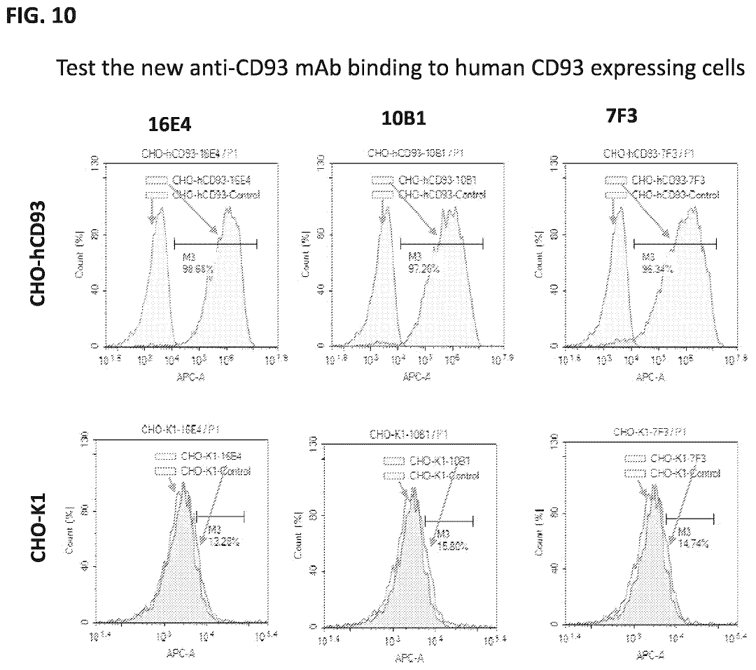

As shown in FIG. 2 and FIG. 10, all fifteen hybridoma clones, as well as commercially available antibody MM01, bind to hCD93 expressing CHO cells (as evidenced by separation of peaks corresponding to anti-CD93 mAbs and control), and there is no binding between CHO-K1 cells and 16E4, 10B1, or 7F3 (as evidenced by no separation of peaks).

### Example 5. IGFBP7/CD93 Blockade Assay in Human CD93 Expressing CHO Cells by Anti-CD93 Antibody Treatment

Human CD93 expressing CHO cells (1×10⁵ per well) were treated with anti-CD93 antibodies or isotype control at a serial concentration for 30 minutes at 4° C. Then the cells were incubated with HIS tagged human IGFBP7 recombinant protein (0.1 μg/ml) for another 30 minutes at 4° C. Then the cells were washed with FACS buffer and incubated with a rabbit anti-IGFBP7 antibody (Sino Biological Inc, Catalog #13100-R003) at 1 μg/ml for 30 minutes at 4° C. After incubation, the cells were washed with FACS buffer and incubated with PE-conjugated anti-rabbit IgG antibody (Biolegend) for 30 minutes in 4° C. After washing by FACS buffer twice, the samples were analyzed and data acquired in NovoCyte Flow.

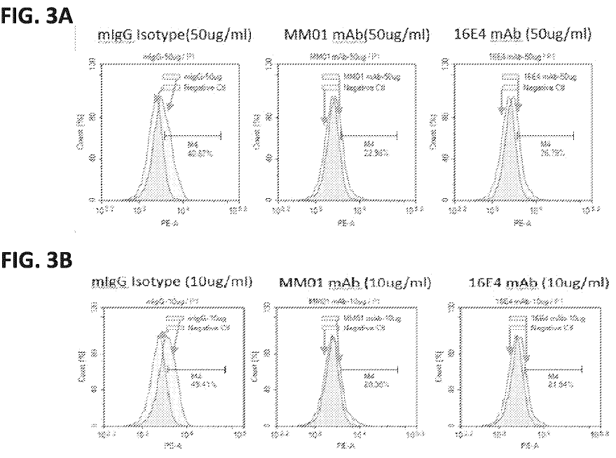

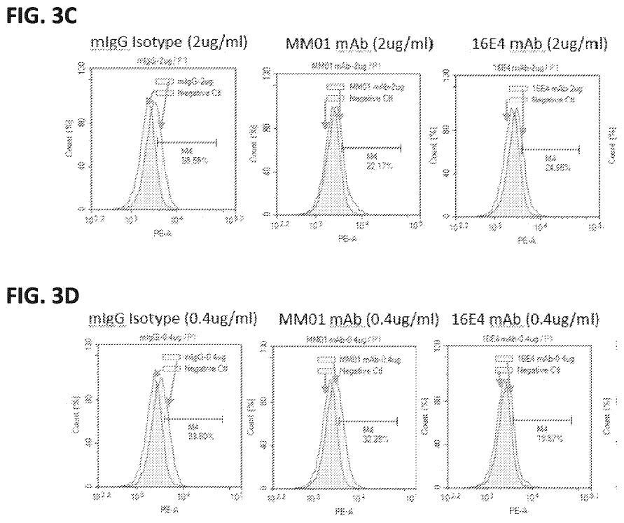

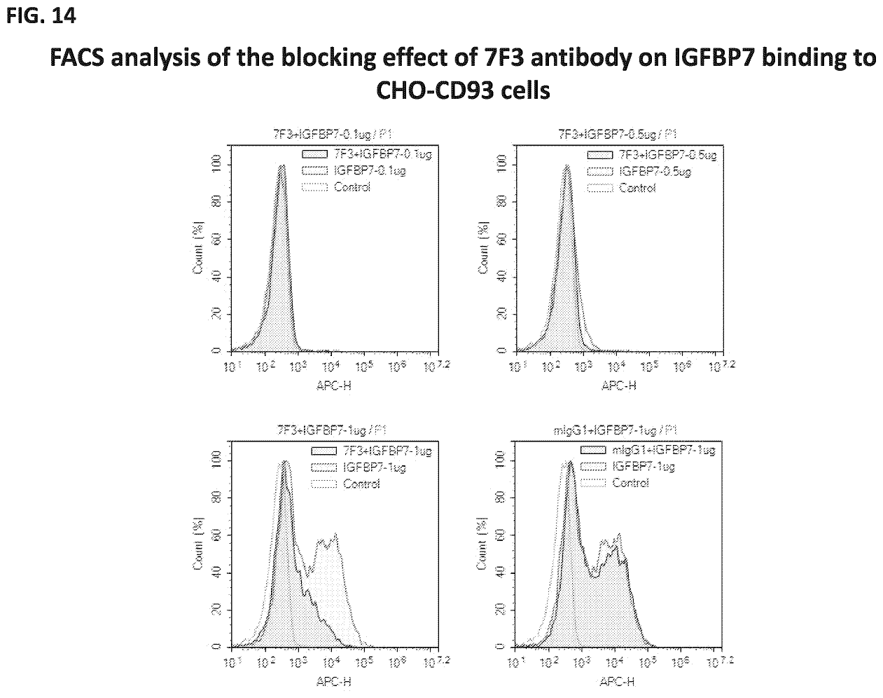

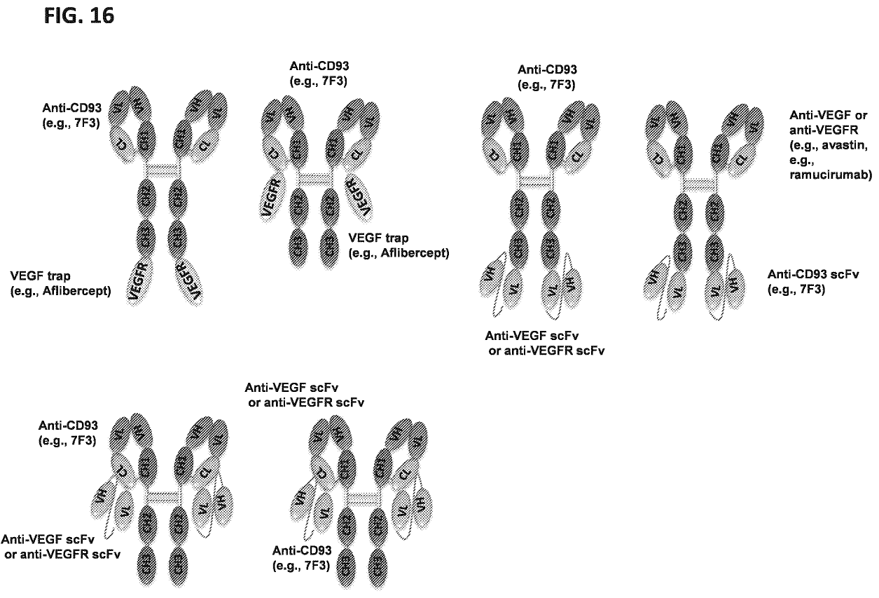

As shown in FIGS. 3A-3D, 16E4 mAb effectively blocks the interaction between CD93 and IGFBP7 at various concentrations, including at the lowest concentration of 0.4 μg/ml (as evidenced by reduction of separation between peaks corresponding to anti-CD93 mAbs and negative controls). FIG. 14 shows that 7F3 effectively blocks the interaction between CD 93 and IGFBP7 at 50 μg/ml (as evidenced by disappearance of the “shoulder” for the control peak).

### Example 6. MMRN2/CD93 Blockade Assay in Human CD93 Expressing CHO Cells by Anti-CD93 Antibody Treatment

Human CD93 expressing CHO cells (1×10⁵ per well) were treated with anti-CD93 antibodies (16E4, 10B1, and 7F3) or isotype control at 50 μg/ml for 30 minutes at 4° C. The cells were then incubated with His-tagged MMRN2 recombinant protein or biotinylated MMRN2 protein (0.1-0.5 μg/ml) for another 30 minutes at 4° C. After incubation, the cells were washed with FACS buffer and incubated with anti-His conjugated APC or streptavidin conjugated APC at a ratio of 1:500 for 30 minutes at 4° C. After washing with FACS buffer twice, the samples were analyzed and data acquired in NovoCyte Flow.

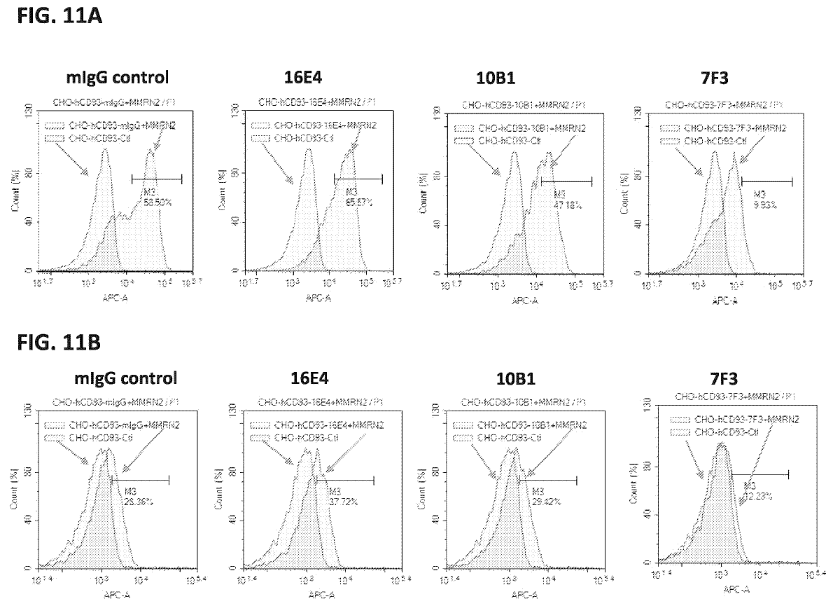

As shown in FIGS. 11A-11B, 7F3 mAb effectively blocks the interaction between MMRN2 and CD93 (as evidenced by reduction of the separation between peaks corresponding to 7F3 mAb and control; FIG. 11A: 0.5 μg/ml of MMRN2; FIG. 11B: 0.1 μg/ml). 16E4 and 10B1 show no significant blockade of the interactions between MMRN2 and CD93.

The blockade of CD93/MMRN2 by 7F3 mIgG1, 5H9 mIgG2a, and 16E4 mIgG2a was further tested as described above at 0.1 μg/ml MMRN2⁴⁹⁵⁻⁶⁷⁴ and 0.5 μg/ml MMRN2⁴⁹⁵⁻⁶⁷⁴ (produced in-house), with IgG2a as negative control.

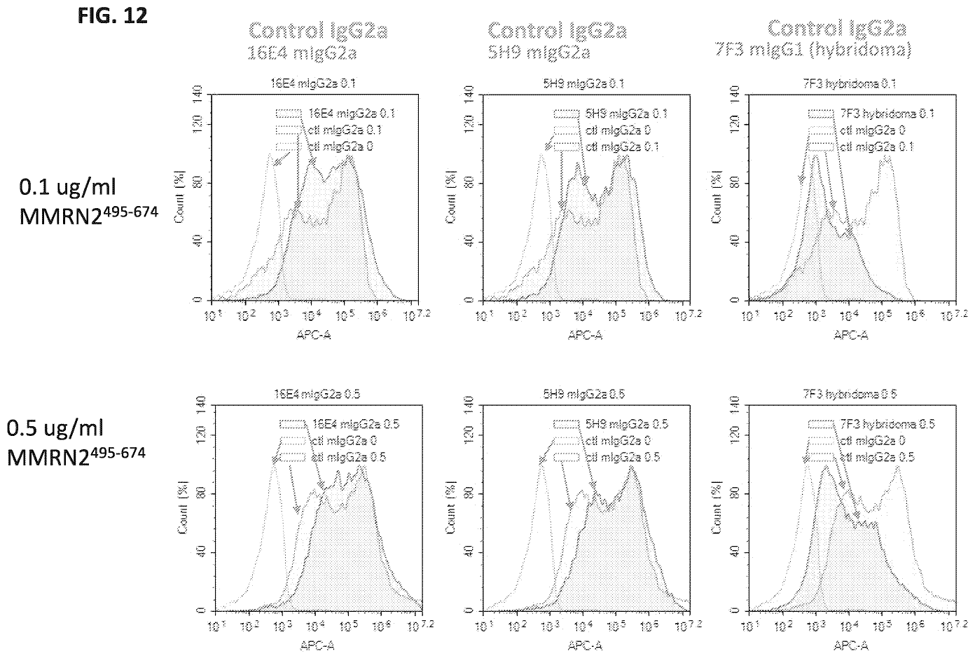

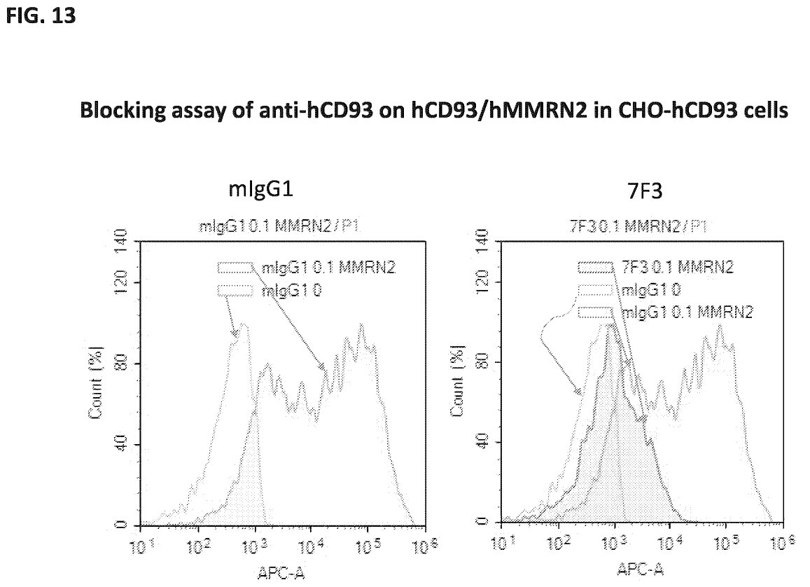

As shown in FIG. 12, 7F3 effectively blocks CD93/MMRN2 interaction at 0.1 μg/ml MMRN2⁴⁹⁵⁻⁴⁷⁴ and as high as 0.5 μg/ml MMRN2⁴⁹⁵⁻⁶⁷⁴ (as evidenced by shift of the 7F3 peak to the left. 7F3 also effectively blocks CD93/MMRN2 interaction at 0.1 μg/ml MMRN2, as shown in FIG. 13 (as evidenced by shift of the 7F3 peak to the left).

### Example 7. HUVEC Tube Forming Inhibition Assay

Human umbilical vein endothelial cells (HUVECs, Thermo Fisher Scientific, Waltham, MA) were cultured in medium 200 supplemented with low serum growth supplement (LSGS, Thermo Fisher Scientific, Waltham, MA) at 37° C. with 5% CO₂. 96 well plates were coated with 50 Al of Geltrex reduced growth factor basement membrane matrix (Thermo Fisher Scientific) and incubated for 30 min at 37° C. To investigate the effects of hybridoma antibodies on tube formation, 2×10⁴ HUVEC cells were seeded onto Matrix-coated plates and incubated in the presence or absence of purified hybridoma antibodies for 18 hours at 37° C. with 5% CO₂. Avastin-IL10 fusion protein was used as a control. Images were obtained using a light microscope.

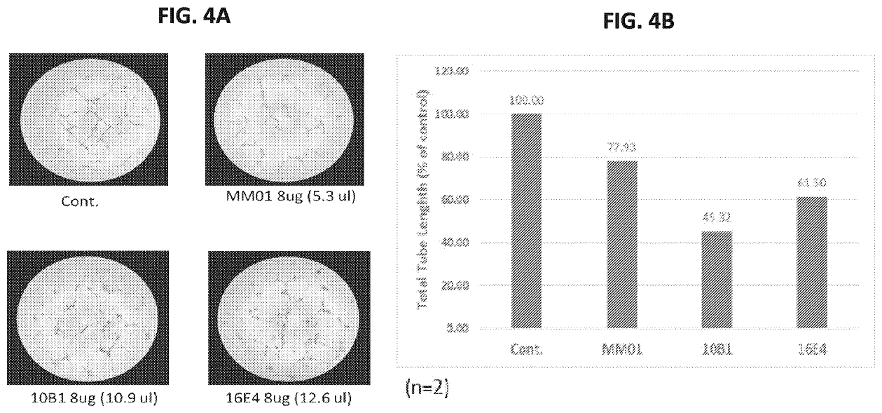

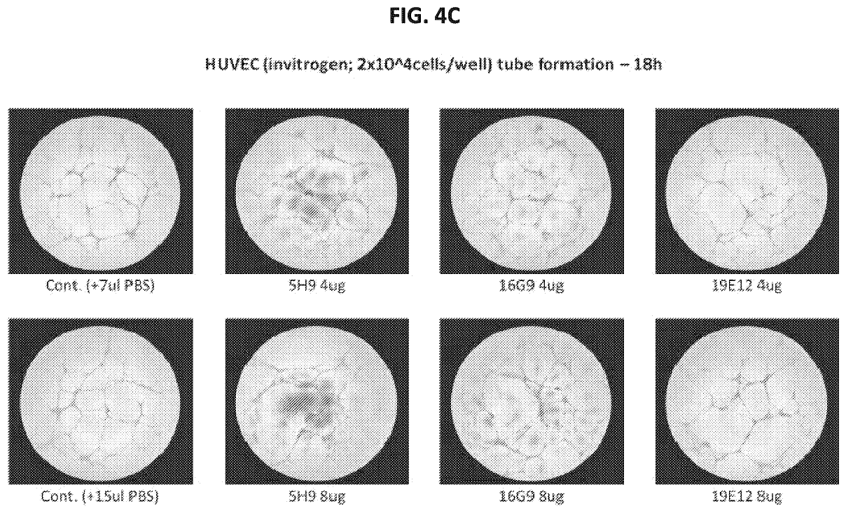

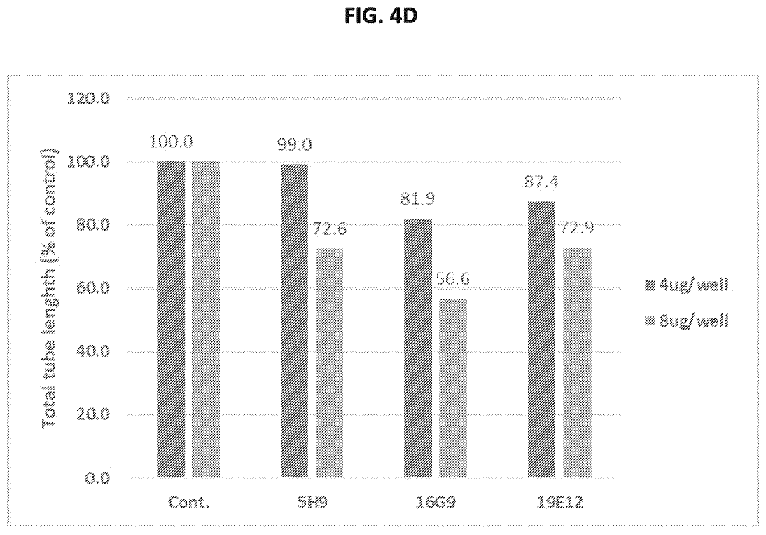

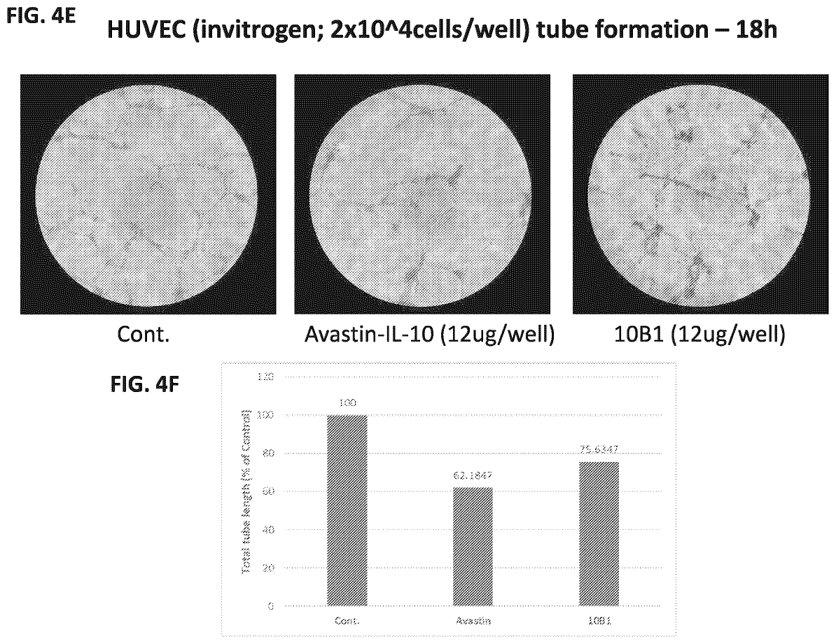

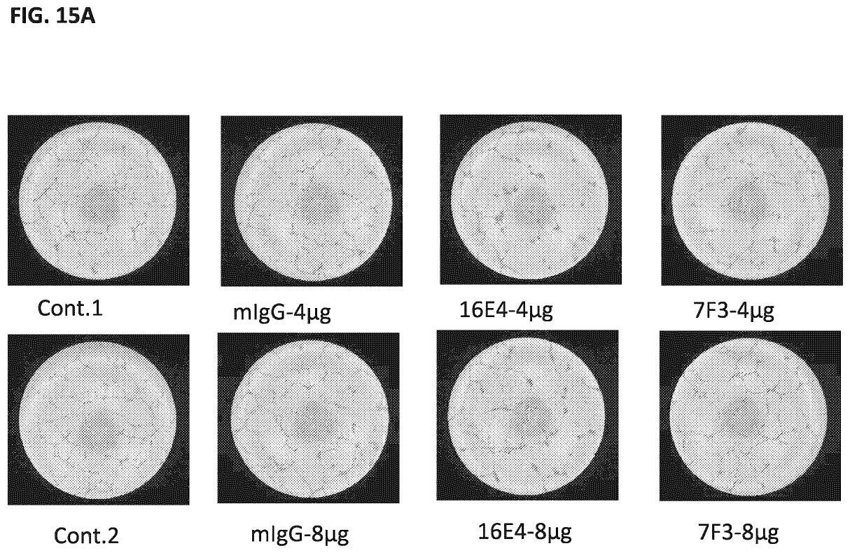

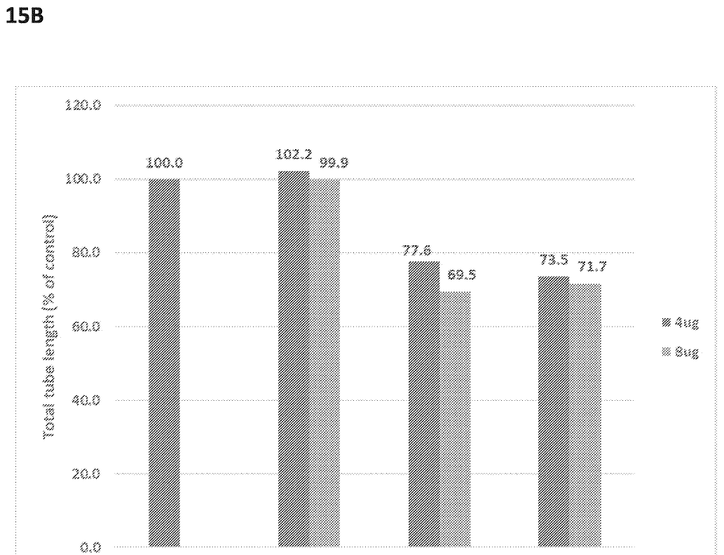

As shown in FIGS. 4A-4F and FIGS. 15A-15B, hybridoma antibodies including 10B1, 16E4, 5H9, 16G9, 19E12 and 7F3 effectively inhibit tube formation at the concentration of 4 μg/ml and/or 8 μg/ml. Specifically, total tube lengths of HUVECs treated with 10B1 or 16E4 decrease to 45% and 61.5% as compared to that of the negative control. Total tube lengths of HUVECs treated with 7F3 at 8 μg/ml decreases to 71.7% as compared to that of the negative control, and to 73.5% at 4 μg/ml. 10B1 achieved a comparable inhibitory effects as Avastin at the same dose.

### Example 8. Epitope Binning Assay of Anti-CD93 Antibodies by Octet Competition

Anti-CD93 antibody epitope bins were determined using Octet QKe (Fortebio). Human CD93 recombinant protein (Sinn Biological Inc, Catalog #12589-H08H) were biotinylated using EZ-LINK NHS-PEG4 biotin (Thermo Fisher Scientific). Streptavidin biosensors tips (Fortebio) were used to capture biotinylated human CD93 protein (300 seconds in 5 μg/ml). The baseline was stabilized for 60 seconds in 1× kinetics buffer (Fortebio) before primary anti-CD93 antibodies (10 μg/ml) were allowed to associate for 300 seconds with captured protein. A panel of secondary anti-CD93 antibodies (10 μg/ml) were then allowed to associate with the antigen and primary antibody complex for additional 300 seconds. Signals were recorded for each binding event and data analysis was performed on ForteBio Data Analysis HT 11.1 software.

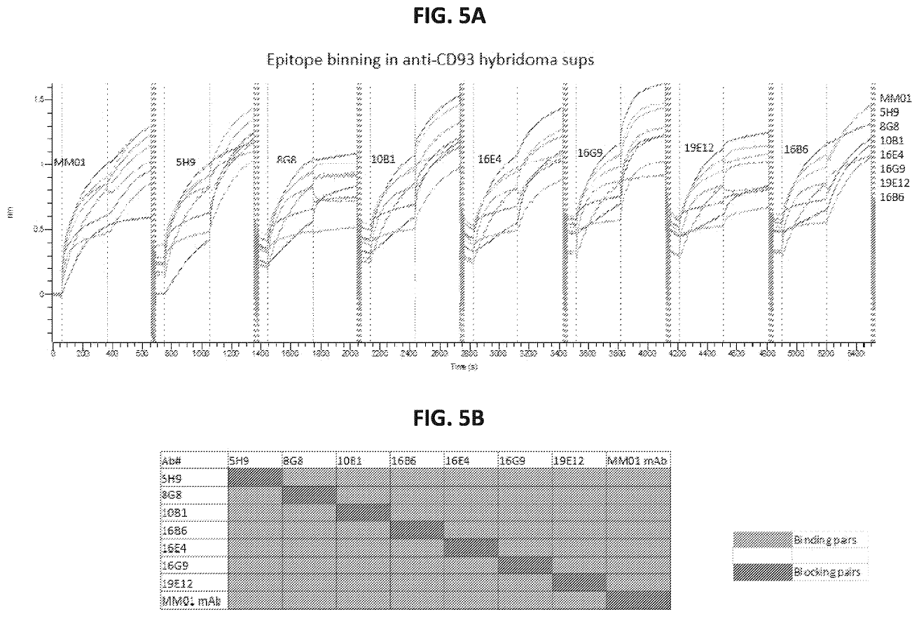

As shown in FIGS. 5A-5B, 5H9, 10B1, 16E4, 16G9, 19E12, 16B6, and MM01 serve as binding pairs among themselves, indicating that they bind to different epitopes on CD93.

### Example 9. Human and Cynomolgus CD93 Antigen Cross-Binding Activities of Anti-CD93 mAbs Measured by Bio-Layer Interferometry (BLI) Assay

The binding affinity of anti-CD93 antibodies were determined with bio-layer interferometry using Octet QKe (Fortebio). Human CD93 recombinant protein (Sino Biological Inc, Catalog #12589-H08H) or cynomolgus CD93 protein (made in-house) were biotinylated using EZ-LINK NHS-PEG4 biotin (Thermo Fisher Scientific). Streptavidin biosensors (Fortebio) were used to load biotinylated CD93 protein (300 seconds in 5 μg/ml). The baseline was stabilized for 60 seconds in 1× kinetics buffer (Fortebio) before anti-CD93 antibodies at a serial dilution were allowed to associate for 300 seconds with captured protein. Then the sensors were dissociated in 1× kinetics buffer for 600 seconds. Data analysis was performed on ForteBio Data Analysis HT 11.1 software.

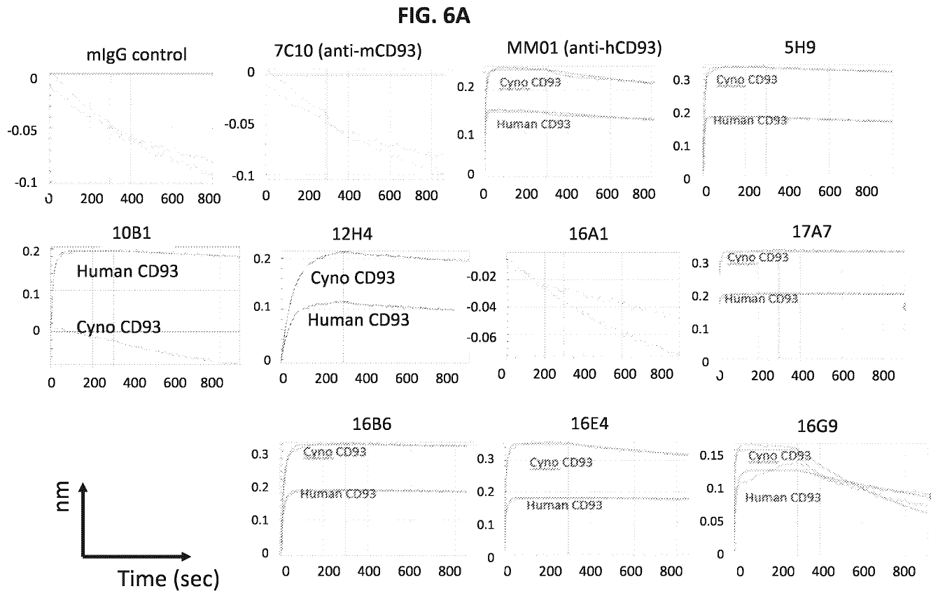

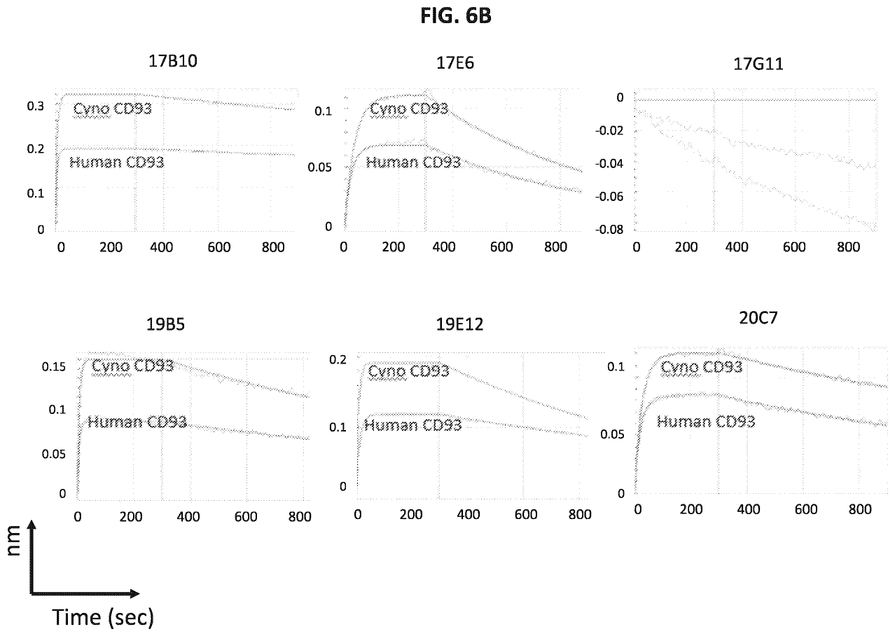

As shown in FIGS. 6A-6B, 5H9, 12H4, 16B6, 16E4, 16G9, 17A7, 17B10, 17E6, 19B5, 19E12, 20C7 as well as MM01 cross-reacted with cynomolgus CD93, while 7C10, 16A1, and 17G11 did not cross-react with cynomolgus CD93.

Table 5 is a summary of the properties of various anti-CD93 antibodies.

TABLE 5
Summary of properties of various anti-CD93 antibodies.

| | | Blocking | Blocking | | |
|---|---|---|---|---|---|
| | | between | between | | |
| | | CD93 and | CD93 and | HUVEC | |
| | | IGFBP7 | MNRN2 | Tube | Cyno Cross |
| Clone Name | Binding | (FACS) | (FACS) | inhibition | (ELISA) |
| 10B1 | +++ | + | − | +++ | + |
| 16E4 | +++ | +++ | − | +++ | + |
| 5H9 | +++ | + | N.D. | + | + |
| 19E12 | ++ | + | N.D. | + | + |
| 16B6 | +++ | − | N.D. | + | + |
| 17G11 | +++ | − | N.D. | ++ | + |
| 20C7 | +++ | − | N.D. | ++ | + |
| 16G9 | ++ | + | N.D. | + | + |
| 12H4 | +++ | + | N.D. | + | + |
| 16A1 | ++ | − | N.D. | + | + |
| 17A7 | +++ | − | N.D. | − | + |
| 17B10 | +++ | + | N.D. | + | + |
| 17E6 | +++ | − | N.D. | ++ | + |
| 19B5 | ++ | − | N.D. | − | + |
| 7F3 | +++ | +++ | +++ | +++ | + |

### Example 10. Humanization of Anti-CD93 Antibodies and Generation of Anti-CD93 Constructs that Inhibit VEGF

Exemplary humanized anti-CD93 heavy chain variable sequences and light chain variable sequences were generated. See SEQ ID NO: 307-324 and 347-365 in Sequence Table. CDR sequences of 16E4, 17B10, 16A1 and 7F3 humanized heavy chain variable region sequences and light chain variable region sequences were analyzed and shown in Tables 6-7.

TABLE 6
Heavy chain CDRs of anti-CD93 antibodies and humanized sequences.

| | | | | HC |
|---|---|---|---|---|
| | | | | variable |
| | | | | region |
| | HC-CDR1 | HC-CDR2 | HC-CDR3 | sequences |
| 16E4 | SYWMH | EIDPSASYTYYNQKFKG | SVYYGNKYFDV | SEQ ID |
| (parental) | (SEQ ID | (SEQ ID NO: 18) | (SEQ ID NO: 19) | NO: 29 |
| | NO: 17) | | | |
| 16E4 | SYWMH | EIDPSASYTYYNQKFKG | SVYYGNKYFDV | SEQ ID |
| VH1 | (SEQ ID | (SEQ ID NO: 18) | (SEQ ID NO: 19) | NO: 307 |
| | NO: 17) | | | |
| 16E4 | SYWMH | EIDPSASYTYYNQKFKG | SVYYGNKYFDV | SEQ ID |
| VH2 | (SEQ ID | (SEQ ID NO: 18) | (SEQ ID NO: 19) | NO: 308 |
| | NO: 17) | | | |
| 16E4 | SYWMH | EIDPSASYTYYNQKFKG | SVYYGNKYFDV | SEQ ID |
| VH3 | (SEQ ID | (SEQ ID NO: 18) | (SEQ ID NO: 19) | NO: 309 |
| | NO: 17) | | | |
| 16E4 | SYWMH | EIDPSASYTYYNQKFKG | SVYYGNKYFDV | SEQ ID |
| VH4 | (SEQ ID | (SEQ ID NO: 18) | (SEQ ID NO: 19) | NO: 310 |
| | NO: 17) | | | |
| 16E4 | SYWIH | EIEPSASYTYYNQKFKG | SVYYGNKYFDV | SEQ ID |
| VH5 | (SEQ ID | (SEQ ID NO: 305) | (SEQ ID NO: 19) | NO: 311 |
| | NO: 304) | | | |
| 16E4 | SYWMH | EIDPSASYTYYNQKFKG | SVYYGNKYFDV | SEQ ID |
| VH6 | (SEQ ID | (SEQ ID NO: 18) | (SEQ ID NO: 19) | NO: 312 |
| | NO: 17) | | | |
| 17B10 | SYWLN | RIYPGDGDTDYNGKFKG | GDGYWAMDY | SEQ ID |
| (parental) | (SEQ ID | (SEQ ID NO: 178) | (SEQ ID NO: 179) | NO: 189 |
| | NO: 177) | | | |
| 17B10 | SYWLN | RIYPGDGDTDYNGKFKG | GDGYWAMDY | SEQ ID |
| VH1 | (SEQ ID | (SEQ ID NO: 178) | (SEQ ID NO: 179) | NO: 347 |
| | NO: 177) | | | |
| 17B10 | SYWLN | RIYPGDGDTDYNGKFKG | GDGYWAMDY | SEQ ID |
| VH2 | (SEQ ID | (SEQ ID NO: 178) | (SEQ ID NO: 179) | NO: 348 |
| | NO: 177) | | | |
| 17B10 | SYWLN | RIYPGDGDTDYNGKFKG | GDGYWAMDY | SEQ ID |
| VH3 | (SEQ ID | (SEQ ID NO: 178) | (SEQ ID NO: 179) | NO: 349 |
| | NO: 177) | | | |
| 16A1 | DHGIH | NISPGNGDIKYNEKFKG | YFVD (SEQ ID | SEQ ID |
| (parental) | (SEQ ID | (SEQ ID NO: 146) | NO: 147) | NO: 157 |
| | NO: 145) | | | |
| 16A1 | DHGIH | NISPGNGDIKYNEKFKG | YFVD (SEQ ID | SEQ ID |
| VH1 | (SEQ ID | (SEQ ID NO: 146) | NO: 147) | NO: 360 |
| | NO: 145) | | | |
| 16A1 | DHGIH | NISPGNGDIKYNEKFKG | YFVD (SEQ ID | SEQ ID |
| VH2 | (SEQ ID | (SEQ ID NO: 146) | NO: 147) | NO: 361 |
| | NO: 145) | | | |
| 16A1 | DHGIH | NISPGNGDIKYNEKFKG | YFVD (SEQ ID | SEQ ID |
| VH3 | (SEQ ID | (SEQ ID NO: 146) | NO: 147) | NO: 362 |
| | NO: 145) | | | |
| 7F3 | DYEMH | GIDPETGDTAYNQNFKG | YGNLYYYAMDY | SEQ ID |
| (parental) | (SEQ ID | (SEQ ID NO: 290) | (SEQ ID NO: 291) | NO: 287 |
| | NO: 289) | | | |
| 7F3 VH1 | DYEMH | GIDPETGDTAYNQNFKG | YGNLYYYAMDY | SEQ ID |
| | (SEQ ID | (SEQ ID NO: 290) | (SEQ ID NO: 291) | NO: 319 |
| | NO: 289) | | | |
| 7F3 VH2 | DYEMH | GIDPETGDTAYNQNFKG | YGNLYYYAMDY | SEQ ID |
| | (SEQ ID | (SEQ ID NO: 290) | (SEQ ID NO: 291) | NO: 320 |
| | NO: 289) | | | |
| 7F3 VH3 | DYEMH | GIDPETGDTAYNQNFKG | YGNLYYYAMDY | SEQ ID |
| | (SEQ ID | (SEQ ID NO: 290) | (SEQ ID NO: 291) | NO: 321 |
| | NO: 289) | | | |

TABLE 7
Light chain CDRs of anti-CD93 antibodies and humanized sequences.

| | | | | LC variable |
|---|---|---|---|---|
| | | | | region |
| | LC-CDR1 | LC-CDR2 | LC-CDR3 | sequences |
| 16E4 | KASQSVDYAGDSYMN | AASNLES | QQTNEDPRT | SEQ ID |
| | (SEQ ID NO: 20) | (SEQ ID NO: 21) | (SEQ ID NO: 22) | NO: 30 |
| 16E4 | KASQSVDYAGDSYLN | AASNLES | QQTNEDPRT | SEQ ID |
| VL1 | (SEQ ID NO: 301) | (SEQ ID NO: 21) | (SEQ ID NO: 22) | NO: 313 |
| 16E4 | RASQSVDYAGDSYMN | AASNLES | QQTNEDPRT | SEQ ID |
| VL2 | (SEQ ID NO: 302) | (SEQ ID NO: 21) | (SEQ ID NO: 22) | NO: 314 |
| 16E4 | RASQSVDYAGDSYLA | AASNLES | QQTNEDPRT | SEQ ID |
| VL3 | (SEQ ID NO: 303) | (SEQ ID NO: 21) | (SEQ ID NO: 22) | NO: 315 |
| 16E4 | RASQSVDYAGDSYMN | AASNLES | QQTNEDPRT | SEQ ID |
| VL4 | (SEQ ID NO: 302) | (SEQ ID NO: 21) | (SEQ ID NO: 22) | NO: 316 |
| 16E4 | RASQSVDYAGDSYLN | AASNLES | QQTNEDPRT | SEQ ID |
| VL5 | (SEQ ID NO: 306) | (SEQ ID NO: 21) | (SEQ ID NO: 22) | NO: 317 |
| 16E4 | KASQSVDYAGDSYMN | AASNLES | QQTNEDPRT | SEQ ID |
| VL6 | (SEQ ID NO: 20) | (SEQ ID NO: 21) | (SEQ ID NO: 22) | NO: 318 |
| 17B10 | RFSKSLLHSNGITYLY | QMSNLAS (SEQ | AQNLELPWT | SEQ ID |
| (parental) | (SEQ ID NO: 180) | ID No: 181) | (SEQ ID NO: | NO: 190 |
| | | | 182) | |
| 17B10 | RFSQSLLHSNGITYLY | QMSNLAS (SEQ | AQNLELPWT | SEQ ID |
| VL1 | (SEQ ID NO: 353) | ID No: 181) | (SEQ ID NO: | NO: 350 |
| | | | 182) | |
| 17B10 | RFSQSLLHSNGITYLY | TMSNLAS (SEQ | AQNLELPWT | SEQ ID |
| VL2 | (SEQ ID NO: 353) | ID No: 354) | (SEQ ID NO: | NO: 351 |
| | | | 182) | |
| 17B10 | RFSKSLLHSNGITYLY | QMSNLAS (SEQ | AQNLELPWT | SEQ ID |
| VL3 | (SEQ ID NO: 180) | ID No: 181) | (SEQ ID NO: | NO: 352 |
| | | | 182) | |
| 16A1 | KSSQSLLNSNNQKNCL | FACTRES (SEQ | QQHCNTPLT | SEQ ID |
| (parental) | A (SEQ ID NO: 148) | ID NO: 149) | (SEQ ID NO: | NO: 158 |
| | | | 150) | |
| 16A1 | KSSQSLLNSNNQKNYL | FASTRES (SEQ | QQHYNTPLT | SEQ ID |
| VL1 | A (SEQ ID NO: 355) | ID NO: 356) | (SEQ ID NO: | NO: 363 |
| | | | 357) | |
| 16A1 | KSSQSLLNSNNQKNSL | FASTRES (SEQ | QQHSNTPLT | SEQ ID |
| VL2 | A (SEQ ID NO: 358) | ID NO: 356) | (SEQ ID NO: | NO: 364 |
| | | | 359) | |
| 16A1 | KSSQSLLNSNNQKNCL | FASTRES (SEQ | QQHCNTPLT | SEQ ID |
| VL3 | A (SEQ ID NO: 148) | ID NO: 356) | (SEQ ID NO: | NO: 365 |
| | | | 150) | |
| 7F3 | RASSSVSSSYLH (SEQ | STSNLAF (SEQ | QQYSGYPLT | SEQ ID |
| (parental) | ID NO: 292) | ID NO: 293) | (SEQ ID NO: | NO: 288 |
| | | | 294) | |
| 7F3 VL1 | RASSSVSSSYLH (SEQ | STSNLAF (SEQ | QQYSGYPLT | SEQ ID |
| | ID NO: 292) | ID NO: 293) | (SEQ ID NO: | NO: 322 |
| | | | 294) | |
| 7F3 VL2 | RASSSVSSSYLH (SEQ | STSNLAF (SEQ | QQYSGYPLT | SEQ ID |
| | ID NO: 292) | ID NO: 293) | (SEQ ID NO: | NO: 323 |
| | | | 294) | |
| 7F3 VL3 | RASSSVSSSYLH (SEQ | STSNLAF (SEQ | QQYSGYPLT | SEQ ID |
| | ID NO: 292) | ID NO: 293) | (SEQ ID NO: | NO: 324 |
| | | | 294) | |

Various humanized 16E4, 17B10, 16A1 and 7F3 were generated by pairing one of the humanized heavy chain variable region sequences with one of the humanized light chain variable region sequences shown in Tables 6 and 7.

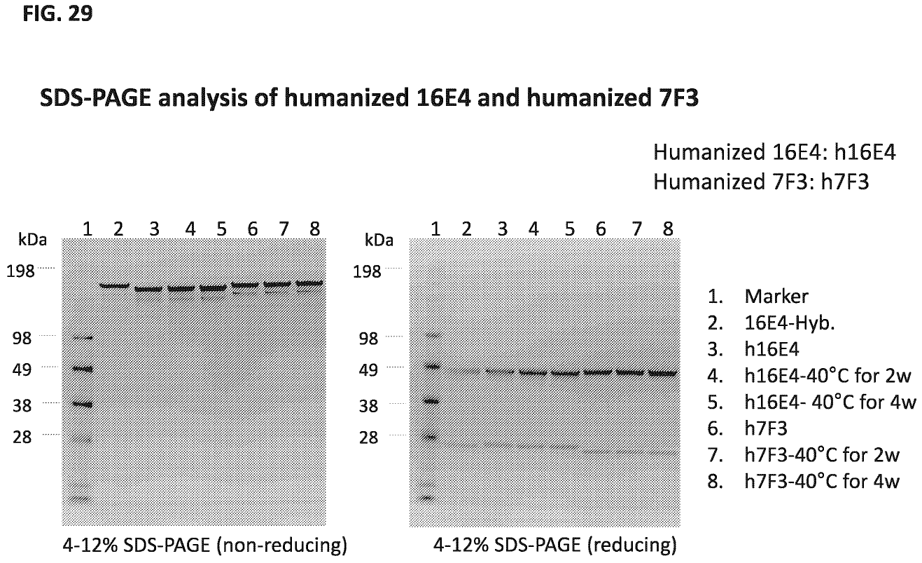

SDS-PAGE stability analysis of humanized 16E4 and 7F3 is shown in FIG. 29. SDS-PAGE was performed under reduced and non-reduced conditions to evaluate the stability of humanized 16E4 and 7F3 antibodies. Humanized 16E4 and 7F3 antibodies were incubated in the dark at 40° C. for two and four weeks. The final samples were run on SDS-PAGE and stained with Coomassie Blue to evaluate any visual changes in the antibodies that could have occurred during the incubation. Parental hybridoma 16E4 was run as a positive control. There was no significant change in the recombinant humanized 16E4 and 7F3 observed by this SDS-PAGE analysis at Day 0, 2 weeks or 4 weeks after incubation.

Anti-CD93 constructs that also target VEGF were designed and generated. See FIG. 16. For example, VEGF-trap (Afibercept, e.g., SEQ ID NO: 325) were fused to C-terminus of two heavy chains of full-length human IgG1 antibody that comprises heavy chain variable region and light chain variable region of any of the 7F3 and its humanized sequences (e.g., SEQ ID NOs: 287, 288 and 319-324) via a linker GSDKTHT (SEQ ID NO: 338). See SEQ ID NOs: 342 and 343 for exemplary heavy chain and light chain sequences. In some embodiments, the heavy chain or light chain further has a signal peptide (such as SEQ ID NO: 344, 345, or 346) fused to the N-terminus of the heavy chain or light chain.

### Example 11. Animal Studies Using 17B10 Antibodies

### 1. Syngeneic B16F10 Model

The anti-tumor effect of the anti-CD93 17B10 antibodies was evaluated in a syngeneic mouse model of B16F10 melanoma at Biocytogen. The 17B10 antibody did not strongly cross-react with mouse CD93 based on Octet and FACS analysis, but did show some binding at high protein concentrations to CD93-HEK cells.

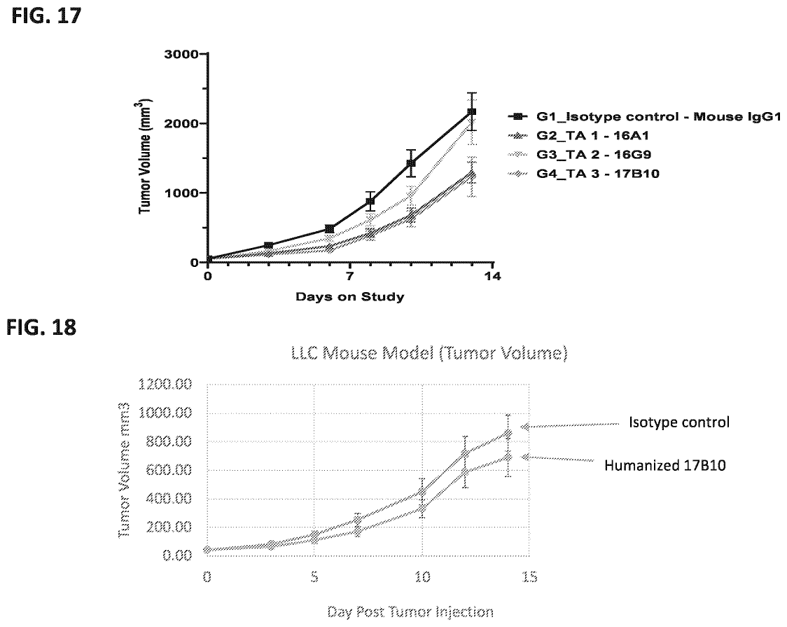

For the syngeneic mouse model, female C57BL/6J mice were implanted with a murine cell line of B16F10 tumor cells (0.2×10⁶) in serum-free media. When tumors reach 40-50 mm³, the mice (n=8 per test article) were randomly assigned to groups. Anti-CD93 antibodies (and isotype control) were dosed at 0.3 mg/mouse intraperitoneally on days 0, 3, 7, and 10. Efficacy was evaluated based on overall tumor volume. Body weight was measured to ensure general health of the animals was not affected by test articles. The 17B10 used in this study was expressed in hybridoma cells and purified over a Protein G column. 16G9 and 16A1 were used as comparisons. Tumor volume in each group is shown in FIG. 17. Mice in 17B10 and 16G9 groups exhibited smaller tumor volume compared to mice in 16A1 group and IgG1 control group, suggesting better anti-tumor effects.

### 2. Lewis Lung Carcinoma

The anti-tumor effect of the humanized anti-CD93 17B10 antibody was evaluated in a syngeneic mouse model of Lewis Lung Carcinoma (LLC). Humanized 17B10 containing a mouse IgG1 Fc was recombinantly produced in ExpiHEK cells. The antibody was purified using a Protein G column, then concentrated and buffer exchanged into 1× PBS. The humanized 17B10 antibody did not strongly cross-react with mouse CD93 based on Octet and FACS analysis, but did show binding at high protein concentrations.

For the syngeneic mouse model, female C57BL/6J mice were implanted with a murine cell line of LLC tumor cells (0.2×10⁶) in serum-free media. When tumors reach 40-50 mm³, the mice (n=7 per test article) were randomly assigned to groups. Anti-CD93 antibodies (and isotype control) were dosed at 0.3 mg/mouse intraperitoneally on days 0, 3, 7, and 10. Efficacy was evaluated based on overall tumor volume. Body weight was measured to ensure general health of the animals was not affected by test articles.

FIG. 18 shows tumor volume+/−SEM from baseline. FIG. 18 demonstrates that mice in 17B10 group exhibited lower tumor volume compared to mice in the isotype control group.

### 3. Knock-In Mouse Model Development

Knock-in mouse model was developed using two methods. The knock-in model was designed to replace the mouse CD93 protein with human CD93 protein.

CRISPR/Cas9 was utilized to make two cuts with a guide RNA #1 targeting near the ATG at the 5′UTR of mouse CD93, and the guide RNA #2 targeting near the beginning of the 3′UTR. Homology directed repair used a donor to fuse in-frame the mouse 5′UTR with the CD93 human cDNA and enable expression from the endogenous CD93 promotor. The repair downstream of the STOP codon ensured that the CD93 hybrid transcript contains the mouse 3′UTR. Pure C57BL/6N mice were used as the background for the knock in model. Embryonic stem cell clones were produced and expanded with the knock-in human CD93 gene. Following sequence confirmation, a blastocyst injection was performed to establish the chimeric founders. Breeding proceeded from there with genotyping to identify heterozygote and homozygote pups.

Alternatively, CRISPR/Cas9 was utilized to remove the mouse exon 1 of CD93 corresponding to the extracellular domain of CD93 (S25-N572). In homology directed repair, the donor DNA contained the human sequence of CD93 from T26-K580. The resulting construct expressed a protein containing the humanized extracellular domain of CD93 with the mouse transmembrane and intracellular domains. C57BL/6 mouse embryonic stem cells were utilized for the knock-in model following sequence confirmation. Ozgene used its proprietary Go-Germiline blastocyst for the injections to establish the chimeric founders. Genotyping and phenotyping was performed to ensure heterozygote and homozygote mice.

### Example 12. Anti-CD93 Antibodies Binding to CD93 Expressing Cells Determined by Flow Cytometry

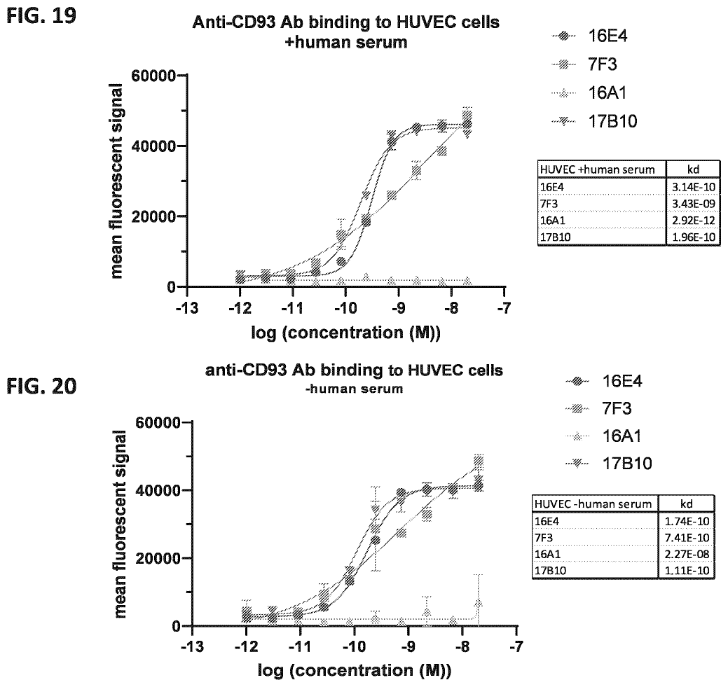

Recombinant parental anti-CD93 antibodies were evaluated for their ability to bind to HUVEC cells in the presence or absence of human serum. The 16E4, 7F3, 16A1, and 17B10 sequences obtained from the hybridoma cells were expressed recombinantly with a human CH1 domain and mouse IgG1 CH2 and CH3 Fc domains. Antibodies were purified using Protein G Sepharose. The resulting antibodies were tested for its binding capacity to a variety of cells that express CD93. HUVEC cells were detached by incubation with TrypLE reagent (Gibco cat #12604-013), which preserves the integrity of CD93 on the cell surface. Cells were quenched with media then counted. Cells were resuspended in FACS buffer (ice cold PBS with 0.5% BSA) and human serum was added to 20% (10% final volume) and put on ice for approximately 20 minutes. 5×10⁴ cells were seeded per well in 100 μL media and incubated with serial diluted anti-CD93 antibodies in 100 μL on ice for 2 hours. Cells were then washed by spinning cells at 1200 rpm for 5 min. Media was discarded and cells were resuspended in 200 μL ice cold FACS buffer. The wash step was repeated and cells were resuspended in 100 μL of secondary antibody, AlexaFluor647 conjugated anti-human IgG or anti-mouse IgG antibodies (Jackson ImmunoResearch), diluted 1:500 in FACS buffer. Plates were blocked from light and incubated 1 hour at 4° C. Cells were then washed again then were resuspended in 200 μL ice cold FACS buffer. Cells were washed again and resuspended in 200 μL fixing solution (PBS with 1% formaldehyde). Samples were stored at 4° C. covered in foil, then were acquired in NovoCyte Flow Cytometer and analyzed by NovoExpress software. Results obtained with serum containing samples are shown in FIG. 19. Results from serum-free samples are shown in FIG. 20.

FIGS. 19 and 20 show that 16E4, 7F3, and 17B10 successfully bound to HUVEC cells under experimental conditions. The serum containing samples (FIG. 19) showed similar binding capacities to those run without serum present (FIG. 20), suggesting that there was little effect of Fc binding for these antibodies on HUVEC cells.

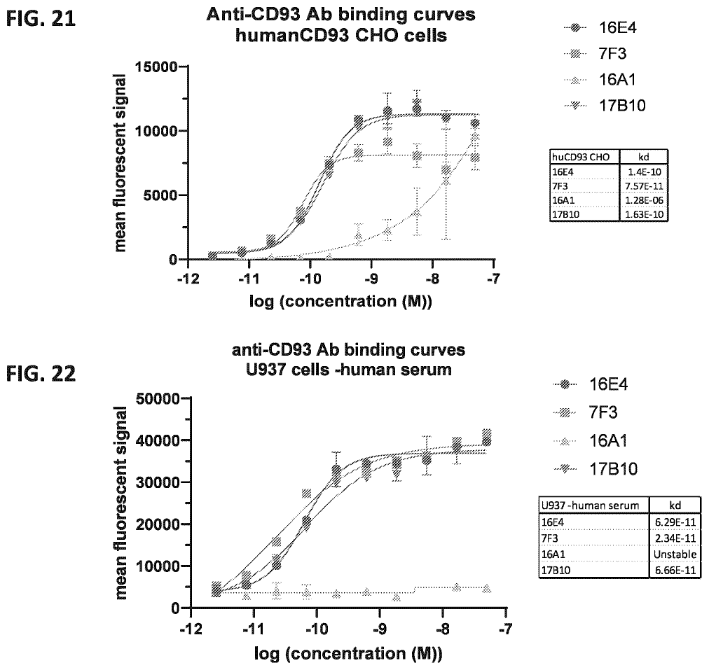

CD93 expressing CHO cells were detached by incubation with TrypLE reagents (Gibco cat #12604-013), which preserves the integrity of CD93 on the cell surface. Cells were quenched with media then counted. Cells were resuspended in FACS buffer (ice cold PBS with 0.5% BSA) and human serum was added to 20% (10% final volume) and put on ice for approximately 20 minutes. 5×10⁴ cells were seeded per well in 100 μL and incubated with serial diluted anti-CD93 antibodies in 100 μL on ice for 2 hours. Samples were then washed by spinning samples at 1200 rpm for 5 minutes. Media was discarded and cells were resuspended in 200 μL ice cold FACS buffer. Cells were washed again and resuspended in 100 μL of secondary Antibody, AlexaFluor647 conjugated anti-human IgG or anti-mouse IgG antibodies (Jackson ImmunoResearch), diluted 1:500 in FACS buffer. Plates were covered with foil to protect from like and incubated for 1 hour on ice. Cells were washed again resuspended in 200 μL ice cold FACS buffer. Cells were washed again and were resuspended in 200 μL fixing solution (PBS with 1% formaldehyde). Samples were stored at 4° C. covered in foil, then were acquired in NovoCyte Flow Cytometer and analyzed by NovoExpress software. Results are shown in FIG. 21.

FIG. 21 shows that 16E4, 7F3, 16A1 and 17B10 successfully bound to human CD93 CHO cells under experimental conditions. 16E4, 7F3, and 17B10 had similar binding affinities to hCD93 CHO cells, while 16A1 had relatively reduced affinity to human CD93 compared to the other antibodies.

U937 cells were detached by incubation with TrypLE reagent (Gibco cat #12604-013), which preserves the integrity of CD93 on the cell surface. Cells were quenched with media then counted. Cells were resuspended in FACS buffer (ice cold PBS with 0.5% BSA) and put on ice ˜20 min. 5×10⁴ cells were seeded per well in 100 μL and incubated with serial diluted anti-CD93 antibodies in 100 μL on ice for 2 hours. Samples were then washed by spinning samples at 1200 rpm for 5 minutes. Media was discarded and cells were resuspended in 200 μL ice cold FACS buffer. Cells were washed again and resuspended in 100 μL of secondary Antibody, AlexaFluor647 conjugated anti-human IgG or anti-mouse IgG antibodies (Jackson ImmunoResearch), diluted 1:500 in FACS buffer. Plates were covered with foil to protect from light and were incubated for 1 hour on ice. Samples were then washed again and resuspended in 200 μL ice cold FACS buffer. Cells were washed again and resuspended in 200 μL fixing solution (PBS with 1% formaldehyde). Samples were stored at 4° C. covered in foil, ands were subsequently acquired in NovoCyte Flow Cytometer and analyzed by NovoExpress software.

FIG. 22 shows that 16E4, 7F3, and 17B10 successfully bound to U937 cells under experimental conditions.

### Example 13. Cell Based Assay Analysis of 17B10 Antibodies

### 1. Binding of Humanized 17B10 to Overexpressing Human CD93 CHO Cells

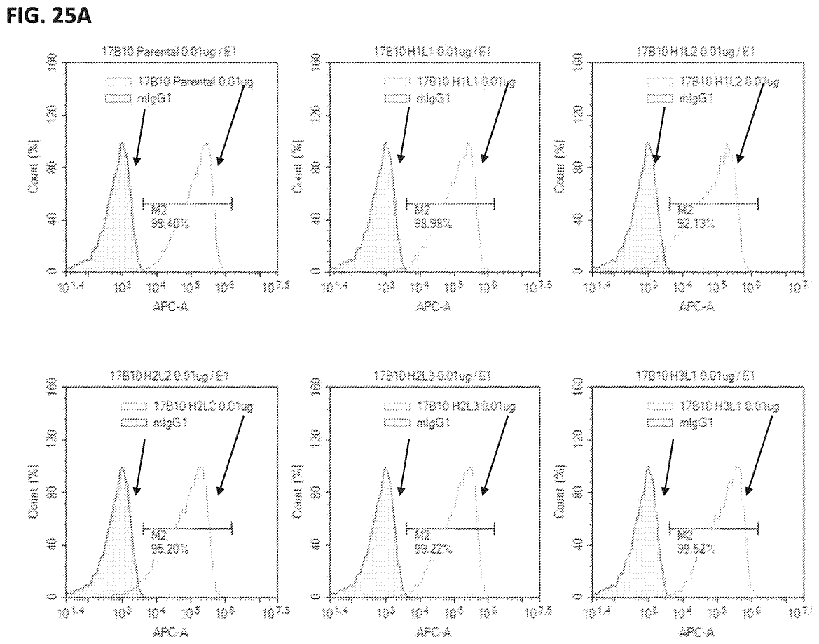

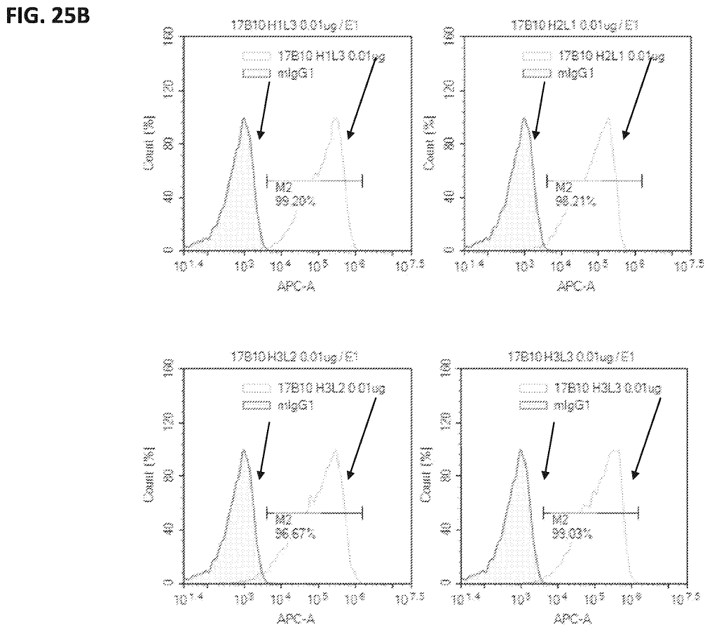

Various humanized 17B10 antibodies comprising a chimeric Fc containing mouse IgG1 CH2 and CH3 domains and human CH1 domains was made in ExpiHEK by combining one of the three humanized heavy chains with one of the three humanized light chains (see Example 10, Tables 6-7). The resulting antibodies were tested for binding to CHO cells overexpressing human CD93 using FACS analysis. The results are shown in FIGS. 25A-25B. As shown, all tested antibodies (i.e., H1L1, H1L2, H1L3, H2L1, H2L2, H2L3, H3L1, H3L2, H3L3) effectively bind to CHO cells overexpressing human CD93.

### 2. Binding of Humanized 17B10 to KG1a and U937 Cells

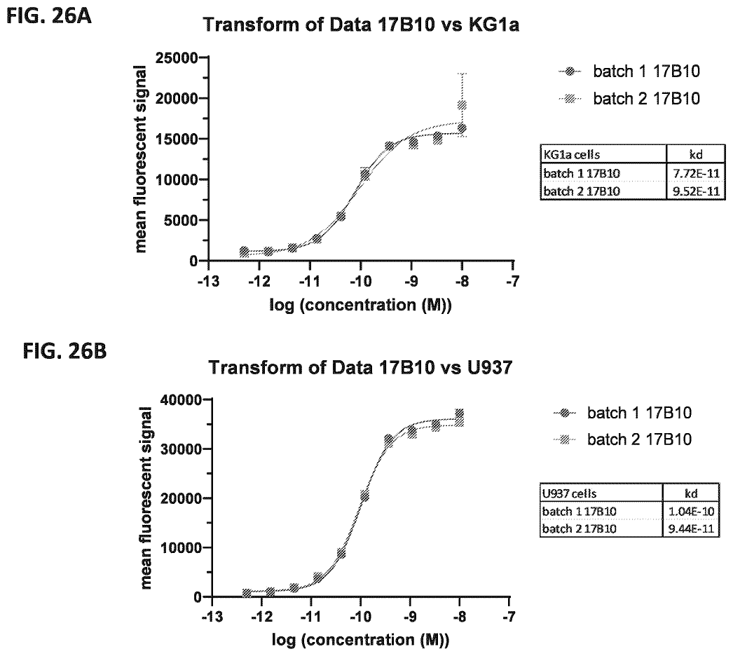

Binding of humanized 17B10 (V_{H}3V_{L}3, i.e., H3L3) to KG1a and U937 cells were tested as described in Example 12. Experiments were repeated using two batches of 17B10 antibody. FIGS. 26A-26B show that 17B10 bound to both KG1a and U937 with high affinity.

3. Binding of Humanized 17B10 (V_{H}3V_{L}3) to Mouse CHO Cells

Parental 17B10 antibody and humanized 17B10 having a V_{H} sequence of SEQ ID NO: 349 and a V_{L} sequence of SEQ ID NO: 352, and a chimeric Fc containing mouse IgG1 CH2 and CH3 domains and human CH1 domains was made in ExpiHEK. Mouse CD93 expressing CHO cells were detached by incubation with TrypLE reagents (Thermo Fisher), which preserved the integrity of CD93 on the cell surface. Then the cells were incubated with parental 17B10 antibody or humanized 17B10 anti-CD93 antibody (50 μg/mL) for 30 minutes at 4° C. After washing with FACS buffer, the cells were incubated with Alexa Fluor 488 conjugated anti-human IgG or anti-mouse IgG antibodies (Jackson ImmunoResearch) for 30 minutes in 4° C. After washing with FACS buffer twice, the samples were acquired in NovoCyte Flow Cytometer and analyzed by NovoExpress software.

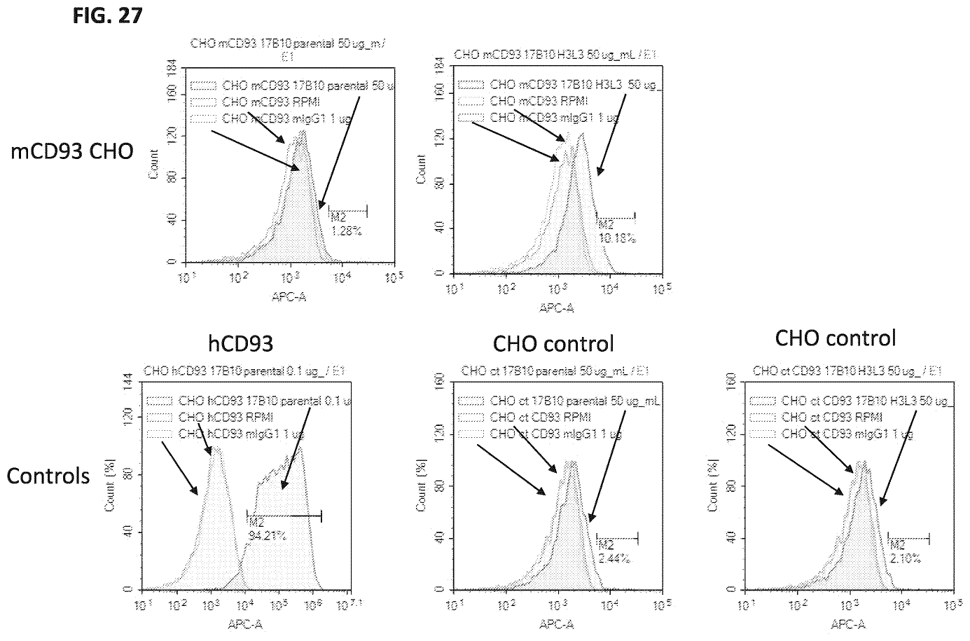

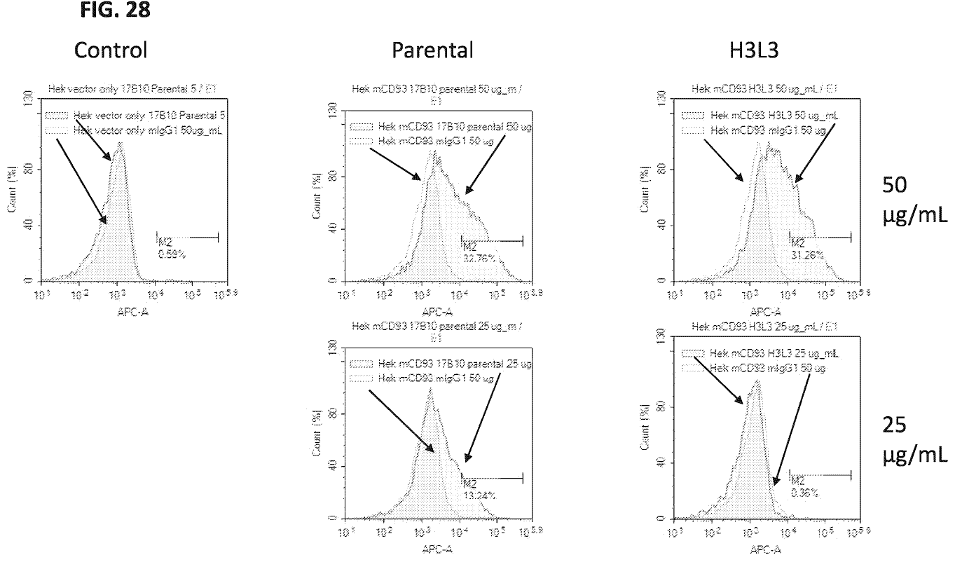

FIG. 27 shows that the humanized 17B10 bound to mouse CD93 expressing cells at 50 μg/mL.

4. Binding of Humanized 17B10 (V_{H}3V_{L}3) to mCD93 HEK

Mouse CD93 expressing HEK cells were detached by incubation with TrypLE reagents (Thermo Fisher), which preserves the integrity of CD93 on the cell surface. Then the cells were incubated with serial diluted parental 17B10 and humanized 17B10 ((1131.3) anti-CD93 antibodies for 30 minutes at 4° C. After washing with FACS buffer, the cells were incubated with Alexa Fluor 488 conjugated anti-human IgG or anti-mouse IgG antibodies (Jackson ImmunoResearch) for 30 minutes in 4° C. After washing with FACS buffer twice, the samples were acquired in NovoCyte Flow Cytometer and analyzed by NovoExpress software.

FIG. 28 shows that both parental 17B10 and humanized 17B10 (11313) bound to mouse CD93 expressing HEK cells at 50 μg/mL.

### 4. HUVEC Tube Formation Assay

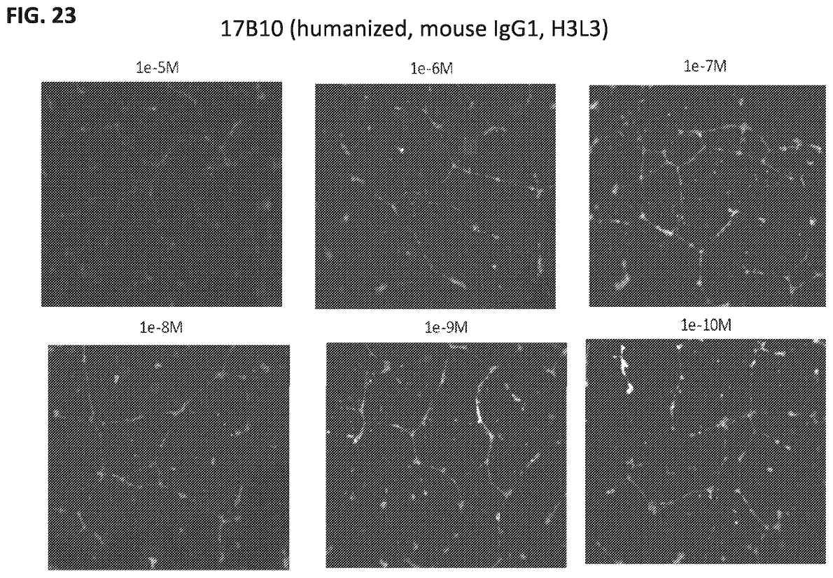

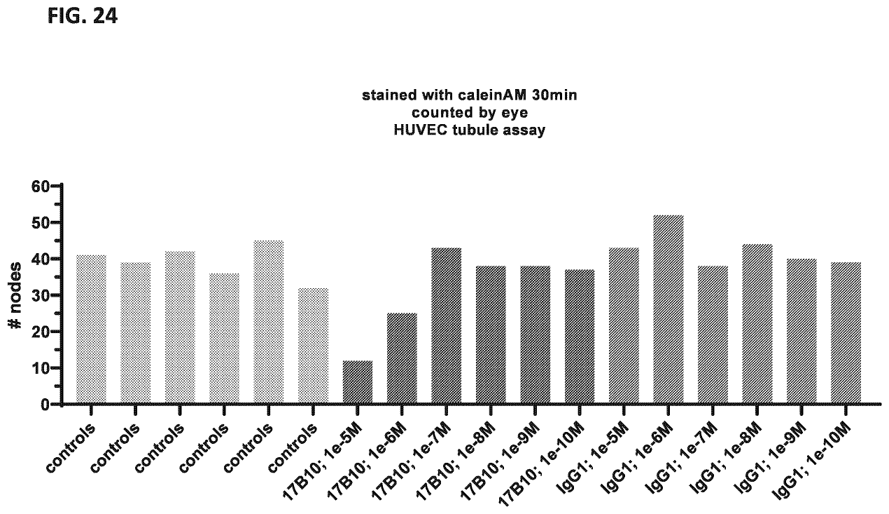

Inhibition of angiogenesis by humanized 17B10 anti-CD93 antibody (11313) was tested in a HUVEC tube formation assay. Human umbilical vein endothelial cell (HUVECs, Thermo Fisher Scientific, Waltham, MA) were cultured in medium 200 supplemented with low serum growth supplement (LSGS, Thermo Fisher Scientific, Waltham, MA) at 37° C. with 5% CO₂. 96 well plates were coated with 50 Al of Geltrex reduced growth factor basement membrane matrix (Thermo Fisher Scientific) and incubated for 30 min at 37° C. To investigate the effects of humanized 17B10 antibody on tube formation, 1×10⁴ HUVEC cells were seeded onto Matrix-coated plates and incubated in the presence or absence of purified antibodies at various concentrations for 18 hours at 37° C. with 5% CO₂. Cells were stained with calcein AM, and images were collected. FIGS. 23-24 show that humanized 17B10 inhibited tube formation at certain concentrations as compared to the controls.

### 4. Blocking Capacities of 17B10 Antibodies

17B10 antibodies (parental and humanized) were tested in cell based assays.

Parental and humanized 17B10 antibodies did not significantly block IGFBP7 binding to CD93 or MMRN2 binding to CD93 (data not shown).

### Example 14. ELISA Binding Analysts of Anti-CD93 Antibodies

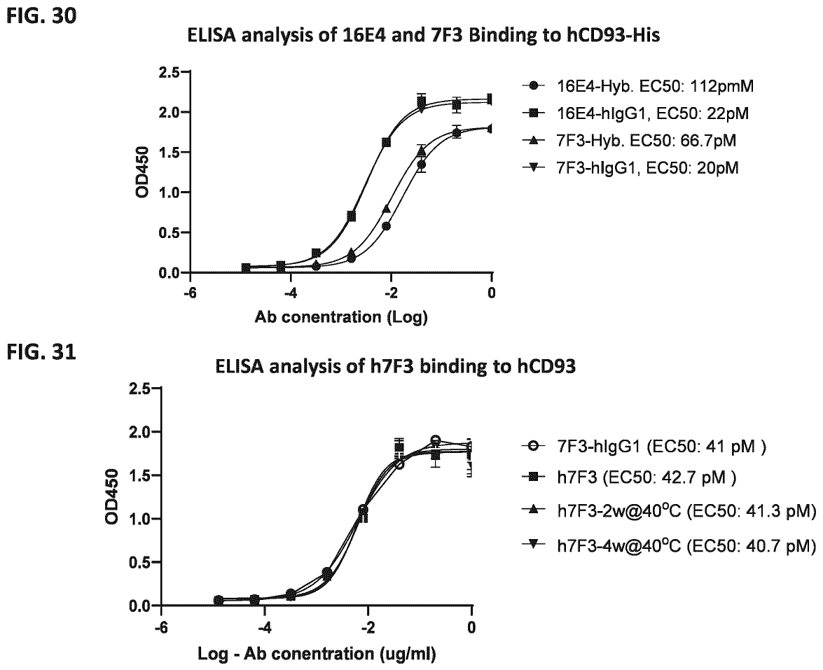

Hybridoma produced parental 16E4 and 7F3 were compared to recombinant, chimeric versions of the antibodies. His-tagged human CD93 was coated onto a 96 well plate at 1 μg/mL in 1×PBS overnight at 4° C. The plate was washed with ELISA wash buffer (Boston BioProduct, Inc.) and the wells were blocked with ELISA blocking buffer for 1 hour at 37° C. Purified antibodies were serially diluted in ELISA blocking buffer (Boston BioProduct, Inc.) and incubated on the receptor for 1 hour at 37° C. The plate was washed with ELISA wash Buffer. HRP conjugated Anti-mouse Fc was diluted in ELISA blocking buffer and added to the wells containing the hybridoma produced 16E4 and 7F3 (16E4-Hyb and 7F3-Hyb in FIG. 30). HRP conjugated Anti-human Fc was added to the well containing the humanized 16E4 and 7F3 antibodies (16E4-hIgG1 and 7F3-hIgG1 in FIG. 30) for one hour at 37° C. The plate was washed with ELISA wash buffer. HRP substrate was added for indirect detection of the antibodies binding to CD93. FIG. 30 shows that recombinant chimeric antibodies had stronger affinity for the CD93 than the parental antibodies under this method.

Humanized 7F3 antibody was stored in the dark at 40° C. for 2 or 4 weeks. His-tagged human CD93 was coated onto a 96 well plate at 1 μg/mL in 1×PBS overnight at 4° C. The plate was washed with ELISA wash buffer (Boston BioProduct, Inc.) and the wells were blocked with ELISA blocking buffer for 1 hour at 37° C. Purified 7F3 antibodies were serially diluted in ELISA blocking buffer (Boston BioProduct, Inc.) and incubated on the receptor for 1 hour at 37° C. The plate was washed with ELISA wash Buffer. HRP-conjugated anti-human Fc antibody was incubated for 1 hour at 37° C. The plate was washed with ELISA wash Buffer. HRP substrate was added for indirect detection of the antibodies binding to CD93. FIG. 31 shows that no difference was observed for any of the treated or untreated samples by ELISA.

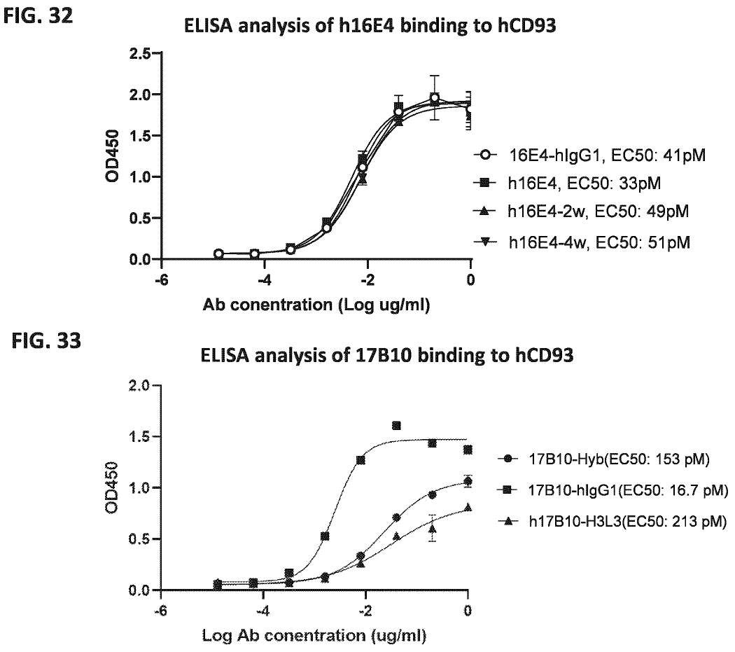

Humanized 16E4 antibody was stored in the dark at 40° C. for 2 or 4 weeks. His-tagged human CD93 was coated onto a 96 well plate at 1 μg/mL in 1×PBS overnight at 4° C. The plate was washed with ELISA wash buffer (Boston BioProduct, Inc.) and the wells were blocked with ELISA blocking buffer for 1 hour at 37° C. Purified 16E4 antibodies were serially diluted in ELISA blocking buffer (Boston BioProduct, Inc.) and incubated on the receptor for 1 hour at 37° C. The plate was washed with ELISA wash Buffer. HRP-conjugated anti-human Fc antibody was incubated for 1 hour at 37° C. The plate was washed with ELISA wash Buffer. HRP substrate was added for indirect detection of the antibodies binding to CD93. FIG. 32 shows that no difference was observed for any of the treated or untreated samples by ELISA.

17B10 antibody produced by hybridoma (17B10-Hyb in FIG. 33) was compared to recombinant parental 17B10-hFc (17B10-hIgG1 in FIG. 33) and humanized 17B10-mFc (h17B10-H3L3 in FIG. 33) to determine the binding to human CD93. His-tagged human CD93 was coated onto a 96 well plate at 1 μg/mL in 1×PBS overnight at 4° C. The plate was washed with ELISA wash buffer (Boston BioProduct, Inc.) and the wells were blocked with ELISA blocking buffer for 1 hour at 37° C. Purified 17B10 antibodies were serially diluted in ELISA blocking buffer (Boston BioProduct, Inc.) and incubated on the receptor for 1 hour at 37° C. The plate was washed with ELISA wash Buffer. HRP conjugated Anti-mouse Fc was diluted in ELISA blocking buffer and added to the wells containing the hybridoma produced 17B10. HRP conjugated anti-human Fc was added to the well containing the recombinant 17B10 antibodies for 1 hour at 37° C. The plate was washed with ELISA wash Buffer. HRP substrate was added for indirect detection of the antibodies binding to CD93. FIG. 33 shows that the mouse Fc containing molecules had weaker binding to the human CD93 than the recombinant parental 17B10 with the human Fc.

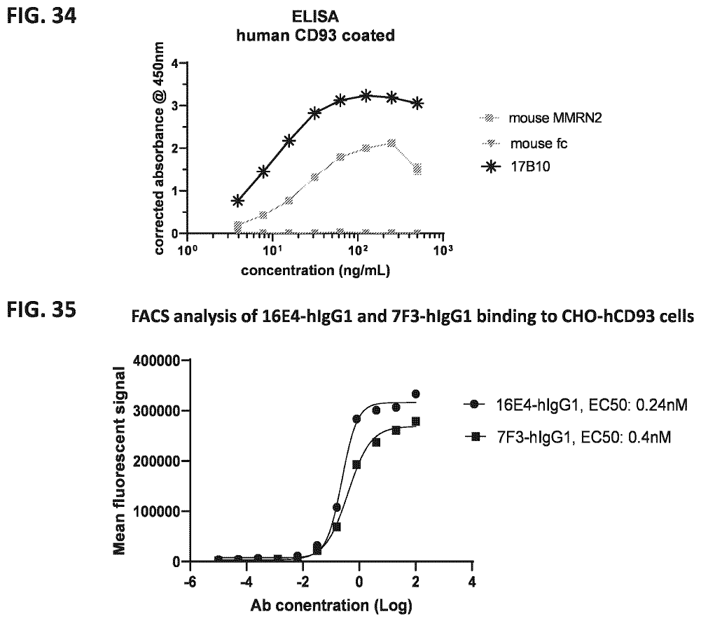

A chimeric 17B10 molecule was made with a humanized CDR and human CH1 domain but mouse IgG1 CH2 and CH3 domains. This molecule was compared to mouse MMRN2-mFc for its ability to bind to human CD93. His-tagged human CD93 was coated onto a 96 well plate at 1 μg/mL in 1×PBS overnight at 4° C. The plate was washed with ELISA wash buffer (PBS with tween; Boston Bioproduct cat #BB-171) 3 times then wells were blocked with 200 μL ELISA blocking buffer (5% BSA (VWR cat #0332) in PBS) for 1 hour at room temp. The plates were then washed 3 times with ELISA wash buffer then purified 17B10 antibody and mouse MMRN2-mFc were serially diluted in ELISA blocking buffer (BSA 5% in PBS) and incubated on the receptor for 2 hours at room temperature on orbital shaker at 100 rpm. The plate was washed 3 times with ELISA wash Buffer then HRP-conjugated anti-mouse Fc antibody (Jackson ImmunoResearch cat #115-035-164) was added to the 17B10 and the mouse MMRN2-mFc for 1 hour at room temperature on orbital shaker at 100 rpm. HRP-conjugated anti-mouse Fc antibody (Jackson ImmunoResearch cat #115-035-164) was added to the wells for 1 hour at room temperature on orbital shaker at 100 rpm. The plates were washed 3 times with ELISA wash Buffer then 100 μL TMB (SeraCare cat #5120-0077) added per well and allowed to mix 1-5 min then stopped by adding 100 μL Sulfuric Acid 1.0N (VWR cat #BDH7232-1). Absorbance measured at 450 nm. Absorbance signals corrected by subtracting averaged background signal from control wells containing secondary HRP Ab only. FIG. 34 shows that 17B10 bound to human CD93-his by ELISA better than mouse MMRN2-mFc.

### Example 15. FACS Cell-Based Binding Analysis of Anti-CD93 Antibodies

Binding of anti-CD93 antibodies 7F3 and 16E4 to cell surface expressing human CD93 CHO cells was determined by fluorescence activated cell sorting (FACS) assay. Human CD93 expressing CHO cells were detached by incubation with TrypLE reagents (Thermo Fisher), which preserves the integrity of CD93 on the cell surface. Then the cells were incubated with serially diluted anti-CD93 antibodies for 30 minutes in 4° C. After washing by FACS buffer, the cells were incubated with Alexa Fluor 647 conjugated anti-human IgG (Jackson ImmunoResearch) for 30 minutes in 4° C. After washing by FACS buffer twice, the samples were acquired in NovoCyte Flow Cytometer and analyzed by NovoExpress software. Recombinant 16E4 bound to the cells with an EC50 of 0.24 nM, while recombinant 7F3 antibody bound with an EC50 of 0.4 nM (FIG. 35).

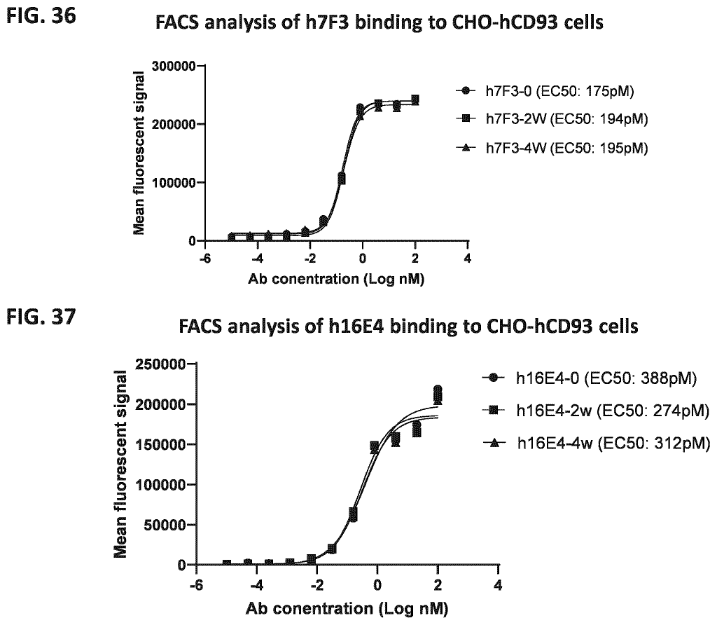

Binding of humanized 7F3 anti-CD93 antibodies to cell surface expressing human CD93 CHO cells was determined by fluorescence activated cell sorting (FACS) assay. Humanized 7F3 antibody was stored in the dark at 40° C. for 2 or 4 weeks. Human CD93 expressing CHO cells were detached by incubation with TrypLE reagents (Thermo Fisher), which preserves the integrity of CD93 on the cell surface. Then the cells were incubated with serial diluted anti-CD93 antibodies for 30 minutes in 4° C. After washing by FACS buffer, the cells were incubated with Alexa Fluor 647 conjugated anti-human IgG (Jackson ImmunoResearch) for 30 minutes at 4° C. After washing by FACS buffer twice, the samples were acquired in NovoCyte Flow Cytometer and analyzed by NovoExpress software. There was no change in the affinity for the 7F3 antibody to CD93 due to the high temperature treatment (FIG. 36).

Humanized 16E4 antibody was stored in the dark at 40′C for 2 or 4 weeks. Human CD93 expressing CHO cells were detached by incubation with TrypLE reagents (Thermo Fisher), which preserves the integrity of CD93 on the cell surface. Then the cells were incubated with serial diluted anti-CD93 antibodies for 30 minutes at 4° C. After washing by FACS buffer, the cells were incubated with Alexa Fluor 647 conjugated anti-human IgG (Jackson ImmunoResearch) for 30 minutes in 4° C. After washing by FACS buffer twice, the samples were acquired in NovoCyte Flow Cytometer and analyzed by NovoExpress software. Incubation of humanized 16E4 at 40° C. did not reduce the binding of the antibodies to the CD93 expressing cells (FIG. 37).

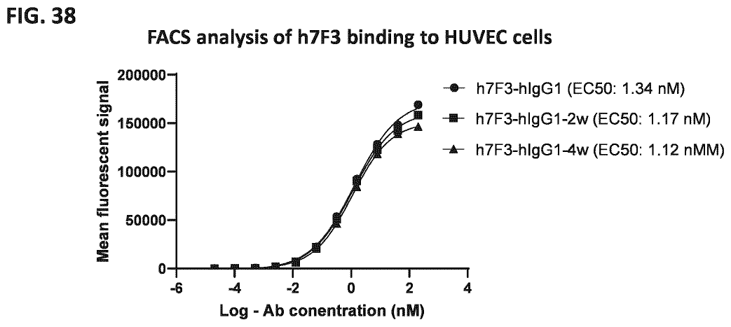

Humanized 7F3 antibody was stored in the dark at 40° C. for 2 or 4 weeks. HUVEC cells were detached by incubation with TrypLE reagents (Thermo Fisher), which preserves the integrity of CD93 on the cell surface. Then the cells were incubated with serial diluted anti-CD93 antibodies for 30 minutes at 4° C. After washing by FACS buffer, the cells were incubated with Alexa Fluor 647 conjugated anti-human IgG (Jackson ImmunoResearch) for 30 minutes in 4° C. After washing by FACS buffer twice, the samples were acquired in NovoCyte Flow Cytometer and analyzed by NovoExpress software. Incubation of humanized 7F3 at 40° C. did not reduce the binding of the antibodies to HUVEC cells (FIG. 38).

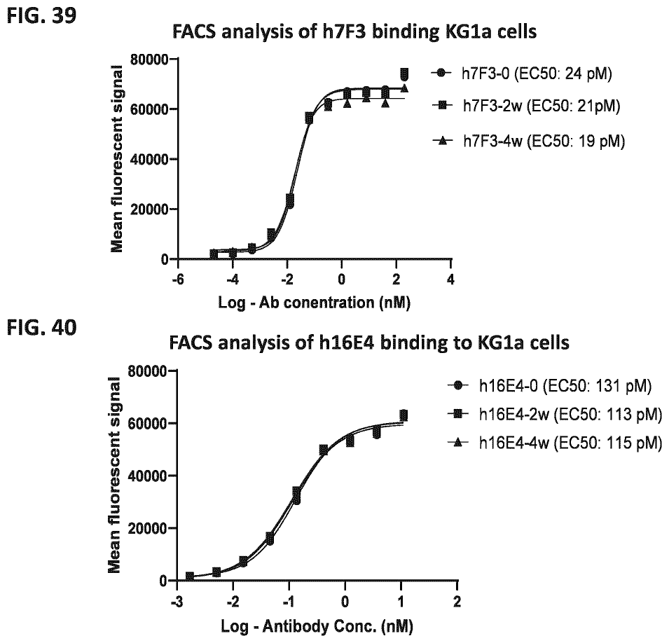

Binding of 7F3 anti-CD93 antibodies to KG1a cells was determined by fluorescence activated cell sorting (FACS) assay. Humanized 7F3 antibody was stored in the dark at 40° C. for 2 or 4 weeks. KG1a cells were detached by incubation with TrypLE reagents (Thermo Fisher), which preserves the integrity of CD93 on the cell surface. Then the cells were incubated with serial diluted anti-CD93 antibodies for 30 minutes at 4° C. After washing by FACS buffer, the cells were incubated with Alexa Fluor 647 conjugated anti-human IgG (Jackson ImmunoResearch) for 30 minutes in 4° C. After washing by FACS buffer twice, the samples were acquired in NovoCyte Flow Cytometer and analyzed by NovoExpress software. Incubation of 7F3 at 40° C. did not reduce the binding of the antibodies to KG1a cells (FIG. 39).

Humanized 16E4 antibody was stored in the dark at 40° C. for 2 or 4 weeks. KG1a cells were detached by incubation with TrypLE reagents (Thermo Fisher), which preserves the integrity of CD93 on the cell surface. Then the cells were incubated with serial diluted anti-CD93 antibodies for 30 minutes at 4° C. After washing by FACS buffer, the cells were incubated with Alexa Fluor 647 conjugated anti-human IgG (Jackson ImmunoResearch) for 30 minutes in 4° C. After washing by FACS buffer twice, the samples were acquired in NovoCyte Flow Cytometer and analyzed by NovoExpress software. Incubation of 16E4 at 40° C. did not reduce the binding of the antibodies to KG1a cells (FIG. 40).

### Example 16. Anti-CD93 Antibody Octet Binding Analysis

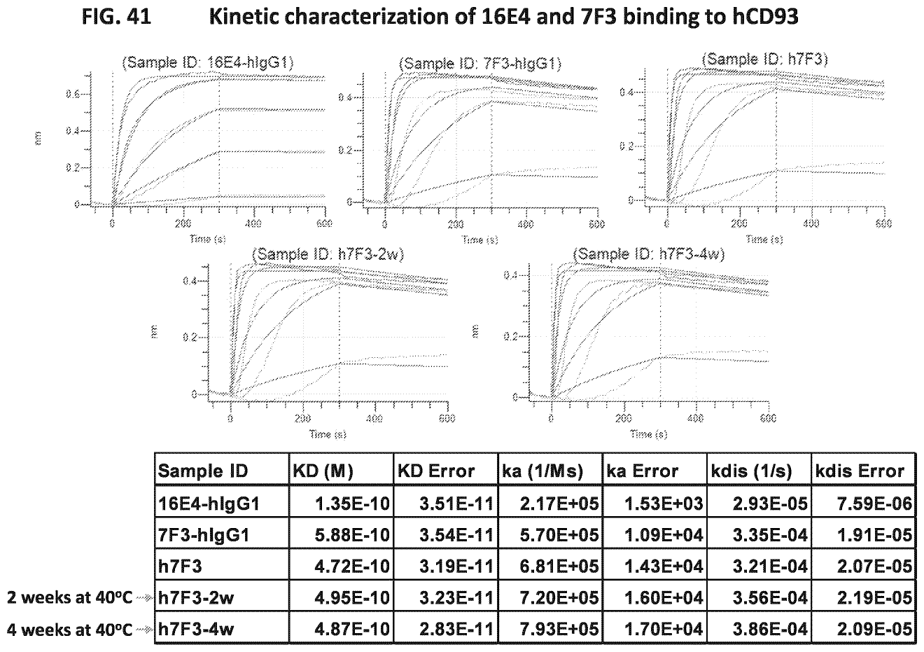

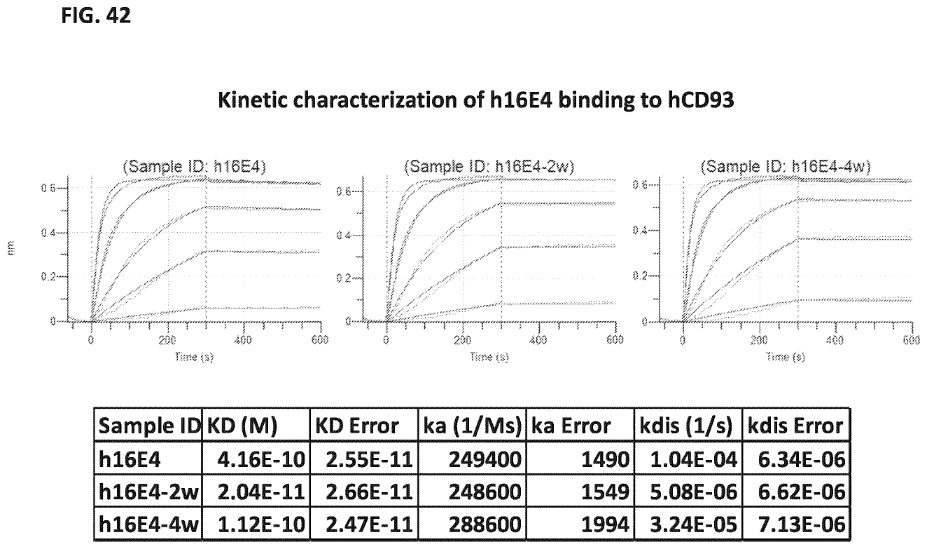

The binding affinity of anti-CD93 antibodies was determined with bio-layer interferometry using Octet QKe (Fortebio). Humanized 7F3 antibody was stored in the dark at 40° C. for 2 or 4 weeks. Human CD93 recombinant protein (Sino Biological Inc, Catalog #12589-H08H) was biotinylated using EZ-LINK NHS-PEG4 biotin (Thermo Fisher Scientific). Streptavidin biosensors (Fortebio) were used to load biotinylated CD93 protein (300 seconds in 5 μg/ml). Baseline was stabilized for 60 seconds in 1×kinetics buffer (Fortebio) before anti-CD93 antibodies, at a serial dilution, were allowed to associate for 300 seconds with captured protein. Then the sensors were dissociated in 1× kinetics buffer for 600 seconds. Data analysis was performed on ForteBio Data Analysis HT 11.1 software. The binding affinity of humanized 7F3 antibody against CD93 was not affected by the incubation at 40° C. (FIG. 41).

The binding affinity of anti-CD93 antibodies was determined with bio-layer interferometry using Octet QKe (Fortebio). Humanized 16E4 antibody was stored in the dark at 40° C. for 2 or 4 weeks. Human CD93 recombinant protein (Sino Biological Inc, Catalog #12589-H08H) was biotinylated using EZ-LINK NHS-PEG4 biotin (Thermo Fisher Scientific). Streptavidin biosensors (Fortebio) were used to load biotinylated CD93 protein (300 seconds in 5 μg/ml). Baseline was stabilized for 60 seconds in 1× kinetics buffer (Fortebio) before anti-CD93 antibodies, at a serial dilution, were allowed to associate for 300 seconds with captured protein. Then the sensors were dissociated in 1× kinetics buffer for 600 seconds. Data analysis was performed on ForteBio Data Analysis HT 11.1 software. The binding affinity of humanized 16E4 antibody against CD93 was not affected by the incubation at 40° C. (FIG. 42).

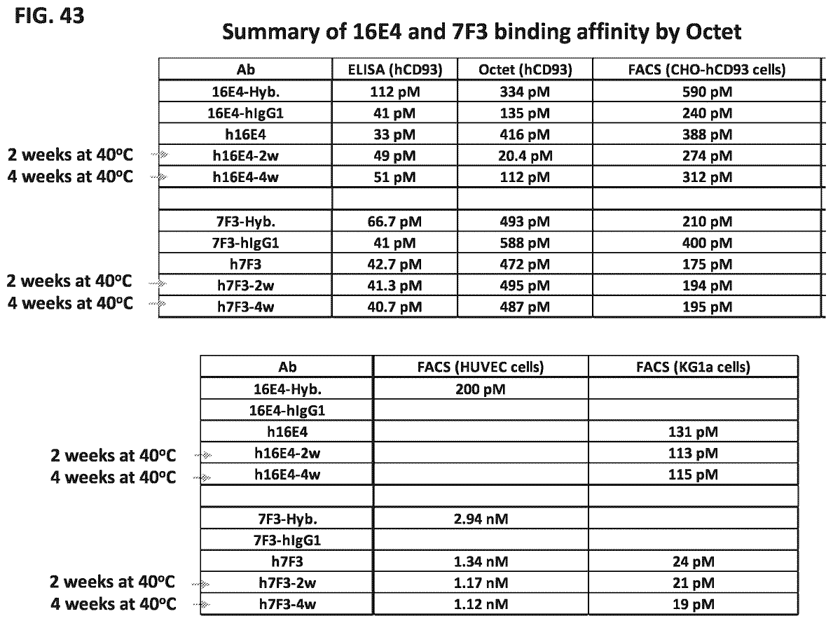

A summary of binding affinity of 16E4 and 7F3 is shown in FIG. 43.

### Example 17. Anti-CD93 Antibody Blocking Function Analysis

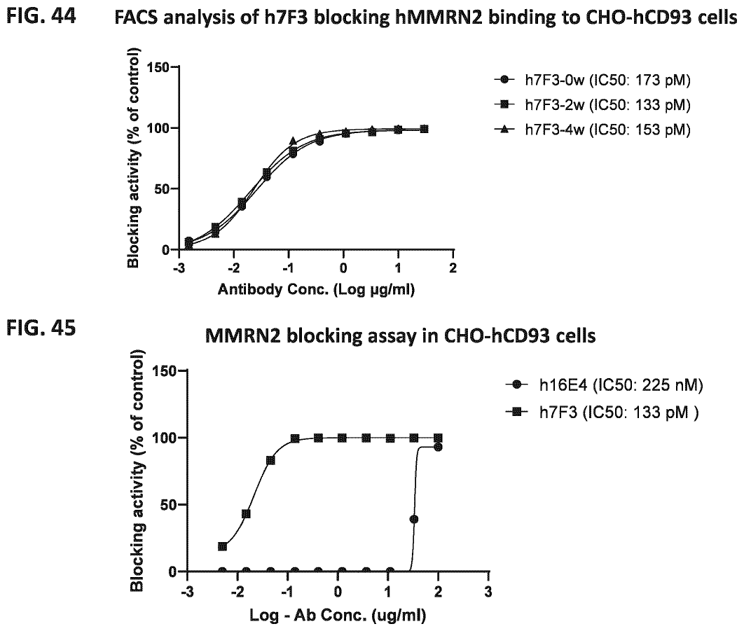

Blocking of MMRN2 binding to cell surface expressed human CD93 CHO cells by the 7F3 anti-CD93 antibody was determined by fluorescence activated cell sorting (FACS) assay. Humanized 7F3 antibody was stored in the dark at 40° C. for 2 or 4 weeks. Human CD93 expressing CHO cells (lx 10⁵ per well) were treated with serially diluted anti-CD93 7F3 antibodies or isotype control for 30 minutes at 4° C. Then the cells were incubated with hMMRN2₄₉₅₋₆₇₄ at 0.1 μg/ml. After incubation, the cells were washed with FACS buffer and incubated with APC-conjugated anti-His tag (BioLegend) for 30 minutes at 4° C. to detect the MMRN2 binding. After washing with FACS buffer twice, the samples were analyzed, and data acquired in NovoCyte Flow. Recombinant his tagged hMMRN2₄₉₅₋₆₇₄ was produced internally in E. Coli following routine procedure. Incubation of 7F3 at 40° C. did not affect the ability of 7F3 to block MMRN2 binding to human CD93 expressing CHO cells (FIG. 44).

Blocking of MMRN2 binding to cell surface expressed human CD93 CHO cells by the humanized 7F3 and 16E4 anti-CD93 antibody was also determined by fluorescence activated cell sorting (FACS) assay. Human CD93 expressing CHO cells (1×10⁵ per well) were treated with serially diluted anti-CD93 7F3 or 16E4 antibodies or isotype control for 30 minutes at 4° C. Then the cells were incubated with hMMRN2₄₉₅₋₆₇₄ at 0.1 μg/ml. APC-conjugated anti-His tag (BioLegend) was used to detect the MMRN2 binding. Then the cells were washed with FACS buffer and incubated with APC-conjugated anti-His tag antibody at 1 μg/ml for 30 minutes at 4° C. After washing with FACS buffer twice, the samples were analyzed and data acquired in NovoCyte Flow. Recombinant his tagged hMMRN2495474 was produced internally in Expi_HEK following routine procedure. Humanized 7F3 was able to block MMRN2 binding to human CD93 expressing CHO cells, but humanized 16E4 was not (FIG. 45).

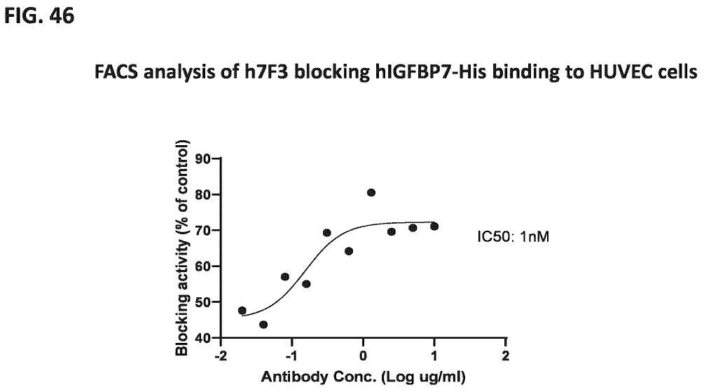

Blocking of IGFBP7 binding to the cell surface of HUVEC cells by humanized 7F3 anti-CD93 antibody was determined by FACS. HUVEC cells (lx 10⁵ per well) were treated with serially diluted humanized anti-CD93 7F3 antibody or isotype control for 30 minutes at 4° C. Then the cells were incubated with His-tagged human IGFBP7 recombinant protein (0.1 μg/ml) for another 30 minutes at 4° C. After incubation, the cells were washed with FACS buffer and incubated with APC-conjugated anti-His tag (BioLegend) for 30 minutes in 4° C. to detect the IGFBP7 binding. After washing with FACS buffer twice, the samples were analyzed and data acquired in NovoCyte Flow. As shown in FIG. 46, 7F3 antibody blocked the binding of IGFBP7 to HUVEC cells.

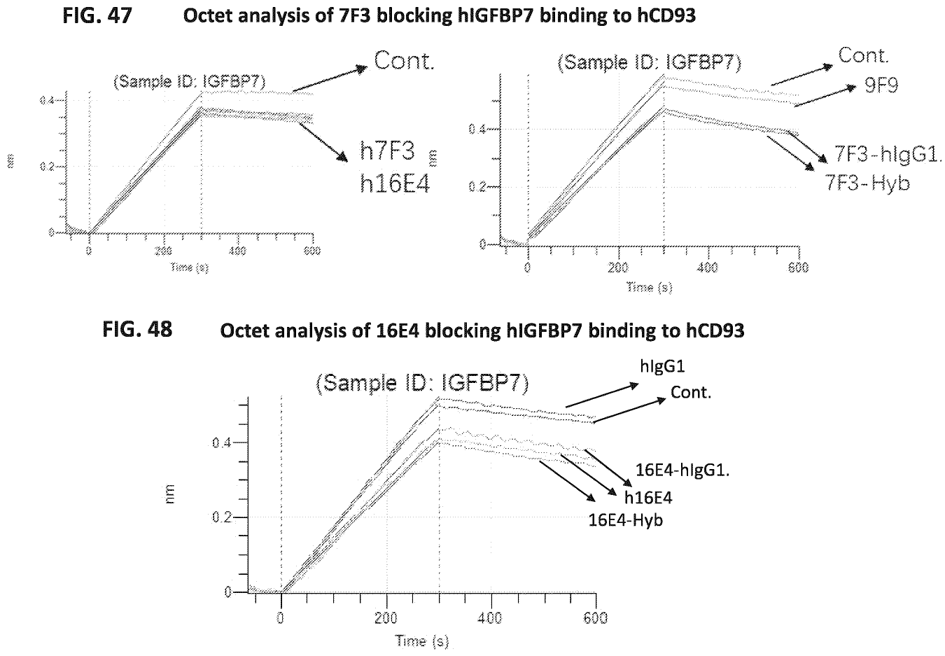

Blocking of IGFBP7 binding to CD93 by 7F3 and 16E4 was determined using bio-layer interferometry (BLI). The blocking of IGFBP7 binding to hCD93 by anti-CD93 antibodies 7F3 and 16E4 was determined with bio-layer interferometry using Octet QKe (Fortebio). Human CD93 recombinant protein (Sino Biological Inc, Catalog #12589-H08H) was biotinylated using EZ-LINK NHS-PEG4 biotin (Thermo Fisher Scientific). Streptavidin biosensors (Fortebio) were used to load biotinylated CD93 protein (300 seconds in 5 μg/ml). Baseline was stabilized for 60 seconds in 1× kinetics buffer (Fortebio) before anti-CD93 antibodies and a negative control antibody (9F9) (90 μg/mL) were allowed to associate for 300 seconds with captured protein. The IGFBP7 was added to associate for 300 seconds. Then the sensors were dissociated in 1× kinetics buffer for 600 seconds. Data analysis was performed on ForteBio Data Analysis HT 11.1 software. Hybridoma and humanized 7F3 and 16E4 antibodies were able to block IGFBP7 association to human CD93 (FIGS. 47 and 48).

### Example 18. Anti-CD93 Antibody Tube Formation Analysis

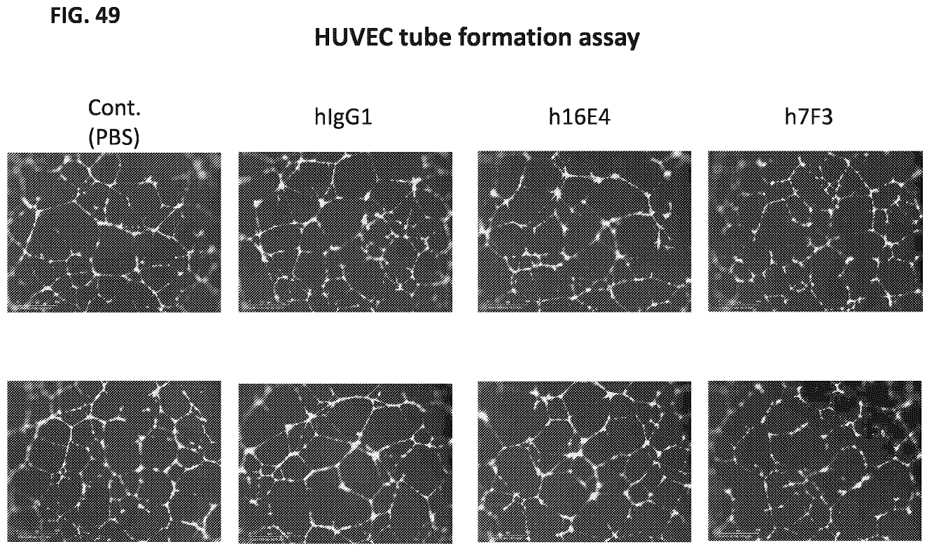

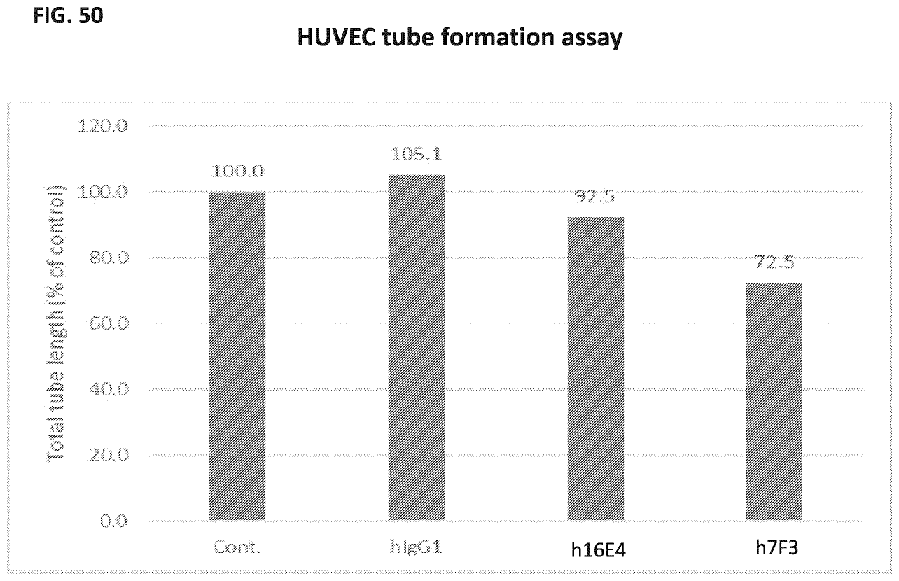

Inhibition of angiogenesis by humanized 7F3 and 16E4 anti-CD93 antibodies was tested in a HUVEC tube formation assay. Human umbilical vein endothelial cell (HUVECs, Thermo Fisher Scientific, Waltham, MA) were cultured in medium 200 supplemented with low serum growth supplement (LSGS, Thermo Fisher Scientific, Waltham, MA) at 37° C. with 5% CO₂. 96 well plates were coated with 50 μl of Geltrex reduced growth factor basement membrane matrix (Thermo Fisher Scientific) and incubated for 30 min at 37° C. To investigate the effects of humanized 7F3 and 16E4 antibodies on tube formation, 2×10⁴ HUVEC cells were seeded onto Matrix-coated plates and incubated in the presence or absence of purified antibodies (40 μg/mL) for 18 hours at 37° C. with 5% CO₂. Cells were stained with calcein AM, and images were collected. FIGS. 49 and 50 show that humanized 16E4 showed 92.5% tube formation, while humanized 7F3 showed 72.5% tube formation compared to the controls.

### Example 19. Anti-Tumor Effect of the CD93 Antibodies in ICI Mouse Model

The anti-tumor effect of the anti-CD93 antibodies was evaluated in a B16F10 melanoma syngeneic hCD93 KI mouse model using conventional technique in the art. The mice used for the study have heterozygous human CD93 knock-in, such that half of the murine CD93 in the mice is completely replaced by the human CD93.

For the syngeneic mouse model, heterozygous human CD93 KI-C57BL/6J mice were implanted with a murine cell line of B16F10 tumor cells (0.2×10⁶) in serum-free media. When tumors reached 40-50 mm³, the mice (n=8 per test article) were randomly assigned to groups. Anti-CD93 antibodies, including h16E4 (humanized 16E4, V_{H}4+V_{L}6), h7F3 (humanized 7F3, V_{H}3+V_{L}3), 17B10 chimeric (m17B10-hIgG1), and an isotype control antibody were dosed at 15 mg/kg mouse intraperitoneally biweekly for 4 weeks. Tumor volume and body weight were measured for each mouse. Upon completion of the study, tumors were surgically removed, weighed, measured, and snap frozen for cell analysis. Anti-tumor efficacy of the anti-CD93 antibodies was evaluated based on overall tumor volume and body weight was measured throughout the study to ensure general health of the animals.

TABLE 8
Anti-B16 tumor effect of CD93 antibodies in a humanized
CD93 knock-in mice model at Day 5 post-injection
Group Mean +/− Standard Error of the Mean, Tumor Volume (mm³)

| Group | 0 day | 5 days |
|---|---|---|
| Group 01: Isotype Control | 50.96 | 405.55 |
| StdErr | 1.75 | 69.44 |
| Group 02: h16E4 | 50.94 | 183.21 |
| StdErr | 1.75 | 24.30 |
| Group 03: h7F3 | 50.93 | 173.51 |
| StdErr | 1.63 | 25.35 |
| Group 04: 17B10 chimeric | 50.92 | 187.43 |
| StdErr | 1.57 | 27.25 |

Mean Growth

Study Days

| Group | 0 | 5 |
|---|---|---|
| Group 01: Isotype Control | 0.00% | 687.41% |
| Group 02: h16E4 | 0.00% | 264.41% |
| Group 03: h7F3 | 0.00% | 244.59% |
| Group 04: 17B10 chimeric | 0.00% | 268.50% |

Mean Growth = mean(T/T0) * 100%
T—current value
T0—initial value

As can be seen from Table 8, all tested CD93 antibodies significantly blocked tumor growth as early as 5 days post-injection, resulting in 2.6˜2.8-fold decrease of tumor growth. No significant difference in the anti-tumor effects of the three tested CD93 antibodies were found.

This study confirms CD93 antibodies of the present disclosure can inhibit tumor in vivo.

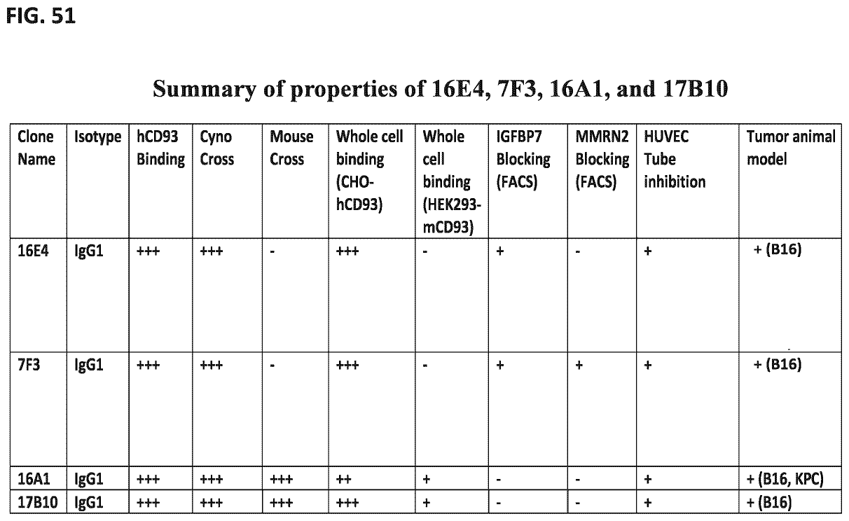

See FIG. 51 for a summary of properties of 16E4, 7F3, 16A1 and 17B10.

### Example 20 Animal Studies in Human CD93 KI Heterozygous Mice Using B16F10 Model

1. Human CD93 Knock-In Mouse Model Development

The knock-in model was designed to replace the mouse CD93 protein with human CD93 protein. Knock-in mouse model was developed using two methods as described below.

CRISPR/Cas9 was utilized to make two cuts with a guide RNA #1 targeting near the ATG at the 5′UTR of mouse CD93, and the guide RNA #2 targeting near the beginning of the 3′UTR. Homology directed repair used a donor to fuse in-frame the mouse 5′UTR with the CD93 human cDNA and enable expression from the endogenous CD93 promotor. The repair downstream of the STOP codon ensured that the CD93 hybrid transcript contains the mouse 3′UTR. Pure C57BL/6N mice were used as the background for the knock in model. Embryonic stem cell clones were produced and expanded with the knock-in human CD93 gene. Following sequence confirmation, a blastocyst injection was performed to establish the chimeric founders. Breeding proceeded from there with genotyping to identify heterozygote and homozygote pups.

Alternatively, CRISPR/Cas9 was utilized to remove the mouse exon 1 of CD93 corresponding to the extracellular domain of CD93 (S25-N572). In homology directed repair, the donor DNA contained the human sequence of CD93 from T26-K580. The resulting construct expressed a protein containing the humanized extracellular domain of CD93 with the mouse transmembrane and intracellular domains. C57BL/6 mouse embryonic stem cells were utilized for the knock-in model following sequence confirmation. Ozgene used its proprietary Go-Germiline blastocyst for the injections to establish the chimeric founders. Genotyping and phenotyping was performed to ensure production of heterozygote and homozygote mice.

2. B16F10 Murine Melanoma

B16F10 (ATCC® CCL-6475™) is a murine melanoma cell line from a C57BL/6J mouse. It is a subclone of the B16 tumor line. B16F10 was generated by injecting mice with B16 tumor cells, collecting and culturing secondary tumor growths, and injecting them into fresh mice for a total of 10 times. The cells are adherent with an epithelial morphology. B16F10 cells are highly metastatic and will form tumors and metastases post implantation into syngeneic C57BL/6 mouse.

The B16F10 cell line was maintained in vitro as monolayer culture in Dulbecco's Modified Eagle's medium (DMEM) with GlutaMAX™ Supplement and 10% Fetal Bovine Serum (FBS) in a humidified incubator at 37° C. in an atmosphere with 5% CO2. The tumor cells were routinely sub-cultured by trypsin-EDTA treatment 2-3 times per week depending on the growth rate and split ratio. For cell inoculation, the cells in an exponential growth phase were harvested and centrifuged at 335 g in a refrigerated centrifuge and the medium aspirated. The cell pellet was re-suspended in 10× volume of serum-free medium and counted. The cell suspension was centrifuged again as above and resuspended in serum-free medium to the final cell concentration of 2.0×10⁶ cells per mL (50% serum free media & 50% GelTrex), each 0.1 mL delivered the number of cells needed per inoculation. Cell suspensions were kept on ice until inoculation.

3. Human CD93 KI Heterozygous B16F10 Mouse Model for Anti-CD93 Antibody Efficacy Evaluation

The anti-tumor effect of the humanized anti-CD93 7F3, 16E4, and 17B10 antibodies were evaluated in the human CD93 KI heterozygous mouse in B16F10 melanoma model. The experimental design is shown in Table 9.

TABLE 9
Experimental design of antibody efficacy evaluation using B16F10 mouse model.

| | | | | | | | Dosing | Survival |
|---|---|---|---|---|---|---|---|---|
| | | | Dose | Dosing | | | Schedule | Bleeds |
| G | Treatment | N | (mg/mouse) | vol (mL) | ROA | Frequency | (Day #) | (Day #) |
| 1 | Isotype control | 8 | 0.3 | 0.3 | IP | Biweekly | Days 0, 5, | 5, 11 |
| | | | | | | (BIW) x4 | 8, 11 | |
| 2 | 7F3 | 8 | 0.3 | 0.3 | IP | BIW x4 | 0, 5, 8, 11 | 5, 11 |
| 3 | 16E4 | 8 | 0.3 | 0.3 | IP | BIW x4 | 0, 5, 8, 11 | 5, 11 |
| 4 | 17B10 chimeric | 8 | 0.3 | 0.3 | IP | BIW x4 | 0, 5, 8, 11 | 5, 11 |

The 17B10 antibody was the same as used in Example 11 (Animal Studies using 17B10 antibodies).

When tumors reach 50-60 mm³, the mice (n=8 mice per group) were randomly assigned to groups. Anti-CD93 antibodies (and isotype control) were dosed at 0.3 mg/mouse intraperitoneally on days 0, 5, 8, 11. Efficacy was evaluated based on overall tumor volume. Body weight was measured to ensure general health of the animals was not affected by the test.

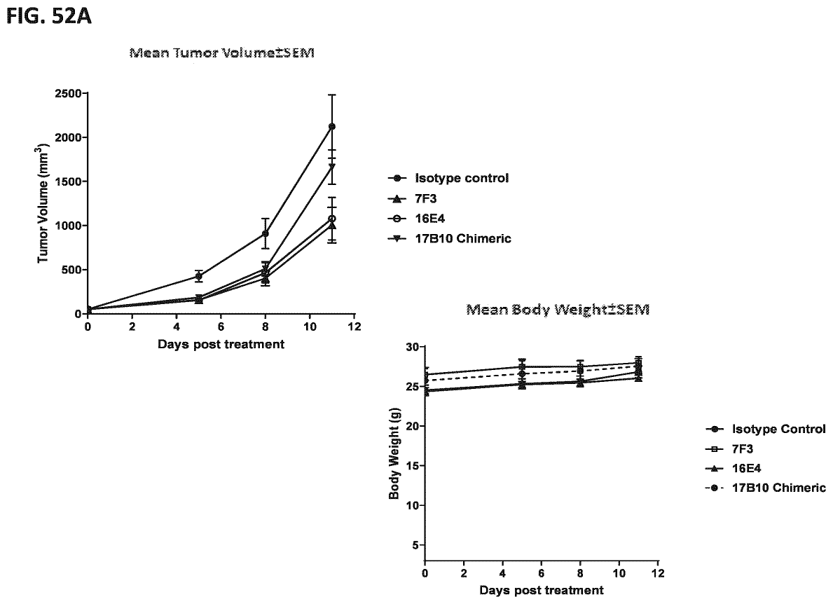

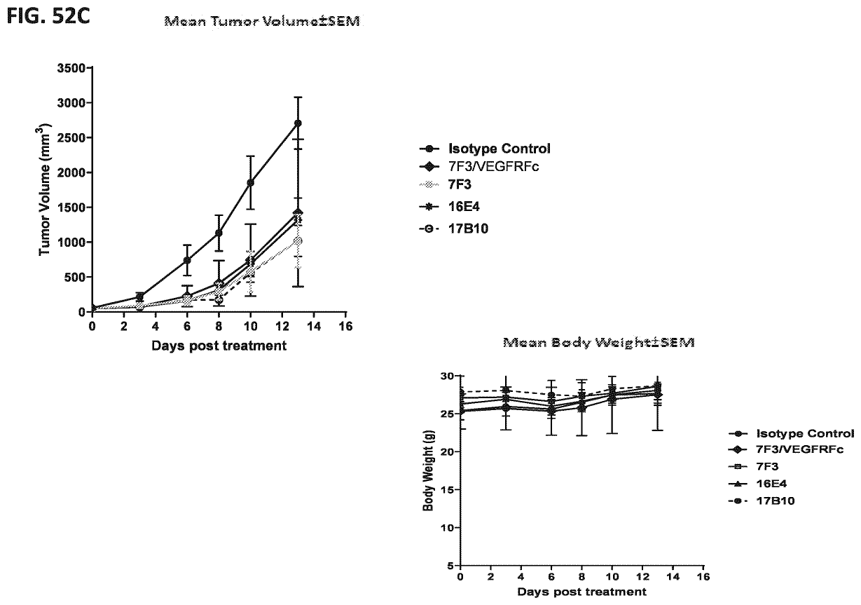

FIG. 52A shows that mice in 7F3 and 16E4 groups exhibited lower tumor volume compared to mice in the 17B10 chimeric group. The mean tumor volumes in 7F3 and 16E4 groups are approximately 50% of the mean tumor volume of the control group, and are approximately 60% of the mean tumor volume of the 17B10 chimeric group. Mice body weights of all tested antibody groups, including the isotype control group, were not affected by test articles.

4. Human CD93 KI Homozygous Mice B16F10 Model for Anti-CD93 Molecule Efficacy Evaluation

The anti-tumor effects of the humanized anti-CD93 7F3. 16E4, and 17B10 antibodies were evaluated in the human CD93 KI homozygous mouse in B16F10 melanoma model.

TABLE 11
Experimental design of antibody-fusion protein
efficacy evaluation using B16F10 mouse model.

| | | | Dose | Dosing | | | Dosing | Survival |
|---|---|---|---|---|---|---|---|---|
| G | Treatment | N | (mg/mouse) | vol (mL) | ROA | Frequency | Schedule | Bleeds |
| 1 | Isotype control | 6 | 0.3 | 0.3 | IP | BIW x4 | 0, 3, 6, 11 | 3, 11 |
| 2 | 7F4 | 6 | 0.3 | 0.3 | IP | BIW x4 | 0, 3, 6, 11 | 3, 11 |
| 3 | 16E4 | 6 | 0.3 | 0.3 | IP | BIW x4 | 0, 3, 6, 11 | 3, 11 |
| 4 | 17B10 | 6 | 0.3 | 0.3 | IP | BIW x4 | 0, 3, 6, 11 | 3, 11 |
| 4 | 7F3/VEGFRFc | 6 | 0.3 | 0.3 | IP | BIW x4 | 0, 3, 6, 11 | 3, 11 |

The 17B10 antibody is the humanized therapeutic antibody

When tumors reach 50-60 mm³, the mice (n=8 per test article) were randomly assigned to groups. Anti-CD93 antibodies (and isotype control) were dosed at 0.3 mg/mouse intraperitoneally on days 0, 3, 6, 11. Efficacy was evaluated based on overall tumor volume. Body weight was measured to ensure that the general health of the animals was not affected by the test.

By showing tumor volume+/−SEM from isotype control baseline, FIG. 52C shows that mice in 7F3, 16E4, 17B10 and 7F3/VEGFRFc exhibited significant inhibition of tumor growth compared to mice in IgG1 isotype control group (p<0.05), suggesting excellent anti-tumor effects. Mice body weights of all tested antibody groups, including the isotype control group, were not affected by the tests.

### Example 21. The Bi-Specific Anti-CD93 Antibody and VEGFR Fusion Protein Generation and Evaluation

1. Bi-Specific Anti-CD93 Antibody and VEGFR Fusion Protein Design

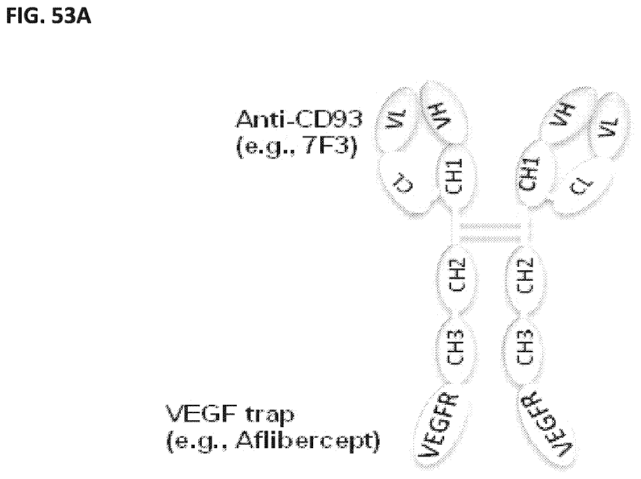

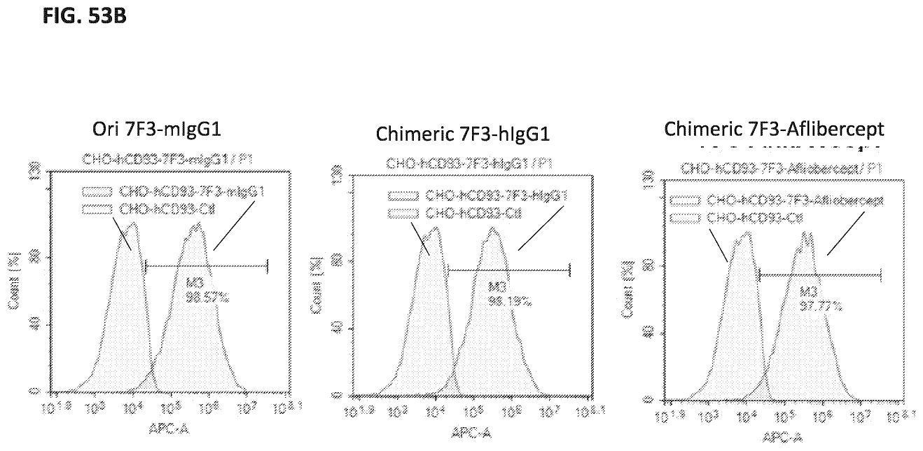

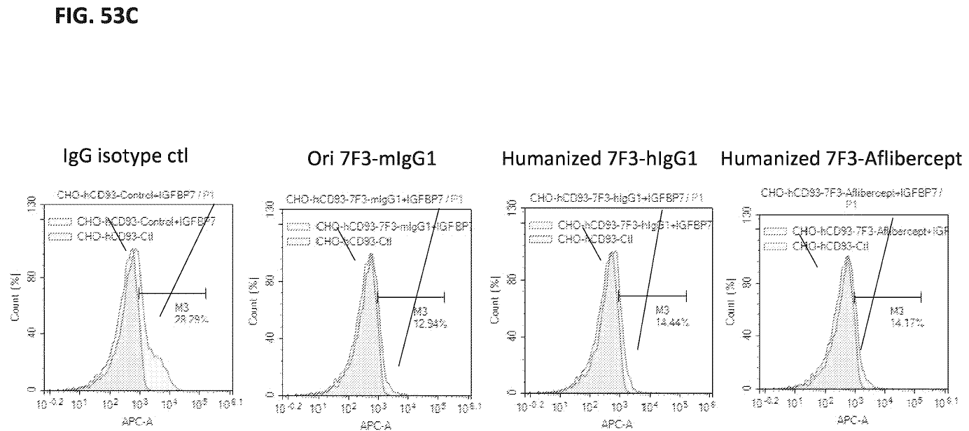

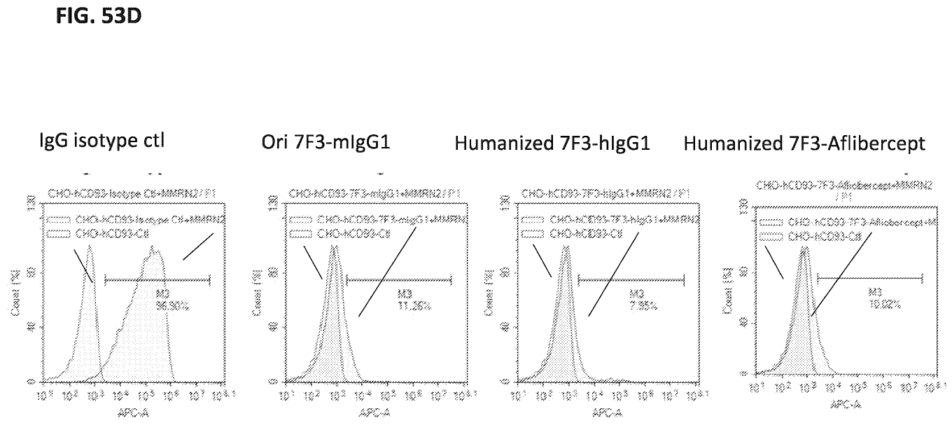

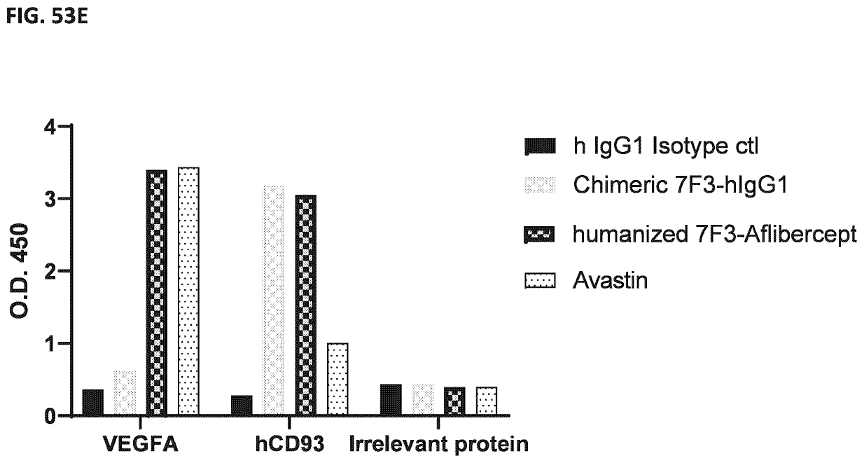

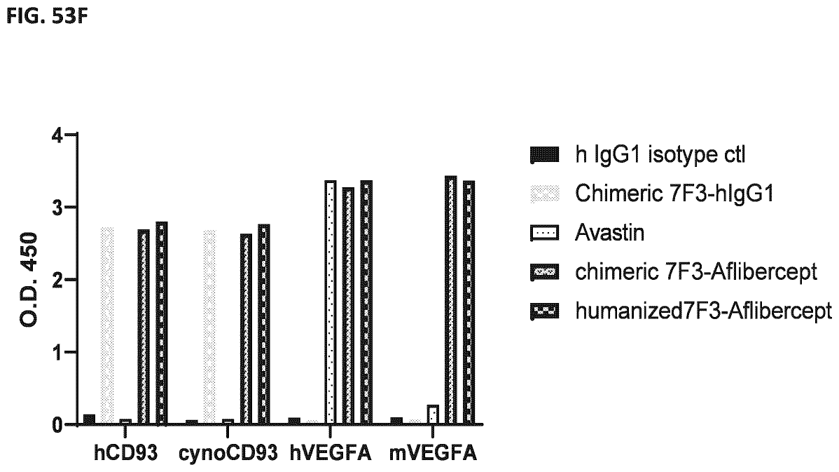

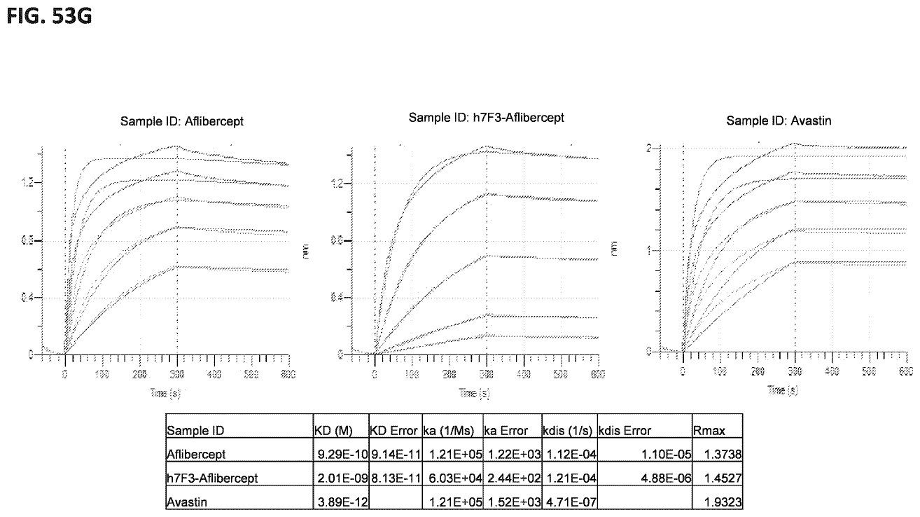

The anti-CD93 constructs that also target VEGF were designed and generated. See FIG. 53A. For example, VEGF-trap (Aflibercept, e.g., SEQ ID NO: 325) were fused to C-terminus of two heavy chains of full-length human IgG1 antibody that comprises heavy chain variable region and light chain variable region of any of the 7F3 and its humanized sequences. An exemplary construct of h7F3/VEGFR having a heavy chain-Aflibercept fusion of SEQ ID NO: 366 and a light chain of SEQ ID NO: 367 was denoted h7F3/VEGFRFc and used for further characterization.

2. Binding of Original Murine 7F3, Humanized 7F3 and Humanized 7F3/VEGFRFc to Cell Surface Expressing Human CD93 CHO Cells Determined by Fluorescence Activated Cell Sorting (FACS) Assay

Human CD93 expressing CHO cells were detached by incubation with TrypLE reagents (Thermos Fisher), which preserves the integrity of CD93 on the cell surface. The cells were then incubated with original murine 7F3, humanized 7F3, and humanized 7F3/VEGFRFc at 10 μg/ml for 30 minutes in 4° C. After washing with FACS buffer, the cells were incubated with Alexa Fluor 488 conjugated anti-human IgG or anti-mouse IgG antibodies (Jackson ImmunoResearch) for 30 minutes at 4° C. After washing with FACS buffer twice, the samples were acquired in NovoCyte Flow Cytometer and analyzed by NovoExpress software.

As shown in FIG. 53B, the original murine 7F3, humanized 7F3, and humanized 7F3/VEGFRFc bi-specific fusion protein all showed strong binding to hCD93 expressing CHO cells. No binding in IgG isotype control was observed.

3. IGFBP7/CD93 Blockade Assay in Human CD93 Expressing CHO Cells by Original Murine 7F3, Humanized 7F3 and Humanized 7F3/VEGFRFc Treatment

Human CD93 expressing CHO cells (1×10⁵ per well) were treated with original murine 7F3, humanized 7F3, and humanized 7F3/VEGFRFc bi-specific fusion protein or isotype control at 50 μg/ml for 30 minutes at 4° C. Subsequently, the cells were incubated with HIS tagged human IGFBP7 recombinant protein (0.5 μg/ml) for another 30 minutes at 4° C. The cells were then washed with FACS buffer and incubated with a rabbit anti-IGFBP7 antibody (Sino Biological Inc, Catalog #13100-R003) at 1 μg/ml for 30 minutes at 4° C. After incubation, the cells were washed with FACS buffer and incubated with PE-conjugated anti-rabbit IgG antibody (Biolegend) for 30 minutes in 4° C. After washing by FACS buffer twice, the samples were analyzed, and data acquired in NovoCyte Flow.

As shown in FIG. 53C, the original murine 7F3, humanized 7F3, and humanized 7F3/VEGFRFc were capable of blocking the interaction between CD93 and IGFBP7. The isotype control antibody was not able to block the interaction between CD93 and IGFBP7.

4. MMRN2/CD93 Blockade Assay in Human CD93 Expressing CHO Cells by Original Murine 7F3, Humanized 7F3 and Humanized 7F3/VEGFRFc Treatment

Human CD93 expressing CHO cells (1×10⁵ per well) were treated with original murine 7F3, humanized 7F3, and humanized 7F3/VEGFRFc bi-specific fusion protein or isotype control at 50 μg/ml for 30 minutes at 4° C. The cells were then incubated with biotinylated MMRN2 protein (0.001 μg/ml) for another 30 minutes at 4° C. After incubation, the cells were washed with FACS buffer and incubated with streptavidin conjugated APC at a ratio of 1:1000 for 30 minutes at 4° C. After washing with FACS buffer twice, the samples were analyzed and data acquired in NovoCyte Flow.

As shown in FIG. 53D, the original murine 7F3, humanized 7F3, and humanized 7F3/VEGFRFc effectively blocked the interaction between MMRN2 and CD93, but isotype control antibody showed no blockade of the interactions between MMRN2 and CD93.

5. ELISA Binding Analyses of Original Murine 7F3, Humanized 7F3 and Humanized 7F3/VEGFRFc

His-tagged human CD93, rh VEGFA (recombinant human VEGFA), or irrelevant His protein were coated onto a 96 well plate at 1 μg/mL in 1× PBS overnight at 4° C. The plate was washed with ELISA wash buffer (Boston BioProduct, Inc.) and the wells were blocked with 3% BSA/PBS ELISA blocking buffer for 1 hour at 37° C. The Avastin, humanized 7F3, and humanized 7F3/VEGFRFc incubated on ice for 1 hour. The plate was washed with ELISA wash Buffer. HRP conjugated anti-human Fc was added and incubated for one hour at 37° C. The plate was washed with ELISA wash buffer and read in a microplate reader at 405 nm.

The results shown in FIG. 53E demonstrates that 7F3/VEGFRFc and Avastin strongly bound to rh VEGFA, whereas chimeric 7F3 and humanized 7F3/VEGFRFc strongly bound to rh CD93. None of Avastin, chimeric 7F3, and humanized 7F3/VEGFRFc showed binding to the irrelevant His protein.

His-tagged human CD93, cyno CD93, human VEGFA, and mouse VEGFA were coated onto a 96 well plate at 1 μg/mL in 1× PBS overnight at 4′C. The plate was washed with ELISA wash buffer (Boston BioProduct, Inc.) and the wells were blocked with 3% BSA/PBS ELISA blocking buffer for 1 hour at 37′C. The Avastin, humanized 7F3, chimeric7F3/VEGFRFc and humanized 7F3/VEGFRFc and control human IgG Fc were incubated on ice for 1 hour. The plate was washed with ELISA wash Buffer. HRP conjugated Anti-human Fc was added and incubated for one hour at 37° C. The plate was washed with ELISA wash buffer and read in a microplate reader at 405 nm.

As shown in FIG. 53F, the chimeric 7F3, chimeric7F3/VEGFRFc and humanized 7F3/VEGFRFc bound to rh CD93 (recombinant human CD93), cyno CD93, rh VEGFA (recombinant human VEGFA), and rm VEGFA (recombinant mouse VEGFA) strongly. The chimeric 7F3 did not show binding to rh VEGFA. Avastin did not show binding to rh CD93 or rm VEGFA. The Avastin, chimeric 7F3, chimeric 7F3/VEGFRFc and humanized 7F3/VEGFRFc did not exhibit binding activity to the control hIgG Fc.

6. Octet Binding Affinity Analysis of Avastin, VEGFRFc, and Humanized 7F3/VEGFRFc

The binding affinities of Avastin, VEGFRFc, and humanized 7F3/VEGFRFc to rh VEGFA were determined with bio-layer interferometry using Octet QKe (Fortebio). The rh VEGFA was made in-house and biotinylated using EZ-LINK NHS-PEG4 biotin (Thermo Fisher Scientific). Streptavidin biosensors (Fortebio) were used to load biotinylated rh VEGFA protein (300 seconds in 5 μg/ml). Baseline was stabilized for 60 seconds in 1× kinetics buffer (Fortebio) before, the Avastin, VEGFRFc, and humanized 7F3/VEGFRFc, at a serial dilution, were allowed to associate for 300 seconds with captured protein. Then the sensors were dissociated in 1× kinetics buffer for 600 seconds. Data analysis was performed on ForteBio Data Analysis HT 11.1 software.

FIG. 53G shows the binding affinities of VEGFRFc and humanized 7F3/VEGFRFc with rh VEGFA protein are similar (VEGFRFc trap is 0.93 nM and 7F3/VEGFRFc is 2 nM).

SEQUENCE TABLE

| SEQ ID | | |
|---|---|---|
| NO. | Description | Nucleotide or Amino Acid Sequence |
| 1. | 10B1 HC- | SFGVN |
| | CDR1 | |
| | (Kabat) | |
| 2. | 10B1 HC- | VIWSGGSTDYNVAFIS |
| | CDR2 | |
| | (Kabat) | |
| 3. | 10B1 HC | NWRYDGYFYAMDY |
| | CDR3 | |
| | (Kabat) | |
| 4. | 10B1 LC- | KASQNVGTNVA |
| | CDR1 | |
| | (Kabat) | |
| 5. | 10B1 LC- | SASYRFI |
| | CDR2 | |
| | (Kabat) | |
| 6. | 10B1 LC- | QQYNRNPIT |
| | CDR3 | |
| | (Kabat) | |
| 7. | 10B1 HC- | DFSLSSFG |
| | CDR1 | |
| | (Vbase2) | |
| 8. | 10B1 HC- | IWSGGST |
| | CDR2 | |
| | (Vbase2) | |
| 9. | 10B1 HC- | ARNWRYDGYFYAMDY |
| | CDR3 | |
| | (Vbase2) | |
| 10. | 10B1 LC- | QNVGTN |
| | CDR1 | |
| | (Vbase2) | |
| 11. | 10B1 LC- | SAS |
| | CDR2 | |
| | (Vbase2) | |
| 12. | 10B1 LC- | QQYNRNPIT |
| | CDR3 | |
| | (Vbase2) | |
| 13. | 10B1 VH | QVQLKQSGPGLVQPSQSLSITCTVSDFSLSSFGVNWV |
| | Amino Acid | RQPPGKGLEWLGVIWSGGSTDYNVAFISRLSISKDNS |
| | Sequence | KSQVFFKMNNLQADDTAIYYCARNWRYDGYFYAM |
| | | DYWGQGTSVTVSS |
| 14. | 10B1 VL | DIVMTQSQKFMSTSTGDRVSVTCKASQNVGTNVAW |
| | Amino Acid | YQQKPGQSPKALIYSASYRFIGVPDRFTGSGSGTDFTL |
| | Sequence | TITNVQSEDLAEYFCQQYNRNPITFGSGTKLEIK |
| 15. | 10B1 VH | CAGGTGCAGCTGAAGCAGTCAGGACCTGGCCTAGT |
| | DNA | GCAGCCCTCACAGAGCCTGTCCATCACCTGCACAG |
| | Sequence | TCTCTGATTTCTCATTATCTAGCTTTGGTGTAAACT |
| | | GGGTTCGCCAGCCTCCAGGAAAGGGTCTGGAGTGG |
| | | CTGGGGGTGATATGGAGTGGTGGAAGTACAGACTA |
| | | TAATGTAGCTTTCATATCCAGACTGAGCATCAGCA |
| | | AGGACAACTCCAAGAGCCAAGTTTTCTTTAAAATG |
| | | AACAATCTGCAAGCTGATGACACAGCCATATACTA |
| | | CTGTGCCAGAAATTGGAGGTATGATGGTTACTTCT |
| | | ATGCTATGGACTACTGGGGTCAAGGAACCTCAGTC |
| | | ACCGTCTCCTCAG |
| 16. | 10B1 VL | GACATTGTGATGACCCAGTCTCAAAAATTCATGTC |
| | DNA | CACATCAACAGGAGACAGGGTCAGCGTCACCTGCA |
| | Sequence | AGGCCAGTCAGAATGTGGGTACTAATGTAGCCTGG |
| | | TATCAACAGAAACCAGGACAGTCTCCTAAAGCACT |
| | | GATTTACTCGGCATCATACCGATTCATTGGAGTCCC |
| | | TGATCGCTTCACAGGCAGTGGATCTGGGACAGATT |
| | | TCACTCTCACCATCACCAATGTGCAGTCTGAAGAC |
| | | TTGGCAGAGTATTTCTGTCAGCAATATAACAGAAA |
| | | TCCTATCACGTTCGGCTCGGGGACAAAGTTGGAAA |
| | | TAAAAC |
| 17. | 16E4 HC- | SYWMH |
| | CDR1 | |
| | (Kabat) | |
| 18. | 16E4 HC- | EIDPSASYTYYNQKFKG |
| | CDR2 | |
| | (Kabat) | |
| 19. | 16E4 HC- | SVYYGNKYFDV |
| | CDR3 | |
| | (Kabat) | |
| 20. | 16E4 LC- | KASQSVDYAGDSYMN |
| | CDR1 | |
| | (Kabat) | |
| 21. | 16E4 LC- | AASNLES |
| | CDR2 | |
| | (Kabat) | |
| 22. | 16E4 LC- | QQTNEDPRT |
| | CDR3 | |
| | (Kabat) | |
| 23. | 16E4 HC- | GYTFTSYW |
| | CDR1 | |
| | (Vbase2) | |
| 24. | 16E4 HC- | IDPSASYT |
| | CDR2 | |
| | (Vbase2) | |
| 25. | 16E4 HC | ARSVYYGNKYFDV |
| | CDR3 | |
| | (Vbase2) | |
| 26. | 16E4 LC- | QSVDYAGDSY |
| | CDR1 | |
| | (Vbase2) | |
| 27. | 16E4 LC- | AAS |
| | CDR2 | |
| | (Vbase2) | |
| 28. | 16E4 LC- | QQTNEDPRT |
| | CDR3 | |
| | (Vbase2) | |
| 29. | 16E4 VH | QVQLQQPGAELVKPGASVKLSCKASGYTFTSYWMH |
| | Amino Acid | WVKQRPGQGLEWIGEIDPSASYTYYNQKFKGKATLT |
| | Sequence | VDKSSSTAYMQLSSLTSEDSAVYYCARSVYYGNKYF |
| | | DVWGAGTTVTVSS |
| 30. | 16E4 VL | DIVLTQSPASLAVSLGQRATISCKASQSVDYAGDSYM |
| | Amino Acid | NWYQQKPGQPPKLLIYAASNLESGIPARFSGSGSGTD |
| | Sequence | FTLNIHPVEEEDAATYYCQQTNEDPRTFGGGTKLEIK |
| 31. | 16E4 VH | CAGGTCCAGCTTCAGCAGCCTGGGGCTGAACTGGT |
| | DNA | GAAGCCTGGGGCTTCAGTGAAGCTGTCCTGCAAGG |
| | Sequence | CTTCTGGATACACCTTCACTAGCTACTGGATGCACT |
| | | GGGTGAAGCAGAGGCCTGGACAAGGCCTTGAGTG |
| | | GATCGGAGAGATTGATCCTTCTGCTAGTTATACTTA |
| | | CTACAATCAAAAGTTCAAGGGCAAGGCCACATTGA |
| | | CTGTAGACAAATCCTCCAGCACAGCCTACATGCAA |
| | | CTCAGCAGCCTGACATCTGAGGACTCTGCGGTCTA |
| | | TTACTGTGCAAGATCGGTCTACTATGGTAACAAGT |
| | | ATTTCGATGTCTGGGGCGCAGGGACCACGGTCACC |
| | | GTCTCCTCA |
| 32. | 16E4 VL | GACATTGTGCTGACCCAATCTCCAGCTTCTTTGGCT |
| | DNA | GTGTCTCTAGGGCAGAGGGCCACCATCTCCTGCAA |
| | Sequence | GGCCAGCCAAAGTGTTGATTATGCCGGTGATAGTT |
| | | ATATGAACTGGTACCAACAGAAACCAGGACAGCC |
| | | ACCCAAACTCCTCATCTATGCTGCATCCAATCTAGA |
| | | ATCTGGGATCCCAGCCAGGTTTAGTGGCAGTGGGT |
| | | CTGGGACAGACTTCACCCTCAACATCCATCCTGTG |
| | | GAGGAGGAGGATGCTGCAACCTATTACTGTCAGCA |
| | | AACTAATGAGGATCCTCGGACGTTCGGTGGAGGCA |
| | | CCAAGCTGGAAATCAAAC |
| 33. | 5H9 HC- | TYWMN |
| | CDR1 | |
| | (Kabat) | |
| 34. | 5H9 HC- | RIFPGDGDANYNGKFKG |
| | CDR2 | |
| | (Kabat) | |
| 35. | 5H9 HC- | TGAAYDFDPFPY |
| | CDR3 | |
| | (Kabat) | |
| 36. | 5H9 LC- | SSSKSLLHSNGVTYLY |
| | CDR1 | |
| | (Kabat) | |
| 37. | 5H9 LC | RMSNLAS |
| | CDR2 | |
| | (Kabat) | |
| 38. | 5H9 LC- | AQMLERPFT |
| | CDR3 | |
| | (Kabat) | |
| 39. | 5H9 HC- | GYAFSTYW |
| | CDR1 | |
| | (Vbase2) | |
| 40. | 5H9 HC- | IFPGDGDA |
| | CDR2 | |
| | (Vbase2) | |
| 41. | 5H9 HC- | TRTGAAYDFDPFPY |
| | CDR3 | |
| | (Vbase2) | |
| 42. | 5H9 LC- | KSLLHSNGVTY |
| | CDR1 | |
| | (Vbase2) | |
| 43. | 5H9 LC- | RMS |
| | CDR2 | |
| | (Vbase2) | |
| 44. | 5H9 LC- | AQMLERPFT |
| | CDR3 | |
| | (Vbase2) | |
| 45. | 5H9 VH | QVQLQQSGPDLVKPGASVKISCKASGYAFSTYWMN |
| | Amino Acid | WVKQRPGKGLEWIGRIFPGDGDANYNGKFKGKATL |
| | Sequence | TADKSSSTAYMQLSSLTSEDSAVYFCTRTGAAYDFDP |
| | | FPYWGQGTLVTVSA |
| 46. | 5H9 VL | DIVMTQAAFSNPVTLGTSASISCSSSKSLLHSNGVTYL |
| | Amino Acid | YWYLQRPGQSPQLLIYRMSNLASGVPDRFSGSGSGT |
| | Sequence | DFTLRISRVEAEDVGIYYCAQMLERPFTFGSGTKLEIK |
| 47. | 5H9 VH | CAGGTTCAGCTGCAGCAGTCTGGACCTGACCTGGT |
| | DNA | GAAGCCTGGGGCCTCAGTGAAGATTTCCTGCAAAG |
| | Sequence | CTTCTGGCTACGCATTCAGTACCTACTGGATGAACT |
| | | GGGTGAAGCAGAGGCCTGGAAAGGGTCTTGAGTG |
| | | GATTGGACGGATTTTTCCTGGAGATGGAGATGCTA |
| | | ACTACAATGGGAAGTTCAAGGGCAAGGCCACACTG |
| | | ACTGCAGACAAATCCTCCAGCACAGCCTACATGCA |
| | | ACTCAGCAGCCTGACATCTGAGGACTCTGCGGTCT |
| | | ACTTCTGTACAAGAACTGGGGCCGCCTATGATTTC |
| | | GACCCTTTTCCTTACTGGGGCCAAGGGACTCTGGTC |
| | | ACTGTCTCTGCAG |
| 48. | 5H9 VL DNA | GATATTGTGATGACGCAGGCTGCATTCTCCAATCC |
| | Sequence | AGTCACTCTTGGAACATCAGCTTCCATCTCTTGCAG |
| | | TTCTAGTAAGAGTCTCCTACATAGTAATGGCGTCA |
| | | CTTATTTGTATTGGTATCTGCAGAGGCCAGGCCAGT |
| | | CTCCTCAGCTCCTGATATATCGGATGTCCAACCTTG |
| | | CCTCAGGAGTCCCAGACAGGTTCAGTGGCAGTGGG |
| | | TCAGGAACTGATTTCACACTGAGAATCAGCAGAGT |
| | | GGAGGCTGAGGATGTGGGTATTTATTACTGTGCTC |
| | | AAATGCTAGAACGCCCATTCACGTTCGGCTCGGGG |
| | | ACAAAGTTGGAAATAAAAC |
| 49. | 16G9 HC- | DYYMN |
| | CDR1 | |
| | (Kabat) | |
| 50. | 16G9 HC- | RVNPNNGGKTYNQKFKG |
| | CDR2 | |
| | (Kabat) | |
| 51. | 16G9 HC- | WRLRPVDYGMDY |
| | CDR3 | |
| | (Kabat) | |
| 52. | 16G9 LC- | RASQSVSTSSYSYMH |
| | CDR1 | |
| | (Kabat) | |
| 53. | 16G9 LC- | YASNLES |
| | CDR2 | |
| | (Kabat) | |
| 54. | 16G9 LC | QHSWEIPFT |
| | CDR3 | |
| | (Kabat) | |
| 55. | 16G9 HC- | GYTFTDYY |
| | CDR1 | |
| | (Vbase2) | |
| 56. | 16G9 HC- | VNPNNGGK |
| | CDR2 | |
| | (Vbase2) | |
| 57. | 16G9 HC- | ARWRLRPVDYGMDY |
| | CDR3 | |
| | (Vbase2) | |
| 58. | 16G9 LC- | QSVSTSSYSY |
| | CDR1 | |
| | (Vbase2) | |
| 59. | 16G9 LC- | YAS |
| | CDR2 | |
| | (Vbase2) | |
| 60. | 16G9 LC- | QHSWEIPFT |
| | CDR3 | |
| | (Vbase2) | |
| 61. | 16G9 VH | EVQLQQSGPELVKPGASVKMSCKASGYTFTDYYMN |
| | Amino Acid | WVKQSHGKSLEWIGRVNPNNGGKTYNQKFKGKATL |
| | Sequence | TVDKSLSTAYMQLNSLTSEDSAVYYCARWRLRPVDY |
| | | GMDYWGQGTSVTVSS |
| 62. | 16G9 VL | DIVLTQSPASLAVSLGQRATISCRASQSVSTSSYSYMH |
| | Amino Acid | WYQQKPGQPPKLLIKYASNLESGVPARFSGSGSGTDF |
| | Sequence | TLNIHPVEEEDTATYYCQHSWEIPFTFGSGTKLEIK |
| 63. | 16G9 VH | GAGGTCCAGCTGCAACAGTCTGGACCTGAGCTGGT |
| | DNA | GAAGCCTGGGGCTTCAGTGAAGATGTCCTGTAAGG |
| | Sequence | CTTCTGGATACACATTCACTGACTACTACATGAACT |
| | | GGGTGAAGCAGAGTCATGGAAAGAGTCTTGAGTG |
| | | GATTGGACGTGTTAATCCTAACAATGGTGGTAAAA |
| | | CCTACAACCAGAAGTTCAAGGGCAAGGCCACATTG |
| | | ACAGTAGACAAATCCCTCAGCACAGCCTACATGCA |
| | | GCTCAACAGCCTGACATCTGAGGACTCTGCGGTCT |
| | | ATTACTGTGCAAGATGGAGGCTACGGCCCGTTGAC |
| | | TATGGTATGGACTACTGGGGTCAAGGAACCTCAGT |
| | | CACCGTCTCCTCAG |
| 64. | 16G9 VL | GACATTGTGCTGACACAGTCTCCTGCTTCCTTGGCT |
| | DNA | GTATCTCTGGGGCAGAGGGCCACCATCTCATGCAG |
| | Sequence | GGCCAGCCAAAGTGTCAGTACATCTAGCTATAGTT |
| | | ATATGCACTGGTACCAACAGAAACCAGGACAGCCA |
| | | CCCAAACTCCTCATCAAGTATGCATCCAACCTAGA |
| | | ATCTGGGGTCCCTGCCAGGTTCAGTGGCAGTGGGT |
| | | CTGGGACAGACTTCACCCTCAACATCCATCCTGTG |
| | | GAGGAGGAGGATACTGCAACATATTACTGTCAGCA |
| | | CAGTTGGGAGATTCCATTCACGTTCGGCTCGGGGA |
| | | CAAAGTTGGAAATAAAAC |
| 65. | 19E12 HC- | DYEMH |
| | CDR1 | |
| | (Kabat) | |
| 66. | 19E12 HC- | GIDPETGGTAYNQKFKG |
| | CDR2 | |
| | (Kabat) | |
| 67. | 19E12 HC- | GAWFAY |
| | CDR3 | |
| | (Kabat) | |
| 68. | 19E12 LC- | RSSTGAVTTSNSAN |
| | CDR1 | |
| | (Kabat) | |
| 69. | 19E12 LC- | GTNNRAP |
| | CDR2 | |
| | (Kabat) | |
| 70. | 19E12 LC- | ALWYNNHFV |
| | CDR3 | |
| | (Kabat) | |
| 71. | 19E12 HC- | GYTFTDYE |
| | CDR1 | |
| | (Vbase2) | |
| 72. | 19E12 HC- | IDPETGGT |
| | CDR2 | |
| | (Vbase2) | |
| 73. | 19E12 HC- | TRGAWFAY |
| | CDR3 | |
| | (Vbase2) | |
| 74. | 19E12 LC- | TGAVTTSNS |
| | CDR1 | |
| | (Vbase2) | |
| 75. | 19E12 LC- | GTN |
| | CDR2 | |
| | (Vbase2) | |
| 76. | 19E12 LC- | ALWYNNHFV |
| | CDR3 | |
| | (Vbase2) | |
| 77. | 19E12 VH | QVQLQQSGAELVRPGASVKLSCKASGYTFTDYEMH |
| | Amino Acid | WVRQTPVHGLEWIGGIDPETGGTAYNQKFKGKATLT |
| | Sequence | ADKSSSTAYMELRSLTSEDSAVYYCTRGAWFAYWG |
| | | QGTLVTVSA |
| 78. | 19E12 VL | QAVVTQESALTTSPGETVTLTCRSSTGAVTTSNSANW |
| | Amino Acid | VQEKPDHLFTGLIGGTNNRAPGVPARFSGSLIGDKAA |
| | Sequence | LTITGAQTEDEAIYFCALWYNNHFVFGGGTKLTVL |
| 79. | 19E12 VH | CAGGTTCAATTGCAGCAGTCTGGGGCTGAGCTGGT |
| | DNA | GAGGCCTGGGGCTTCAGTGAAGCTGTCCTGCAAGG |
| | Sequence | CTTCGGGCTATACATTTACTGACTATGAAATGCACT |
| | | GGGTGAGGCAGACACCTGTGCATGGCCTGGAATGG |
| | | ATTGGAGGTATTGATCCTGAAACTGGTGGTACTGC |
| | | CTACAATCAGAAGTTCAAGGGCAAGGCCACACTGA |
| | | CTGCAGACAAATCCTCCAGCACAGCCTACATGGAG |
| | | CTCCGCAGCCTGACATCTGAGGACTCTGCCGTCTAT |
| | | TACTGTACACGAGGGGCCTGGTTTGCTTACTGGGG |
| | | CCAAGGGACTCTGGTCACTGTCTCTGCAG |
| 80. | 19E12 VL | CAGGCTGTTGTGACTCAGGAATCTGCACTCACCAC |
| | DNA | ATCACCTGGTGAAACAGTCACACTCACTTGTCGCT |
| | Sequence | CAAGTACTGGGGCTGTTACAACTAGTAACTCTGCC |
| | | AACTGGGTCCAAGAAAAACCAGATCATTTATTCAC |
| | | TGGTCTAATCGGTGGTACCAACAACCGAGCTCCAG |
| | | GTGTTCCTGCCAGATTCTCAGGCTCCCTGATTGGAG |
| | | ACAAGGCTGCCCTCACCATCACAGGGGCACAGACT |
| | | GAGGATGAGGCAATATATTTCTGTGCTCTATGGTA |
| | | CAACAACCATTTCGTGTTCGGTGGAGGCACCAAAC |
| | | TGACTGTCCTAG |
| 81. | 17G11 HC- | SYWMH |
| | CDR1 | |
| | (Kabat) | |
| 82. | 17G11 HC- | AIYPGNSDTSYNQKFKG |
| | CDR2 | |
| | (Kabat) | |
| 83. | 17G11 HC- | GGFDYSNYWFAY |
| | CDR3 | |
| | (Kabat) | |
| 84. | 17G11 LC- | KASQSVSNDVA |
| | CDR1 | |
| | (Kabat) | |
| 85. | 17G11 LC- | YASNRYT |
| | CDR2 | |
| | (Kabat) | |
| 86. | 17G11 LC- | QQDYSSYT |
| | CDR3 | |
| | (Kabat) | |
| 87. | 17G11 HC- | GYTFTSYW |
| | CDR1 | |
| | (Vbase2) | |
| 88. | 17G11 HC- | IYPGNSDT |
| | CDR2 | |
| | (Vbase2) | |
| 89. | 17G11 HC- | TRGGFDYSNYWFAY |
| | CDR3 | |
| | (Vbase2) | |
| 90. | 17G11 LC- | QSVSND |
| | CDR1 | |
| | (Vbase2) | |
| 91. | 17G11 LC- | YAS |
| | CDR2 | |
| | (Vbase2) | |
| 92. | 17G11 LC- | QQDYSSYT |
| | CDR3 | |
| | (Vbase2) | |
| 93. | 17G11 VH | EVQLQQSGTVLARPGASVKMSCKASGYTFTSYWMH |
| | Amino Acid | WVKQRPGQGLEWIGAIYPGNSDTSYNQKFKGKAKLT |
| | Sequence | AVTSASTAYMELSSLTNEDSAVYYCTRGGFDYSNYW |
| | | FAYWGQGTLVTVSA |
| 94. | 17G11 VL | SIVMTQTPKFLLVSAGDRVTITCKASQSVSNDVAWY |
| | Amino Acid | QQKPGQSPKLLIYYASNRYTGVPDRFTGSGYGTDFTF |
| | Sequence | TISTVQAEDLAVYFCQQDYSSYTFGGGTKLEIK |
| 95. | 17G11 VH | GAGGTTCAGCTCCAGCAGTCTGGGACTGTGCTGGC |
| | DNA | AAGGCCTGGGGCTTCAGTGAAGATGTCCTGCAAGG |
| | Sequence | CTTCTGGCTACACCTTTACCAGCTACTGGATGCACT |
| | | GGGTAAAACAGAGGCCTGGACAGGGTCTGGAATG |
| | | GATTGGCGCTATTTATCCTGGAAATAGTGATACTA |
| | | GCTACAACCAGAAGTTCAAGGGCAAGGCCAAACT |
| | | GACTGCAGTCACATCTGCCAGCACTGCCTACATGG |
| | | AGCTCAGCAGCCTGACAAATGAGGACTCTGCGGTC |
| | | TATTACTGTACAAGAGGAGGATTTGACTATAGTAA |
| | | CTACTGGTTTGCTTACTGGGGCCAAGGGACTCTGG |
| | | TCACTGTCTCTGCA |
| 96. | 17G11 VL | AGTATTGTGATGACCCAGACTCCCAAATTCCTGCTT |
| | DNA | GTATCAGCAGGAGACAGGGTTACCATAACCTGCAA |
| | Sequence | GGCCAGTCAGAGTGTGAGTAATGATGTAGCTTGGT |
| | | ACCAACAGAAGCCAGGGCAGTCTCCTAAACTGCTG |
| | | ATATACTATGCATCCAATCGCTACACTGGAGTCCCT |
| | | GATCGCTTCACTGGCAGTGGATATGGGACGGATTT |
| | | CACTTTCACCATCAGCACTGTGCAGGCTGAAGACC |
| | | TGGCAGTTTATTTCTGTCAGCAGGATTATAGCTCGT |
| | | ACACGTTCGGAGGGGGGACCAAGCTGGAAATAAA |
| | | AC |
| 97. | 16B6 HC- | RSWMN |
| | CDR1 | |
| | (Kabat) | |
| 98. | 16B6 HC- | WIYPGDGDTNYNGKFKG |
| | CDR2 | |
| | (Kabat) | |
| 99. | 16B6 HC- | SATLPYWYFDV |
| | CDR3 | |
| | (Kabat) | |
| 100. | 16B6 LC- | KASQDIKSYLS |
| | CDR1 | |
| | (Kabat) | |
| 101. | 16B6 LC- | YATNLAD |
| | CDR2 | |
| | (Kabat) | |
| 102. | 16B6 LC- | LQHVESPWT |
| | CDR3 | |
| | (Kabat) | |
| 103. | 16B6 HC- | GYAFSRSW |
| | CDR1 | |
| | (Vbase2) | |
| 104. | 16B6 HC- | IYPGDGDT |
| | CDR2 | |
| | (Vbase2) | |
| 105. | 16B6 HC- | ARSATLPYWYFDV |
| | CDR3 | |
| | (Vbase2) | |
| 106 | 16B6 LC- | QDIKSY |
| | CDR1 | |
| | (Vbase2) | |
| 107. | 16B6 LC- | YAT |
| | CDR2 | |
| | (Vbase2) | |
| 108. | 16B6 LC- | LQHVESPWT |
| | CDR3 | |
| | (Vbase2) | |
| 109. | 16B6 VH | QVQLQQSGPELVKPGASVKISCKASGYAFSRSWMNW |
| | Amino Acid | VKQRPGKGLEWIGWIYPGDGDTNYNGKFKGKATLT |
| | Sequence | ADKSSSTAYMQLSSLTSEDSAAYFCARSATLPYWYF |
| | | DVWGAGTTVTVSS |
| 110. | 16B6 VL | DIKMTQSPSSMYASLGERVTITCKASQDIKSYLSWYQ |
| | Amino Acid | QKPWKSPKTLIYYATNLADGVPSRFSGSGSGQDYSLT |
| | Sequence | ISSLGSDDTATYYCLQHVESPWTFGGGTKLEIK |
| 111. | 16B6 VH | CAGGTCCAGCTGCAGCAGTCTGGACCTGAGCTGGT |
| | DNA | GAAGCCTGGGGCCTCAGTGAAGATTTCCTGCAAAG |
| | Sequence | CTTCTGGCTATGCATTCAGTCGCTCCTGGATGAACT |
| | | GGGTAAAGCAGAGGCCTGGAAAGGGTCTTGAGTG |
| | | GATTGGATGGATTTATCCTGGAGATGGTGATACTA |
| | | ACTACAATGGAAAGTTCAAGGGCAAGGCCACACTG |
| | | ACTGCAGACAAATCCTCAAGCACAGCCTACATGCA |
| | | GCTCAGCAGCCTGACATCTGAGGACTCTGCGGCCT |
| | | ATTTCTGTGCAAGGTCGGCTACCCTACCTTACTGGT |
| | | ACTTCGATGTCTGGGGCGCAGGGACCACGGTCACC |
| | | GTCTCCTCAG |
| 112. | 16B6 VL | GACATCAAGATGACCCAGTCTCCATCCTCCATGTA |
| | DNA | TGCATCGCTGGGAGAGAGAGTCACTATCACTTGCA |
| | Sequence | AGGCGAGTCAGGACATTAAAAGCTATTTAAGTTGG |
| | | TACCAGCAGAAACCATGGAAATCTCCTAAGACCCT |
| | | GATCTATTATGCAACAAACTTGGCAGATGGGGTCC |
| | | CATCAAGATTCAGTGGCAGTGGATCTGGGCAGGAT |
| | | TATTCTCTAACCATCAGCAGCCTGGGGTCTGACGA |
| | | TACAGCAACTTATTACTGTCTACAGCATGTTGAGA |
| | | GCCCGTGGACGTTCGGTGGAGGCACCAAGCTGGAA |
| | | ATCAAAC |
| 113. | 20C7 HC- | AYVMH |
| | CDR1 | |
| | (Kabat) | |
| 114. | 20C7 HC- | YIFPYNDGTEYNEKFKG |
| | CDR2 | |
| | (Kabat) | |
| 115. | 20C7 HC- | RTDGNPYTMDY |
| | CDR3 | |
| | (Kabat) | |
| 116. | 20C7 LC- | KASQDVSTAVA |
| | CDR1 | |
| | (Kabat) | |
| 117. | 20C7 LC- | SASYRYT |
| | CDR2 | |
| | (Kabat) | |
| 118. | 20C7 LC- | QQHYSTPFT |
| | CDR3 | |
| | (Kabat) | |
| 119. | 20C7 HC- | GYTFTAYV |
| | CDR1 | |
| | (Vbase2) | |
| 120. | 20C7 HC- | IFPYNDGT |
| | CDR2 | |
| | (Vbase2) | |
| 121. | 20C7 HC- | ARRTDGNPYTMDY |
| | CDR3 | |
| | (Vbase2) | |
| 122. | 20C7 LC- | QDVSTA |
| | CDR1 | |
| | (Vbase2) | |
| 123. | 20C7 LC- | SAS |
| | CDR2 | |
| | (Vbase2) | |
| 124. | 20C7 LC- | QQHYSSPFT |
| | CDR3 | |
| | (Vbase2) | |
| 125. | 20C7 VH | EVQLQQSGPELVNPGASVKMSCKASGYTFTAYVMH |
| | Amino Acid | WVKQKPGQGLEWIGYIFPYNDGTEYNEKFKGKATLT |
| | Sequence | SDKSSSTAYMELSSLTSEDSAVYYCARRTDGNPYTM |
| | | DYWGQGTSVTVSS |
| 126. | 20C7 VL | DIVMTQSHKFMSTSVGDRVSITCKASQDVSTAVAWY |
| | Amino Acid | QQKPGQSPKLLIHSASYRYTGVPDRFTGRGSGTDFTF |
| | Sequence | TISSVQAEDLAVYYCQQHYSTPFTFGSGTKLEIK |
| 127. | 20C7 VH | GAGGTCCAGCTGCAGCAGTCTGGACCTGAGTTGGT |
| | DNA | AAATCCTGGGGCTTCAGTGAAGATGTCCTGCAAGG |
| | Sequence | CTTCTGGATACACATTCACTGCCTATGTTATGCACT |
| | | GGGTGAAACAGAAGCCTGGGCAGGGCCTTGAGTG |
| | | GATTGGATATATTTTTCCTTACAATGATGGTACTGA |
| | | GTACAATGAGAAGTTCAAAGGCAAGGCCACACTG |
| | | ACTTCAGACAAATCCTCCAGCACAGCCTACATGGA |
| | | GCTCAGCAGCCTGACCTCTGAGGACTCTGCGGTCT |
| | | ATTACTGTGCAAGGAGGACAGATGGTAACCCCTAT |
| | | ACTATGGACTATTGGGGTCAAGGAACCTCAGTCAC |
| | | CGTCTCCTCAG |
| 128. | 20C7 VL | GACATTGTGATGACCCAGTCTCACAAATTCATGTC |
| | DNA | CACATCAGTAGGAGACAGGGTCAGCATCACCTGCA |
| | Sequence | AGGCCAGTCAGGATGTGAGTACTGCTGTAGCCTGG |
| | | TATCAACAGAAACCAGGACAATCTCCTAAACTACT |
| | | GATTCATTCGGCATCCTACCGGTACACTGGAGTCC |
| | | CTGATCGCTTCACTGGCAGAGGATCTGGGACGGAT |
| | | TTCACTTTCACCATCAGCAGTGTGCAGGCTGAAGA |
| | | CCTGGCAGTTTATTACTGTCAGCAACATTATAGTAC |
| | | TCCATTCACGTTCGGCTCGGGGACAAAGTTGGAAA |
| | | TAAAAC |
| 129. | 12H4 HC- | DYYIH |
| | CDR1 | |
| | (Kabat) | |
| 130. | 12H4 HC- | EIYPGSDDAYYNEKFKG |
| | CDR2 | |
| | (Kabat) | |
| 131. | 12H4 HC- | ETTATAY |
| | CDR3 | |
| | (Kabat) | |
| 132. | 12H4 LC- | SASSSVSLIY |
| | CDR1 | |
| | (Kabat) | |
| 133. | 12H4 LC- | STSNLAS |
| | CDR2 | |
| | (Kabat) | |
| 134. | 12H4 LC- | QQRSGYPPT |
| | CDR3 | |
| | (Kabat) | |
| 135. | 12H4 HC- | GYTFTDYY |
| | CDR1 | |
| | (Vbase2) | |
| 136. | 12H4 HC- | IYPGSDDA |
| | CDR2 | |
| | (Vbase2) | |
| 137. | 12H4 HC- | TRETTATAY |
| | CDR3 | |
| | (Vbase2) | |
| 138. | 12H4 LC- | SSVSL |
| | CDR1 | |
| | (Vbase2) | |
| 139. | 12H4 LC- | STS |
| | CDR2 | |
| | (Vbase2) | |
| 140. | 12H4 LC- | QQRSGYPPT |
| | CDR3 | |
| | (Vbase2) | |
| 141. | 12H4 VH | EVQLQQSGPELVKPGASVKVSCKASGYTFTDYYIHW |
| | Amino Acid | VKQRPGQGLEWIGEIYPGSDDAYYNEKFKGKATLTA |
| | Sequence | DKSSSTAYMQLSSLTSEDSAVYFCTRETTATAYWGQ |
| | | GTLVTVSA |
| 142. | 12H4 VL | QIVLTQSPAIMSASPGEKVTITCSASSSVSLIYWFQQKP |
| | Amino Acid | GTSPKLWIYSTSNLASGVPARFSGSGSGTSYSLTISRM |
| | Sequence | EAEDAATYYCQQRSGYPPTFGGGTKLEIK |
| 143. | 12H4 VH | CTGAGGTCCAGCTGCAGCAGTCTGGACCTGAGCTG |
| | DNA | GTTAAGCCTGGGGCTTCAGTGAAGGTATCCTGCAA |
| | Sequence | GGCCTCTGGATACACATTCACTGACTACTATATAC |
| | | ACTGGGTGAAGCAGAGGCCTGGGCAGGGCCTTGA |
| | | GTGGATTGGAGAGATTTATCCTGGAAGTGATGATG |
| | | CTTACTACAATGAGAAATTCAAGGGCAAGGCCACA |
| | | CTGACTGCAGACAAATCCTCCAGCACAGCCTACAT |
| | | GCAGCTCAGCAGCCTGACATCTGAGGACTCTGCAG |
| | | TCTATTTCTGTACAAGAGAGACTACGGCTACGGCT |
| | | TACTGGGGCCAAGGGACTCTGGTCACTGTCTCTGC |
| | | AG |
| 144. | 12H4 VL | CAAATTGTTCTCACCCAGTCTCCAGCAATCATGTCT |
| | DNA | GCATCTCCAGGGGAGAAGGTCACCATAACCTGCAG |
| | Sequence | TGCCAGCTCAAGTGTAAGTCTCATTTACTGGTTCCA |
| | | GCAGAAGCCAGGCACTTCTCCCAAACTCTGGATTT |
| | | ATAGCACATCCAACCTGGCTTCTGGAGTCCCTGCTC |
| | | GCTTCAGTGGCAGTGGATCTGGGACCTCTTACTCTC |
| | | TCACAATCAGCCGAATGGAGGCTGAAGATGCTGCC |
| | | ACTTATTACTGCCAGCAAAGGAGTGGTTACCCACC |
| | | CACGTTCGGAGGGGGGACCAAGCTGGAAATAAAA |
| | | C |
| 145. | 16A1 HC- | DHGIH |
| | CDR1 | |
| | (Kabat) | |
| 146. | 16A1 HC- | NISPGNGDIKYNEKFKG |
| | CDR2 | |
| | (Kabat) | |
| 147. | 16A1 HC- | YFVD |
| | CDR3 | |
| | (Kabat) | |
| 148. | 16A1 LC- | KSSQSLLNSNNQKNCLA |
| | CDR1 | |
| | (Kabat) | |
| 149. | 16A1 LC- | FACTRES |
| | CDR2 | |
| | (Kabat) | |
| 150. | 16A1 LC- | QQHCNTPLT |
| | CDR3 | |
| | (Kabat) | |
| 151. | 16A1 HC- | GYTFTDHG |
| | CDR1 | |
| | (Vbase2) | |
| 152. | 16A1 HC- | ISPGNGDI |
| | CDR2 | |
| | (Vbase2) | |
| 153. | 16A1 HC- | TTYFVD |
| | CDR3 | |
| | (Vbase2) | |
| 154. | 16A1 LC- | QSLLNSNNQKNC |
| | CDR1 | |
| | (Vbase2) | |
| 155. | 16A1 LC- | FAC |
| | CDR2 | |
| | (Vbase2) | |
| 156. | 16A1 LC | QQHCNTPLT |
| | CDR3 | |
| | (Vbase2) | |
| 157. | 16A1 VH | QVQLQQSDAELVKPGTSVKISCKASGYTFTDHGIHW |
| | Amino Acid | VKQRPERGLEWIGNISPGNGDIKYNEKFKGKATLTAD |
| | Sequence | KSSSTVYMQVNSLTSEDSAVYFCTTYFVDWGRGTLV |
| | | TVSA |
| 158. | 16A1 VL | DIVMTQSPSSLAMSIGQRVTMSCKSSQSLLNSNNQKN |
| | Amino Acid | CLAWYQQKPGQSPRLLIYFACTRESGVPDRFIGSGSG |
| | Sequence | TDFTLTISSVQAEDLAYYFCQQHCNTPLTFGAGTKLE |
| | | LK |
| 159. | 16A1 VH | CAGGTTCAGCTGCAACAGTCTGACGCTGAGTTGGT |
| | DNA | GAAACCTGGGACTTCAGTGAAGATATCCTGCAAGG |
| | Sequence | CTTCTGGCTACACCTTCACTGACCATGGTATTCACT |
| | | GGGTGAAACAGAGGCCTGAACGGGGCCTGGAATG |
| | | GATTGGAAATATTTCTCCCGGAAATGGTGATATTA |
| | | AGTATAATGAGAAGTTCAAGGGCAAGGCCACGCTG |
| | | ACTGCAGACAAATCCTCCAGCACTGTCTACATGCA |
| | | GGTCAACAGCCTGACATCTGAGGATTCTGCAGTGT |
| | | ATTTCTGTACAACCTATTTTGTTGACTGGGGCCGGG |
| | | GGACTCTGGTCACTGTCTCTGCAG |
| 160. | 16A1 VL | GACATTGTGATGACACAGTCTCCATCCTCCCTGGCT |
| | DNA | ATGTCAATTGGACAGAGGGTCACTATGAGCTGCAA |
| | Sequence | GTCCAGTCAGAGCCTTTTAAATAGTAACAATCAAA |
| | | AGAACTGTTTGGCCTGGTACCAGCAGAAACCAGGA |
| | | CAGTCTCCTAGACTTCTGATTTACTTTGCATGTACT |
| | | AGGGAATCGGGGGTCCCTGATCGCTTCATTGGCAG |
| | | TGGATCTGGGACAGATTTCACCCTTACCATCAGCA |
| | | GTGTGCAGGCTGAAGACCTGGCATATTACTTCTGT |
| | | CAGCAACATTGTAACACTCCGCTCACGTTCGGTGC |
| | | TGGGACCAAGCTGGAGCTGAAAC |
| 161. | 17A7 HC- | TYWMN |
| | CDR1 | |
| | (Kabat) | |
| 162. | 17A7 HC- | RIFPGDGDTDYDGKFKG |
| | CDR2 | |
| | (Kabat) | |
| 163. | 17A7 HC | TGAAYEFDPFPY |
| | CDR3 | |
| | (Kabat) | |
| 164. | 17A7 LC- | SSTKSLLHSSGITYLY |
| | CDR1 | |
| | (Kabat) | |
| 165. | 17A7 LC- | RMSNLAS |
| | CDR2 | |
| | (Kabat) | |
| 166. | 17A7 LC- | AQMLERPFT |
| | CDR3 | |
| | (Kabat) | |
| 167. | 17A7 HC- | GYAFSTYW |
| | CDR1 | |
| | (Vbase2) | |
| 168. | 17A7 HC- | IFPGDGDT |
| | CDR2 | |
| | (Vbase2) | |
| 169. | 17A7 HC | ARTGAAYEFDPFPY |
| | CDR3 | |
| | (Vbase2) | |
| 170. | 17A7 LC- | KSLLHSSGITY |
| | CDR1 | |
| | (Vbase2) | |
| 171. | 17A7 LC- | RMS |
| | CDR2 | |
| | (Vbase2) | |
| 172. | 17A7 LC- | AQMLERPFT |
| | CDR3 | |
| | (Vbase2) | |
| 173. | 17A7 VH | QVQLQQSGPELVKPGASVKISCKGSGYAFSTYWMN |
| | Amino Acid | WVKQRPGKGLEWIGRIFPGDGDTDYDGKFKGKATLT |
| | Sequence | ADKSSNTAYMQLSSLTSEDSAVYFCARTGAAYEFDP |
| | | FPYWGQGTLVTVSA |
| 174. | 17A7 VL | DIVMTQAAFSNPVTLGTSASISCSSTKSLLHSSGITYLY |
| | Amino Acid | WYLQRPGQSPQLLIYRMSNLASGVPDRFSGSGSGTDF |
| | Sequence | TLRISRVEAEDVGVYYCAQMLERPFTFGSGTKLEIK |
| 175. | 17A7 VH | CAGGTTCAGCTGCAGCAGTCTGGACCTGAGCTGGT |
| | DNA | GAAGCCTGGGGCCTCAGTGAAGATTTCCTGCAAAG |
| | Sequence | GTTCTGGCTACGCATTCAGTACCTACTGGATGAACT |
| | | GGGTGAAGCAGAGGCCTGGAAAGGGTCTTGAGTG |
| | | GATTGGACGGATTTTTCCTGGAGATGGAGATACAG |
| | | ATTACGATGGGAAGTTCAAGGGCAAGGCCACACTG |
| | | ACTGCAGACAAATCCTCCAACACAGCCTACATGCA |
| | | ACTCAGCAGCCTGACATCTGAAGACTCTGCGGTCT |
| | | ACTTCTGTGCAAGAACTGGGGCCGCCTATGAATTC |
| | | GACCCTTTTCCTTACTGGGGCCAAGGGACTCTGGTC |
| | | ACTGTCTCTGCAG |
| 176. | 17A7 VL | GATATTGTGATGACGCAGGCTGCATTCTCCAATCC |
| | DNA | AGTCACTCTTGGAACATCAGCTTCCATCTCTTGCAG |
| | Sequence | TTCTACTAAGAGTCTCCTACATAGTAGCGGCATCA |
| | | CTTATCTGTATTGGTATCTGCAGAGGCCAGGCCAG |
| | | TCTCCTCAGCTCCTGATATATCGGATGTCCAACCTT |
| | | GCCTCAGGAGTCCCAGACAGGTTCAGTGGCAGTGG |
| | | GTCAGGAACTGATTTCACACTGAGAATCAGCAGAG |
| | | TGGAGGCTGAGGATGTGGGTGTTTATTACTGTGCT |
| | | CAAATGCTAGAACGCCCATTCACGTTCGGCTCGGG |
| | | GACAAAGTTGGAAATAAAAC |
| 177. | 17B10 HC- | SYWLN |
| | CDR1 | |
| | (Kabat) | |
| 178. | 17B10 HC- | RIYPGDGDTDYNGKFKG |
| | CDR2 | |
| | (Kabat) | |
| 179. | 17B10 HC- | GDGYWAMDY |
| | CDR3 | |
| | (Kabat) | |
| 180. | 17B10 LC- | RFSKSLLHSNGITYLY |
| | CDR1 | |
| | (Kabat) | |
| 181. | 17B10 LC- | QMSNLAS |
| | CDR2 | |
| | (Kabat) | |
| 182. | 17B10 LC- | AQNLELPWT |
| | CDR3 | |
| | (Kabat) | |
| 183. | 17B10 HC- | GYAFSSYW |
| | CDR1 | |
| | (Vbase2) | |
| 184. | 17B10 HC- | IYPGDGDT |
| | CDR2 | |
| | (Vbase2) | |
| 185. | 17B10 HC- | VRGDGYWAMDY |
| | CDR3 | |
| | (Vbase2) | |
| 186. | 17B10 LC- | KSLLHSNGITY |
| | CDR1 | |
| | (Vbase2) | |
| 187. | 17B10 LC- | QMS |
| | CDR2 | |
| | (Vbase2) | |
| 188. | 17B10 LC- | AQNLELPWT |
| | CDR3 | |
| | (Vbase2) | |
| 189. | 17B10 VH | QVQLQQSGPELVKPGASVKISCKASGYAFSSYWLNW |
| | Amino Acid | VKQRPGKGLEWFGRIYPGDGDTDYNGKFKGKATLT |
| | Sequence | ADKSSSTAYMQLRSLTSEDSAVYFCVRGDGYWAMD |
| | | YWGQGTSVTVSS |
| 190. | 17B10 VL | DIVMTQAAFSNPVTLGTSASISCRFSKSLLHSNGITYL |
| | Amino Acid | YWYLQKPGQSPQLLIYQMSNLASGVPDRFSSSGSGTD |
| | Sequence | FTLRISRVEAEDVGVYYCAQNLELPWTFGGGTKLEIK |
| 191. | 17B10 VH | CAGGTTCAGCTGCAGCAGTCTGGACCTGAGCTGGT |
| | DNA | GAAGCCTGGGGCCTCGGTGAAGATTTCCTGCAAAG |
| | Sequence | CTTCTGGCTACGCATTCAGTAGCTACTGGCTGAACT |
| | | GGGTGAAGCAGAGGCCTGGAAAGGGTCTTGAGTG |
| | | GTTTGGACGGATTTATCCTGGAGATGGAGATACTG |
| | | ACTACAATGGGAAGTTCAAGGGCAAGGCCACACTG |
| | | ACTGCAGACAAATCCTCCAGCACAGCCTACATGCA |
| | | ACTCAGAAGCCTGACATCTGAGGACTCTGCGGTCT |
| | | ACTTCTGTGTAAGAGGTGATGGTTACTGGGCTATG |
| | | GACTACTGGGGTCAAGGAACCTCAGTCACCGTCTC |
| | | CTCAG |
| 192. | 17B10 VL | GATATTGTGATGACGCAGGCTGCATTCTCCAATCC |
| | DNA | AGTCACTCTTGGAACATCAGCTTCCATCTCCTGCAG |
| | Sequence | GTTTAGTAAGAGTCTCCTACATAGTAATGGCATCA |
| | | CTTATTTGTATTGGTATCTGCAGAAGCCAGGCCAGT |
| | | CTCCTCAGCTCCTGATTTATCAGATGTCCAACCTTG |
| | | CCTCAGGAGTCCCAGACAGGTTCAGTAGCAGTGGG |
| | | TCAGGAACTGATTTCACACTGAGAATCAGCAGAGT |
| | | GGAGGCTGAGGATGTGGGTGTTTATTACTGTGCTC |
| | | AAAATCTAGAACTTCCGTGGACGTTCGGTGGAGGC |
| | | ACCAAGCTGGAAATCAAAC |
| 193. | 19B5 HC- | NYYMS |
| | CDR1 | |
| | (Kabat) | |
| 194. | 19B5 HC- | TISNNGDSTYYLDTVKG |
| | CDR2 | |
| | (Kabat) | |
| 195. | 19B5 HC- | VGTGFTY |
| | CDR3 | |
| | (Kabat) | |
| 196. | 19B5 LC- | RASQSINNYLH |
| | CDR1 | |
| | (Kabat) | |
| 197. | 19B5 LC- | FASQSIS |
| | CDR2 | |
| | (Kabat) | |
| 198. | 19B5 LC- | QQSNSWPLT |
| | CDR3 | |
| | (Kabat) | |
| 199. | 19B5 HC- | GFTFSNYY |
| | CDR1 | |
| | (Vbase2) | |
| 200. | 19B5 HC- | ISNNGDST |
| | CDR2 | |
| | (Vbase2) | |
| 201 | 19B5 HC- | TRVGTGFTY |
| | CDR3 | |
| | (Vbase2) | |
| 202. | 19B5 LC- | QSINNY |
| | CDR1 | |
| | (Vbase2) | |
| 203. | 19B5 LC- | FAS |
| | CDR2 | |
| | (Vbase2) | |
| 204. | 19B5 LC- | QQSNSWPLT |
| | CDR3 | |
| | (Vbase2) | |
| 205 | 19B5 VH | DVNLVESGGGLVKLGGSLKLSCAASGFTFSNYYMSW |
| | Amino Acid | VRQSPEKRLEWVATISNNGDSTYYLDTVKGRFTISRD |
| | Sequence | SAENTLYLQMSSLISEDTAVYYCTRVGTGFTYWGQG |
| | | TLVTVSA |
| 206. | 19B5 VL | DIVLTQSPATLSVTPGDSVSLSCRASQSINNYLHWYQ |
| | Amino Acid | QRSHESPRLLIKFASQSISDIPSRFSGSGSGTDFTLSINSI |
| | Sequence | ETEDFGMYFCQQSNSWPLTFGAGTKLELK |
| 207. | 19B5 VH | GACGTGAACCTCGTGGAGTCTGGGGGAGGCTTAGT |
| | DNA | GAAGCTTGGAGGGTCCCTGAAACTCTCCTGTGCAG |
| | Sequence | CCTCTGGATTCACTTTCAGTAACTACTACATGTCTT |
| | | GGGTTCGCCAGAGTCCGGAGAAGAGGCTGGAGTG |
| | | GGTCGCAACCATTAGTAATAATGGTGATAGCACCT |
| | | ACTATCTAGACACTGTGAAGGGCCGATTCACCATC |
| | | TCCAGAGACAGTGCCGAGAACACCCTGTACCTGCA |
| | | AATGAGCAGTCTGATTTCTGAGGACACAGCCGTGT |
| | | ATTACTGTACAAGAGTTGGGACGGGGTTTACTTAC |
| | | TGGGGCCAAGGGACTCTGGTCACTGTCTCTGCAG |
| 208. | 19B5 VL | GATATTGTGCTAACTCAGTCTCCAGCCACCCTGTCT |
| | DNA | GTGACTCCAGGAGATAGCGTCAGTCTTTCCTGCAG |
| | Sequence | GGCCAGCCAAAGTATTAACAACTACCTACACTGGT |
| | | ATCAACAAAGATCACATGAGTCTCCAAGGCTTCTC |
| | | ATCAAGTTTGCTTCCCAGTCCATCTCTGACATCCCC |
| | | TCCAGGTTCAGTGGCAGTGGATCAGGGACAGATTT |
| | | CACTCTCAGTATCAACAGTATAGAGACTGAAGATT |
| | | TTGGAATGTATTTCTGTCAACAGAGTAACAGCTGG |
| | | CCGCTCACGTTCGGTGCTGGGACCAAGCTGGAGCT |
| | | GAAAC |
| 209. | 17E6 HC- | SYVIH |
| | CDR1 | |
| | (Kabat) | |
| 210. | 17E6 HC- | YINPYSDYTQYNEKFKG |
| | CDR2 | |
| | (Kabat) | |
| 211. | 17E6 HC- | RADGNPYAMDY |
| | CDR3 | |
| | (Kabat) | |
| 212. | 17E6 LC- | KASQDVSTAVV |
| | CDR1 | |
| | (Kabat) | |
| 213. | 17E6 LC- | SASYRYT |
| | CDR2 | |
| | (Kabat) | |
| 214. | 17E6 LC- | QQHYSTPFT |
| | CDR3 | |
| | (Kabat) | |
| 215. | 17E6 HC- | GYTFTSYV |
| | CDR1 | |
| | (Vbase2) | |
| 216. | 17E6 HC- | INPYSDYT |
| | CDR2 | |
| | (Vbase2) | |
| 217. | 17E6 HC- | ARRADGNPYAMDY |
| | CDR3 | |
| | (Vbase2) | |
| 218. | 17E6 LC- | QDVSTA |
| | CDR1 | |
| | (Vbase2) | |
| 219. | 17E6 LC- | SAS |
| | CDR2 | |
| | (Vbase2) | |
| 220. | 17E6 LC- | QQHYSTPFT |
| | CDR3 | |
| | (Vbase2) | |
| 221. | 17E6 VH | EVQLQQSGPELVKPGASVKMSCKASGYTFTSYVIHW |
| | Amino Acid | VKQKPGQGLEWIGYINPYSDYTQYNEKFKGKATLTS |
| | Sequence | DKSSSTAYMELSSLTSEDSAVYSCARRADGNPYAMD |
| | | YWGQGTSVTVSS |
| 222. | 17E6 VL | DIVMTQSHKFMSTSVGDRVSTTCKASQDVSTAVVW |
| | Amino Acid | YQQKPGQSPKLLIYSASYRYTGVPDRFTGSGSGTDFT |
| | Sequence | FTITSVQAEDLAVYYCQQHYSTPFTFGSGTKLEIK |
| 223. | 17E6 VH | GAGGTCCAGCTACAGCAGTCTGGACCTGAGCTGGT |
| | DNA | AAAGCCTGGGGCTTCAGTGAAGATGTCCTGCAAGG |
| | Sequence | CTTCTGGATACACATTCACTAGCTATGTTATTCACT |
| | | GGGTAAAGCAGAAGCCTGGGCAGGGCCTTGAGTG |
| | | GATTGGATATATTAATCCTTACAGTGATTATACTCA |
| | | GTACAATGAGAAGTTCAAAGGCAAGGCCACACTG |
| | | ACTTCAGACAAATCCTCCAGCACAGCCTACATGGA |
| | | GCTCAGCAGCCTGACCTCTGAGGACTCTGCGGTCT |
| | | ATTCCTGTGCAAGGAGGGCAGATGGTAACCCCTAT |
| | | GCTATGGACTACTGGGGTCAAGGAACCTCAGTCAC |
| | | CGTCTCCTCAG |
| 224. | 17E6 VL | GACATTGTGATGACCCAGTCTCACAAATTCATGTC |
| | DNA | CACATCAGTAGGAGACAGGGTCAGCACCACCTGCA |
| | Sequence | AGGCCAGTCAGGATGTGAGTACTGCTGTAGTCTGG |
| | | TATCAACAGAAACCAGGACAATCTCCTAAACTACT |
| | | GATTTACTCGGCATCCTACCGGTACACTGGAGTCC |
| | | CTGATCGCTTCACTGGCAGTGGATCTGGGACGGAT |
| | | TTCACTTTCACCATCACCAGTGTGCAGGCTGAAGA |
| | | CCTGGCAGTTTATTACTGTCAGCAACATTATAGTAC |
| | | TCCATTCACGTTCGGCTCGGGGACAAAGTTGGAAA |
| | | TAAAAC |

Exemplary linkers

| SEQ ID | | |
|---|---|---|
| NO. | Description | Nucleotide or Amino Acid Sequence |
| 225. | Linker | (G)ₙ, n > = 1 |
| 226. | Linker | (GS)ₙ, 8 > = n > = 1 |
| 227. | Linker | (GSGGS)ₙ, 8 > = n > = 1 |
| 228. | Linker | (GGGGS)ₙ, 8 > = n > = 1 |
| 229. | Linker | (GGGS)ₙ, 8 > = n > = 1 |
| 230. | Linker | (GGGGS)₃ |
| 231. | Linker | (GGGGS)₆ |
| 232. | Linker | (GSTSGSGKPGSGEGS)ₙ |
| | | 3 > = n > = 1 |

Exemplary consensus sequence of anti-CD93 antibodies

| | | |
|---|---|---|
| 233. | CDRH2 | RIFPGDGDX₁X₂YX₃GKFKG |
| | (5H9/17A7) | X₁X₂ = AN or TD, X₃ = N or D |
| 234. | CDRH3 | TGAAYX₁FDPFPY |
| | (5H9/17A7) | X₁ = D or E |
| 235. | CDRL1 | SSX₁KSLLHSX₂GX₃TYLY |
| | (5H9/17A7) | X₁ = S or T, X₂ = N or S, X₃ = V or I |
| 236. | CDRH1 | X₁YWX₂N |
| | (5H9/17A7/ | X₁ = S or T, X₂ = L or M |
| | 17B10) | |
| 237. | CDRH2 | RIX₁PGDGDX₂X₃YX₄GKFKG |
| | (5H9/17A7/ | X₁ = Y or F, X₂X₃ = TD or AN, X₄ = N or D |
| | 17B10) | |
| 238. | CDRL1 | X₁X₂X₃KSLLHSX₄GX₅TYLY |
| | (5H9/17A7/ | X₁X₂X₃ = SSS, SST, or RFS, X₄ = N or S, |
| | 17B10) | X₅ = V or I |
| 239. | CDRL2 | X₁MSNLAS |
| | (5H9/17A7/ | X₁ = R or Q |
| | 17B10) | |
| 240. | CDRL3 | AQX₁LEX₂PX₃T |
| | (5H9/17A7/ | X₁ = M or N, X₂ = R or L, X₃ = F or W |
| | 17B10) | |
| 241. | CDRH1 | X₁YVX₂H |
| | (20C7/17E6) | X₁ = A or S, X₂ = M or I |
| 242. | CDRH2 | YIX₁PYX₂DX₃TX₄YNEKFKG |
| | (20C7/17E6) | X₁ = F or N, X₂ = N or S, X₃ = G or Y, |
| | | X₄ = E or Q |
| 243. | CDRH3 | RX₁DGNPYX₂MDY |
| | (20C7/17E6) | X₁ = T or A, X₂ = T or A |
| 244. | CDRL1 | KASQDVSTAVX₁ |
| | (20C7/17E6) | X₁ = A or V |
| 245. | CDRL1 | KASQX₁VX₂TX₃VX₄ |
| | (10B1/20C7/ | X₁ = N or D, X₂ = G or S, X₃ = N or A, |
| | 17E6) | X₄ = A or V |
| 246. | CDRL2 | SASYRX₁X₂ |
| | (10B1/20C7/ | X₁X₂ = FI or YT |
| | 17E6) | |
| 247. | CDRL3 | QQX₁X₂X₃X₄PX₅T |
| | (10B1/20C7/ | X₁X₂X₃X₄ = YNRN or HYST, X₅ = I or F |
| | 17E6) | |
| 248. | CDRL1 | X₁ASQSVX₂X₃X₄X₅X₆SYMX₇ |
| | (16E4/16G9) | X₁ = K or R, X₂X₃X₄X₅X₆ = DYAGD or STSSY, |
| | | X₇ = N or H |
| 249. | CDRL2 | X₁ASNLES |
| | (16E4/16G9) | X₁ = A or Y |
| 250. | CDRL3 | QX₁X₂X₃X₄X₅PX₆T |
| | (16E4/16G9) | X₁X₂X₃X₄X₅ = QTNED or HSWEI, X₆ = R or F |

Exemplary anti-PD-L1 antibody moiety sequences

| | | |
|---|---|---|
| 251. | HC-CDR1 | DTYMY |
| 252. | HC-CDR2 | RIDPANDNTKYAQKFQG |
| 253. | HC-CDR3 | AKNLLNYFDY |
| 254. | LC-CDR1 | RASQEISGYLS |
| 255. | LC-CDR2 | ATSTLQS |
| 256. | LC-CDR3 | LQYAIYPLT |

Exemplary anti-PD-1 antibody moiety sequences

| | | |
|---|---|---|
| 257. | Ab1 HC- | GFTFSSYT |
| | CDR1 | |
| | (Vbase2) | |
| 258. | Ab1 HC- | ISHGGGDT |
| | CDR2 | |
| | (Vbase2) | |
| 259. | Ab1 HC- | ARHSGYERGYYYVMDY |
| | CDR3 | |
| | (Vbase2) | |
| 260. | Ab1 LC- | ESVDYYGFSF |
| | CDR1 | |
| | (Vbase2) | |
| 261. | Ab1 LC- | AAS |
| | CDR2 | |
| | (Vbase2) | |
| 262. | Ab1 LC- | QQSKEVPW |
| | CDR3 | |
| | (Vbase2) | |
| 263. | Ab2 HC- | GYTFTSYT |
| | CDR1 | |
| | (Vbase2) | |
| 264. | Ab2 HC- | INPTTGYT |
| | CDR2 | |
| | (Vbase2) | |
| 265. | Ab2 HC- | ARDDAYYSGY |
| | CDR3 | |
| | (Vbase2) | |
| 266. | Ab2 LC- | ENIYSNL |
| | CDR1 | |
| | (Vbase2) | |
| 267. | Ab2 LC- | AAK |
| | CDR2 | |
| | (Vbase2) | |
| 268. | Ab2 LC- | QHFWGTPWT |
| | CDR3 | |
| | (Vbase2) | |
| 269. | Ab3 HC- | GFAFSSYD |
| | CDR1 | |
| | (Vbase2) | |
| 270. | Ab3 HC- | ITIGGGTT |
| | CDR2 | |
| | (Vbase2) | |
| 271. | Ab3 HC- | ARHRYDYFAMDN |
| | CDR3 | |
| | (Vbase2) | |
| 272. | Ab3 LC- | ENVDNYGINF |
| | CDR1 | |
| | (Vbase2) | |
| 273. | Ab3 LC- | VSS |
| | CDR2 | |
| | (Vbase2) | |
| 274. | Ab3 LC- | QQSKDVPW |
| | CDR3 | |
| | (Vbase2) | |
| 275. | Murine Ab1 | SQVQLQQSGAELARPGASVKMSCKASGYTFTSYTMH |
| | VH | WVKQRPGQGLEWIGYINPTTGYTNYNQKFKDKANPT |
| | | TGYTNYNQKFKDKATLTADKSSSTAYMQLSSLTSED |
| | | SAVYYCARDDAYYSGYWGQGTTLTVSS |
| 276. | Murine Ab1 | DIQMTQSPASLSVSVGETVTITCRASENIYSNLAWYR |
| | VL | QKQGKSPQLLVYAAKNLADGVPSRFSGSGSGTQYSL |
| | | KINSLQSEDFGSYYCQHFWGTPWTFGGGTKLEIKR |
| 277. | Murine Ab2 | VQLVESGGGLVKPGGSLKLSCAASGFAFSSYDMSWV |
| | VH | RQTPEKRLVWVAYITIGGGTTYYSDTVKRLVWVAYI |
| | | TIGGGTTYYSDTVKGRFTISRDNAKNTLYLQMSSLKS |
| | | EDTAMYYCARHRYDYFAMDNWGHGTSVTVSS |
| 278. | Murine Ab2 | DIVLTQSPASLAVSLEHRATISCQASENVDNYGINFM |
| | VI | NWFQHKPAQPPQLLIYVSSNLGSGVPAKFSGSGSGTD |
| | | FSLNIHPMEEDDTAMYFCQQSKDVPWTFSGGTKLEIK |
| | | R |
| 279. | Murine Ab3 | EVKLVESGGGLVQPGGSLKLSCAASGFTFSSYTMSWI |
| | VH | RQTPEKRLEWVAYISHGGGDTYYPDTVKGRFTISRD |
| | | NAKNTLYLQMSSLKSEDTAMYYCARHSGYERGYYY |
| | | VMDYWGQGTSVTVSS |
| 280. | Murine Ab3 | DIVLTQFPTSLAVSLGQRATISCRASESVDYYGFSFIN |
| | VL | WFQQKPGQPPKLLIYAASNQGSGVPARFGGSGSGTD |
| | | FSLNIHPMEEDDTAMYFCQQSKEVPWTFGGGTKLEIK |

Additional Exemplary anti-PD-L1 antibody moiety sequences

| | | |
|---|---|---|
| 281. | Humanized | QVQLVQSGAEVKKPGASVKVSCKASGFNIKDTYMY |
| | VH 1 | WVRQAPGQGLEWMGRIDPANDNTKYAQKFQGRVTI |
| | | TADTSTSTAYMELSSLRSEDTAVYYCARAKNLLNYF |
| | | DYWGQGTLVTVSS |
| 282. | Humanized | QVQLVQSGAEVKKPGASVKVSCKASGFNIKDTYMY |
| | VH 2 | WVRQAPGQGLEWIGRIDPANDNTKYAPKFQGRVTIT |
| | | ADTSTNTAYMELSSLRSEDTAVYYCARAKNLLNYFD |
| | | YWGQGTLVTVSS |
| 283. | Humanized | EVQLVQSGAEVKKPGASVKVSCKASGFNIKDTYMY |
| | VH3 | WVRQAPGQGLEWMGRIDPANDNTKYAQKFQGRVTI |
| | | TADTSTNTAYMELSSLRSEDTAVYYCARAKNLLNYF |
| | | DYWGQGTLVTVSS |
| 284. | Humanized | DIQMTQSPSSLSASVGDRVTITCRASQEISGYLSWYQ |
| | VL 1 | QKPGKAPKRLIYATSTLDSGVPSRFSGSGSGTDFTLTI |
| | | SSLQPEDFATYYCLQYAIYPLTFGQGTKLEIKR |
| 285. | Humanized | DIQMTQSPSSLSASVGDRVTITCRASQEISGYLSWLQQ |
| | VL 2 | KPGKAPKRLIYATSTLQSGVPSRFSGSRSGTDYTLTIS |
| | | SLQPEDFATYYCLQYAIYPLTFGQGTKLEIKR |
| 286. | Humanized | DIQMTQSPSSLSASVGDRVTITCRASQEISGYLSWYQ |
| | VL 3 | QKPGKAPKRLIYATSTLDSGVPSRFSGSRSGSDYTLTI |
| | | SSLQPEDFATYYCLQYAIYPLTFGQGTKLEIKR |

Additional exemplary anti-CD93 antibody sequences

| | | |
|---|---|---|
| 287. | 7F3 Heavy | QVQLQQSGADLVRPGASVKLSCKASGYTFTDYEMH |
| | chain | WVKQTPVYGLEWIGGIDPETGDTAYNQNFKGKATLT |
| | | ADKSSSAAYMELRSLTSEDSAVYYCTNYGNLYYYA |
| | | MDYWGQGTSVTVSS |
| 288. | 7F3 Light | ENVLTQSPAIMSASPGEKVTMTCRASSSVSSSYLHWY |
| | chain | QQKSGASPKLWIYSTSNLAFGVPARFSGSGSGTSYSL |
| | | TISSVEAEDAATYYCQQYSGYPLTFGSGTKLEIK |
| 289. | 7F3 HC | DYEMH |
| | CDR1 | |
| | (Kabat) | |
| 290. | 7F3 HC- | GIDPETGDTAYNQNFKG |
| | CDR2 | |
| | (Kabat) | |
| 291. | 7F3 HC- | YGNLYYYAMDY |
| | CDR3 | |
| | (Kabat) | |
| 292. | 7F3 LC- | RASSSVSSSYLH |
| | CDR1 | |
| | (Kabat) | |
| 293. | 7F3 LC- | STSNLAF |
| | CDR2 | |
| | (Kabat) | |
| 294. | 7F3 LC- | QQYSGYPLT |
| | CDR3 | |
| | (Kabat) | |
| 295 | 7F3 HC | GYTFTDYE |
| | CDR1 | |
| | (Vbase2) | |
| 296. | 7F3 HC- | IDPETGDT |
| | CDR2 | |
| | (Vbase2) | |
| 297. | 7F3 HC- | TNYGNLYYYAMDY |
| | CDR3 | |
| | (Vbase2) | |
| 298. | 7F3 LC- | SSVSSSY |
| | CDR1 | |
| | (Vbase2) | |
| 299. | 7F3 LC- | STS |
| | CDR2 | |
| | (Vbase2) | |
| 300. | 7F3 LC- | QQYSGYPLT |
| | CDR3 | |
| | (Vbase2) | |
| 301. | 16E4 VL1 | KASQSVDYAGDSYLN |
| | LC-CDR1 | |
| | (Kabat) | |
| 302. | 16E4 | RASQSVDYAGDSYMN |
| | VL2/16E4 | |
| | VLA LC- | |
| | CDR1 | |
| | (Kabat) | |
| 303. | 16E4 VL3 | RASQSVDYAGDSYLA |
| | LC-CDR1 | |
| | (Kabat) | |
| 304. | 16E4 VH5 | SYWIH |
| | HC-CDR1 | |
| | (Kabat) | |
| 305. | 16E4 VH5 | EIEPSASYTYYNQKFKG |
| | HC-CDR2 | |
| | (Kabat) | |
| 306. | 16E4 VL5 | RASQSVDYAGDSYLN |
| | LC-CDR1 | |
| | (Kabat) | |
| 307. | 16E4 VH-1 | QVQLVESGAEVKKPGASVKLSCKASGYTFTSYWMH |
| | | WVRQAPGQRLEWMGEIDPSASYTYYNQKFKGRVTIT |
| | | VDKSASTAYMELSSLRSEDTAVYYCARSVYYGNKYF |
| | | DVWGPGTTVTVSS |
| 308. | 16E4 VH-2 | QVQLVQSGAEVKKPGASVKVSCKASGYTFTSYWMH |
| | | WVRQAPGQGLEWMGEIDPSASYTYYNQKFKGRVTM |
| | | TRDKSISTAYMELNSLTSDDSAVYYCARSVYYGNKY |
| | | FDVWGAGTTVTVSS |
| 309. | 16E4 VH-3 | QVQLVQSGAEVRKPGASVKVSCKASGYTFTSYWMH |
| | | WVRQAPGQGLEWVGEIDPSASYTYYNQKFKGRVTIT |
| | | ADKSTSTAYMELSSLRSEDTDVYYCARSVYYGNKYF |
| | | DVWGQGTTVTVSS |
| 310. | 16E4 VH-4 | QVQLVQSGAEVKKPGASVKVSCKASGYTFTSYWMH |
| | | WVRQAPGQGLEWMGEIDPSASYTYYNQKFKGRVTM |
| | | TRDKSSSTVYMELSSLTSEDSAVYYCARSVYYGNKY |
| | | FDVWGAGTTVTVSS |
| 311. | 16E4 VH-5 | QVQLVQSGAEVKKPGASVKVSCRASGYTFTSYWIH |
| | | WVRQAPGQGLEWIGEIEPSASYTYYNQKFKGRVTMT |
| | | RDKSSSTVYMELSSLTSEDSAVYYCARSVYYGNKYF |
| | | DVWGAGTTVTVSS |
| 312. | 16E4 VH-6 | QVQLQQSGAEVKKPGASVKVSCKASGYTFTSYWMH |
| | | WVRQAPGQGLEWIGEIDPSASYTYYNQKFKGRVTMT |
| | | RDKSTSTVYMQLSSLTSEDTAVYYCARSVYYGNKYF |
| | | DVWGAGTTVTVSS |
| 313. | 16E4 VL-1 | DIVMTQSPDSLAVSLGERATINCKASQSVDYAGDSYL |
| | | NWYQQKPGQPPKLLIYAASNLESGVPDRFSGSGSGTD |
| | | FTLTISSLQAEDVAVYYCQQTNEDPRTFGGGTKVEIK |
| 314. | 16E4 VL-2 | DIVLTQSPSSLSASVGQRVTITCRASQSVDYAGDSYM |
| | | NWYQQKPGKAPKLLIYAASNLESGVPSRFSGSGSGTD |
| | | FTLTVSSLEDEDFATYYCQQTNEDPRTFGGGTKVEIK |
| 315. | 16E4 VL-3 | EIVLTQSPATLSLSPGQRATLSCRASQSVDYAGDSYL |
| | | AWYQQKPGQAPRLLIYAASNLESGIPARFSGSGSGTD |
| | | FTLTIRPLEEEDAAVYYCQQTNEDPRTFGGGTKLEIK |
| 316. | 16E4 VL-4 | DIQMTQSPSSLSASVGDRVTITCRASQSVDYAGDSYM |
| | | NWYQQKPGKAPKLLIYAASNLESGVPSRFSGSGSGTD |
| | | FTLTISSLEDEDFATYYCQQTNEDPRTFGGGTKLEIK |
| 317. | 16E4 VL-5 | DIVLTQSPSSLSASVGQRVTITCRASQSVDYAGDSYL |
| | | NWYQQKPGKAPKLLIYAASNLESGIPSRFSGSGSGTD |
| | | FTLTISSLEDEDFATYYCQQTNEDPRTFGGGTKLEIK |
| 318. | 16E4 VL-6 | DIQMTQSPSTLSASVGDRVTITCKASQSVDYAGDSYM |
| | | NWYQQKPGKAPKLLIYAASNLESGVPSRFSGSGSGTE |
| | | FTLTISSLQPDDFATYYCQQTNEDPRTFGGGTKLEIK |
| 319. | 7F3 VH-1 | QVQLVQSGAEMVKPGASVKISCKASGYTFTDYEMH |
| | | WVRQTPVYGLEWIGGIDPETGDTAYNQNFKGRVTM |
| | | TRDTSISTAYMELSRLTSDDTAVYYCTNYGNLYYYA |
| | | MDYWGQGTLVTVSS |
| 320. | 7F3 VH-2 | QVQLQQSGAEVKKPGSSVKVSCKASGYTFTDYEMH |
| | | WVRQTPVYGLEWMGGIDPETGDTAYNQNFKGRVTI |
| | | TADKSTSTAYMELSSLRSEDTAVYYCTNYGNLYYYA |
| | | MDYWGQGTTVTVSS |
| 321. | 7F3 VH-3 | QVQLVQSGAEVKKPGASVKVSCKASGYTFTDYEMH |
| | | WVRQAPGQGLEWMGGIDPETGDTAYNQNFKGRVT |
| | | MTTDTSTSTAYMELRSLTSDDTAVYYCTNYGNLYYY |
| | | AMDYWGQGTSVTVSS |
| 322. | 7F3 VL-1 | EIVLTQSPATLSLSPGERATLSCRASSSVSSSYLHWYQ |
| | | QKSGASPRLLIYSTSNLAFGIPARFSGSGSGTDYTLTIS |
| | | SLEAEDVAVYYCQQYSGYPLTFGGGTKVEIK |
| 323. | 7F3 VL-2 | EIVMTQSPATLSVSPGERATLSCRASSSVSSSYLHWY |
| | | QQKSGASPRLWIYSTSNLAFGIPARFSGSGSGTEYTLT |
| | | ISSLQSEDFAAYYCQQYSGYPLTFGGGTKVEIK |
| 324. | 7F3 VL-3 | EIVLTQSPSSLSASVGDRVTITCRASSSVSSSYLHWYQ |
| | | QKPGKAPKLLIYSTSNLAFGVPSRFSGSGSGTSYTFTIS |
| | | SLQPEDIATYYCQQYSGYPLTFGSGTKLEIK |

Exemplary anti-VEGF sequences

| | | |
|---|---|---|
| 325. | Afibercept | SDTGRPFVEMYSEIPEIIHMTEGRELVIPCRVTSPNITV |
| | | TLKKFPLDTLIPDGKRIIWDSRKGFIISNATYKEIGLLT |
| | | CEATVNGHLYKTNYLTHRQTNTIIDVVLSPSHGIELSV |
| | | GEKLVLNCTARTELNVGIDFNWEYPSSKHQHKKLVN |
| | | RDLKTQSGSEMKKFLSTLTIDGVTRSDQGLYTCAASS |
| | | GLMTKKNSTFVRVH |
| 326. | Avastin HC- | GYTFTNYGMN |
| | CDR1 | |
| | (Kabat) | |
| 327. | Avastin HC- | WINTYTGEPTYAADFKR |
| | CDR2 | |
| | (Kabat) | |
| 328. | Avastin HC- | YPHYYGSSHWYFDV |
| | CDR3 | |
| | (Kabat) | |
| 329. | Avastin LC- | SASQDISNYLN |
| | CDR1 | |
| | (Kabat) | |
| 330. | Avastin LC- | FTSSLHS |
| | CDR2 | |
| | (Kabat) | |
| 331. | Avastin LC- | QQYSTVPWT |
| | CDR3 | |
| | (Kabat) | |
| 332. | Ramucirumab | SYSMN |
| | HC-CDR1 | |
| | (Kabat) | |
| 333. | Ramucirumab | SISSSSSYIYYADSVKG |
| | HC-CDR2 | |
| | (Kabat) | |
| 334. | Ramucirumab | VTDAFDI |
| | HC-CDR3 | |
| | (Kabat) | |
| 335. | Ramucirumab | RASQGIDNWLG |
| | LC-CDR1 | |
| | (Kabat) | |
| 336. | Ramucirumab | DASNLDT |
| | LC-CDR2 | |
| | (Kabat) | |
| 337. | Ramucirumab | QQAKAFPPT |
| | LC-CDR3 | |
| | (Kabat) | |

Additional sequences

| | | |
|---|---|---|
| 338. | Exemplary | GSDKTHT |
| | Linker | |
| 339. | hIgG1 CH1 | ASTKGPSVFPLAPSSKSTSGGTAALGCLVKDYFPEPV |
| | | TVSWNSGALTSGVHTFPAVLQSSGLYSLSSVVTVPSS |
| | | SLGTQTYICNVNHKPSNTKVDKKV |
| 340. | hIgG1 Fc | EPKSCDKTHTCPPCPAPELLGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALPAPIEKTISKAKGQPREPQVYTLPPSRDELTKN |
| | | QVSLTCLVKGFYPSDIAVEWESNGQPENNYKTTPPVL |
| | | DSDGSFFLYSKLTVDKSRWQQGNVFSCSVMHEALHN |
| | | HYTQKSLSLSPGK |
| 341. | Human kappa | RTVAAPSVFIFPPSDEQLKSGTASVVCLLNNFYPREAK |
| | CL | VQWKVDNALQSGNSQESVTEQDSKDSTYSLSSTLTL |
| | | SKADYEKHKVYACEVTHQGLSSPVTKSFNRGEC |
| 342. | 7F3-HC- | QVQLQQSGADLVRPGASVKLSCKASGYTFTDYEMH |
| | Aflibercept | WVKQTPVYGLEWIGGIDPETGDTAYNQNFKGKATLT |
| | fusion | ADKSSSAAYMELRSLTSEDSAVYYCTNYGNLYYYA |
| | (without | MDYWGQGTSVTVSSASTKGPSVFPLAPSSKSTSGGTA |
| | signal | ALGCLVKDYFPEPVTVSWNSGALTSGVHTFPAVLQS |
| | peptide) | SGLYSLSSVVTVPSSSLGTQTYICNVNHKPSNTKVDK |
| | | KVEPKSCDKTHTCPPCPAPELLGGPSVFLFPPKPKDTL |
| | | MISRTPEVTCVVVDVSHEDPEVKFNWYVDGVEVHN |
| | | AKTKPREEQYNSTYRVVSVLTVLHQDWLNGKEYKC |
| | | KVSNKALPAPIEKTISKAKGQPREPQVYTLPPSRDELT |
| | | KNQVSLTCLVKGFYPSDIAVEWESNGQPENNYKTTPP |
| | | VLDSDGSFFLYSKLTVDKSRWQQGNVFSCSVMHEAL |
| | | HNHYTQKSLSLSPGKGSDKTHTSDTGRPFVEMYSEIP |
| | | EIIHMTEGRELVIPCRVTSPNITVTLKKFPLDTLIPDGK |
| | | RIIWDSRKGFIISNATYKEIGLLTCEATVNGHLYKTNY |
| | | LTHRQTNTIIDVVLSPSHGIELSVGEKLVLNCTARTEL |
| | | NVGIDFNWEYPSSKHQHKKLVNRDLKTQSGSEMKKF |
| | | LSTLTIDGVTRSDQGLYTCAASSGLMTKKNSTFVRVH |
| 343. | 7F3 LC | ENVLTQSPAIMSASPGEKVTMTCRASSSVSSSYLHWY |
| | (without | QQKSGASPKLWIYSTSNLAFGVPARFSGSGSGTSYSL |
| | signal | TISSVEAEDAATYYCQQYSGYPLTFGSGTKLEIKRTV |
| | peptide) | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |

Exemplary signaling peptides

| | | |
|---|---|---|
| 344. | Signaling | MGWTLVFLFLLSVTAGVHS |
| | peptide | |
| 345. | Signaling | MVSSAQFLGLLLLCFQGTRC |
| | peptide | |
| 346. | Signaling | MGWSCIILFLVATATGVHS |
| | peptide | |

Additional humanized anti-CD93 antibody sequence

| | | |
|---|---|---|
| 347. | 17B10 VH1 | QVQLVQSGAEVKKPGSSVKVSCKASGYAFSSYWLN |
| | | WVRQAPGQGLEWFGRIYPGDGDTDYNGKFKGRVTL |
| | | TADKSTSTAYMELSSLRSEDTAVYFCVRGDGYWAM |
| | | DYWGQGTTVTVSS |
| 348. | 17B10 VH2 | QVQLVQSGAEVVKSGASVKVSCKASGYAFSSYWLN |
| | | WVRQAPGQGLEWFGRIYPGDGDTDYNGKFKGRVTLI |
| | | RDTSTSTVYMELTSLTSEDTAVYYCVRGDGYWAMD |
| | | YWGQGTLVTVSS |
| 349. | 17B10 VH3 | QVQLVQSGPEVKKPGESLKISCKASGYAFSSYWLNW |
| | | VRQMPGKGLEWMGRIYPGDGDTDYNGKFKGQVTIS |
| | | ADKSSGTAYLQLSSLKASDTAVYFCVRGDGYWAMD |
| | | YWGQGTLVTVSS |
| 350. | 17B10 VL1 | DIVMTQSPLSLPVTPGEPASISCRFSQSLLHSNGITYLY |
| | | WYLQKPGQSPQLLIYQMSNLASGVPDRFSGSGSGTDF |
| | | TLKISRVEAEDVGVYYCAQNLELPWTFGGGTKLEIK |
| 351. | 17B10 VL2 | DIVMTQTPLSLPVTPGEPASISCRFSQSLLHSNGITYLY |
| | | WYLQKPGQSPQLLIYTMSNLASGVPDRFSGSGSGTDF |
| | | TLKISRVEAEDVGVYYCAQNLELPWTFGGGTKLEIK |
| 352. | 17B10 VL3 | DIVMTQSPDSLAVSLGERATINCRFSKSLLHSNGITYL |
| | | YWYQQKPGQPPKLLIYQMSNLASGVPDRFSGSGSGT |
| | | DFTLTISSLQAEDVAVYYCAQNLELPWTFGGGTKLEI |
| | | K |
| 353. | 17B10 VL1 | RFSQSLLHSNGITYLY |
| | and VL2 LC- | |
| | CDR1 | |
| | (Kabat) | |
| 354. | 17B10 and | TMSNLAS |
| | VL2 LC- | |
| | CDR2 | |
| | (Kabat) | |
| 355. | 16A1 VL1 | KSSQSLLNSNNQKNYLA |
| | LC-CDR1 | |
| | (Kabat) | |
| 356. | 16A1 VL1 | FASTRES |
| | LC-CDR2 | |
| | (Kabat) | |
| 357. | 16A1 VL1 | QQHYNTPLT |
| | LC-CDR3 | |
| | (Kabat) | |
| 358. | 16A1 VL2 | KSSQSLLNSNNQKNSLA |
| | LC-CDR1 | |
| | (Kabat) | |
| 359. | 16A1 VL2 | QQHSNTPLT |
| | LC-CDR3 | |
| | (Kabat) | |
| 360. | 16A1_VH1 | EVQLVQSGAEVKKPGTTVKIACKVSGYTFTDHGIHW |
| | | VQQAPGKGLEWMGNISPGNGDIKYNEKFKGRVTLTA |
| | | DKSSDTAYMELNTLRSEDTAIYFCTTYFVDWGRGTL |
| | | VTVSS |
| 361. | 16A1_VH2 | QVQLQQSGAEVKKPGASVKVSCKASGYTFTDHGIH |
| | | WVRQAPGRGLEWLGNISPGNGDIKYNEKFKGRVTM |
| | | TRDTSTSTVYMELSSLTSEDTAVYFCTTYFVDWGRG |
| | | TLVTVSS |
| 362. | 16A1_VH3 | QVQLLESGAEAKKPGASVKLSCKASGYTFTDHGIHW |
| | | VHQAPGQRLEWIGNISPGNGDIKYNEKFKGRVTITVD |
| | | KSASTAYMEVSSLRSEDTAVYFCTTYFVDWGRGTLV |
| | | TVSS |
| 363. | 16A1_VL1 | DIVMTQSPSSLAVSLGERATLNCKSSQSLLNSNNQKN |
| | | YLAWYQQKPGQPPKLLIYFASTRESGVPDRFSGSGSG |
| | | TDFTLTISSVQAEDVAYYFCQQHYNTPLTFGQGTKLE |
| | | IK |
| 364. | 16A1_VL2 | DIVMTQSPDSLAVSLGERATINCKSSQSLLNSNNQKN |
| | | SLAWYQQKPGQSPKLLIYFASTRESGVPDRFSGSGSG |
| | | TDFTLTISSLQAEDVAYYFCQQHSNTPLTFGGGTKVEI |
| | | K |
| 365. | 16A1_VL3 | EIVMTQSPATLSVSPGERATLSCKSSQSLLNSNNQKN |
| | | CLAWYQQKPGQAPRLLIYFASTRESGIPARFSGSGSG |
| | | TEFTLTISSLQSEDFAYYFCQQHCNTPLTFGGGTKVEI |
| | | K |

Additional humanized anti-CD93 antibody fusion protein sequence

| | | |
|---|---|---|
| 366. | h7F3-HC- | QVQLVQSGAEVKKPGASVKVSCKASGYTFTDYEM |
| | Aflibercept | HWVRQAPGQGLEWMGGIDPETGDTAYNQNFKGR |
| | fusion A | VTMTTDTSTSTAYMELRSLTSDDTAVYYCTNYGN |
| | (without | LYYYAMDYWGQGTSVTVSSASTKGPSVFPLAPSSKS |
| | signal | TSGGTAALGCLVKDYFPEPVTVSWNSGALTSGVHTF |
| | peptide) | PAVLQSSGLYSLSSVVTVPSSSLGTQTYICNVNHKPSN |
| | | TKVDKKVEPKSCDKTHTCPPCPAPELLGGPSVFLFPP |
| | | KPKDTLMISRTPEVTCVVVDVSHEDPEVKFNWYVDG |
| | | VEVHNAKTKPREEQYNSTYRVVSVLTVLHQDWLNG |
| | | KEYKCKVSNKALPAPIEKTISKAKGQPREPQVYTLPPS |
| | | RDELTKNQVSLTCLVKGFYPSDIAVEWESNGQPENN |
| | | YKTTPPVLDSDGSFFLYSKLTVDKSRWQQGNVFSCS |
| | | VMHEALHNHYTQKSLSLSPGKGSDKTHTSDTGRPFV |
| | | EMYSEIPEIIHMTEGRELVIPCRVTSPNITVTLKKFPLD |
| | | TLIPDGKRIIWDSRKGFIISNATYKEIGLLTCEATVNGH |
| | | LYKTNYLTHRQTNTIIDVVLSPSHGIELSVGEKLVLNC |
| | | TARTELNVGIDFNWEYPSSKHQHKKLVNRDLKTQSG |
| | | SEMKKFLSTLTIDGVTRSDQGLYTCAASSGLMTKKNS |
| | | TFVRVH |
| 367. | h7F3 LC | EIVLTQSPSSLSASVGDRVTITCRASSSVSSSYLHWYQ |
| | ( without | QKPGKAPKLLIYSTSNLAFGVPSRFSGSGSGTSYTFTIS |
| | signal | SLQPEDIATYYCQQYSGYPLTFGSGTKLEIKRTVAAPS |
| | peptide) | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |

1. An anti-CD93 construct comprising an antibody moiety comprising a heavy chain variable region (V_{H}) and a light chain variable region (V_{L}), wherein the antibody moiety competes for a binding epitope of CD93 with an antibody or antibody fragment comprising a second heavy chain variable region (V_{H-2}) and a second light chain variable region (V_{L-2}), wherein:
a) the V_{H-2} comprises the HC-CDR1 comprising the amino acid sequence of SEQ ID NO:289, the HC-CDR2 comprising the amino acid sequence of SEQ ID NO: 290, and the HC-CDR3 comprising the amino acid sequence of SEQ ID NO: 291, and the V_{L-2} comprises the LC-CDR1 comprising the amino acid sequence of SEQ ID NO: 292, the LC-CDR2 comprising the amino acid sequence of SEQ ID NO: 293, and the LC-CDR3 comprising the amino acid sequence of SEQ ID NO:294;
b) the V_{H-2} comprises the HC-CDR1 comprising the amino acid sequence of SEQ ID NO: 17, the HC-CDR2 comprising the amino acid sequence of SEQ ID NO: 18, and the HC-CDR3 comprising the amino acid sequence of SEQ ID NO: 19, and the V_{L-2} comprises the LC-CDR1 comprising the amino acid sequence of SEQ ID NO: 20, the LC-CDR2 comprising the amino acid sequence of SEQ ID NO: 21, and the LC-CDR3 comprising the amino acid sequence of SEQ ID NO: 22;
c) the V_{H-2} comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the V_{L-2} comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38;
d) the V_{H-2} comprises the HC-CDR1 comprising the amino acid sequence of SEQ ID NO: 49, the HC-CDR2 comprising the amino acid sequence of SEQ ID NO: 50, and the HC-CDR3 comprising the amino acid sequence of SEQ ID NO: 51, and the V_{L-2} comprises the LC-CDR1 comprising the amino acid sequence of SEQ ID NO: 52, the LC-CDR2 comprising the amino acid sequence of SEQ ID NO: 53, and the LC-CDR3 comprising the amino acid sequence of SEQ ID NO: 54;
e) the V_{H-2} comprises the HC-CDR1 comprising the amino acid sequence of SEQ ID NO: 65, the HC-CDR2 comprising the amino acid sequence of SEQ ID NO: 66, and the HC-CDR3 comprising the amino acid sequence of SEQ ID NO: 67, and the V_{L-2} comprises the LC-CDR1 comprising the amino acid sequence of SEQ ID NO: 68, the LC-CDR2 comprising the amino acid sequence of SEQ ID NO: 69, and the LC-CDR3 comprising the amino acid sequence of SEQ ID NO: 70;
f) the V_{H-2} comprises the HC-CDR1 comprising the amino acid sequence of SEQ ID NO: 81, the HC-CDR2 comprising the amino acid sequence of SEQ ID NO: 82, and the HC-CDR3 comprising the amino acid sequence of SEQ ID NO: 83, and the V_{L-2} comprises the LC-CDR1 comprising the amino acid sequence of SEQ ID NO: 84, the LC-CDR2 comprising the amino acid sequence of SEQ ID NO: 85, and the LC-CDR3 comprising the amino acid sequence of SEQ ID NO: 86;
g) the V_{H-2} comprises the HC-CDR1 comprising the amino acid sequence of SEQ ID NO: 97, the HC-CDR2 comprising the amino acid sequence of SEQ ID NO: 98, and the HC-CDR3 comprising the amino acid sequence of SEQ ID NO: 99, and the V_{L-2} comprises the LC-CDR1 comprising the amino acid sequence of SEQ ID NO: 100, the LC-CDR2 comprising the amino acid sequence of SEQ ID NO: 101, and the LC-CDR3 comprising the amino acid sequence of SEQ ID NO: 102;
h) the V_{H-2} comprises the HC-CDR1 comprising the amino acid sequence of SEQ ID NO: 113, the HC-CDR2 comprising the amino acid sequence of SEQ ID NO: 114, and the HC-CDR3 comprising the amino acid sequence of SEQ ID NO: 115, and the V_{L-2} comprises the LC-CDR1 comprising the amino acid sequence of SEQ ID NO: 116, the LC-CDR2 comprising the amino acid sequence of SEQ ID NO: 117, and the LC-CDR3 comprising the amino acid sequence of SEQ ID NO: 118;
i) the V_{H-2} comprises the HC-CDR1 comprising the amino acid sequence of SEQ ID NO: 129, the HC-CDR2 comprising the amino acid sequence of SEQ ID NO: 130, and the HC-CDR3 comprising the amino acid sequence of SEQ ID NO: 131, and the V_{L-2} comprises the LC-CDR1 comprising the amino acid sequence of SEQ ID NO: 132, the LC-CDR2 comprising the amino acid sequence of SEQ ID NO: 133, and the LC-CDR3 comprising the amino acid sequence of SEQ ID NO: 134;
j) the V_{H-2} comprises the HC-CDR1 comprising the amino acid sequence of SEQ ID NO: 145, the HC-CDR2 comprising the amino acid sequence of SEQ ID NO: 146, and the HC-CDR3 comprising the amino acid sequence of SEQ ID NO: 147, and the V_{L-2} comprises the LC-CDR1 comprising the amino acid sequence of SEQ ID NO: 148, 355, or 358, the LC-CDR2 comprising the amino acid sequence of SEQ ID NO: 149 or 356, and the LC-CDR3 comprising the amino acid sequence of SEQ ID NO: 150, 357 or 359;
k) the V_{H-2} comprises the HC-CDR1 comprising the amino acid sequence of SEQ ID NO: 161, the HC-CDR2 comprising the amino acid sequence of SEQ ID NO: 162, and the HC-CDR3 comprising the amino acid sequence of SEQ ID NO: 163, and the V_{L-2} comprises the LC-CDR1 comprising the amino acid sequence of SEQ ID NO: 164, the LC-CDR2 comprising the amino acid sequence of SEQ ID NO: 165, and the LC-CDR3 comprising the amino acid sequence of SEQ ID NO: 166;
l) the V_{H-2} comprises the HC-CDR1 comprising the amino acid sequence of SEQ ID NO: 177, the HC-CDR2 comprising the amino acid sequence of SEQ ID NO: 178, and the HC-CDR3 comprising the amino acid sequence of SEQ ID NO: 179, and the V_{L-2} comprises the LC-CDR1 comprising the amino acid sequence of SEQ ID NO: 180 or 353, the LC-CDR2 comprising the amino acid sequence of SEQ ID NO: 181 or 354, and the LC-CDR3 comprising the amino acid sequence of SEQ ID NO: 182;
m) the V_{H-2} comprises the HC-CDR1 comprising the amino acid sequence of SEQ ID NO: 193, the HC-CDR2 comprising the amino acid sequence of SEQ ID NO: 194, and the HC-CDR3 comprising the amino acid sequence of SEQ ID NO: 195, and the V_{L-2} comprises the LC-CDR1 comprising the amino acid sequence of SEQ ID NO: 196, the LC-CDR2 comprising the amino acid sequence of SEQ ID NO: 197, and the LC-CDR3 comprising the amino acid sequence of SEQ ID NO: 198;
n) the V_{H-2} comprises the HC-CDR1 comprising the amino acid sequence of SEQ ID NO: 209, the HC-CDR2 comprising the amino acid sequence of SEQ ID NO: 210, and the HC-CDR3 comprising the amino acid sequence of SEQ ID NO: 211, and the V_{L-2} comprises the LC-CDR1 comprising the amino acid sequence of SEQ ID NO: 212, the LC-CDR2 comprising the amino acid sequence of SEQ ID NO: 213, and the LC-CDR3 comprising the amino acid sequence of SEQ ID NO: 214;
o) the V_{H-2} comprising the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the V_{L-2} comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6; or
p) the V_{H-2} comprises the HC-CDR1 comprising the amino acid sequence of SEQ ID NO: 17 or 304, the HC-CDR2 comprising the amino acid sequence of SEQ ID NO: 18 or 305, and the HC-CDR3 comprising the amino acid sequence of SEQ ID NO: 19, and the V_{L-2} comprises the LC-CDR1 comprising the amino acid sequence of SEQ ID NO: 20, 301, 302, 303, or 306, the LC-CDR2 comprising the amino acid sequence of SEQ ID NO: 21, and the LC-CDR3 comprising the amino acid sequence of SEQ ID NO:22.

2. The anti-CD93 construct of claim 1, wherein:
a) the V_{H} comprises i) the HC-CDR1 comprising the amino acid sequence of SEQ ID NO: 289, ii) the HC-CDR2 comprising the amino acid sequence of SEQ ID NO: 290, and iii) the HC-CDR3 comprising the amino acid sequence of SEQ ID NO: 291, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 292, ii) the LC-CDR2 comprising the amino acid sequence of SEQ ID NO: 293, and iii) the LC-CDR3 comprising the amino acid sequence of SEQ ID NO: 294, or a variant thereof comprising up to 5, 4, 3, 2, or 1 amino acid substitutions in the LC-CDRs,
b) the V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22, or a variant thereof comprising up to 5, 4, 3, 2, or 1 amino acid substitutions in the LC-CDRs,
c) the V_{H} comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38, or a variant thereof comprising up to 5, 4, 3, 2, or 1 amino acid substitutions in the LC-CDRs,
d) the V_{H} comprises i) the HC-CDR1 comprising the amino acid sequence of SEQ ID NO: 49, ii) the HC-CDR2 comprising the amino acid sequence of SEQ ID NO: 50, and iii) the HC-CDR3 comprising the amino acid sequence of SEQ ID NO: 51, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 52, ii) the LC-CDR2 comprising the amino acid sequence of SEQ ID NO: 53, and iii) the LC-CDR3 comprising the amino acid sequence of SEQ ID NO: 54, or a variant thereof comprising up to 5, 4, 3, 2, or 1 amino acid substitutions in the LC-CDRs,
e) the V_{H} comprises i) the HC-CDR1 comprising the amino acid sequence of SEQ ID NO: 65, ii) the HC-CDR2 comprising the amino acid sequence of SEQ ID NO: 66, and iii) the HC-CDR3 comprising the amino acid sequence of SEQ ID NO: 67, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 68, ii) the LC-CDR2 comprising the amino acid sequence of SEQ ID NO: 69, and iii) the LC-CDR3 comprising the amino acid sequence of SEQ ID NO: 70, or a variant thereof comprising up to 5, 4, 3, 2, or 1 amino acid substitutions in the LC-CDRs,
f) the V_{H} comprises i) the HC-CDR1 comprising the amino acid sequence of SEQ ID NO: 81, ii) the HC-CDR2 comprising the amino acid sequence of SEQ ID NO: 82, and iii) the HC-CDR3 comprising the amino acid sequence of SEQ ID NO: 83, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 84, ii) the LC-CDR2 comprising the amino acid sequence of SEQ ID NO: 85, and iii) the LC-CDR3 comprising the amino acid sequence of SEQ ID NO: 86, or a variant thereof comprising up to 5, 4, 3, 2, or 1 amino acid substitutions in the LC-CDRs,
g) the V_{H} comprises i) the HC-CDR1 comprising the amino acid sequence of SEQ ID NO: 97, ii) the HC-CDR2 comprising the amino acid sequence of SEQ ID NO: 98, and iii) the HC-CDR3 comprising the amino acid sequence of SEQ ID NO: 99, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 100, ii) the LC-CDR2 comprising the amino acid sequence of SEQ ID NO: 101, and iii) the LC-CDR3 comprising the amino acid sequence of SEQ ID NO: 102, or a variant thereof comprising up to 5, 4, 3, 2, or 1 amino acid substitutions in the LC-CDRs,
h) the V_{H} comprises i) the HC-CDR1 comprising the amino acid sequence of SEQ ID NO: 113, ii) the HC-CDR2 comprising the amino acid sequence of SEQ ID NO: 114, and iii) the HC-CDR3 comprising the amino acid sequence of SEQ ID NO: 115, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 116, ii) the LC-CDR2 comprising the amino acid sequence of SEQ ID NO: 117, and iii) the LC-CDR3 comprising the amino acid sequence of SEQ ID NO: 118, or a variant thereof comprising up to 5, 4, 3, 2, or 1 amino acid substitutions in the LC-CDRs,
i) the V_{H} comprises i) the HC-CDR1 comprising the amino acid sequence of SEQ ID NO: 129, ii) the HC-CDR2 comprising the amino acid sequence of SEQ ID NO: 130, and iii) the HC-CDR3 comprising the amino acid sequence of SEQ ID NO: 131, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 132, ii) the LC-CDR2 comprising the amino acid sequence of SEQ ID NO: 133, and iii) the LC-CDR3 comprising the amino acid sequence of SEQ ID NO: 134, or a variant thereof comprising up to 5, 4, 3, 2, or 1 amino acid substitutions in the LC-CDRs,
j) the V_{H} comprises i) the HC-CDR1 comprising the amino acid sequence of SEQ ID NO: 145, ii) the HC-CDR2 comprising the amino acid sequence of SEQ ID NO: 146, and iii) the HC-CDR3 comprising the amino acid sequence of SEQ ID NO: 147, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 148, 355, or 358, ii) the LC-CDR2 comprising the amino acid sequence of SEQ ID NO: 149 or 356, and iii) the LC-CDR3 comprising the amino acid sequence of SEQ ID NO: 150, 357 or 359, or a variant thereof comprising up to 5, 4, 3, 2, or 1 amino acid substitutions in the LC-CDRs,
k) the V_{H} comprises i) the HC-CDR1 comprising the amino acid sequence of SEQ ID NO: 161, ii) the HC-CDR2 comprising the amino acid sequence of SEQ ID NO: 162, and iii) the HC-CDR3 comprising the amino acid sequence of SEQ ID NO: 163, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 164, ii) the LC-CDR2 comprising the amino acid sequence of SEQ ID NO: 165, and iii) the LC-CDR3 comprising the amino acid sequence of SEQ ID NO: 166, or a variant thereof comprising up to 5, 4, 3, 2, or 1 amino acid substitutions in the LC-CDRs,
l) the V_{H} comprises i) the HC-CDR1 comprising the amino acid sequence of SEQ ID NO: 177, ii) the HC-CDR2 comprising the amino acid sequence of SEQ ID NO: 178, and iii) the HC-CDR3 comprising the amino acid sequence of SEQ ID NO: 179, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 180 or 353, ii) the LC-CDR2 comprising the amino acid sequence of SEQ ID NO: 181 or 354, and iii) the LC-CDR3 comprising the amino acid sequence of SEQ ID NO: 182, or a variant thereof comprising up to 5, 4, 3, 2, or 1 amino acid substitutions in the LC-CDRs,
m) the V_{H} comprises i) the HC-CDR1 comprising the amino acid sequence of SEQ ID NO: 193, ii) the HC-CDR2 comprising the amino acid sequence of SEQ ID NO: 194, and iii) the HC-CDR3 comprising the amino acid sequence of SEQ ID NO: 195, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 196, ii) the LC-CDR2 comprising the amino acid sequence of SEQ ID NO: 197, and iii) the LC-CDR3 comprising the amino acid sequence of SEQ ID NO: 198, or a variant thereof comprising up to 5, 4, 3, 2, or 1 amino acid substitutions in the LC-CDRs, or
n) the V_{H} comprises i) the HC-CDR1 comprising the amino acid sequence of SEQ ID NO: 209, ii) the HC-CDR2 comprising the amino acid sequence of SEQ ID NO: 210, and iii) the HC-CDR3 comprising the amino acid sequence of SEQ ID NO: 211, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 212, ii) the LC-CDR2 comprising the amino acid sequence of SEQ ID NO: 213, and iii) the LC-CDR3 comprising the amino acid sequence of SEQ ID NO: 214, or a variant thereof comprising up to 5, 4, 3, 2, or 1 amino acid substitutions in the LC-CDRs,
o) the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 5, 4, 3, 2, or 1 amino acid substitutions in the LC-CDRs, or
p) the V_{H} comprises the HC-CDR1 comprising the amino acid sequence of SEQ ID NO: 17 or 304, the HC-CDR2 comprising the amino acid sequence of SEQ ID NO: 18 or 305, and the HC-CDR3 comprising the amino acid sequence of SEQ ID NO: 19, and the V_{L} comprises the LC-CDR1 comprising the amino acid sequence of SEQ ID NO: 20, 301, 302, 303, or 306, the LC-CDR2 comprising the amino acid sequence of SEQ ID NO: 21, and the LC-CDR3 comprising the amino acid sequence of SEQ ID NO:22.

3. The anti-CD93 construct of claim 2, wherein the V_{H} comprises i) the HC-CDR1 comprising the amino acid sequence of SEQ ID NO: 289, ii) the HC-CDR2 comprising the amino acid sequence of SEQ ID NO: 290, and iii) the HC-CDR3 comprising the amino acid sequence of SEQ ID NO: 291, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 292, ii) the LC-CDR2 comprising the amino acid sequence of SEQ ID NO: 293, and iii) the LC-CDR3 comprising the amino acid sequence of SEQ ID NO: 294, or a variant thereof comprising up to 5, 4, 3, 2, or 1 amino acid substitutions in the LC-CDRs.

4. The anti-CD93 construct of claim 2, wherein the V_{H} comprises i) the HC-CDR1 comprising the amino acid sequence of SEQ ID NO: 17 or 304, ii) the HC-CDR2 comprising the amino acid sequence of SEQ ID NO: 18 or 305, and iii) the HC-CDR3 comprising the amino acid sequence of SEQ ID NO: 19, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 20, 301, 302, 303, or 306, ii) the LC-CDR2 comprising the amino acid sequence of SEQ ID NO: 21, and iii) the LC-CDR3 comprising the amino acid sequence of SEQ ID NO: 22, or a variant thereof comprising up to 5, 4, 3, 2, or 1 amino acid substitutions in the LC-CDRs.

5. The anti-CD93 construct of claim 2, wherein the V_{H} comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38, or a variant thereof comprising up to 5, 4, 3, 2, or 1 amino acid substitutions in the LC-CDRs.

6. The anti-CD93 construct of claim 2, wherein the V_{H} comprises i) the HC-CDR1 comprising the amino acid sequence of SEQ ID NO: 49, ii) the HC-CDR2 comprising the amino acid sequence of SEQ ID NO: 50, and iii) the HC-CDR3 comprising the amino acid sequence of SEQ ID NO: 51, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 52, ii) the LC-CDR2 comprising the amino acid sequence of SEQ ID NO: 53, and iii) the LC-CDR3 comprising the amino acid sequence of SEQ ID NO: 54, or a variant thereof comprising up to 5, 4, 3, 2, or 1 amino acid substitutions in the LC-CDRs.

7. The anti-CD93 construct of claim 2, wherein the V_{H} comprises i) the HC-CDR1 comprising the amino acid sequence of SEQ ID NO: 65, ii) the HC-CDR2 comprising the amino acid sequence of SEQ ID NO: 66, and iii) the HC-CDR3 comprising the amino acid sequence of SEQ ID NO: 67, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 68, ii) the LC-CDR2 comprising the amino acid sequence of SEQ ID NO: 69, and iii) the LC-CDR3 comprising the amino acid sequence of SEQ ID NO: 70, or a variant thereof comprising up to 5, 4, 3, 2, or 1 amino acid substitutions in the LC-CDRs.

8. The anti-CD93 construct of claim 2, wherein the V_{H} comprises i) the HC-CDR1 comprising the amino acid sequence of SEQ ID NO: 81, ii) the HC-CDR2 comprising the amino acid sequence of SEQ ID NO: 82, and iii) the HC-CDR3 comprising the amino acid sequence of SEQ ID NO: 83, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 84, ii) the LC-CDR2 comprising the amino acid sequence of SEQ ID NO: 85, and iii) the LC-CDR3 comprising the amino acid sequence of SEQ ID NO: 86, or a variant thereof comprising up to 5, 4, 3, 2, or 1 amino acid substitutions in the LC-CDRs.

9. The anti-CD93 construct of claim 2, wherein the V_{H} comprises i) the HC-CDR1 comprising the amino acid sequence of SEQ ID NO: 97, ii) the HC-CDR2 comprising the amino acid sequence of SEQ ID NO: 98, and iii) the HC-CDR3 comprising the amino acid sequence of SEQ ID NO: 99, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 100, ii) the LC-CDR2 comprising the amino acid sequence of SEQ ID NO: 101, and iii) the LC-CDR3 comprising the amino acid sequence of SEQ ID NO: 102, or a variant thereof comprising up to 5, 4, 3, 2, or 1 amino acid substitutions in the LC-CDRs.

10. The anti-CD93 construct of claim 2, wherein the V_{H} comprises i) the HC-CDR1 comprising the amino acid sequence of SEQ ID NO: 113, ii) the HC-CDR2 comprising the amino acid sequence of SEQ ID NO: 114, and iii) the HC-CDR3 comprising the amino acid sequence of SEQ ID NO: 115, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 116, ii) the LC-CDR2 comprising the amino acid sequence of SEQ ID NO: 117, and iii) the LC-CDR3 comprising the amino acid sequence of SEQ ID NO: 118, or a variant thereof comprising up to 5, 4, 3, 2, or 1 amino acid substitutions in the LC-CDRs.

11. The anti-CD93 construct of claim 2, wherein the V_{H} comprises i) the HC-CDR1 comprising the amino acid sequence of SEQ ID NO: 129, ii) the HC-CDR2 comprising the amino acid sequence of SEQ ID NO: 130, and iii) the HC-CDR3 comprising the amino acid sequence of SEQ ID NO: 131, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 132, ii) the LC-CDR2 comprising the amino acid sequence of SEQ ID NO: 133, and iii) the LC-CDR3 comprising the amino acid sequence of SEQ ID NO: 134, or a variant thereof comprising up to 5, 4, 3, 2, or 1 amino acid substitutions in the LC-CDRs.

12. The anti-CD93 construct of claim 2, wherein the V_{H} comprises i) the HC-CDR1 comprising the amino acid sequence of SEQ ID NO: 145, ii) the HC-CDR2 comprising the amino acid sequence of SEQ ID NO: 146, and iii) the HC-CDR3 comprising the amino acid sequence of SEQ ID NO: 147, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 148, 355, or 358, ii) the LC-CDR2 comprising the amino acid sequence of SEQ ID NO: 149 or 356, and iii) the LC-CDR3 comprising the amino acid sequence of SEQ ID NO: 150, 357 or 359, or a variant thereof comprising up to 5, 4, 3, 2, or 1 amino acid substitutions in the LC-CDRs.

13. The anti-CD93 construct of claim 2, wherein the V_{H} comprises i) the HC-CDR1 comprising the amino acid sequence of SEQ ID NO: 161, ii) the HC-CDR2 comprising the amino acid sequence of SEQ ID NO: 162, and iii) the HC-CDR3 comprising the amino acid sequence of SEQ ID NO: 163, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 164, ii) the LC-CDR2 comprising the amino acid sequence of SEQ ID NO: 165, and iii) the LC-CDR3 comprising the amino acid sequence of SEQ ID NO: 166, or a variant thereof comprising up to 5, 4, 3, 2, or 1 amino acid substitutions in the LC-CDRs.

14. The anti-CD93 construct of claim 2, wherein the V_{H} comprises i) the HC-CDR1 comprising the amino acid sequence of SEQ ID NO: 177, ii) the HC-CDR2 comprising the amino acid sequence of SEQ ID NO: 178, and iii) the HC-CDR3 comprising the amino acid sequence of SEQ ID NO: 179, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 180 or 353, ii) the LC-CDR2 comprising the amino acid sequence of SEQ ID NO: 181 or 354, and iii) the LC-CDR3 comprising the amino acid sequence of SEQ ID NO: 182, or a variant thereof comprising up to 5, 4, 3, 2, or 1 amino acid substitutions in the LC-CDRs.

15. The anti-CD93 construct of claim 2, wherein the V_{H} comprises i) the HC-CDR1 comprising the amino acid sequence of SEQ ID NO: 193, ii) the HC-CDR2 comprising the amino acid sequence of SEQ ID NO: 194, and iii) the HC-CDR3 comprising the amino acid sequence of SEQ ID NO: 195, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 196, ii) the LC-CDR2 comprising the amino acid sequence of SEQ ID NO: 197, and iii) the LC-CDR3 comprising the amino acid sequence of SEQ ID NO: 198, or a variant thereof comprising up to 5, 4, 3, 2, or 1 amino acid substitutions in the LC-CDRs.

16. The anti-CD93 construct of claim 2, wherein the V_{H} comprises i) the HC-CDR1 comprising the amino acid sequence of SEQ ID NO: 209, ii) the HC-CDR2 comprising the amino acid sequence of SEQ ID NO: 210, and iii) the HC-CDR3 comprising the amino acid sequence of SEQ ID NO: 211, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 212, ii) the LC-CDR2 comprising the amino acid sequence of SEQ ID NO: 213, and iii) the LC-CDR3 comprising the amino acid sequence of SEQ ID NO: 214, or a variant thereof comprising up to 5, 4, 3, 2, or 1 amino acid substitutions in the LC-CDRs.

17. The anti-CD93 construct of claim 2, wherein the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 5, 4, 3, 2, or 1 amino acid substitutions in the HC-CDRs; and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 5, 4, 3, 2, or 1 amino acid substitutions in the LC-CDRs.

18. An anti-CD93 construct comprising an antibody moiety that specifically binds to CD93, comprising:
a) a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 287 and 319-321, and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 288, and 322-324;
b) a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 29 and 307-312, and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 30, and 313-318;
c) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 45, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 46;
d) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 61, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 62;
e) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 77, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 78;
f) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 93, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 94;
g) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 109, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 110;
h) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 125, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 126;
i) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 141, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 142;
j) a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 157 and 360-362, and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 158, and 363-365;
k) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 173, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 174;
l) a HC-CDR1, a HC-CDR2, and a HC-CDR3, respectively comprising the amino acid sequences of a CDR1, a CDR2, and a CDR3 within a V_{H} chain region having the sequence set forth in any of SEQ ID NO: 189 and 347-349, and a LC-CDR1, a LC-CDR2, and a LC-CDR3, respectively comprising the amino acid sequences of a CDR1, a CDR2, and a CDR3 within a V_{L} chain region having the sequence set forth in any of SEQ ID NO: 190, and 350-352;
m) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 205, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 206;
n) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 221, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 222;
o) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 13, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 14;
p) a HC-CDR1, a HC-CDR2, and a HC-CDR3, respectively comprising the amino acid sequences of a CDR1, a CDR2, and a CDR3 within a V_{H} chain region having the sequence set forth in any one of SEQ ID NOs: 307-312, and a LC-CDR1, a LC-CDR2, and a LC-CDR3, respectively comprising the amino acid sequences of a CDR1, a CDR2, and a CDR3 within a V_{L} chain region having the sequence set forth in any one of SEQ ID NOs: 313-318; or
q) a HC-CDR1, a HC-CDR2, and a HC-CDR3, respectively comprising the amino acid sequences of a CDR1, a CDR2, and a CDR3 within a V_{H} chain region having the sequence set forth in any one of SEQ ID NOs: 319-321, and a LC-CDR1, a LC-CDR2, and a LC-CDR3, respectively comprising the amino acid sequences of a CDR1, a CDR2, and a CDR3 within a V_{L} chain region having the sequence set forth in any one of SEQ ID NOs: 322-324.

19. The anti-CD93 construct of any one of claims 1-18, wherein the V_{H} comprises an amino acid sequence of any one of SEQ ID NOs: 13, 29, 45, 61, 77, 93, 109, 125, 141, 157, 173, 189, 205, 221, 287, 307-312 and 319-321 or a variant comprising an amino acid sequence having at least about 80% sequence identity; and/or wherein the V_{L} comprises an amino acid sequence of any one of SEQ ID NOs: 14, 30, 46, 62, 78, 94, 110, 126, 142, 158, 174, 190, 206, 222, 288, 313-318 and 322-324, or a variant comprising an amino acid sequence having at least about 80% sequence identity.

20. The anti-CD93 construct of claim 19, wherein:
a) the V_{H} comprises an amino acid sequence of any of SEQ ID NO: 287 and 319-321, or a variant comprising an amino acid sequence having at least about 80% sequence identity; and the V_{L} comprises an amino acid sequence of any of SEQ ID NO: 288, and 322-324, or a variant comprising an amino acid sequence having at least about 80% sequence identity,
b) the V_{H} comprises an amino acid sequence of any of SEQ ID NO: 29 and 307-312, or a variant comprising an amino acid sequence having at least about 80% sequence identity; and the V_{L} comprises an amino acid sequence of any of SEQ ID NO: 30, and 313-318, or a variant comprising an amino acid sequence having at least about 80% sequence identity,
c) the V_{H} comprises an amino acid sequence of SEQ ID NO: 45, or a variant comprising an amino acid sequence having at least about 80% sequence identity; and the V_{L} comprises an amino acid sequence of SEQ ID NO: 46, or a variant comprising an amino acid sequence having at least about 80% sequence identity,
d) the V_{H} comprises an amino acid sequence of SEQ ID NO: 61, or a variant comprising an amino acid sequence having at least about 80% sequence identity; and the V_{L} comprises an amino acid sequence of SEQ ID NO: 62, or a variant comprising an amino acid sequence having at least about 80% sequence identity,
e) the V_{H} comprises an amino acid sequence of SEQ ID NO: 77, or a variant comprising an amino acid sequence having at least about 80% sequence identity; and the V_{L} comprises an amino acid sequence of SEQ ID NO: 78, or a variant comprising an amino acid sequence having at least about 80% sequence identity,
f) the V_{H} comprises an amino acid sequence of SEQ ID NO: 93, or a variant comprising an amino acid sequence having at least about 80% sequence identity; and the V_{L} comprises an amino acid sequence of SEQ ID NO: 94, or a variant comprising an amino acid sequence having at least about 80% sequence identity,
g) the V_{H} comprises an amino acid sequence of SEQ ID NO: 109, or a variant comprising an amino acid sequence having at least about 80% sequence identity; and the V_{L} comprises an amino acid sequence of SEQ ID NO: 110, or a variant comprising an amino acid sequence having at least about 80% sequence identity,
h) the V_{H} comprises an amino acid sequence of SEQ ID NO: 125, or a variant comprising an amino acid sequence having at least about 80% sequence identity; and the V_{L} comprises an amino acid sequence of SEQ ID NO: 126, or a variant comprising an amino acid sequence having at least about 80% sequence identity,
i) the V_{H} comprises an amino acid sequence of SEQ ID NO: 141, or a variant comprising an amino acid sequence having at least about 80% sequence identity; and the V_{L} comprises an amino acid sequence of SEQ ID NO: 142, or a variant comprising an amino acid sequence having at least about 80% sequence identity,
j) the V_{H} comprises an amino acid sequence of SEQ ID NO: 157, or a variant comprising an amino acid sequence having at least about 80% sequence identity; and the V_{L} comprises an amino acid sequence of SEQ ID NO: 158, or a variant comprising an amino acid sequence having at least about 80% sequence identity,
k) the V_{H} comprises an amino acid sequence of SEQ ID NO: 173, or a variant comprising an amino acid sequence having at least about 80% sequence identity; and the V_{L} comprises an amino acid sequence of SEQ ID NO: 174, or a variant comprising an amino acid sequence having at least about 80% sequence identity,
l) the V_{H} comprises an amino acid sequence of any of SEQ ID NO: 189 and 347-349, or a variant comprising an amino acid sequence having at least about 80% sequence identity; and the V_{L} comprises an amino acid sequence of any of SEQ ID NO: 190, and 350-352, or a variant comprising an amino acid sequence having at least about 80% sequence identity,
m) the V_{H} comprises an amino acid sequence of SEQ ID NO: 205, or a variant comprising an amino acid sequence having at least about 80% sequence identity; and the V_{L} comprises an amino acid sequence of SEQ ID NO: 206, or a variant comprising an amino acid sequence having at least about 80% sequence identity,
n) the V_{H} comprises an amino acid sequence of SEQ ID NO: 221, or a variant comprising an amino acid sequence having at least about 80% sequence identity; and the V_{L} comprises an amino acid sequence of SEQ ID NO: 222, or a variant comprising an amino acid sequence having at least about 80% sequence identity,
o) the V_{H} comprises an amino acid sequence of SEQ ID NO: 13, or a variant comprising an amino acid sequence having at least about 80% sequence identity; and the V_{L} comprises an amino acid sequence of SEQ ID NO: 14, or a variant comprising an amino acid sequence having at least about 80% sequence identity,
p) the V_{H} comprises an amino acid sequence of any one of SEQ ID NOs: 307-312, or a variant comprising an amino acid sequence having at least about 80% sequence identity, and the V_{L} comprises an amino acid sequence of any one of SEQ ID NOs: 313-318, or a variant comprising an amino acid sequence having at least about 80% sequence identity, or
q) the V_{H} comprises an amino acid sequence of any one of SEQ ID NOs: 319-321, or a variant comprising an amino acid sequence having at least about 80% sequence identity, and the V_{L} comprises an amino acid sequence of any one of SEQ ID NOs: 322-324, or a variant comprising an amino acid sequence having at least about 80% sequence identity.

21. The anti-CD93 construct of any one of claims 1-20, wherein the antibody moiety is an antibody or antigen-binding fragment thereof selected from the group consisting of a full-length antibody, a bispecific antibody, a single-chain Fv (scFv) fragment, a Fab fragment, a Fab′ fragment, a F(ab′)2, an Fv fragment, a disulfide stabilized Fv fragment (dsFv), a (dsFv)₂, a Fv-Fc fusion, a scFv-Fc fusion, a scFv-Fv fusion, a diabody, a tribody, and a tetrabody.

22. The anti-CD93 construct of claim 21, wherein the antibody moiety is a full-length antibody.

23. The anti-CD93 construct of any one of claims 1-22, wherein the antibody moiety has an Fc fragment is selected from the group consisting of Fc fragments form IgG, IgA, IgD, IgE, IgM, and combinations and hybrids thereof.

24. The anti-CD93 construct of claim 23, wherein the Fc fragment is selected from the group consisting of Fc fragments from IgG1, IgG2, IgG3, IgG4, and combinations and hybrids thereof.

25. The anti-CD93 construct of claim 23 or claim 24, wherein the Fc fragment has a reduced effector function as compared to the corresponding wildtype Fc fragment.

26. The anti-CD93 construct of claim 23 or claim 24, wherein the Fc fragment has an enhanced effector function as compared to the corresponding wildtype Fc fragment.

27. The anti-CD93 construct of any one of claims 1-26, wherein the antibody moiety blocks the binding of CD93 to IGFBP7.

28. The anti-CD93 construct of any one of claims 1-27, wherein the antibody moiety blocks the binding of CD93 to MMRN2.

29. The anti-CD93 construct of any one of claims 1-22, wherein the CD93 is a human CD93.

30. An anti-CD93 construct comprising a first moiety that binds to CD93 and a second moiety that binds to VEGF, wherein the first moiety comprises an anti-CD93 antibody moiety comprising a heavy chain variable region (V_{H}) and a light chain variable region (V_{L}), wherein the V_{H} comprises i) a HC-CDR1 comprising the amino acid sequence of SEQ ID NO: 289, ii) a HC-CDR2 comprising the amino acid sequence of SEQ ID NO: 290, and iii) a HC-CDR3 comprising the amino acid sequence of SEQ ID NO: 291; and the V_{L} comprises i) a LC-CDR1 comprising the amino acid sequence of SEQ ID NO: 292, ii) a LC-CDR2 comprising the amino acid sequence of SEQ ID NO: 293, and iii) a LC-CDR3 comprising the amino acid sequence of SEQ ID NO: 294.

31. The anti-CD93 construct of claim 30, wherein the V_{H} comprises the amino acid sequence set forth in any one of SEQ ID NOs: 287 and 319-321, or a variant comprising an amino acid sequence having at least about 80% sequence identity, and wherein the V_{L} comprises the amino acid sequence set forth in any one of SEQ ID NOs: 288 and 322-324, or a variant comprising an amino acid sequence having at least about 80% sequence identity.

32. The anti-CD93 construct of claim 30 or claim 31, wherein the anti-CD93 antibody moiety is an anti-CD93 full-length antibody comprising two heavy chains and two light chains, and wherein the second moiety is fused to C-terminus of both of the heavy chains of the anti-CD93 full-length antibody.

33. The anti-CD93 construct of claim 32, wherein the two heavy chains fused with the second moiety each comprises the amino acid sequence set forth in SEQ ID NO: 342 or 366, or a variant comprising an amino acid sequence having at least about 80% sequence identity, and wherein the two light chains each comprise the amino acid sequences set forth in SEQ ID NO: 343 or 367, or a variant comprising an amino acid sequence having at least about 80% sequence identity.

34. A pharmaceutical composition comprising the anti-CD93 construct of any one of claims 1-33, and a pharmaceutical acceptable carrier.

35. An isolated nucleic acid encoding the anti-CD93 construct of any one of claims 1-33 or a portion thereof.

36. A vector comprising the isolated nucleic acid of claim 35.

37. An isolated host cell comprising the isolated nucleic acid of claim 35, or the vector of claim 36.

38. An immunoconjugate comprising the anti-CD93 construct of any one of claims 1-33, linked to a therapeutic agent or a label.

39. A method of producing an anti-CD93 construct comprising:
a) culturing the isolated host cell of claim 37 under conditions effective to express the anti-CD93 construct; and
b) obtaining the expressed anti-CD93 construct from the host cell.

40. A method of treating a disease or condition in an individual, comprising administering to the individual an effective mount of the anti-CD93 construct of any one of claims 1-33, or the pharmaceutical composition of claim 34.

41. The method of claim 40, wherein the disease or condition is associated with an abnormal vascular structure.

42. The method of claim 40 or claim 41, wherein the disease or condition is a cancer.

43. The method of claim 42, wherein the cancer is a solid tumor.

44. The method of claim 42 or claim 43, wherein the cancer comprises CD93+ endothelial cells.

45. The method of any one of claims 42-44, wherein the cancer comprises IGFBP7+ blood vessels.

46. The method of any one of claims 42-45, wherein the cancer comprises MMRN2+ blood vessels.

47. The method of any one of claims 42-46, wherein the cancer is characterized by tumor hypoxia.

48. The method of any one of claims 42-47, wherein the cancer is a locally advanced or metastatic cancer.

49. The method of any one of claims 42-48, wherein the cancer is selected from the group consisting of a lymphoma, colon cancer, brain cancer, breast cancer, ovarian cancer, endometrial cancer, esophageal cancer, prostate cancer, cervical cancer, renal cancer, bladder cancer, gastric cancer, non-small cell lung cancer, melanoma, and pancreatic cancer.

50. The method of any one of claims 40-49, wherein the anti-CD93 construct is administered parenterally into the individual.

51. The method of any one of claims 40-50, wherein the method further comprises administering a second therapy.

52. The method of claim 51, wherein the second therapy is selected from the group consisting of surgery, radiation, gene therapy, immunotherapy, bone marrow transplantation, stem cell transplantation, hormone therapy, targeted therapy, cryotherapy, ultrasound therapy, photodynamic therapy, and chemotherapy.

53. The method of claim 52, wherein the second therapy is an immunotherapy.

54. The method of claim 53, wherein the immunotherapy comprises administering an immunomodulatory agent.

55. The method of claim 54, wherein the immunomodulatory agent is an immune checkpoint inhibitor.

56. The method of claim 55, wherein the immune checkpoint inhibitor comprises an anti-PD-L1 antibody or an anti-PD-1 antibody.

57. The method of any one of claims 40-56, wherein the individual is a human.


SEQUENCE LISTING (422 sequences)

SEQ ID NO: 1 - protein, 5 aa, Artificial Sequence
  Synthetic Construct
SFGVN

SEQ ID NO: 2 - protein, 16 aa, Artificial Sequence
  Synthetic Construct
VIWSGGSTDYNVAFIS

SEQ ID NO: 3 - protein, 13 aa, Artificial Sequence
  Synthetic Construct
NWRYDGYFYAMDY

SEQ ID NO: 4 - protein, 11 aa, Artificial Sequence
  Synthetic Construct
KASQNVGTNVA

SEQ ID NO: 5 - protein, 7 aa, Artificial Sequence
  Synthetic Construct
SASYRFI

SEQ ID NO: 6 - protein, 9 aa, Artificial Sequence
  Synthetic Construct
QQYNRNPIT

SEQ ID NO: 7 - protein, 8 aa, Artificial Sequence
  Synthetic Construct
DFSLSSFG

SEQ ID NO: 8 - protein, 7 aa, Artificial Sequence
  Synthetic Construct
IWSGGST

SEQ ID NO: 9 - protein, 15 aa, Artificial Sequence
  Synthetic Construct
ARNWRYDGYFYAMDY

SEQ ID NO: 10 - protein, 6 aa, Artificial Sequence
  Synthetic Construct
QNVGTN

SEQ ID NO: 11 - protein, 3 aa, Artificial Sequence
  Synthetic Construct
SAS

SEQ ID NO: 12 - protein, 9 aa, Artificial Sequence
  Synthetic Construct
QQYNRNPIT

SEQ ID NO: 13 - protein, 121 aa, Artificial Sequence
  Synthetic Construct
QVQLKQSGPGLVQPSQSLSITCTVSDFSLSSFGVNWVRQPPGKGLEWLGVIWSGGSTDYNVAFISRLSISKDNSKSQVFFKMNNLQADDTAIYYCARNWRYDGYFYAMDYWGQGTSVTVSS

SEQ ID NO: 14 - protein, 107 aa, Artificial Sequence
  Synthetic Construct
DIVMTQSQKFMSTSTGDRVSVTCKASQNVGTNVAWYQQKPGQSPKALIYSASYRFIGVPDRFTGSGSGTDFTLTITNVQSEDLAEYFCQQYNRNPITFGSGTKLEIK

SEQ ID NO: 15 - DNA, 364 bp, Artificial Sequence
  Synthetic Construct
caggtgcagctgaagcagtcaggacctggcctagtgcagccctcacagagcctgtccatcacctgcacagtctctgatttctcattatctagctttggtgtaaactgggttcgccagcctccaggaaagggtctggagtggctgggggtgatatggagtggtggaagtacagactataatgtagctttcatatccagactgagcatcagcaaggacaactccaagagccaagttttctttaaaatgaacaatctgcaagctgatgacacagccatatactactgtgccagaaattggaggtatgatggttacttctatgctatggactactggggtcaaggaacctcagtcaccgtctcctcag

SEQ ID NO: 16 - DNA, 322 bp, Artificial Sequence
  Synthetic Construct
gacattgtgatgacccagtctcaaaaattcatgtccacatcaacaggagacagggtcagcgtcacctgcaaggccagtcagaatgtgggtactaatgtagcctggtatcaacagaaaccaggacagtctcctaaagcactgatttactcggcatcataccgattcattggagtccctgatcgcttcacaggcagtggatctgggacagatttcactctcaccatcaccaatgtgcagtctgaagacttggcagagtatttctgtcagcaatataacagaaatcctatcacgttcggctcggggacaaagttggaaataaaac

SEQ ID NO: 17 - protein, 5 aa, Artificial Sequence
  Synthetic Construct
SYWMH

SEQ ID NO: 18 - protein, 17 aa, Artificial Sequence
  Synthetic Construct
EIDPSASYTYYNQKFKG

SEQ ID NO: 19 - protein, 11 aa, Artificial Sequence
  Synthetic Construct
SVYYGNKYFDV

SEQ ID NO: 20 - protein, 15 aa, Artificial Sequence
  Synthetic Construct
KASQSVDYAGDSYMN

SEQ ID NO: 21 - protein, 7 aa, Artificial Sequence
  Synthetic Construct
AASNLES

SEQ ID NO: 22 - protein, 9 aa, Artificial Sequence
  Synthetic Construct
QQTNEDPRT

SEQ ID NO: 23 - protein, 8 aa, Artificial Sequence
  Synthetic Construct
GYTFTSYW

SEQ ID NO: 24 - protein, 8 aa, Artificial Sequence
  Synthetic Construct
IDPSASYT

SEQ ID NO: 25 - protein, 13 aa, Artificial Sequence
  Synthetic Construct
ARSVYYGNKYFDV

SEQ ID NO: 26 - protein, 10 aa, Artificial Sequence
  Synthetic Construct
QSVDYAGDSY

SEQ ID NO: 27 - protein, 3 aa, Artificial Sequence
  Synthetic Construct
AAS

SEQ ID NO: 28 - protein, 9 aa, Artificial Sequence
  Synthetic Construct
QQTNEDPRT

SEQ ID NO: 29 - protein, 120 aa, Artificial Sequence
  Synthetic Construct
QVQLQQPGAELVKPGASVKLSCKASGYTFTSYWMHWVKQRPGQGLEWIGEIDPSASYTYYNQKFKGKATLTVDKSSSTAYMQLSSLTSEDSAVYYCARSVYYGNKYFDVWGAGTTVTVSS

SEQ ID NO: 30 - protein, 111 aa, Artificial Sequence
  Synthetic Construct
DIVLTQSPASLAVSLGQRATISCKASQSVDYAGDSYMNWYQQKPGQPPKLLIYAASNLESGIPARFSGSGSGTDFTLNIHPVEEEDAATYYCQQTNEDPRTFGGGTKLEIK

SEQ ID NO: 31 - DNA, 360 bp, Artificial Sequence
  Synthetic Construct
caggtccagcttcagcagcctggggctgaactggtgaagcctggggcttcagtgaagctgtcctgcaaggcttctggatacaccttcactagctactggatgcactgggtgaagcagaggcctggacaaggccttgagtggatcggagagattgatccttctgctagttatacttactacaatcaaaagttcaagggcaaggccacattgactgtagacaaatcctccagcacagcctacatgcaactcagcagcctgacatctgaggactctgcggtctattactgtgcaagatcggtctactatggtaacaagtatttcgatgtctggggcgcagggaccacggtcaccgtctcctca

SEQ ID NO: 32 - DNA, 334 bp, Artificial Sequence
  Synthetic Construct
gacattgtgctgacccaatctccagcttctttggctgtgtctctagggcagagggccaccatctcctgcaaggccagccaaagtgttgattatgccggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaactaatgaggatcctcggacgttcggtggaggcaccaagctggaaatcaaac

SEQ ID NO: 33 - protein, 5 aa, Artificial Sequence
  Synthetic Construct
TYWMN

SEQ ID NO: 34 - protein, 17 aa, Artificial Sequence
  Synthetic Construct
RIFPGDGDANYNGKFKG

SEQ ID NO: 35 - protein, 12 aa, Artificial Sequence
  Synthetic Construct
TGAAYDFDPFPY

SEQ ID NO: 36 - protein, 16 aa, Artificial Sequence
  Synthetic Construct
SSSKSLLHSNGVTYLY

SEQ ID NO: 37 - protein, 7 aa, Artificial Sequence
  Synthetic Construct
RMSNLAS

SEQ ID NO: 38 - protein, 9 aa, Artificial Sequence
  Synthetic Construct
AQMLERPFT

SEQ ID NO: 39 - protein, 8 aa, Artificial Sequence
  Synthetic Construct
GYAFSTYW

SEQ ID NO: 40 - protein, 8 aa, Artificial Sequence
  Synthetic Construct
IFPGDGDA

SEQ ID NO: 41 - protein, 14 aa, Artificial Sequence
  Synthetic Construct
TRTGAAYDFDPFPY

SEQ ID NO: 42 - protein, 11 aa, Artificial Sequence
  Synthetic Construct
KSLLHSNGVTY

SEQ ID NO: 43 - protein, 3 aa, Artificial Sequence
  Synthetic Construct
RMS

SEQ ID NO: 44 - protein, 9 aa, Artificial Sequence
  Synthetic Construct
AQMLERPFT

SEQ ID NO: 45 - protein, 121 aa, Artificial Sequence
  Synthetic Construct
QVQLQQSGPDLVKPGASVKISCKASGYAFSTYWMNWVKQRPGKGLEWIGRIFPGDGDANYNGKFKGKATLTADKSSSTAYMQLSSLTSEDSAVYFCTRTGAAYDFDPFPYWGQGTLVTVSA

SEQ ID NO: 46 - protein, 112 aa, Artificial Sequence
  Synthetic Construct
DIVMTQAAFSNPVTLGTSASISCSSSKSLLHSNGVTYLYWYLQRPGQSPQLLIYRMSNLASGVPDRFSGSGSGTDFTLRISRVEAEDVGIYYCAQMLERPFTFGSGTKLEIK

SEQ ID NO: 47 - DNA, 364 bp, Artificial Sequence
  Synthetic Construct
caggttcagctgcagcagtctggacctgacctggtgaagcctggggcctcagtgaagatttcctgcaaagcttctggctacgcattcagtacctactggatgaactgggtgaagcagaggcctggaaagggtcttgagtggattggacggatttttcctggagatggagatgctaactacaatgggaagttcaagggcaaggccacactgactgcagacaaatcctccagcacagcctacatgcaactcagcagcctgacatctgaggactctgcggtctacttctgtacaagaactggggccgcctatgatttcgacccttttccttactggggccaagggactctggtcactgtctctgcag

SEQ ID NO: 48 - DNA, 337 bp, Artificial Sequence
  Synthetic Construct
gatattgtgatgacgcaggctgcattctccaatccagtcactcttggaacatcagcttccatctcttgcagttctagtaagagtctcctacatagtaatggcgtcacttatttgtattggtatctgcagaggccaggccagtctcctcagctcctgatatatcggatgtccaaccttgcctcaggagtcccagacaggttcagtggcagtgggtcaggaactgatttcacactgagaatcagcagagtggaggctgaggatgtgggtatttattactgtgctcaaatgctagaacgcccattcacgttcggctcggggacaaagttggaaataaaac

SEQ ID NO: 49 - protein, 5 aa, Artificial Sequence
  Synthetic Construct
DYYMN

SEQ ID NO: 50 - protein, 17 aa, Artificial Sequence
  Synthetic Construct
RVNPNNGGKTYNQKFKG

SEQ ID NO: 51 - protein, 12 aa, Artificial Sequence
  Synthetic Construct
WRLRPVDYGMDY

SEQ ID NO: 52 - protein, 15 aa, Artificial Sequence
  Synthetic Construct
RASQSVSTSSYSYMH

SEQ ID NO: 53 - protein, 7 aa, Artificial Sequence
  Synthetic Construct
YASNLES

SEQ ID NO: 54 - protein, 9 aa, Artificial Sequence
  Synthetic Construct
QHSWEIPFT

SEQ ID NO: 55 - protein, 8 aa, Artificial Sequence
  Synthetic Construct
GYTFTDYY

SEQ ID NO: 56 - protein, 8 aa, Artificial Sequence
  Synthetic Construct
VNPNNGGK

SEQ ID NO: 57 - protein, 14 aa, Artificial Sequence
  Synthetic Construct
ARWRLRPVDYGMDY

SEQ ID NO: 58 - protein, 10 aa, Artificial Sequence
  Synthetic Construct
QSVSTSSYSY

SEQ ID NO: 59 - protein, 3 aa, Artificial Sequence
  Synthetic Construct
YAS

SEQ ID NO: 60 - protein, 9 aa, Artificial Sequence
  Synthetic Construct
QHSWEIPFT

SEQ ID NO: 61 - protein, 121 aa, Artificial Sequence
  Synthetic Construct
EVQLQQSGPELVKPGASVKMSCKASGYTFTDYYMNWVKQSHGKSLEWIGRVNPNNGGKTYNQKFKGKATLTVDKSLSTAYMQLNSLTSEDSAVYYCARWRLRPVDYGMDYWGQGTSVTVSS

SEQ ID NO: 62 - protein, 111 aa, Artificial Sequence
  Synthetic Construct
DIVLTQSPASLAVSLGQRATISCRASQSVSTSSYSYMHWYQQKPGQPPKLLIKYASNLESGVPARFSGSGSGTDFTLNIHPVEEEDTATYYCQHSWEIPFTFGSGTKLEIK

SEQ ID NO: 63 - DNA, 364 bp, Artificial Sequence
  Synthetic Construct
gaggtccagctgcaacagtctggacctgagctggtgaagcctggggcttcagtgaagatgtcctgtaaggcttctggatacacattcactgactactacatgaactgggtgaagcagagtcatggaaagagtcttgagtggattggacgtgttaatcctaacaatggtggtaaaacctacaaccagaagttcaagggcaaggccacattgacagtagacaaatccctcagcacagcctacatgcagctcaacagcctgacatctgaggactctgcggtctattactgtgcaagatggaggctacggcccgttgactatggtatggactactggggtcaaggaacctcagtcaccgtctcctcag

SEQ ID NO: 64 - DNA, 334 bp, Artificial Sequence
  Synthetic Construct
gacattgtgctgacacagtctcctgcttccttggctgtatctctggggcagagggccaccatctcatgcagggccagccaaagtgtcagtacatctagctatagttatatgcactggtaccaacagaaaccaggacagccacccaaactcctcatcaagtatgcatccaacctagaatctggggtccctgccaggttcagtggcagtgggtctgggacagacttcaccctcaacatccatcctgtggaggaggaggatactgcaacatattactgtcagcacagttgggagattccattcacgttcggctcggggacaaagttggaaataaaac

SEQ ID NO: 65 - protein, 5 aa, Artificial Sequence
  Synthetic Construct
DYEMH

SEQ ID NO: 66 - protein, 17 aa, Artificial Sequence
  Synthetic Construct
GIDPETGGTAYNQKFKG

SEQ ID NO: 67 - protein, 6 aa, Artificial Sequence
  Synthetic Construct
GAWFAY

SEQ ID NO: 68 - protein, 14 aa, Artificial Sequence
  Synthetic Construct
RSSTGAVTTSNSAN

SEQ ID NO: 69 - protein, 7 aa, Artificial Sequence
  Synthetic Construct
GTNNRAP

SEQ ID NO: 70 - protein, 9 aa, Artificial Sequence
  Synthetic Construct
ALWYNNHFV

SEQ ID NO: 71 - protein, 8 aa, Artificial Sequence
  Synthetic Construct
GYTFTDYE

SEQ ID NO: 72 - protein, 8 aa, Artificial Sequence
  Synthetic Construct
IDPETGGT

SEQ ID NO: 73 - protein, 8 aa, Artificial Sequence
  Synthetic Construct
TRGAWFAY

SEQ ID NO: 74 - protein, 9 aa, Artificial Sequence
  Synthetic Construct
TGAVTTSNS

SEQ ID NO: 75 - protein, 3 aa, Artificial Sequence
  Synthetic Construct
GTN

SEQ ID NO: 76 - protein, 9 aa, Artificial Sequence
  Synthetic Construct
ALWYNNHFV

SEQ ID NO: 77 - protein, 115 aa, Artificial Sequence
  Synthetic Construct
QVQLQQSGAELVRPGASVKLSCKASGYTFTDYEMHWVRQTPVHGLEWIGGIDPETGGTAYNQKFKGKATLTADKSSSTAYMELRSLTSEDSAVYYCTRGAWFAYWGQGTLVTVSA

SEQ ID NO: 78 - protein, 109 aa, Artificial Sequence
  Synthetic Construct
QAVVTQESALTTSPGETVTLTCRSSTGAVTTSNSANWVQEKPDHLFTGLIGGTNNRAPGVPARFSGSLIGDKAALTITGAQTEDEAIYFCALWYNNHFVFGGGTKLTVL

SEQ ID NO: 79 - DNA, 346 bp, Artificial Sequence
  Synthetic Construct
caggttcaattgcagcagtctggggctgagctggtgaggcctggggcttcagtgaagctgtcctgcaaggcttcgggctatacatttactgactatgaaatgcactgggtgaggcagacacctgtgcatggcctggaatggattggaggtattgatcctgaaactggtggtactgcctacaatcagaagttcaagggcaaggccacactgactgcagacaaatcctccagcacagcctacatggagctccgcagcctgacatctgaggactctgccgtctattactgtacacgaggggcctggtttgcttactggggccaagggactctggtcactgtctctgcag

SEQ ID NO: 80 - DNA, 328 bp, Artificial Sequence
  Synthetic Construct
caggctgttgtgactcaggaatctgcactcaccacatcacctggtgaaacagtcacactcacttgtcgctcaagtactggggctgttacaactagtaactctgccaactgggtccaagaaaaaccagatcatttattcactggtctaatcggtggtaccaacaaccgagctccaggtgttcctgccagattctcaggctccctgattggagacaaggctgccctcaccatcacaggggcacagactgaggatgaggcaatatatttctgtgctctatggtacaacaaccatttcgtgttcggtggaggcaccaaactgactgtcctag

SEQ ID NO: 81 - protein, 5 aa, Artificial Sequence
  Synthetic Construct
SYWMH

SEQ ID NO: 82 - protein, 17 aa, Artificial Sequence
  Synthetic Construct
AIYPGNSDTSYNQKFKG

SEQ ID NO: 83 - protein, 12 aa, Artificial Sequence
  Synthetic Construct
GGFDYSNYWFAY

SEQ ID NO: 84 - protein, 11 aa, Artificial Sequence
  Synthetic Construct
KASQSVSNDVA

SEQ ID NO: 85 - protein, 7 aa, Artificial Sequence
  Synthetic Construct
YASNRYT

SEQ ID NO: 86 - protein, 8 aa, Artificial Sequence
  Synthetic Construct
QQDYSSYT

SEQ ID NO: 87 - protein, 8 aa, Artificial Sequence
  Synthetic Construct
GYTFTSYW

SEQ ID NO: 88 - protein, 8 aa, Artificial Sequence
  Synthetic Construct
IYPGNSDT

SEQ ID NO: 89 - protein, 14 aa, Artificial Sequence
  Synthetic Construct
TRGGFDYSNYWFAY

SEQ ID NO: 90 - protein, 6 aa, Artificial Sequence
  Synthetic Construct
QSVSND

SEQ ID NO: 91 - protein, 3 aa, Artificial Sequence
  Synthetic Construct
YAS

SEQ ID NO: 92 - protein, 8 aa, Artificial Sequence
  Synthetic Construct
QQDYSSYT

SEQ ID NO: 93 - protein, 121 aa, Artificial Sequence
  Synthetic Construct
EVQLQQSGTVLARPGASVKMSCKASGYTFTSYWMHWVKQRPGQGLEWIGAIYPGNSDTSYNQKFKGKAKLTAVTSASTAYMELSSLTNEDSAVYYCTRGGFDYSNYWFAYWGQGTLVTVSA

SEQ ID NO: 94 - protein, 106 aa, Artificial Sequence
  Synthetic Construct
SIVMTQTPKFLLVSAGDRVTITCKASQSVSNDVAWYQQKPGQSPKLLIYYASNRYTGVPDRFTGSGYGTDFTFTISTVQAEDLAVYFCQQDYSSYTFGGGTKLEIK

SEQ ID NO: 95 - DNA, 363 bp, Artificial Sequence
  Synthetic Construct
gaggttcagctccagcagtctgggactgtgctggcaaggcctggggcttcagtgaagatgtcctgcaaggcttctggctacacctttaccagctactggatgcactgggtaaaacagaggcctggacagggtctggaatggattggcgctatttatcctggaaatagtgatactagctacaaccagaagttcaagggcaaggccaaactgactgcagtcacatctgccagcactgcctacatggagctcagcagcctgacaaatgaggactctgcggtctattactgtacaagaggaggatttgactatagtaactactggtttgcttactggggccaagggactctggtcactgtctctgca

SEQ ID NO: 96 - DNA, 319 bp, Artificial Sequence
  Synthetic Construct
agtattgtgatgacccagactcccaaattcctgcttgtatcagcaggagacagggttaccataacctgcaaggccagtcagagtgtgagtaatgatgtagcttggtaccaacagaagccagggcagtctcctaaactgctgatatactatgcatccaatcgctacactggagtccctgatcgcttcactggcagtggatatgggacggatttcactttcaccatcagcactgtgcaggctgaagacctggcagtttatttctgtcagcaggattatagctcgtacacgttcggaggggggaccaagctggaaataaaac

SEQ ID NO: 97 - protein, 5 aa, Artificial Sequence
  Synthetic Construct
RSWMN

SEQ ID NO: 98 - protein, 17 aa, Artificial Sequence
  Synthetic Construct
WIYPGDGDTNYNGKFKG

SEQ ID NO: 99 - protein, 11 aa, Artificial Sequence
  Synthetic Construct
SATLPYWYFDV

SEQ ID NO: 100 - protein, 11 aa, Artificial Sequence
  Synthetic Construct
KASQDIKSYLS

SEQ ID NO: 101 - protein, 7 aa, Artificial Sequence
  Synthetic Construct
YATNLAD

SEQ ID NO: 102 - protein, 9 aa, Artificial Sequence
  Synthetic Construct
LQHVESPWT

SEQ ID NO: 103 - protein, 8 aa, Artificial Sequence
  Synthetic Construct
GYAFSRSW

SEQ ID NO: 104 - protein, 8 aa, Artificial Sequence
  Synthetic Construct
IYPGDGDT

SEQ ID NO: 105 - protein, 13 aa, Artificial Sequence
  Synthetic Construct
ARSATLPYWYFDV

SEQ ID NO: 106 - protein, 6 aa, Artificial Sequence
  Synthetic Construct
QDIKSY

SEQ ID NO: 107 - protein, 3 aa, Artificial Sequence
  Synthetic Construct
YAT

SEQ ID NO: 108 - protein, 9 aa, Artificial Sequence
  Synthetic Construct
LQHVESPWT

SEQ ID NO: 109 - protein, 120 aa, Artificial Sequence
  Synthetic Construct
QVQLQQSGPELVKPGASVKISCKASGYAFSRSWMNWVKQRPGKGLEWIGWIYPGDGDTNYNGKFKGKATLTADKSSSTAYMQLSSLTSEDSAAYFCARSATLPYWYFDVWGAGTTVTVSS

SEQ ID NO: 110 - protein, 107 aa, Artificial Sequence
  Synthetic Construct
DIKMTQSPSSMYASLGERVTITCKASQDIKSYLSWYQQKPWKSPKTLIYYATNLADGVPSRFSGSGSGQDYSLTISSLGSDDTATYYCLQHVESPWTFGGGTKLEIK

SEQ ID NO: 111 - DNA, 361 bp, Artificial Sequence
  Synthetic Construct
caggtccagctgcagcagtctggacctgagctggtgaagcctggggcctcagtgaagatttcctgcaaagcttctggctatgcattcagtcgctcctggatgaactgggtaaagcagaggcctggaaagggtcttgagtggattggatggatttatcctggagatggtgatactaactacaatggaaagttcaagggcaaggccacactgactgcagacaaatcctcaagcacagcctacatgcagctcagcagcctgacatctgaggactctgcggcctatttctgtgcaaggtcggctaccctaccttactggtacttcgatgtctggggcgcagggaccacggtcaccgtctcctcag

SEQ ID NO: 112 - DNA, 322 bp, Artificial Sequence
  Synthetic Construct
gacatcaagatgacccagtctccatcctccatgtatgcatcgctgggagagagagtcactatcacttgcaaggcgagtcaggacattaaaagctatttaagttggtaccagcagaaaccatggaaatctcctaagaccctgatctattatgcaacaaacttggcagatggggtcccatcaagattcagtggcagtggatctgggcaggattattctctaaccatcagcagcctggggtctgacgatacagcaacttattactgtctacagcatgttgagagcccgtggacgttcggtggaggcaccaagctggaaatcaaac

SEQ ID NO: 113 - protein, 5 aa, Artificial Sequence
  Synthetic Construct
AYVMH

SEQ ID NO: 114 - protein, 17 aa, Artificial Sequence
  Synthetic Construct
YIFPYNDGTEYNEKFKG

SEQ ID NO: 115 - protein, 11 aa, Artificial Sequence
  Synthetic Construct
RTDGNPYTMDY

SEQ ID NO: 116 - protein, 11 aa, Artificial Sequence
  Synthetic Construct
KASQDVSTAVA

SEQ ID NO: 117 - protein, 7 aa, Artificial Sequence
  Synthetic Construct
SASYRYT

SEQ ID NO: 118 - protein, 9 aa, Artificial Sequence
  Synthetic Construct
QQHYSTPFT

SEQ ID NO: 119 - protein, 8 aa, Artificial Sequence
  Synthetic Construct
GYTFTAYV

SEQ ID NO: 120 - protein, 8 aa, Artificial Sequence
  Synthetic Construct
IFPYNDGT

SEQ ID NO: 121 - protein, 13 aa, Artificial Sequence
  Synthetic Construct
ARRTDGNPYTMDY

SEQ ID NO: 122 - protein, 6 aa, Artificial Sequence
  Synthetic Construct
QDVSTA

SEQ ID NO: 123 - protein, 3 aa, Artificial Sequence
  Synthetic Construct
SAS

SEQ ID NO: 124 - protein, 9 aa, Artificial Sequence
  Synthetic Construct
QQHYSSPFT

SEQ ID NO: 125 - protein, 120 aa, Artificial Sequence
  Synthetic Construct
EVQLQQSGPELVNPGASVKMSCKASGYTFTAYVMHWVKQKPGQGLEWIGYIFPYNDGTEYNEKFKGKATLTSDKSSSTAYMELSSLTSEDSAVYYCARRTDGNPYTMDYWGQGTSVTVSS

SEQ ID NO: 126 - protein, 107 aa, Artificial Sequence
  Synthetic Construct
DIVMTQSHKFMSTSVGDRVSITCKASQDVSTAVAWYQQKPGQSPKLLIHSASYRYTGVPDRFTGRGSGTDFTFTISSVQAEDLAVYYCQQHYSTPFTFGSGTKLEIK

SEQ ID NO: 127 - DNA, 361 bp, Artificial Sequence
  Synthetic Construct
gaggtccagctgcagcagtctggacctgagttggtaaatcctggggcttcagtgaagatgtcctgcaaggcttctggatacacattcactgcctatgttatgcactgggtgaaacagaagcctgggcagggccttgagtggattggatatatttttccttacaatgatggtactgagtacaatgagaagttcaaaggcaaggccacactgacttcagacaaatcctccagcacagcctacatggagctcagcagcctgacctctgaggactctgcggtctattactgtgcaaggaggacagatggtaacccctatactatggactattggggtcaaggaacctcagtcaccgtctcctcag

SEQ ID NO: 128 - DNA, 322 bp, Artificial Sequence
  Synthetic Construct
gacattgtgatgacccagtctcacaaattcatgtccacatcagtaggagacagggtcagcatcacctgcaaggccagtcaggatgtgagtactgctgtagcctggtatcaacagaaaccaggacaatctcctaaactactgattcattcggcatcctaccggtacactggagtccctgatcgcttcactggcagaggatctgggacggatttcactttcaccatcagcagtgtgcaggctgaagacctggcagtttattactgtcagcaacattatagtactccattcacgttcggctcggggacaaagttggaaataaaac

SEQ ID NO: 129 - protein, 5 aa, Artificial Sequence
  Synthetic Construct
DYYIH

SEQ ID NO: 130 - protein, 17 aa, Artificial Sequence
  Synthetic Construct
EIYPGSDDAYYNEKFKG

SEQ ID NO: 131 - protein, 7 aa, Artificial Sequence
  Synthetic Construct
ETTATAY

SEQ ID NO: 132 - protein, 10 aa, Artificial Sequence
  Synthetic Construct
SASSSVSLIY

SEQ ID NO: 133 - protein, 7 aa, Artificial Sequence
  Synthetic Construct
STSNLAS

SEQ ID NO: 134 - protein, 9 aa, Artificial Sequence
  Synthetic Construct
QQRSGYPPT

SEQ ID NO: 135 - protein, 8 aa, Artificial Sequence
  Synthetic Construct
GYTFTDYY

SEQ ID NO: 136 - protein, 8 aa, Artificial Sequence
  Synthetic Construct
IYPGSDDA

SEQ ID NO: 137 - protein, 9 aa, Artificial Sequence
  Synthetic Construct
TRETTATAY

SEQ ID NO: 138 - protein, 5 aa, Artificial Sequence
  Synthetic Construct
SSVSL

SEQ ID NO: 139 - protein, 3 aa, Artificial Sequence
  Synthetic Construct
STS

SEQ ID NO: 140 - protein, 9 aa, Artificial Sequence
  Synthetic Construct
QQRSGYPPT

SEQ ID NO: 141 - protein, 116 aa, Artificial Sequence
  Synthetic Construct
EVQLQQSGPELVKPGASVKVSCKASGYTFTDYYIHWVKQRPGQGLEWIGEIYPGSDDAYYNEKFKGKATLTADKSSSTAYMQLSSLTSEDSAVYFCTRETTATAYWGQGTLVTVSA

SEQ ID NO: 142 - protein, 106 aa, Artificial Sequence
  Synthetic Construct
QIVLTQSPAIMSASPGEKVTITCSASSSVSLIYWFQQKPGTSPKLWIYSTSNLASGVPARFSGSGSGTSYSLTISRMEAEDAATYYCQQRSGYPPTFGGGTKLEIK

SEQ ID NO: 143 - DNA, 351 bp, Artificial Sequence
  Synthetic Construct
ctgaggtccagctgcagcagtctggacctgagctggttaagcctggggcttcagtgaaggtatcctgcaaggcctctggatacacattcactgactactatatacactgggtgaagcagaggcctgggcagggccttgagtggattggagagatttatcctggaagtgatgatgcttactacaatgagaaattcaagggcaaggccacactgactgcagacaaatcctccagcacagcctacatgcagctcagcagcctgacatctgaggactctgcagtctatttctgtacaagagagactacggctacggcttactggggccaagggactctggtcactgtctctgcag

SEQ ID NO: 144 - DNA, 319 bp, Artificial Sequence
  Synthetic Construct
caaattgttctcacccagtctccagcaatcatgtctgcatctccaggggagaaggtcaccataacctgcagtgccagctcaagtgtaagtctcatttactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtggttacccacccacgttcggaggggggaccaagctggaaataaaac

SEQ ID NO: 145 - protein, 5 aa, Artificial Sequence
  Synthetic Construct
DHGIH

SEQ ID NO: 146 - protein, 17 aa, Artificial Sequence
  Synthetic Construct
NISPGNGDIKYNEKFKG

SEQ ID NO: 147 - protein, 4 aa, Artificial Sequence
  Synthetic Construct
YFVD

SEQ ID NO: 148 - protein, 17 aa, Artificial Sequence
  Synthetic Construct
KSSQSLLNSNNQKNCLA

SEQ ID NO: 149 - protein, 7 aa, Artificial Sequence
  Synthetic Construct
FACTRES

SEQ ID NO: 150 - protein, 9 aa, Artificial Sequence
  Synthetic Construct
QQHCNTPLT

SEQ ID NO: 151 - protein, 8 aa, Artificial Sequence
  Synthetic Construct
GYTFTDHG

SEQ ID NO: 152 - protein, 8 aa, Artificial Sequence
  Synthetic Construct
ISPGNGDI

SEQ ID NO: 153 - protein, 6 aa, Artificial Sequence
  Synthetic Construct
TTYFVD

SEQ ID NO: 154 - protein, 12 aa, Artificial Sequence
  Synthetic Construct
QSLLNSNNQKNC

SEQ ID NO: 155 - protein, 3 aa, Artificial Sequence
  Synthetic Construct
FAC

SEQ ID NO: 156 - protein, 9 aa, Artificial Sequence
  Synthetic Construct
QQHCNTPLT

SEQ ID NO: 157 - protein, 113 aa, Artificial Sequence
  Synthetic Construct
QVQLQQSDAELVKPGTSVKISCKASGYTFTDHGIHWVKQRPERGLEWIGNISPGNGDIKYNEKFKGKATLTADKSSSTVYMQVNSLTSEDSAVYFCTTYFVDWGRGTLVTVSA

SEQ ID NO: 158 - protein, 113 aa, Artificial Sequence
  Synthetic Construct
DIVMTQSPSSLAMSIGQRVTMSCKSSQSLLNSNNQKNCLAWYQQKPGQSPRLLIYFACTRESGVPDRFIGSGSGTDFTLTISSVQAEDLAYYFCQQHCNTPLTFGAGTKLELK

SEQ ID NO: 159 - DNA, 340 bp, Artificial Sequence
  Synthetic Construct
caggttcagctgcaacagtctgacgctgagttggtgaaacctgggacttcagtgaagatatcctgcaaggcttctggctacaccttcactgaccatggtattcactgggtgaaacagaggcctgaacggggcctggaatggattggaaatatttctcccggaaatggtgatattaagtataatgagaagttcaagggcaaggccacgctgactgcagacaaatcctccagcactgtctacatgcaggtcaacagcctgacatctgaggattctgcagtgtatttctgtacaacctattttgttgactggggccgggggactctggtcactgtctctgcag

SEQ ID NO: 160 - DNA, 340 bp, Artificial Sequence
  Synthetic Construct
gacattgtgatgacacagtctccatcctccctggctatgtcaattggacagagggtcactatgagctgcaagtccagtcagagccttttaaatagtaacaatcaaaagaactgtttggcctggtaccagcagaaaccaggacagtctcctagacttctgatttactttgcatgtactagggaatcgggggtccctgatcgcttcattggcagtggatctgggacagatttcacccttaccatcagcagtgtgcaggctgaagacctggcatattacttctgtcagcaacattgtaacactccgctcacgttcggtgctgggaccaagctggagctgaaac

SEQ ID NO: 161 - protein, 5 aa, Artificial Sequence
  Synthetic Construct
TYWMN

SEQ ID NO: 162 - protein, 17 aa, Artificial Sequence
  Synthetic Construct
RIFPGDGDTDYDGKFKG

SEQ ID NO: 163 - protein, 12 aa, Artificial Sequence
  Synthetic Construct
TGAAYEFDPFPY

SEQ ID NO: 164 - protein, 16 aa, Artificial Sequence
  Synthetic Construct
SSTKSLLHSSGITYLY

SEQ ID NO: 165 - protein, 7 aa, Artificial Sequence
  Synthetic Construct
RMSNLAS

SEQ ID NO: 166 - protein, 9 aa, Artificial Sequence
  Synthetic Construct
AQMLERPFT

SEQ ID NO: 167 - protein, 8 aa, Artificial Sequence
  Synthetic Construct
GYAFSTYW

SEQ ID NO: 168 - protein, 8 aa, Artificial Sequence
  Synthetic Construct
IFPGDGDT

SEQ ID NO: 169 - protein, 14 aa, Artificial Sequence
  Synthetic Construct
ARTGAAYEFDPFPY

SEQ ID NO: 170 - protein, 11 aa, Artificial Sequence
  Synthetic Construct
KSLLHSSGITY

SEQ ID NO: 171 - protein, 3 aa, Artificial Sequence
  Synthetic Construct
RMS

SEQ ID NO: 172 - protein, 9 aa, Artificial Sequence
  Synthetic Construct
AQMLERPFT

SEQ ID NO: 173 - protein, 121 aa, Artificial Sequence
  Synthetic Construct
QVQLQQSGPELVKPGASVKISCKGSGYAFSTYWMNWVKQRPGKGLEWIGRIFPGDGDTDYDGKFKGKATLTADKSSNTAYMQLSSLTSEDSAVYFCARTGAAYEFDPFPYWGQGTLVTVSA

SEQ ID NO: 174 - protein, 112 aa, Artificial Sequence
  Synthetic Construct
DIVMTQAAFSNPVTLGTSASISCSSTKSLLHSSGITYLYWYLQRPGQSPQLLIYRMSNLASGVPDRFSGSGSGTDFTLRISRVEAEDVGVYYCAQMLERPFTFGSGTKLEIK

SEQ ID NO: 175 - DNA, 364 bp, Artificial Sequence
  Synthetic Construct
caggttcagctgcagcagtctggacctgagctggtgaagcctggggcctcagtgaagatttcctgcaaaggttctggctacgcattcagtacctactggatgaactgggtgaagcagaggcctggaaagggtcttgagtggattggacggatttttcctggagatggagatacagattacgatgggaagttcaagggcaaggccacactgactgcagacaaatcctccaacacagcctacatgcaactcagcagcctgacatctgaagactctgcggtctacttctgtgcaagaactggggccgcctatgaattcgacccttttccttactggggccaagggactctggtcactgtctctgcag

SEQ ID NO: 176 - DNA, 337 bp, Artificial Sequence
  Synthetic Construct
gatattgtgatgacgcaggctgcattctccaatccagtcactcttggaacatcagcttccatctcttgcagttctactaagagtctcctacatagtagcggcatcacttatctgtattggtatctgcagaggccaggccagtctcctcagctcctgatatatcggatgtccaaccttgcctcaggagtcccagacaggttcagtggcagtgggtcaggaactgatttcacactgagaatcagcagagtggaggctgaggatgtgggtgtttattactgtgctcaaatgctagaacgcccattcacgttcggctcggggacaaagttggaaataaaac

SEQ ID NO: 177 - protein, 5 aa, Artificial Sequence
  Synthetic Construct
SYWLN

SEQ ID NO: 178 - protein, 17 aa, Artificial Sequence
  Synthetic Construct
RIYPGDGDTDYNGKFKG

SEQ ID NO: 179 - protein, 9 aa, Artificial Sequence
  Synthetic Construct
GDGYWAMDY

SEQ ID NO: 180 - protein, 16 aa, Artificial Sequence
  Synthetic Construct
RFSKSLLHSNGITYLY

SEQ ID NO: 181 - protein, 7 aa, Artificial Sequence
  Synthetic Construct
QMSNLAS

SEQ ID NO: 182 - protein, 9 aa, Artificial Sequence
  Synthetic Construct
AQNLELPWT

SEQ ID NO: 183 - protein, 8 aa, Artificial Sequence
  Synthetic Construct
GYAFSSYW

SEQ ID NO: 184 - protein, 8 aa, Artificial Sequence
  Synthetic Construct
IYPGDGDT

SEQ ID NO: 185 - protein, 11 aa, Artificial Sequence
  Synthetic Construct
VRGDGYWAMDY

SEQ ID NO: 186 - protein, 11 aa, Artificial Sequence
  Synthetic Construct
KSLLHSNGITY

SEQ ID NO: 187 - protein, 3 aa, Artificial Sequence
  Synthetic Construct
QMS

SEQ ID NO: 188 - protein, 9 aa, Artificial Sequence
  Synthetic Construct
AQNLELPWT

SEQ ID NO: 189 - protein, 118 aa, Artificial Sequence
  Synthetic Construct
QVQLQQSGPELVKPGASVKISCKASGYAFSSYWLNWVKQRPGKGLEWFGRIYPGDGDTDYNGKFKGKATLTADKSSSTAYMQLRSLTSEDSAVYFCVRGDGYWAMDYWGQGTSVTVSS

SEQ ID NO: 190 - protein, 112 aa, Artificial Sequence
  Synthetic Construct
DIVMTQAAFSNPVTLGTSASISCRFSKSLLHSNGITYLYWYLQKPGQSPQLLIYQMSNLASGVPDRFSSSGSGTDFTLRISRVEAEDVGVYYCAQNLELPWTFGGGTKLEIK

SEQ ID NO: 191 - DNA, 355 bp, Artificial Sequence
  Synthetic Construct
caggttcagctgcagcagtctggacctgagctggtgaagcctggggcctcggtgaagatttcctgcaaagcttctggctacgcattcagtagctactggctgaactgggtgaagcagaggcctggaaagggtcttgagtggtttggacggatttatcctggagatggagatactgactacaatgggaagttcaagggcaaggccacactgactgcagacaaatcctccagcacagcctacatgcaactcagaagcctgacatctgaggactctgcggtctacttctgtgtaagaggtgatggttactgggctatggactactggggtcaaggaacctcagtcaccgtctcctcag

SEQ ID NO: 192 - DNA, 337 bp, Artificial Sequence
  Synthetic Construct
gatattgtgatgacgcaggctgcattctccaatccagtcactcttggaacatcagcttccatctcctgcaggtttagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttccgtggacgttcggtggaggcaccaagctggaaatcaaac

SEQ ID NO: 193 - protein, 5 aa, Artificial Sequence
  Synthetic Construct
NYYMS

SEQ ID NO: 194 - protein, 17 aa, Artificial Sequence
  Synthetic Construct
TISNNGDSTYYLDTVKG

SEQ ID NO: 195 - protein, 7 aa, Artificial Sequence
  Synthetic Construct
VGTGFTY

SEQ ID NO: 196 - protein, 11 aa, Artificial Sequence
  Synthetic Construct
RASQSINNYLH

SEQ ID NO: 197 - protein, 7 aa, Artificial Sequence
  Synthetic Construct
FASQSIS

SEQ ID NO: 198 - protein, 9 aa, Artificial Sequence
  Synthetic Construct
QQSNSWPLT

SEQ ID NO: 199 - protein, 8 aa, Artificial Sequence
  Synthetic Construct
GFTFSNYY

SEQ ID NO: 200 - protein, 8 aa, Artificial Sequence
  Synthetic Construct
ISNNGDST

SEQ ID NO: 201 - protein, 9 aa, Artificial Sequence
  Synthetic Construct
TRVGTGFTY

SEQ ID NO: 202 - protein, 6 aa, Artificial Sequence
  Synthetic Construct
QSINNY

SEQ ID NO: 203 - protein, 3 aa, Artificial Sequence
  Synthetic Construct
FAS

SEQ ID NO: 204 - protein, 9 aa, Artificial Sequence
  Synthetic Construct
QQSNSWPLT

SEQ ID NO: 205 - protein, 116 aa, Artificial Sequence
  Synthetic Construct
DVNLVESGGGLVKLGGSLKLSCAASGFTFSNYYMSWVRQSPEKRLEWVATISNNGDSTYYLDTVKGRFTISRDSAENTLYLQMSSLISEDTAVYYCTRVGTGFTYWGQGTLVTVSA

SEQ ID NO: 206 - protein, 107 aa, Artificial Sequence
  Synthetic Construct
DIVLTQSPATLSVTPGDSVSLSCRASQSINNYLHWYQQRSHESPRLLIKFASQSISDIPSRFSGSGSGTDFTLSINSIETEDFGMYFCQQSNSWPLTFGAGTKLELK

SEQ ID NO: 207 - DNA, 349 bp, Artificial Sequence
  Synthetic Construct
gacgtgaacctcgtggagtctgggggaggcttagtgaagcttggagggtccctgaaactctcctgtgcagcctctggattcactttcagtaactactacatgtcttgggttcgccagagtccggagaagaggctggagtgggtcgcaaccattagtaataatggtgatagcacctactatctagacactgtgaagggccgattcaccatctccagagacagtgccgagaacaccctgtacctgcaaatgagcagtctgatttctgaggacacagccgtgtattactgtacaagagttgggacggggtttacttactggggccaagggactctggtcactgtctctgcag

SEQ ID NO: 208 - DNA, 322 bp, Artificial Sequence
  Synthetic Construct
gatattgtgctaactcagtctccagccaccctgtctgtgactccaggagatagcgtcagtctttcctgcagggccagccaaagtattaacaactacctacactggtatcaacaaagatcacatgagtctccaaggcttctcatcaagtttgcttcccagtccatctctgacatcccctccaggttcagtggcagtggatcagggacagatttcactctcagtatcaacagtatagagactgaagattttggaatgtatttctgtcaacagagtaacagctggccgctcacgttcggtgctgggaccaagctggagctgaaac

SEQ ID NO: 209 - protein, 5 aa, Artificial Sequence
  Synthetic Construct
SYVIH

SEQ ID NO: 210 - protein, 17 aa, Artificial Sequence
  Synthetic Construct
YINPYSDYTQYNEKFKG

SEQ ID NO: 211 - protein, 11 aa, Artificial Sequence
  Synthetic Construct
RADGNPYAMDY

SEQ ID NO: 212 - protein, 11 aa, Artificial Sequence
  Synthetic Construct
KASQDVSTAVV

SEQ ID NO: 213 - protein, 7 aa, Artificial Sequence
  Synthetic Construct
SASYRYT

SEQ ID NO: 214 - protein, 9 aa, Artificial Sequence
  Synthetic Construct
QQHYSTPFT

SEQ ID NO: 215 - protein, 8 aa, Artificial Sequence
  Synthetic Construct
GYTFTSYV

SEQ ID NO: 216 - protein, 8 aa, Artificial Sequence
  Synthetic Construct
INPYSDYT

SEQ ID NO: 217 - protein, 13 aa, Artificial Sequence
  Synthetic Construct
ARRADGNPYAMDY

SEQ ID NO: 218 - protein, 6 aa, Artificial Sequence
  Synthetic Construct
QDVSTA

SEQ ID NO: 219 - protein, 3 aa, Artificial Sequence
  Synthetic Construct
SAS

SEQ ID NO: 220 - protein, 9 aa, Artificial Sequence
  Synthetic Construct
QQHYSTPFT

SEQ ID NO: 221 - protein, 120 aa, Artificial Sequence
  Synthetic Construct
EVQLQQSGPELVKPGASVKMSCKASGYTFTSYVIHWVKQKPGQGLEWIGYINPYSDYTQYNEKFKGKATLTSDKSSSTAYMELSSLTSEDSAVYSCARRADGNPYAMDYWGQGTSVTVSS

SEQ ID NO: 222 - protein, 107 aa, Artificial Sequence
  Synthetic Construct
DIVMTQSHKFMSTSVGDRVSTTCKASQDVSTAVVWYQQKPGQSPKLLIYSASYRYTGVPDRFTGSGSGTDFTFTITSVQAEDLAVYYCQQHYSTPFTFGSGTKLEIK

SEQ ID NO: 223 - DNA, 361 bp, Artificial Sequence
  Synthetic Construct
gaggtccagctacagcagtctggacctgagctggtaaagcctggggcttcagtgaagatgtcctgcaaggcttctggatacacattcactagctatgttattcactgggtaaagcagaagcctgggcagggccttgagtggattggatatattaatccttacagtgattatactcagtacaatgagaagttcaaaggcaaggccacactgacttcagacaaatcctccagcacagcctacatggagctcagcagcctgacctctgaggactctgcggtctattcctgtgcaaggagggcagatggtaacccctatgctatggactactggggtcaaggaacctcagtcaccgtctcctcag

SEQ ID NO: 224 - DNA, 322 bp, Artificial Sequence
  Synthetic Construct
gacattgtgatgacccagtctcacaaattcatgtccacatcagtaggagacagggtcagcaccacctgcaaggccagtcaggatgtgagtactgctgtagtctggtatcaacagaaaccaggacaatctcctaaactactgatttactcggcatcctaccggtacactggagtccctgatcgcttcactggcagtggatctgggacggatttcactttcaccatcaccagtgtgcaggctgaagacctggcagtttattactgtcagcaacattatagtactccattcacgttcggctcggggacaaagttggaaataaaac

SEQ ID NO: 225 - protein, 1 aa, Artificial Sequence
  Synthetic Construct
  VARIANT 1: Can be present in repeats of any integer
G

SEQ ID NO: 226 - protein, 16 aa, Artificial Sequence
  Synthetic Construct
  VARIANT (3)..(16): Can be present or absent
GSGSGSGSGSGSGSGS

SEQ ID NO: 227 - protein, 40 aa, Artificial Sequence
  Synthetic Construct
  VARIANT (6)..(40): Can be present or absent
GSGGSGSGGSGSGGSGSGGSGSGGSGSGGSGSGGSGSGGS

SEQ ID NO: 228 - protein, 40 aa, Artificial Sequence
  Synthetic Construct
  VARIANT (6)..(40): Can be present or absent
GGGGSGGGGSGGGGSGGGGSGGGGSGGGGSGGGGSGGGGS

SEQ ID NO: 229 - protein, 32 aa, Artificial Sequence
  Synthetic Construct
  VARIANT (5)..(32): Can be present or absent
GGGSGGGSGGGSGGGSGGGSGGGSGGGSGGGS

SEQ ID NO: 230 - protein, 15 aa, Artificial Sequence
  Synthetic Construct
GGGGSGGGGSGGGGS

SEQ ID NO: 231 - protein, 30 aa, Artificial Sequence
  Synthetic Construct
GGGGSGGGGSGGGGSGGGGSGGGGSGGGGS

SEQ ID NO: 232 - protein, 60 aa, Artificial Sequence
  Synthetic Construct
  VARIANT (16)..(60): Can be present or absent
GSTSGSGKPGSGEGSGSTSGSGKPGSGEGSGSTSGSGKPGSGEGSGSTSGSGKPGSGEGS

SEQ ID NO: 233 - protein, 17 aa, Artificial Sequence
  Synthetic Construct
  VARIANT 9, 10: Xaa = AN or TD
  VARIANT 12: Xaa = N or D
RIFPGDGDXXYXGKFKG

SEQ ID NO: 234 - protein, 12 aa, Artificial Sequence
  Synthetic Construct
  VARIANT 6: Xaa = D or E
TGAAYXFDPFPY

SEQ ID NO: 235 - protein, 16 aa, Artificial Sequence
  Synthetic Construct
  VARIANT 3: Xaa = S or T
  VARIANT 10: Xaa = N or S
  VARIANT 12: Xaa = V or I
SSXKSLLHSXGXTYLY

SEQ ID NO: 236 - protein, 5 aa, Artificial Sequence
  Synthetic Construct
  VARIANT 1: Xaa = S or T
  VARIANT 4: Xaa = L or M
XYWXN

SEQ ID NO: 237 - protein, 17 aa, Artificial Sequence
  Synthetic Construct
  VARIANT 3: Xaa = Y or F
  VARIANT 9, 10: Xaa = AN or TD
  VARIANT 12: Xaa = N or D
RIXPGDGDXXYXGKFKG

SEQ ID NO: 238 - protein, 16 aa, Artificial Sequence
  Synthetic Construct
  VARIANT 1, 2, 3: Xaa = SSS, SST, or RFS
  VARIANT 10: Xaa = N or S
  VARIANT 12: Xaa = V or I
XXXKSLLHSXGXTYLY

SEQ ID NO: 239 - protein, 7 aa, Artificial Sequence
  Synthetic Construct
  VARIANT 1: Xaa = R or Q
XMSNLAS

SEQ ID NO: 240 - protein, 9 aa, Artificial Sequence
  Synthetic Construct
  VARIANT 3: Xaa = M or N
  VARIANT 6: Xaa = R or L
  VARIANT 8: Xaa = F or W
AQXLEXPXT

SEQ ID NO: 241 - protein, 5 aa, Artificial Sequence
  Synthetic Construct
  VARIANT 1: Xaa = A or S
  VARIANT 4: Xaa = M or I
XYVXH

SEQ ID NO: 242 - protein, 17 aa, Artificial Sequence
  Synthetic Construct
  VARIANT 3: Xaa = F or N
  VARIANT 6: Xaa = N or S
  VARIANT 8: Xaa = G or Y
  VARIANT 10: Xaa = E or Q
YIXPYXDXTXYNEKFKG

SEQ ID NO: 243 - protein, 11 aa, Artificial Sequence
  Synthetic Construct
  VARIANT 2, 8: Xaa = T or A
RXDGNPYXMDY

SEQ ID NO: 244 - protein, 11 aa, Artificial Sequence
  Synthetic Construct
  VARIANT 11: Xaa = A or V
KASQDVSTAVX

SEQ ID NO: 245 - protein, 11 aa, Artificial Sequence
  Synthetic Construct
  VARIANT 5: Xaa = N or D
  VARIANT 7: Xaa = G or S
  VARIANT 9: Xaa = N or A
  VARIANT 11: Xaa = A or V
KASQXVXTXVX

SEQ ID NO: 246 - protein, 7 aa, Artificial Sequence
  Synthetic Construct
  VARIANT 6, 7: Xaa = FI or YT
SASYRXX

SEQ ID NO: 247 - protein, 9 aa, Artificial Sequence
  Synthetic Construct
  VARIANT 3, 4, 5, 6: Xaa = YNRN or HYST
  VARIANT 8: Xaa = I or F
QQXXXXPXT

SEQ ID NO: 248 - protein, 15 aa, Artificial Sequence
  Synthetic Construct
  VARIANT 1: Xaa = K or R
  VARIANT 7, 8, 9, 10, 11: Xaa = DYAGD or STSSY
  VARIANT 15: Xaa = N or H
XASQSVXXXXXSYMX

SEQ ID NO: 249 - protein, 7 aa, Artificial Sequence
  Synthetic Construct
  VARIANT 1: Xaa = A or Y
XASNLES

SEQ ID NO: 250 - protein, 9 aa, Artificial Sequence
  Synthetic Construct
  VARIANT 2, 3, 4, 5, 6: Xaa = QTNED or HSWEI
  VARIANT 8: Xaa = R or F
QXXXXXPXT

SEQ ID NO: 251 - protein, 5 aa, Artificial Sequence
  Synthetic Construct
DTYMY

SEQ ID NO: 252 - protein, 17 aa, Artificial Sequence
  Synthetic Construct
RIDPANDNTKYAQKFQG

SEQ ID NO: 253 - protein, 10 aa, Artificial Sequence
  Synthetic Construct
AKNLLNYFDY

SEQ ID NO: 254 - protein, 11 aa, Artificial Sequence
  Synthetic Construct
RASQEISGYLS

SEQ ID NO: 255 - protein, 7 aa, Artificial Sequence
  Synthetic Construct
ATSTLQS

SEQ ID NO: 256 - protein, 9 aa, Artificial Sequence
  Synthetic Construct
LQYAIYPLT

SEQ ID NO: 257 - protein, 8 aa, Artificial Sequence
  Synthetic Construct
GFTFSSYT

SEQ ID NO: 258 - protein, 8 aa, Artificial Sequence
  Synthetic Construct
ISHGGGDT

SEQ ID NO: 259 - protein, 16 aa, Artificial Sequence
  Synthetic Construct
ARHSGYERGYYYVMDY

SEQ ID NO: 260 - protein, 10 aa, Artificial Sequence
  Synthetic Construct
ESVDYYGFSF

SEQ ID NO: 261 - protein, 3 aa, Artificial Sequence
  Synthetic Construct
AAS

SEQ ID NO: 262 - protein, 8 aa, Artificial Sequence
  Synthetic Construct
QQSKEVPW

SEQ ID NO: 263 - protein, 8 aa, Artificial Sequence
  Synthetic Construct
GYTFTSYT

SEQ ID NO: 264 - protein, 8 aa, Artificial Sequence
  Synthetic Construct
INPTTGYT

SEQ ID NO: 265 - protein, 10 aa, Artificial Sequence
  Synthetic Construct
ARDDAYYSGY

SEQ ID NO: 266 - protein, 7 aa, Artificial Sequence
  Synthetic Construct
ENIYSNL

SEQ ID NO: 267 - protein, 3 aa, Artificial Sequence
  Synthetic Construct
AAK

SEQ ID NO: 268 - protein, 9 aa, Artificial Sequence
  Synthetic Construct
QHFWGTPWT

SEQ ID NO: 269 - protein, 8 aa, Artificial Sequence
  Synthetic Construct
GFAFSSYD

SEQ ID NO: 270 - protein, 8 aa, Artificial Sequence
  Synthetic Construct
ITIGGGTT

SEQ ID NO: 271 - protein, 12 aa, Artificial Sequence
  Synthetic Construct
ARHRYDYFAMDN

SEQ ID NO: 272 - protein, 10 aa, Artificial Sequence
  Synthetic Construct
ENVDNYGINF

SEQ ID NO: 273 - protein, 3 aa, Artificial Sequence
  Synthetic Construct
VSS

SEQ ID NO: 274 - protein, 8 aa, Artificial Sequence
  Synthetic Construct
QQSKDVPW

SEQ ID NO: 275 - protein, 135 aa, Mus musculus
SQVQLQQSGAELARPGASVKMSCKASGYTFTSYTMHWVKQRPGQGLEWIGYINPTTGYTNYNQKFKDKANPTTGYTNYNQKFKDKATLTADKSSSTAYMQLSSLTSEDSAVYYCARDDAYYSGYWGQGTTLTVSS

SEQ ID NO: 276 - protein, 108 aa, Mus musculus
DIQMTQSPASLSVSVGETVTITCRASENIYSNLAWYRQKQGKSPQLLVYAAKNLADGVPSRFSGSGSGTQYSLKINSLQSEDFGSYYCQHFWGTPWTFGGGTKLEIKR

SEQ ID NO: 277 - protein, 140 aa, Mus musculus
VQLVESGGGLVKPGGSLKLSCAASGFAFSSYDMSWVRQTPEKRLVWVAYITIGGGTTYYSDTVKRLVWVAYITIGGGTTYYSDTVKGRFTISRDNAKNTLYLQMSSLKSEDTAMYYCARHRYDYFAMDNWGHGTSVTVSS

SEQ ID NO: 278 - protein, 112 aa, Mus musculus
DIVLTQSPASLAVSLEHRATISCQASENVDNYGINFMNWFQHKPAQPPQLLIYVSSNLGSGVPAKFSGSGSGTDFSLNIHPMEEDDTAMYFCQQSKDVPWTFSGGTKLEIKR

SEQ ID NO: 279 - protein, 123 aa, Mus musculus
EVKLVESGGGLVQPGGSLKLSCAASGFTFSSYTMSWIRQTPEKRLEWVAYISHGGGDTYYPDTVKGRFTISRDNAKNTLYLQMSSLKSEDTAMYYCARHSGYERGYYYVMDYWGQGTSVTVSS

SEQ ID NO: 280 - protein, 111 aa, Mus musculus
DIVLTQFPTSLAVSLGQRATISCRASESVDYYGFSFINWFQQKPGQPPKLLIYAASNQGSGVPARFGGSGSGTDFSLNIHPMEEDDTAMYFCQQSKEVPWTFGGGTKLEIK

SEQ ID NO: 281 - protein, 119 aa, Artificial Sequence
  Synthetic Construct
QVQLVQSGAEVKKPGASVKVSCKASGFNIKDTYMYWVRQAPGQGLEWMGRIDPANDNTKYAQKFQGRVTITADTSTSTAYMELSSLRSEDTAVYYCARAKNLLNYFDYWGQGTLVTVSS

SEQ ID NO: 282 - protein, 119 aa, Artificial Sequence
  Synthetic Construct
QVQLVQSGAEVKKPGASVKVSCKASGFNIKDTYMYWVRQAPGQGLEWIGRIDPANDNTKYAPKFQGRVTITADTSTNTAYMELSSLRSEDTAVYYCARAKNLLNYFDYWGQGTLVTVSS

SEQ ID NO: 283 - protein, 119 aa, Artificial Sequence
  Synthetic Construct
EVQLVQSGAEVKKPGASVKVSCKASGFNIKDTYMYWVRQAPGQGLEWMGRIDPANDNTKYAQKFQGRVTITADTSTNTAYMELSSLRSEDTAVYYCARAKNLLNYFDYWGQGTLVTVSS

SEQ ID NO: 284 - protein, 108 aa, Artificial Sequence
  Synthetic Construct
DIQMTQSPSSLSASVGDRVTITCRASQEISGYLSWYQQKPGKAPKRLIYATSTLDSGVPSRFSGSGSGTDFTLTISSLQPEDFATYYCLQYAIYPLTFGQGTKLEIKR

SEQ ID NO: 285 - protein, 108 aa, Artificial Sequence
  Synthetic Construct
DIQMTQSPSSLSASVGDRVTITCRASQEISGYLSWLQQKPGKAPKRLIYATSTLQSGVPSRFSGSRSGTDYTLTISSLQPEDFATYYCLQYAIYPLTFGQGTKLEIKR

SEQ ID NO: 286 - protein, 108 aa, Artificial Sequence
  Synthetic Construct
DIQMTQSPSSLSASVGDRVTITCRASQEISGYLSWYQQKPGKAPKRLIYATSTLDSGVPSRFSGSRSGSDYTLTISSLQPEDFATYYCLQYAIYPLTFGQGTKLEIKR

SEQ ID NO: 287 - protein, 120 aa, Artificial Sequence
  Synthetic Construct
QVQLQQSGADLVRPGASVKLSCKASGYTFTDYEMHWVKQTPVYGLEWIGGIDPETGDTAYNQNFKGKATLTADKSSSAAYMELRSLTSEDSAVYYCTNYGNLYYYAMDYWGQGTSVTVSS

SEQ ID NO: 288 - protein, 108 aa, Artificial Sequence
  Synthetic Construct
ENVLTQSPAIMSASPGEKVTMTCRASSSVSSSYLHWYQQKSGASPKLWIYSTSNLAFGVPARFSGSGSGTSYSLTISSVEAEDAATYYCQQYSGYPLTFGSGTKLEIK

SEQ ID NO: 289 - protein, 5 aa, Artificial Sequence
  Synthetic Construct
DYEMH

SEQ ID NO: 290 - protein, 17 aa, Artificial Sequence
  Synthetic Construct
GIDPETGDTAYNQNFKG

SEQ ID NO: 291 - protein, 11 aa, Artificial Sequence
  Synthetic Construct
YGNLYYYAMDY

SEQ ID NO: 292 - protein, 12 aa, Artificial Sequence
  Synthetic Construct
RASSSVSSSYLH

SEQ ID NO: 293 - protein, 7 aa, Artificial Sequence
  Synthetic Construct
STSNLAF

SEQ ID NO: 294 - protein, 9 aa, Artificial Sequence
  Synthetic Construct
QQYSGYPLT

SEQ ID NO: 295 - protein, 8 aa, Artificial Sequence
  Synthetic Construct
GYTFTDYE

SEQ ID NO: 296 - protein, 8 aa, Artificial Sequence
  Synthetic Construct
IDPETGDT

SEQ ID NO: 297 - protein, 13 aa, Artificial Sequence
  Synthetic Construct
TNYGNLYYYAMDY

SEQ ID NO: 298 - protein, 7 aa, Artificial Sequence
  Synthetic Construct
SSVSSSY

SEQ ID NO: 299 - protein, 3 aa, Artificial Sequence
  Synthetic Construct
STS

SEQ ID NO: 300 - protein, 9 aa, Artificial Sequence
  Synthetic Construct
QQYSGYPLT

SEQ ID NO: 301 - protein, 15 aa, Artificial Sequence
  Synthetic Construct
KASQSVDYAGDSYLN

SEQ ID NO: 302 - protein, 15 aa, Artificial Sequence
  Synthetic Construct
RASQSVDYAGDSYMN

SEQ ID NO: 303 - protein, 15 aa, Artificial Sequence
  Synthetic Construct
RASQSVDYAGDSYLA

SEQ ID NO: 304 - protein, 5 aa, Artificial Sequence
  Synthetic Construct
SYWIH

SEQ ID NO: 305 - protein, 17 aa, Artificial Sequence
  Synthetic Construct
EIEPSASYTYYNQKFKG

SEQ ID NO: 306 - protein, 15 aa, Artificial Sequence
  Synthetic Construct
RASQSVDYAGDSYLN

SEQ ID NO: 307 - protein, 120 aa, Artificial Sequence
  Synthetic Construct
QVQLVESGAEVKKPGASVKLSCKASGYTFTSYWMHWVRQAPGQRLEWMGEIDPSASYTYYNQKFKGRVTITVDKSASTAYMELSSLRSEDTAVYYCARSVYYGNKYFDVWGPGTTVTVSS

SEQ ID NO: 308 - protein, 120 aa, Artificial Sequence
  Synthetic Construct
QVQLVQSGAEVKKPGASVKVSCKASGYTFTSYWMHWVRQAPGQGLEWMGEIDPSASYTYYNQKFKGRVTMTRDKSISTAYMELNSLTSDDSAVYYCARSVYYGNKYFDVWGAGTTVTVSS

SEQ ID NO: 309 - protein, 120 aa, Artificial Sequence
  Synthetic Construct
QVQLVQSGAEVRKPGASVKVSCKASGYTFTSYWMHWVRQAPGQGLEWVGEIDPSASYTYYNQKFKGRVTITADKSTSTAYMELSSLRSEDTDVYYCARSVYYGNKYFDVWGQGTTVTVSS

SEQ ID NO: 310 - protein, 120 aa, Artificial Sequence
  Synthetic Construct
QVQLVQSGAEVKKPGASVKVSCKASGYTFTSYWMHWVRQAPGQGLEWMGEIDPSASYTYYNQKFKGRVTMTRDKSSSTVYMELSSLTSEDSAVYYCARSVYYGNKYFDVWGAGTTVTVSS

SEQ ID NO: 311 - protein, 120 aa, Artificial Sequence
  Synthetic Construct
QVQLVQSGAEVKKPGASVKVSCRASGYTFTSYWIHWVRQAPGQGLEWIGEIEPSASYTYYNQKFKGRVTMTRDKSSSTVYMELSSLTSEDSAVYYCARSVYYGNKYFDVWGAGTTVTVSS

SEQ ID NO: 312 - protein, 120 aa, Artificial Sequence
  Synthetic Construct
QVQLQQSGAEVKKPGASVKVSCKASGYTFTSYWMHWVRQAPGQGLEWIGEIDPSASYTYYNQKFKGRVTMTRDKSTSTVYMQLSSLTSEDTAVYYCARSVYYGNKYFDVWGAGTTVTVSS

SEQ ID NO: 313 - protein, 111 aa, Artificial Sequence
  Synthetic Construct
DIVMTQSPDSLAVSLGERATINCKASQSVDYAGDSYLNWYQQKPGQPPKLLIYAASNLESGVPDRFSGSGSGTDFTLTISSLQAEDVAVYYCQQTNEDPRTFGGGTKVEIK

SEQ ID NO: 314 - protein, 111 aa, Artificial Sequence
  Synthetic Construct
DIVLTQSPSSLSASVGQRVTITCRASQSVDYAGDSYMNWYQQKPGKAPKLLIYAASNLESGVPSRFSGSGSGTDFTLTVSSLEDEDFATYYCQQTNEDPRTFGGGTKVEIK

SEQ ID NO: 315 - protein, 111 aa, Artificial Sequence
  Synthetic Construct
EIVLTQSPATLSLSPGQRATLSCRASQSVDYAGDSYLAWYQQKPGQAPRLLIYAASNLESGIPARFSGSGSGTDFTLTIRPLEEEDAAVYYCQQTNEDPRTFGGGTKLEIK

SEQ ID NO: 316 - protein, 111 aa, Artificial Sequence
  Synthetic Construct
DIQMTQSPSSLSASVGDRVTITCRASQSVDYAGDSYMNWYQQKPGKAPKLLIYAASNLESGVPSRFSGSGSGTDFTLTISSLEDEDFATYYCQQTNEDPRTFGGGTKLEIK

SEQ ID NO: 317 - protein, 111 aa, Artificial Sequence
  Synthetic Construct
DIVLTQSPSSLSASVGQRVTITCRASQSVDYAGDSYLNWYQQKPGKAPKLLIYAASNLESGIPSRFSGSGSGTDFTLTISSLEDEDFATYYCQQTNEDPRTFGGGTKLEIK

SEQ ID NO: 318 - protein, 111 aa, Artificial Sequence
  Synthetic Construct
DIQMTQSPSTLSASVGDRVTITCKASQSVDYAGDSYMNWYQQKPGKAPKLLIYAASNLESGVPSRFSGSGSGTEFTLTISSLQPDDFATYYCQQTNEDPRTFGGGTKLEIK

SEQ ID NO: 319 - protein, 120 aa, Artificial Sequence
  Synthetic Construct
QVQLVQSGAEMVKPGASVKISCKASGYTFTDYEMHWVRQTPVYGLEWIGGIDPETGDTAYNQNFKGRVTMTRDTSISTAYMELSRLTSDDTAVYYCTNYGNLYYYAMDYWGQGTLVTVSS

SEQ ID NO: 320 - protein, 120 aa, Artificial Sequence
  Synthetic Construct
QVQLQQSGAEVKKPGSSVKVSCKASGYTFTDYEMHWVRQTPVYGLEWMGGIDPETGDTAYNQNFKGRVTITADKSTSTAYMELSSLRSEDTAVYYCTNYGNLYYYAMDYWGQGTTVTVSS

SEQ ID NO: 321 - protein, 120 aa, Artificial Sequence
  Synthetic Construct
QVQLVQSGAEVKKPGASVKVSCKASGYTFTDYEMHWVRQAPGQGLEWMGGIDPETGDTAYNQNFKGRVTMTTDTSTSTAYMELRSLTSDDTAVYYCTNYGNLYYYAMDYWGQGTSVTVSS

SEQ ID NO: 322 - protein, 108 aa, Artificial Sequence
  Synthetic Construct
EIVLTQSPATLSLSPGERATLSCRASSSVSSSYLHWYQQKSGASPRLLIYSTSNLAFGIPARFSGSGSGTDYTLTISSLEAEDVAVYYCQQYSGYPLTFGGGTKVEIK

SEQ ID NO: 323 - protein, 108 aa, Artificial Sequence
  Synthetic Construct
EIVMTQSPATLSVSPGERATLSCRASSSVSSSYLHWYQQKSGASPRLWIYSTSNLAFGIPARFSGSGSGTEYTLTISSLQSEDFAAYYCQQYSGYPLTFGGGTKVEIK

SEQ ID NO: 324 - protein, 108 aa, Artificial Sequence
  Synthetic Construct
EIVLTQSPSSLSASVGDRVTITCRASSSVSSSYLHWYQQKPGKAPKLLIYSTSNLAFGVPSRFSGSGSGTSYTFTISSLQPEDIATYYCQQYSGYPLTFGSGTKLEIK

SEQ ID NO: 325 - protein, 203 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVH

SEQ ID NO: 326 - protein, 10 aa, Artificial Sequence
  Synthetic Construct
GYTFTNYGMN

SEQ ID NO: 327 - protein, 17 aa, Artificial Sequence
  Synthetic Construct
WINTYTGEPTYAADFKR

SEQ ID NO: 328 - protein, 14 aa, Artificial Sequence
  Synthetic Construct
YPHYYGSSHWYFDV

SEQ ID NO: 329 - protein, 11 aa, Artificial Sequence
  Synthetic Construct
SASQDISNYLN

SEQ ID NO: 330 - protein, 7 aa, Artificial Sequence
  Synthetic Construct
FTSSLHS

SEQ ID NO: 331 - protein, 9 aa, Artificial Sequence
  Synthetic Construct
QQYSTVPWT

SEQ ID NO: 332 - protein, 5 aa, Artificial Sequence
  Synthetic Construct
SYSMN

SEQ ID NO: 333 - protein, 17 aa, Artificial Sequence
  Synthetic Construct
SISSSSSYIYYADSVKG

SEQ ID NO: 334 - protein, 7 aa, Artificial Sequence
  Synthetic Construct
VTDAFDI

SEQ ID NO: 335 - protein, 11 aa, Artificial Sequence
  Synthetic Construct
RASQGIDNWLG

SEQ ID NO: 336 - protein, 7 aa, Artificial Sequence
  Synthetic Construct
DASNLDT

SEQ ID NO: 337 - protein, 9 aa, Artificial Sequence
  Synthetic Construct
QQAKAFPPT

SEQ ID NO: 338 - protein, 7 aa, Artificial Sequence
  Synthetic Construct
GSDKTHT

SEQ ID NO: 339 - protein, 98 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 340 - protein, 232 aa, Artificial Sequence
  Synthetic Construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 341 - protein, 107 aa, Homo sapiens [sequence omitted]

SEQ ID NO: 342 - protein, 660 aa, Artificial Sequence
  Synthetic Construct
QVQLQQSGADLVRPGASVKLSCKASGYTFTDYEMHWVKQTPVYGLEWIGGIDPETGDTAYNQNFKGKATLTADKSSSAAYMELRSLTSEDSAVYYCTNYGNLYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSDKTHTSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVH

SEQ ID NO: 343 - protein, 215 aa, Artificial Sequence
  Synthetic Construct
ENVLTQSPAIMSASPGEKVTMTCRASSSVSSSYLHWYQQKSGASPKLWIYSTSNLAFGVPARFSGSGSGTSYSLTISSVEAEDAATYYCQQYSGYPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 344 - protein, 19 aa, Artificial Sequence
  Synthetic Construct
MGWTLVFLFLLSVTAGVHS

SEQ ID NO: 345 - protein, 20 aa, Artificial Sequence
  Synthetic Construct
MVSSAQFLGLLLLCFQGTRC

SEQ ID NO: 346 - protein, 19 aa, Artificial Sequence
  Synthetic Construct
MGWSCIILFLVATATGVHS

SEQ ID NO: 347 - protein, 118 aa, Artificial Sequence
  Synthetic Construct
QVQLVQSGAEVKKPGSSVKVSCKASGYAFSSYWLNWVRQAPGQGLEWFGRIYPGDGDTDYNGKFKGRVTLTADKSTSTAYMELSSLRSEDTAVYFCVRGDGYWAMDYWGQGTTVTVSS

SEQ ID NO: 348 - protein, 118 aa, Artificial Sequence
  Synthetic Construct
QVQLVQSGAEVVKSGASVKVSCKASGYAFSSYWLNWVRQAPGQGLEWFGRIYPGDGDTDYNGKFKGRVTLIRDTSTSTVYMELTSLTSEDTAVYYCVRGDGYWAMDYWGQGTLVTVSS

SEQ ID NO: 349 - protein, 118 aa, Artificial Sequence
  Synthetic Construct
QVQLVQSGPEVKKPGESLKISCKASGYAFSSYWLNWVRQMPGKGLEWMGRIYPGDGDTDYNGKFKGQVTISADKSSGTAYLQLSSLKASDTAVYFCVRGDGYWAMDYWGQGTLVTVSS

SEQ ID NO: 350 - protein, 112 aa, Artificial Sequence
  Synthetic Construct
DIVMTQSPLSLPVTPGEPASISCRFSQSLLHSNGITYLYWYLQKPGQSPQLLIYQMSNLASGVPDRFSGSGSGTDFTLKISRVEAEDVGVYYCAQNLELPWTFGGGTKLEIK

SEQ ID NO: 351 - protein, 112 aa, Artificial Sequence
  Synthetic Construct
DIVMTQTPLSLPVTPGEPASISCRFSQSLLHSNGITYLYWYLQKPGQSPQLLIYTMSNLASGVPDRFSGSGSGTDFTLKISRVEAEDVGVYYCAQNLELPWTFGGGTKLEIK

SEQ ID NO: 352 - protein, 112 aa, Artificial Sequence
  Synthetic Construct
DIVMTQSPDSLAVSLGERATINCRFSKSLLHSNGITYLYWYQQKPGQPPKLLIYQMSNLASGVPDRFSGSGSGTDFTLTISSLQAEDVAVYYCAQNLELPWTFGGGTKLEIK

SEQ ID NO: 353 - protein, 16 aa, Artificial Sequence
  Synthetic Construct
RFSQSLLHSNGITYLY

SEQ ID NO: 354 - protein, 7 aa, Artificial Sequence
  Synthetic Construct
TMSNLAS

SEQ ID NO: 355 - protein, 17 aa, Artificial Sequence
  Synthetic Construct
KSSQSLLNSNNQKNYLA

SEQ ID NO: 356 - protein, 7 aa, Artificial Sequence
  Synthetic Construct
FASTRES

SEQ ID NO: 357 - protein, 9 aa, Artificial Sequence
  Synthetic Construct
QQHYNTPLT

SEQ ID NO: 358 - protein, 17 aa, Artificial Sequence
  Synthetic Construct
KSSQSLLNSNNQKNSLA

SEQ ID NO: 359 - protein, 9 aa, Artificial Sequence
  Synthetic Construct
QQHSNTPLT

SEQ ID NO: 360 - protein, 113 aa, Artificial Sequence
  Synthetic Construct
EVQLVQSGAEVKKPGTTVKIACKVSGYTFTDHGIHWVQQAPGKGLEWMGNISPGNGDIKYNEKFKGRVTLTADKSSDTAYMELNTLRSEDTAIYFCTTYFVDWGRGTLVTVSS

SEQ ID NO: 361 - protein, 113 aa, Artificial Sequence
  Synthetic Construct
QVQLQQSGAEVKKPGASVKVSCKASGYTFTDHGIHWVRQAPGRGLEWLGNISPGNGDIKYNEKFKGRVTMTRDTSTSTVYMELSSLTSEDTAVYFCTTYFVDWGRGTLVTVSS

SEQ ID NO: 362 - protein, 113 aa, Artificial Sequence
  Synthetic Construct
QVQLLESGAEAKKPGASVKLSCKASGYTFTDHGIHWVHQAPGQRLEWIGNISPGNGDIKYNEKFKGRVTITVDKSASTAYMEVSSLRSEDTAVYFCTTYFVDWGRGTLVTVSS

SEQ ID NO: 363 - protein, 113 aa, Artificial Sequence
  Synthetic Construct
DIVMTQSPSSLAVSLGERATLNCKSSQSLLNSNNQKNYLAWYQQKPGQPPKLLIYFASTRESGVPDRFSGSGSGTDFTLTISSVQAEDVAYYFCQQHYNTPLTFGQGTKLEIK

SEQ ID NO: 364 - protein, 113 aa, Artificial Sequence
  Synthetic Construct
DIVMTQSPDSLAVSLGERATINCKSSQSLLNSNNQKNSLAWYQQKPGQSPKLLIYFASTRESGVPDRFSGSGSGTDFTLTISSLQAEDVAYYFCQQHSNTPLTFGGGTKVEIK

SEQ ID NO: 365 - protein, 113 aa, Artificial Sequence
  Synthetic Construct
EIVMTQSPATLSVSPGERATLSCKSSQSLLNSNNQKNCLAWYQQKPGQAPRLLIYFASTRESGIPARFSGSGSGTEFTLTISSLQSEDFAYYFCQQHCNTPLTFGGGTKVEIK

SEQ ID NO: 366 - protein, 660 aa, Artificial Sequence
  Synthetic Construct
QVQLVQSGAEVKKPGASVKVSCKASGYTFTDYEMHWVRQAPGQGLEWMGGIDPETGDTAYNQNFKGRVTMTTDTSTSTAYMELRSLTSDDTAVYYCTNYGNLYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSDKTHTSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVH

SEQ ID NO: 367 - protein, 215 aa, Artificial Sequence
  Synthetic Construct
EIVLTQSPSSLSASVGDRVTITCRASSSVSSSYLHWYQQKPGKAPKLLIYSTSNLAFGVPSRFSGSGSGTSYTFTISSLQPEDIATYYCQQYSGYPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 368-392 - skipped (25 entries)

SEQ ID NO: 393 - protein, 121 aa, Artificial Sequence; identical to SEQ ID NO: 13
  Synthetic Construct

SEQ ID NO: 394 - protein, 116 aa, Artificial Sequence; identical to SEQ ID NO: 205
  Synthetic Construct

SEQ ID NO: 395 - protein, 121 aa, Artificial Sequence; identical to SEQ ID NO: 61
  Synthetic Construct

SEQ ID NO: 396 - protein, 113 aa, Artificial Sequence; identical to SEQ ID NO: 157
  Synthetic Construct

SEQ ID NO: 397 - protein, 120 aa, Artificial Sequence; identical to SEQ ID NO: 125
  Synthetic Construct

SEQ ID NO: 398 - protein, 120 aa, Artificial Sequence; identical to SEQ ID NO: 221
  Synthetic Construct

SEQ ID NO: 399 - protein, 120 aa, Artificial Sequence; identical to SEQ ID NO: 29
  Synthetic Construct

SEQ ID NO: 400 - protein, 116 aa, Artificial Sequence; identical to SEQ ID NO: 141
  Synthetic Construct

SEQ ID NO: 401 - protein, 121 aa, Artificial Sequence; identical to SEQ ID NO: 45
  Synthetic Construct

SEQ ID NO: 402 - protein, 121 aa, Artificial Sequence; identical to SEQ ID NO: 173
  Synthetic Construct

SEQ ID NO: 403 - protein, 120 aa, Artificial Sequence; identical to SEQ ID NO: 109
  Synthetic Construct

SEQ ID NO: 404 - protein, 118 aa, Artificial Sequence; identical to SEQ ID NO: 189
  Synthetic Construct

SEQ ID NO: 405 - protein, 115 aa, Artificial Sequence; identical to SEQ ID NO: 77
  Synthetic Construct

SEQ ID NO: 406 - protein, 121 aa, Artificial Sequence; identical to SEQ ID NO: 93
  Synthetic Construct

SEQ ID NO: 407 - protein, 109 aa, Artificial Sequence; identical to SEQ ID NO: 78
  Synthetic Construct

SEQ ID NO: 408 - protein, 107 aa, Artificial Sequence; identical to SEQ ID NO: 206
  Synthetic Construct

SEQ ID NO: 409 - protein, 112 aa, Artificial Sequence; identical to SEQ ID NO: 46
  Synthetic Construct

SEQ ID NO: 410 - protein, 112 aa, Artificial Sequence; identical to SEQ ID NO: 174
  Synthetic Construct

SEQ ID NO: 411 - protein, 112 aa, Artificial Sequence; identical to SEQ ID NO: 190
  Synthetic Construct

SEQ ID NO: 412 - protein, 113 aa, Artificial Sequence; identical to SEQ ID NO: 158
  Synthetic Construct

SEQ ID NO: 413 - protein, 106 aa, Artificial Sequence; identical to SEQ ID NO: 94
  Synthetic Construct

SEQ ID NO: 414 - protein, 107 aa, Artificial Sequence; identical to SEQ ID NO: 14
  Synthetic Construct

SEQ ID NO: 415 - protein, 107 aa, Artificial Sequence; identical to SEQ ID NO: 126
  Synthetic Construct

SEQ ID NO: 416 - protein, 107 aa, Artificial Sequence; identical to SEQ ID NO: 222
  Synthetic Construct

SEQ ID NO: 417 - protein, 107 aa, Artificial Sequence; identical to SEQ ID NO: 110
  Synthetic Construct

SEQ ID NO: 418 - protein, 106 aa, Artificial Sequence; identical to SEQ ID NO: 142
  Synthetic Construct

SEQ ID NO: 419 - protein, 111 aa, Artificial Sequence; identical to SEQ ID NO: 30
  Synthetic Construct

SEQ ID NO: 420 - protein, 111 aa, Artificial Sequence; identical to SEQ ID NO: 62
  Synthetic Construct

SEQ ID NO: 421 - protein, 6 aa, Artificial Sequence
  Synthetic Construct
SGTSTD

SEQ ID NO: 422 - protein, 6 aa, Artificial Sequence
  Synthetic Construct
TGTSDA